(19) United States
(12) Patent Application Publication
(10) Pub. No.: US 2005/0288273 A1
(43) Pub. Date: Dec. 29, 2005

(54) Antibacterial agents

(76) Inventors: Frederick Earl Boyer JR., Canton Township, MI (US); Allison Choy, Ann Arbor, MI (US); Louis S. Chupak, Old Saybrook, CT (US); Susan Hagen, Canton, MI (US); Joysula Venkata Nagendra Vara Prasad, Ann Arbor, MI (US); Takushi Kaneko, Guilford, CT (US); Ji-Young Kim, Ann Arbor, MI (US)

Correspondence Address: WARNER-LAMBERT COMPANY, 2800 PLYMOUTH RD, ANN ARBOR, MI, 48105, US

(21) Appl. No.: 10/774,241

(22) Filed: Feb. 6, 2004

Related U.S. Application Data
(60) Provisional application No. 60/445,821, filed on Feb. 7, 2003.

(51) Int. Cl. A61K 31/55

(52) U.S. Cl. 514/214.01; 540/579

(57) ABSTRACT

Compounds of formula I and methods for their preparation are disclosed. Further disclosed are methods of making biologically active compounds of formula I as well as pharmaceutically acceptable compositions comprising compounds of formula I. Compounds of formula I as disclosed herein can be used in a variety of applications including use as antibacterial agents.

This Regular Application claims benefits of U.S. Provisional Application No. 60/445,821, filed on Feb. 7, 2003.

### FIELD OF THE INVENTION

The invention relates to compounds bearing an oxazolidinone core structure which exhibit antibacterial activity, methods for their preparation, as well as pharmaceutically acceptable compositions comprising such compounds.

### BACKGROUND OF THE INVENTION

The oxazolidinones form a novel class of antibacterial agents with potent activity against a number of human and veterinary pathogens, including gram-positive aerobic bacteria such as multiply-resistant staphylococci and streptococci, anaerobic organisms such as bacteroides and clostridia species, and acid-fast organisms such as Mycobacterium tuberculosis and Mycobacterium. However, oxazolidinones generally do not demonstrate useful activity levels against aerobic gram-negative organisms. As a result, the use of oxazolidinones is limited to infections due to gram-positive bacteria. Accordingly, there is a need for oxazolidinones that have broader antibacterial activity, including activity against gram-negative as well as gram positive organisms.

### SUMMARY OF THE INVENTION

These and other needs are met by the present invention, which is directed to a compound of formula I: 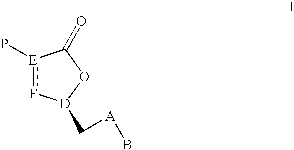
or a pharmaceutically acceptable salt thereof wherein:
   A is O,
      NH, or
      S;
   B is
      C(═O)R₁,
      C(═S)R₁,
      heterocylco,
      heteroaryl,
      C(═O)-heterocyclo, or
      C(═O)-heteteroaryl;
   D is N when E is C and F is CH when “- - - - - -” is a bond, or D is CH when E is N and F is CH₂ when “- - - - - -” is absent;
   P is 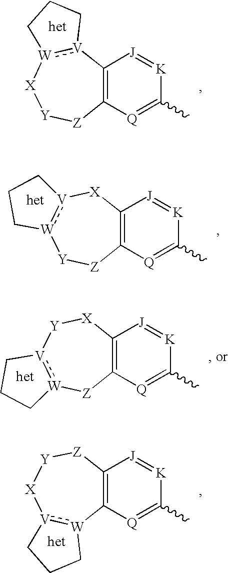
   wherein [symbol] indicates the point of attachment; and 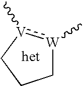
   is 5-membered heterocyclo or heteroaryl, wherein “ [symbol] ” indicates points of attachment, and wherein the 5-membered heterocyclo or heteroaryl is optionally substituted with one or more group selected from aryl, heteroaryl, heterocyclo, OR₅, OC(═O)R₁, NR₆R₇, NR₅, N(C═O)R₅, NH(C═O)OR₅, NHSO₂R₅, NHSO₂NR₅, aryl, heteroaryl, heterocyclo, wherein aryl or heteroaryl is optionally substituted with one or more halo, OH, CF₃, CN, NO₂, (C₁-C₈)alkyl, (C₃-C₆)cycloalkyl, S(C₁-C₄)alkyl, C(═O)R₁, OR₅, OC(═O)R₁, NR₆R₇, NHR₅, N(C═O)R₅, NH(C═O)OR₅, NHSO₂R₅, NHSO₂NR₅;
   J, K, Q independently are CR₂ or N, with the proviso that when any one of J, K, or Q is N, then the other two are CR₂;
   X, Y, Z independently are C═C—R₅, O═C,
      CH₂,
      CHR₃,
      CHR₄,
      CR₃R₄,
      CH(OR₅), or
      CHNR₆R₇;
   R₁ is H,
      (C₁-C₈)alkyl,
      (C₃-C₆)cycloalkyl,
      O—(C₁-C₄)alkyl,
      O—(C₃-C₆)cycloalkyl,
      S—(C₁-C₄) alkyl,
      S—(C₃-C₆)cycloalkyl,
      NH₂,
      NH(C₁-C₄)alkyl,
      N((C₁-C₄)alkyl)₂, or
      NH—(C₃-C₆)cycloalkyl,
   R₂ is H,
      halo,
      (C₁-C₈)alkyl,
      (C₃-C₆)cycloalkyl,
      O—(C₁-C₄)alkyl,
      O—(C₃-C₆)cycloalkyl,
      S—(C₁-C₄) alkyl,
      S—(C₃-C₆)cycloalkyl,
      NH₂,
      NH(C₁-C₄)alkyl,
      N((C₁-C₄)alkyl)₂, or
      NH—(C₃-C₆)cycloalkyl;
   R₃ and R₄ independently are halo,
      (C₁-C₈)alkyl,
      (C₃-C₆)cycloalkyl,
      O—(C₁-C₄)alkyl,
      O—(C₃-C₆)cycloalkyl,
      S—(C₁-C₄)alkyl,
      S—(C₃-C₆)cycloalkyl,
      NH₂,
      NH(C₁-C₄)alkyl,
      N((C₁-C₄)alkyl)₂,
      NH—(C₃-C₆)cycloalkyl;
      aryl,
      (CH₂)ₙ-aryl,
      heterocyclo,
      (CH₂)ₙ-heterocyclo,
      heteroaryl, or
      (CH₂)ₙ-heteroaryl;
   wherein n is 0, 1, 2, or 3;
   R₅ is H,
      (C₁-C₈)alkyl,
      (C₃-C₆)cycloalkyl,
      aryl,
      (CH₂)ₙ-aryl,
      heterocyclo,
      (CH₂)ₙ-heterocyclo,
      heteroaryl, or
      (CH₂)ₙ-heteroaryl;
   wherein n is as defined above;
   R₆ and R₇ independently are H,
      (C₁-C₈)alkyl,
      (C₃-C₆)cycloalkyl,
      aryl,
      (CH₂)ₙ-aryl,
      heterocyclo,
      (CH₂)ₙ-heterocyclo,
      heteroaryl, or
      (CH₂)ₙ-heteroaryl;
   wherein n is 0, 1, 2, or 3; or R₆ and R₇ together can form a 5-7-membered ring containing 1, 2, or 3 heteroatoms which are N or S.

What is also provided is a compound f formula II 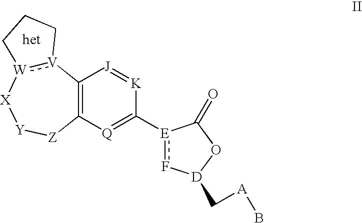
or a pharmaceutically acceptable salt thereof wherein
   A is O,
      NH, or
      S;
   B is
      C(═O)R₁,
      C(═S)R₁,
      heterocylco,
      heteroaryl,
      C(═O)-heterocyclo, or
      C(═O)-heteteroaryl;
   D is N when E is C and F is CH when “- - - - - -” is a bond, or D is CH when E is N and F is CH₂ when “- - - - - -”is absent; 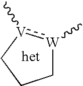
   is 5-membered heterocyclo or heteroaryl, wherein [symbol] indicates points of attachment, and wherein the 5-membered heterocyclo or heteroaryl is optionally substituted with one or more group selected from aryl, heteroaryl, heterocyclo, OR₅, OC(═O)R₁, NR₆R₇, NR₅, N(C═O)R₅, NH(C═O)OR₅, NHSO₂R₅, NHSO₂NR₅, aryl, heteroaryl, heterocyclo, wherein aryl or heteroaryl is optionally substituted with one or more halo, OH, CF₃, CN, NO₂, (C₁-C₈)alkyl, (C₃-C₆)cycloalkyl, S(C₁-C₄)alkyl, C(═O)R₁, OR₅, OC(═O)R₁, NR₆R₇, NHR₅, N(C═O)R₅, NH(C═O)OR₅, NHSO₂R₅, NHSO₂NR₅;
   J, K, Q independently are CR₂ or N, with the proviso that when any one of J, K, or Q is N, then the other two are CR₂;
   X, Y, Z independently are C═C—R₅, O═C,
      CH₂,
      CHR₃,
      CHR₄,
      CR₃R₄,
      CH(OR₅), or
      CHNR₆R₇;
   R₁ is H,
      (C₁-C₈)alkyl,
      (C₃-C₆)cycloalkyl,
      O—(C₁-C₄)alkyl,
      O—(C₃-C₆)cycloalkyl,
      S—(C₁-C₄) alkyl,
      S—(C₃-C₆)cycloalkyl,
      NH₂,
      NH(C₁-C₄)alkyl,
      N((C₁-C₄)alkyl)₂, or
      NH—(C₃-C₆)cycloalkyl,
   R₂ is H,
      halo,
      (C₁-C₈)alkyl,
      (C₃-C₆)cycloalkyl,
      O—(C₁-C₄)alkyl,
      O—(C₃-C₆)cycloalkyl,
      S—(C₁-C₄) alkyl,
      S—(C₃-C₆)cycloalkyl,
      NH₂,
      NH(C₁-C₄)alkyl,
      N((C₁-C₄)alkyl)₂, or
      NH—(C₃-C₆)cycloalkyl;
   R₃ and R₄ independently are H, halo,
      (C₁-C₈)alkyl,
      (C₃-C₆)cycloalkyl,
      O—(C₁-C₄)alkyl,
      O—(C₃-C₆)cycloalkyl,
      S—(C₁-C₄) alkyl,
      S—(C₃-C₆)cycloalkyl,
      NH₂,
      NH(C₁-C₄)alkyl,
      N((C₁-C₄)alkyl)₂,
      NH—(C₃-C₆)cycloalkyl;
      aryl,
      (CH₂)ₙ-aryl,
      heterocyclo,
      (CH₂)ₙ-heterocyclo,
      heteroaryl, or
      (CH₂)ₙ-heteroaryl;
   wherein n is 0, 1, 2, or 3;
   R₅ is H,
      (C₁-C₈)alkyl,
      (C₃-C₆)cycloalkyl,
      aryl,
      (CH₂)ₙ-aryl,
      heterocyclo,
      (CH₂)ₙ-heterocyclo,
      heteroaryl, or
      (CH₂)ₙ-heteroaryl;
   wherein n is 0, 1, 2, or 3;
   R₆ and R₇ independently are H;
      (C₁-C₈)alkyl,
      (C₃-C₆)cycloalkyl,
      aryl,
      (CH₂)ₙ-aryl,
      heterocyclo,
      (CH₂)ₙ-heterocyclo,
      heteroaryl, or
      (CH₂)ₙ-heteroaryl;
   wherein n is 0, 1, 2, or 3; or R₆ and R₇ together can form a 5-7-membered ring containing 1, 2, or 3 heteroatoms which are N or S.

What is also provided is a compound of formula III 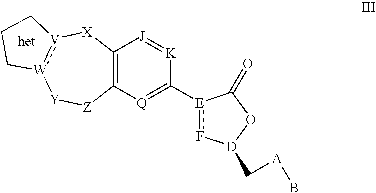
or a pharmaceutically acceptable salt thereof wherein:
   A is O,
      NH, or
      S;
   B is
      C(═O)R₁,
      C(═S)R₁,
      heterocylco,
      heteroaryl,
      C(═O)-heterocyclo, or
      C(═O)-heteteroaryl;
   D is N when E is C and F is CH when “- - - - - -” is a bond, or D is CH when E is N and F is CH₂ when “- - - - - -” is absent; 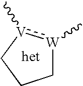
   is 5-membered heterocyclo or heteroaryl, wherein [symbol] indicates points of attachment, and wherein the 5-membered heterocyclo or heteroaryl is optionally substituted with one or more group selected from aryl, heteroaryl, heterocyclo, OR₅, OC(═O)R₁, NR₆R₇, NR₅, N(C═O)R₅, NH(C═O)OR₅, NHSO₂R₅, NHSO₂NR₅, aryl, heteroaryl, heterocyclo, wherein aryl or heteroaryl is optionally substituted with one or more halo, OH, CF₃, CN, NO₂, (C₁-C₈)alkyl, (C₃-C₆)cycloalkyl, S(C₁-C₄)alkyl, C(═O)R₁, OR₅, OC(═O)R₁, NR₆R₇, NHR₅, N(C═O)R₅, NH(C═O)OR₅, NHSO₂R₅, NHSO₂NR₅;
   J, K, Q independently are CR₂ or N, with the proviso that when any one of J, K, or Q is N, then the other two are CR₂;
   X, Y, Z independently are C═C—R₅, O═C,
      CH₂,
      CHR₃,
      CHR₄,
      CR₃R₄,
      CH(OR₅), or
      CHNR₆R₇;
   R₁ is H,
      (C₁-C₈)alkyl,
      (C₃-C₆)cycloalkyl,
      O—(C₁-C₄)alkyl,
      O—(C₃-C₆)cycloalkyl,
      S—(C₁-C₄) alkyl,
      S—(C₃-C₆)cycloalkyl,
      NH₂,
      NH(C₁-C₄)alkyl,
      N((C₁-C₄)alkyl)₂, or
      NH—(C₃-C₆)cycloalkyl,
   R₂ is H,
      halo,
      (C₁-C₈)alkyl,
      (C₃-C₆)cycloalkyl,
      O—(C₁-C₄)alkyl,
      O—(C₃-C₆)cycloalkyl,
      S—(C₁-C₄) alkyl,
      S—(C₃-C₆)cycloalkyl,
      NH₂,
      NH(C₁-C₄)alkyl,
      N((C₁-C₄)alkyl)₂, or
      NH—(C₃-C₆)cycloalkyl;
   R₃ and R₄ independently are halo,
      (C₁-C₈)alkyl,
      (C₃-C₆)cycloalkyl,
      O—(C₁-C₄)alkyl,
      O—(C₃-C₆)cycloalkyl,
      S—(C₁-C₄) alkyl,
      S—(C₃-C₆)cycloalkyl,
      NH₂,
      NH(C₁-C₄)alkyl,
      N((C₁-C₄)alkyl)₂,
      NH—(C₃-C₆)cycloalkyl;
      aryl,
      (CH₂)ₙ-aryl,
      heterocyclo,
      (CH₂)ₙ-heterocyclo,
      heteroaryl, or
      (CH₂)ₙ-heteroaryl;
   wherein n is 0, 1, 2, or 3;
   R₅ is H,
      (C₁-C₈)alkyl,
      (C₃-C₆)cycloalkyl,
      aryl,
      (CH₂)ₙ-aryl,
      heterocyclo,
      (CH₂)ₙ-heterocyclo,
      heteroaryl, or
      (CH₂)ₙ-heteroaryl;
   wherein n is 0, 1, 2, or 3;
   R₆ and R₇ independently are H,
      (C₁-C₈)alkyl,
      (C₃-C₆)cycloalkyl,
      aryl,
      (CH₂)ₙ-aryl,
      heterocyclo,
      (CH₂)ₙ-heterocyclo,
      heteroaryl, or
      (CH₂)ₙ-heteroaryl;
wherein n is 0, 1, 2, or 3; or R₆ and R₇ together can form a 5-7-membered ring containing 1, 2, or 3 heteroatoms which are N or S.

What is also provided is a compound of formula IV 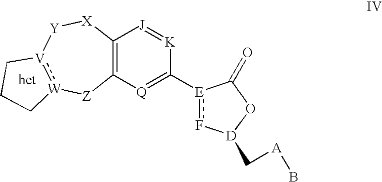
or a pharmaceutically acceptable salt thereof wherein:
   A is O,
      NH, or
      S;
   B is
      C(═O)R₁,
      C(═S)R₁,
      heterocylco,
      heteroaryl,
      C(═O)-heterocyclo, or
      C(═O)-heteteroaryl;
   D is N when E is C and F is CH when “- - - - - -” is a bond, or D is CH when E is N and F is CH₂ when “- - - - - -” is absent; 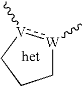
   is 5-membered heterocyclo or heteroaryl, wherein [symbol] indicates points of attachment, and wherein the 5-membered heterocyclo or heteroaryl is optionally substituted with one or more group selected from aryl, heteroaryl, heterocyclo, OR₅, OC(═O)R₁, NR₆R₇, NR₅, N(C═O)R₅, NH(C═O)OR₅, NHSO₂R₅, NHSO₂NR₅, aryl, heteroaryl, heterocyclo, wherein aryl or heteroaryl is optionally substituted with one or more halo, OH, CF₃, CN, NO₂, (C₁-C₈)alkyl, (C₃-C₆)cycloalkyl, S(C₁-C₄)alkyl, C(═O)R₁, OR₅, OC(═O)R₁, NR₆R₇, NHR₅, N(C═O)R₅, NH(C═O)OR₅, NHSO₂R₅, NHSO₂NR₅;
   J, K, Q independently are CR₂ or N, with the proviso that when any one of J, K, or Q is N, then the other two are CR₂;
   X, Y, Z independently are C═C—R₅, O═C,
      CH₂,
      CHR₃,
      CHR₄,
      CR₃R₄,
      CH(OR₅), or
      CHNR₆R₇;
   R₁ is H,
      (C₁-C₈)alkyl,
      (C₃-C₆)cycloalkyl,
      O—(C₁-C₄)alkyl,
      O—(C₃-C₆)cycloalkyl,
      S—(C₁-C₄) alkyl,
      S—(C₃-C₆)cycloalkyl,
      NH₂,
      NH(C₁-C₄)alkyl,
      N((C₁-C₄)alkyl)₂, or
      NH—(C₃-C₆)cycloalkyl,
   R₂ is H,
      halo,
      (C₁-C₈)alkyl,
      (C₃-C₆)cycloalkyl,
      O—(C₁-C₄)alkyl,
      O—(C₃-C₆)cycloalkyl,
      S—(C₁-C₄) alkyl,
      S—(C₃-C₆)cycloalkyl,
      NH₂,
      NH(C₁-C₄)alkyl,
      N((C₁-C₄)alkyl)₂, or
      NH—(C₃-C₆)cycloalkyl;
   R₃ and R₄ independently are halo,
      (C₁-C₈)alkyl,
      (C₃-C₆)cycloalkyl,
      O—(C₁-C₄)alkyl,
      O—(C₃-C₆)cycloalkyl,
      S—(C₁-C₄) alkyl,
      S—(C₃-C₆)cycloalkyl,
      NH₂,
      NH(C₁-C₄)alkyl,
      N((C₁-C₄)alkyl)₂,
      NH—(C₃-C₆)cycloalkyl;
      aryl,
      (CH₂)ₙ-aryl,
      heterocyclo,
      (CH₂)ₙ-heterocyclo,
      heteroaryl, or
      (CH₂)ₙ-heteroaryl;
      wherein n is 0, 1, 2, or 3;
   R₅ is H,
      (C₁-C₈)alkyl,
      (C₃-C₆)cycloalkyl,
      aryl,
      (CH₂)ₙ-aryl,
      heterocyclo,
      (CH₂)ₙ-heterocyclo,
      heteroaryl, or
      (CH₂)ₙ-heteroaryl;
      wherein n is 0, 1, 2, or 3;
   R₆ and R₇ independently are H,
      (C₁-C₈)alkyl,
      (C₃-C₆)cycloalkyl,
      aryl,
      (CH₂)ₙ-aryl,
      heterocyclo,
      (CH₂)ₙ-heterocyclo,
      heteroaryl, or
      (CH₂)ₙ-heteroaryl;
      wherein n is 0, 1, 2, or 3; or
   R₆ and R₇ together can form a 5-7-membered ring containing 1, 2, or 3 heteroatoms which are N or S.

What is also provided is a compound of formula V 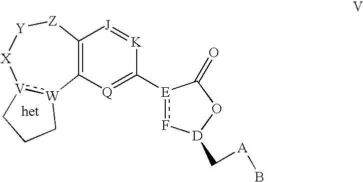
or a pharmaceutically acceptable salt thereof wherein:
   A is O,
      NH, or
      S;
   B is
      C(═O)R₁,
      C(═S)R₁,
      heterocylco,
      heteroaryl,
      C(═O)-heterocyclo, or
      C(═O)-heteteroaryl;
   D is N when E is C and F is CH when “- - - - - -” is a bond, or D is CH when E is N and F is CH₂ when “- - - - - -” is absent; 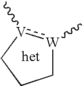
   is 5-membered heterocyclo or heteroaryl, wherein [symbol] indicates points of attachment, and wherein the 5-membered heterocyclo or heteroaryl is optionally substituted with one or more group selected from aryl, heteroaryl, heterocyclo, OR₅, OC(═O)R₁, NR₆R₇, NR₅, N(C═O)R₅, NH(C═O)OR₅, NHSO₂R₅, NHSO₂NR₅, aryl, heteroaryl, heterocyclo, wherein aryl or heteroaryl is optionally substituted with one or more halo, OH, CF₃, CN, NO₂, (C₁-C₈)alkyl, (C₃-C₆)cycloalkyl, S(C₁-C₄)alkyl, C(═O)R₁, OR₅, OC(═O)R₁, NR₆R₇, NHR₅, N(C═O)R₅, NH(C═O)OR₅, NHSO₂R₅, NHSO₂NR₅;
   J, K, Q independently are CR₂ or N, with the proviso that when any one of J, K, or Q is N, then the other two are CR₂;
   X, Y, Z independently are C═C—R₅, O═C,
      CH₂,
      CHR₃,
      CHR₄,
      CR₃R₄,
      CH(OR₅), or
      CHNR₆R₇;
   R₁ is H,
      (C₁-C₈)alkyl,
      (C₃-C₆)cycloalkyl,
      O—(C₁-C₄)alkyl,
      O—(C₃-C₆)cycloalkyl,
      S—(C₁-C₄) alkyl,
      S—(C₃-C₆)cycloalkyl,
      NH₂,
      NH(C₁-C₄)alkyl,
      N((C₁-C₄)alkyl)₂, or
      NH—(C₃-C₆)cycloalkyl,
   R₂ is H,
      halo,
      (C₁-C₈)alkyl,
      (C₃-C₆)cycloalkyl,
      O—(C₁-C₄)alkyl,
      O—(C₃-C₆)cycloalkyl,
      S—(C₁-C₄) alkyl,
      S—(C₃-C₆)cycloalkyl,
      NH₂,
      NH(C₁-C₄)alkyl,
      N((C₁-C₄)alkyl)₂, or
      NH—(C₃-C₆)cycloalkyl;
   R₃ and R₄ independently are halo,
      (C₁-C₈)alkyl,
      (C₃-C₆)cycloalkyl,
      O—(C₁-C₄)alkyl,
      O—(C₃-C₆)cycloalkyl,
      S—(C₁-C₄) alkyl,
      S—(C₃-C₆)cycloalkyl,
      NH₂,
      NH(C₁-C₄)alkyl,
      N((C₁-C₄)alkyl)₂,
      NH—(C₃-C₆)cycloalkyl;
      aryl,
      (CH₂)ₙ-aryl,
      heterocyclo,
      (CH₂)ₙ-heterocyclo,
      heteroaryl, or
      (CH₂)ₙ-heteroaryl;
      wherein n is 0, 1, 2, or 3;
   R₅ is H,
      (C₁-C₈)alkyl,
      (C₃-C₆)cycloalkyl,
      aryl,
      (CH₂)ₙ-aryl,
      heterocyclo,
      (CH₂)ₙ-heterocyclo,
      heteroaryl, or
      (CH₂)ₙ-heteroaryl;
      wherein n is 0, 1, 2, or 3;
   R₆ and R₇ independently are H,
      (C₁-C₈)alkyl,
      (C₃-C₆)cycloalkyl,
      aryl,
      (CH₂)ₙ-aryl,
      heterocyclo,
      (CH₂)ₙ-heterocyclo,
      heteroaryl, or
      (CH₂)ₙ-heteroaryl;
      wherein n is 0, 1, 2, or 3; or
   R₆ and R₇ together can form a 5-7-membered ring containing 1, 2, or 3 heteroatoms which are N or S.

What is also provided is a compound which is:
(S)-N-[2-Oxo-3-(1,4,5,6-tetrahydro-1,2-diaza-benzo[e]azulen-8-yl)-oxazolidin-5-ylmethyl]-acetamide;
(S)-N-[2-Oxo-3-(3-phenyl-1,4,5,6-tetrahydro-1,2-diaza-benzo[e]azulen-8-yl)-oxazolidin-5-ylmethyl]-acetamide;
(S)-N-{3-[3-(2-Hydroxy-phenyl)-1,4,5,6-tetrahydro-1,2-diaza-benzo[e]azulen-8-yl]-2-oxo-oxazolidin-5-ylmethyl}-acetamide;
(S)-N-{3-[3-(2-Methoxy-phenyl)-1,4,5,6-tetrahydro-1,2-diaza-benzo[e]azulen-8-yl]-2-oxo-oxazolidin-5-ylmethyl}-acetamide;
(S)-N-{2-Oxo-3-[3-(2-trifluoromethoxy-phenyl)-1,4,5,6-tetrahydro-1,2-diaza-benzo[e]azulen-8-yl]-oxazolidin-5-ylmethyl}-acetamide;
(S)-N-{2-Oxo-3-[3-(2-trifluoromethyl-phenyl)-1,4,5,6-tetrahydro-1,2-diaza-benzo[e]azulen-8-yl]-oxazolidin-5-ylmethyl}-acetamide;
(S)-N-{3-[3-(2-Fluoro-phenyl)-1,4,5,6-tetrahydro-1,2-diaza-benzo[e]azulen-8-yl]-2-oxo-oxazolidin-5-ylmethyl}-acetamide;
(S)-N-{3-[3-(3-Hydroxy-phenyl)-1,4,5,6-tetrahydro-1,2-diaza-benzo[e]azulen-8-yl]-2-oxo-oxazolidin-5-ylmethyl}-acetamide;
(S)-N-{3-[3-(3-Methroxy-phenyl)-1,4,5,6-tetrahydro-1,2-diaza-benzo[e]azulen-8-yl]-2-oxo-oxazolidin-5-ylmethyl}-acetamide;
(S)-N-{2-Oxo-3-[3-(3-trifluoromethoxy-phenyl)-1,4,5,6-tetrahydro-1,2-diaza-benzo[e]azulen-8-yl]-oxazolidin-5-ylmethyl}-acetamide;
(S)-N-{2-Oxo-3-[3-(3-trifluormethyl-phenyl)-1,4,5,6-tetrahydro-1,2-diaza-benzo[e]azulen-8-yl]-oxazolidin-5-ylmethyl}-acetamide;
(S)-N-{3-[3-(3-Fluoro-phenyl)-1,4,5,6-tetrahydro-1,2-diaza-benzo[e]azulen-8-yl]-2-oxo-oxazolidin-5-ylmethyl}-acetamide;
(S)-N-{3-[3-(4-Hydroxy-phenyl)-1,4,5,6-tetrahydro-1,2-diaza-benzo[e]azulen-8-yl]-2-oxo-oxazolidin-5-ylmethyl}-acetamide;
(S)-N-{3-[3-(4-Methoxy-phenyl)-1,4,5,6-tetrahydro-1,2-diaza-benzo[e]azulen-8-yl]-2-oxo-oxazolidin-5-ylmethyl}-acetamide;
(S)-N-{2-Oxo-3-[3-(4-trifluoromethoxy-phenyl)-1,4,5,6-tetrahydro-1,2-diaza-benzo[e]azulen-8-yl]-oxazolidin-5-ylmethyl}-acetamide;
(S)-N-{2-Oxo-3-[3-(4-trifluoromethyl-phenyl)-1,4,5,6-tetrahydro-1,2-diaza-benzo[e]azulen-8-yl]-oxazolidin-5-ylmethyl}-acetamide;
(S)-N-{3-[3-(4-Fluoro-phenyl)-1,4,5,6-tetrahydro-1,2-diaza-benzo[e]azulen-8-yl]-2-oxo-oxazolidin-5-ylmethyl}-acetamide;
(S)-N-[2-Oxo-3-(3-thiophen-3-yl-1,4,5,6-tetrahydro-1,2-diaza-benzo[e]azulen-8-yl)-oxazolidin-5-ylmethyl]-acetamide;
(S)-N-{3-[3-(4-Hydroxy-thiophen-3-yl)-1,4,5,6-tetrahydro-1,2-diaza-benzo[e]azulen-8-yl]-2-oxo-oxazolidin-5-ylmethyl}-acetamide;
(S)-N-{3-[3-(4-Methoxy-thiophen-3-yl)-1,4,5,6-tetrahydro-1,2-diaza-benzo[e]azulen-8-yl]-2-oxo-oxazolidin-5-ylmethyl}-acetamide;
(S)-N-{2-Oxo-3-[3-(4-trifluoromethoxy-thiophen-3-yl)-1,4,5,6-tetrahydro-1,2-diaza-benzo[e]azulen-8-yl]-oxazolidin-5-ylmethyl}-acetamide;
(S)-N-{2-Oxo-3-[3-(4-trifluoromethyl-thiophen-3-yl)-1,4,5,6-tetrahydro-1,2-diaza-benzo[e]azulen-8-yl]-oxazolidin-5-ylmethyl}-acetamide;
(S)-N-{3-[3-(4-Fluoro-thiophen-3-yl)-1,4,5,6-tetrahydro-1,2-diaza-benzo[e]azulen-8-yl]-2-oxo-oxazolidin-5-ylmethyl}-acetamide;
(S)-N-{3-[3-(5-Hydroxy-thiophen-3-yl)-1,4,5,6-tetrahydro-1,2-diaza-benzo[e]azulen-8-yl]-2-oxo-oxazolidin-5-ylmethyl}-acetamide;
(S)-N-{3-[3-(5-Methoxy-thiophen-3-yl)-1,4,5,6-tetrahydro-1,2-diaza-benzo[e]azulen-8-yl]-2-oxo-oxazolidin-5-ylmethyl}-acetamide;
(S)-N-{2-Oxo-3-[3-(5-trifluoromethoxy-thiophen-3-yl)-1,4,5,6-tetrahydro-1,2-diaza-benzo[e]azulen-8-yl]-oxazolidin-5-ylmethyl}-acetamide;
(S)-N-{2-Oxo-3-[3-(5-trifluoromethyl-thiophen-3-yl)-1,4,5,6-tetrahydro-1,2-diaza-benzo[e]azulen-8-yl]-oxazolidin-5-ylmethyl}-acetamide;
(S)-N-{3-[3-(5-Fluoro-thiophen-3-yl)-1,4,5,6-tetrahydro-1,2-diaza-benzo[e]azulen-8-yl]-2-oxo-oxazolidin-5-ylmethyl}-acetamide;
(S)-N-[3-(3-Furan-3-yl-1,4,5,6-tetrahydro-1,2-diaza-benzo[e]azulen-8-yl)-2-oxo-oxazolidin-5-ylmethyl]-acetamide;
(S)-N-{3-[3-(4-Hydroxy-furan-3-yl)-1,4,5,6-tetrahydro-1,2-diaza-benzo[e]azulen-8-yl]-2-oxo-oxazolidin-5-ylmethyl}-acetamide;
(S)-N-{3-[3-(4-Methoxy-furan-3-yl)-1,4,5,6-tetrahydro-1,2-diaza-benzo[e]azulen-8-yl]-2-oxo-oxazolidin-5-ylmethyl}-acetamide;
(S)-N-{2-Oxo-3-[3-(4-trifluoromethoxy-furan-3-yl)-1,4,5,6-tetrahydro-1,2-diaza-benzo[e]azulen-8-yl]-oxazolidin-5-ylmethyl}-acetamide;
(S)-N-{2-Oxo-3-[3-(4-trifluoromethyl-furan-3-yl)-1,4,5,6-tetrahydro-1,2-diaza-benzo[e]azulen-8-yl]-oxazolidin-5-ylmethyl}-acetamide;
(S)-N-{3-[3-(4-Fluoro-furan-3-yl)-1,4,5,6-tetrahydro-1,2-diaza-benzo[e]azulen-8-yl]-2-oxo-oxazolidin-5-ylmethyl}-acetamide;
(S)-N-{3-[3-(5-Hydroxy-furan-3-yl)-1,4,5,6-tetrahydro-1,2-diaza-benzo[e]azulen-8-yl]-2-oxo-oxazolidin-5-ylmethyl}-acetamide;
(S)-N-{3-[3-(5-Methoxy-furan-3-yl)-1,4,5,6-tetrahydro-1,2-diaza-benzo[e]azulen-8-yl]-2-oxo-oxazolidin-5-ylmethyl}-acetamide;
(S)-N-{2-Oxo-3-[3-(5-trifluoromethoxy-furan-3-yl)-1,4,5,6-tetrahydro-1,2-diaza-benzo[e]azulen-8-yl]-oxazolidin-5-ylmethyl}-acetamide;
(S)-N-{2-Oxo-3-[3-(5-trifluoromethyl-furan-3-yl)-1,4,5,6-tetrahydro-1,2-diaza-benzo[e]azulen-8-yl]-oxazolidin-5-ylmethyl}-acetamide;
(S)-N-{3-[3-(5-Fluoro-furan-3-yl)-1,4,5,6-tetrahydro-1,2-diaza-benzo[e]azulen-8-yl]-2-oxo-oxazolidin-5-ylmethyl}-acetamide;
(S)-N-[2-Oxo-3-(3-pyridin-4-yl-1,4,5,6-tetrahydro-1,2-diaza-benzo[e]azulen-8-yl)-oxazolidin-5-ylmethyl]-acetamide;
(S)-N-{3-[3-(3-Hydroxy-pyridin-4-yl)-1,4,5,6-tetrahydro-1,2-diaza-benzo[e]azulen-8-yl]-2-oxo-oxazolidin-5-ylmethyl}-acetamide;
(S)-N-{3-[3-(3-Methoxy-pyridin-4-yl)-1,4,5,6-tetrahydro-1,2-diaza-benzo[e]azulen-8-yl]-2-oxo-oxazolidin-5-ylmethyl}-acetamide;
(S)-N-{2-Oxo-3-[3-(3-trifluoromethoxy-pyridin-4-yl)-1,4,5,6-tetrahydro-1,2-diaza-benzo[e]azulen-8-yl]-oxazolidin-5-ylmethyl}-acetamide;
(S)-N-{2-Oxo-3-[3-(3-trifluoromethyl-pyridin-4-yl)-1,4,5,6-tetrahydro-1,2-diaza-benzo[e]azulen-8-yl]-oxazolidin-5-ylmethyl}-acetamide;
(S)-N-{3-[3-(3-Fluoro-pyridin-4-yl)-1,4,5,6-tetrahydro-1,2-diaza-benzo[e]azulen-8-yl]-2-oxo-oxazolidin-5-ylmethyl}-acetamide;
(S)-N-{3-[3-(2-Hydroxy-pyridin-4-yl)-1,4,5,6-tetrahydro-1,2-diaza-benzo[e]azulen-8-yl]-2-oxo-oxazolidin-5-ylmethyl}-acetamide;
(S)-N-{3-[3-(2-Methoxy-pyridin-4-yl)-1,4,5,6-tetrahydro-1,2-diaza-benzo[e]azulen-8-yl]-2-oxo-oxazolidin-5-ylmethyl}-acetamide;
(S)-N-{2-Oxo-3-[3-(2-trifluoromethoxy-pyridin-4-yl)-1,4,5,6-tetrahydro-1,2-diaza-benzo[e]azulen-8-yl]-oxazolidin-5-ylmethyl}-acetamide;
(S)-N-{2-Oxo-3-[3-(2-trifluoromethyl-pyridin-4-yl)-1,4,5,6-tetrahydro-1,2-diaza-benzo[e]azulen-8-yl]-oxazolidin-5-ylmethyl}-acetamide;
(S)-N-{3-[3-(2-Fluoro-pyridin-4-yl)-1,4,5,6-tetrahydro-1,2-diaza-benzo[e]azulen-8-yl]-2-oxo-oxazolidin-5-ylmethyl}-acetamide;
(S)-N-[3-(5,6-Dihydro-4H-1-oxa-2-aza-benzo[e]azulen-8-yl)-2-oxo-oxazolidin-5-ylmethyl]-acetamide;
(S)-N-[2-Oxo-3-(3-phenyl-5,6-dihydro-4H-1-oxa-2-aza-benzo[e]azulen-8-yl)-oxazolidin-5-ylmethyl]-acetamide;
(S)-N-{3-[3-(2-Hydroxy-phenyl)-5,6-dihydro-4H-1-oxa-2-aza-benzo[e]azulen-8-yl]-2-oxo-oxazolidin-5-ylmethyl}-acetamide;
(S)-N-{3-[3-(2-Methoxy-phenyl)-5,6-dihydro-4H-1-oxa-2-aza-benzo[e]azulen-8-yl]-2-oxo-oxazolidin-5-ylmethyl}-acetamide;
(S)-N-{2-Oxo-3-[3-(2-trifluoromethoxy-phenyl)-5,6-dihydro-4H-1-oxa-2-aza-benzo[e]azulen-8-yl]-oxazolidin-5-ylmethyl}-acetamide;
(S)-N-{2-Oxo-3-[3-(2-trifluoromethyl-phenyl)-5,6-dihydro-4H-1-oxa-2-aza-benzo[e]azulen-8-yl]-oxazolidin-5-ylmethyl}-acetamide;
(S)-N-{3-[3-(2-Fluoro-phenyl)-5,6-dihydro-4H-1-oxa-2-aza-benzo[e]azulen-8-yl]-2-oxo-oxazolidin-5-ylmethyl}-acetamide;
(S)-N-{3-[3-(3-Hydroxy-phenyl)-5,6-dihydro-4H-1-oxa-2-aza-benzo[e]azulen-8-yl]-2-oxo-oxazolidin-5-ylmethyl}-acetamide;
(S)-N-{3-[3-(3-Methoxy-phenyl)-5,6-dihydro-4H-1-oxa-2-aza-benzo[e]azulen-8-yl]-2-oxo-oxazolidin-5-ylmethyl}-acetamide;
(S)-N-{2-Oxo-3-[3-(3-trifluoromethoxy-phenyl)-5,6-dihydro-4H-1-oxa-2-aza-benzo[e]azulen-8-yl]-oxazolidin-5-ylmethyl}-acetamide;
(S)-N-{2-Oxo-3-[3-(3-trifluoromethyl-phenyl)-5,6-dihydro-4H-1-oxa-2-aza-benzo[e]azulen-8-yl]-oxazolidin-5-ylmethyl}-acetamide;
(S)-N-{2-Oxo-3-[3-(3-trifluoromethyl-phenyl)-5,6-dihydro-4H-1-oxa-2-aza-benzo[e]azulen-8-yl]-oxazolidin-5-ylmethyl}-acetamide;
(S)-N-{3-[3-(3-Fluoro-phenyl)-5,6-dihydro-4H-1-oxa-2-aza-benzo[e]azulen-8-yl]-2-oxo-oxazolidin-5-ylmethyl}-acetamide;
(S)-N-{3-[3-(4-Hydroxy-phenyl)-5,6-dihydro-4H-1-oxa-2-aza-benzo[e]azulen-8-yl]-2-oxo-oxazolidin-5-ylmethyl}-acetamide;
(S)-N-{3-[3-(4-methoxy-phenyl)-5,6-dihydro-4H-1-oxa-2-aza-benzo[e]azulen-8-yl]-2-oxo-oxazolidin-5-ylmethyl}-acetamide;
(S)-N-{2-Oxo-3-[3-(4-trifluoromethoxy-phenyl)-5,6-dihydro-4H-1-oxa-2-aza-benzo[e]azulen-8-yl]-oxazolidin-5-ylmethyl}-acetamide;
(S)-N-{2-Oxo-3-[4-(3-trifluoromethyl-phenyl)-5,6-dihydro-4H-1-oxa-2-aza-benzo[e]azulen-8-yl]-oxazolidin-5-ylmethyl}-acetamide;
(S)-N-{3-[3-(4-Fluoro-phenyl)-5,6-dihydro-4H-1-oxa-2-aza-benzo[e]azulen-8-yl]-2-oxo-oxazolidin-5-ylmethyl}-acetamide;
(S)-N-[2-Oxo-3-(3-thiophen-3-yl-5,6-dihydro-4H-1-oxa-2-aza-benzo[e]azulen-8-yl)-oxazolidin-5-ylmethyl]-acetamide;
(S)-N-{3-[3-(4-Hydroxy-thiophen-3-yl)-5,6-dihydro-4H-1-oxa-2-aza-benzo[e]azulen-8-yl]-2-oxo-oxazolidin-5-ylmethyl}-acetamide;
(S)-N-{3-[3-(4-Methoxy-thiophen-3-yl)-5,6-dihydro-4H-1-oxa-2-aza-benzo[e]azulen-8-yl]-2-oxo-oxazolidin-5-ylmethyl}-acetamide;
(S)-N-{2-Oxo-3-[3-(4-trifluoromethoxy-thiophen-3-yl)-5,6-dihydro-4H-1-oxa-2-aza-benzo[e]azulen-8-yl]-oxazolidin-5-ylmethyl}-acetamide;
(S)-N-{2-Oxo-3-[3-(4-trifluoromethyl-thiophen-3-yl)-5,6-dihydro-4H-1-oxa-2-aza-benzo[e]azulen-8-yl]-oxazolidin-5-ylmethyl}-acetamide;
(S)-N-{3-[3-(4-Fluoro-thiophen-3-yl)-5,6-dihydro-4H-1-oxa-2-aza-benzo[e]azulen-8-yl]-2-oxo-oxazolidin-5-ylmethyl}-acetamide;
(S)-N-{3-[3-(5-Hydroxy-thiophen-3-yl)-5,6-dihydro-4H-1-oxa-2-aza-benzo[e]azulen-8-yl]-2-oxo-oxazolidin-5-ylmethyl}-acetamide;
(S)-N-{3-[3-(5-Methoxy-thiophen-3-yl)-5,6-dihydro-4H-1-oxa-2-aza-benzo[e]azulen-8-yl]-2-oxo-oxazolidin-5-ylmethyl}-acetamide;
(S)-N-{2-Oxo-3-[3-(5-trifluoromethoxy-thiophen-3-yl)-5,6-dihydro-4H-1-oxa-2-aza-benzo[e]azulen-8-yl]-oxazolidin-5-ylmethyl}-acetamide;
(S)-N-{2-Oxo-3-[3-(5-trifluoromethyl-thiophen-3-yl)-5,6-dihydro-4H-1-oxa-2-aza-benzo[e]azulen-8-yl]-oxazolidin-5-ylmethyl}-acetamide;
(S)-N-{3-[3-(5-Fluoro-thiophen-3-yl)-5,6-dihydro-4H-1-oxa-2-aza-benzo[e]azulen-8-yl]-2-oxo-oxazolidin-5-ylmethyl}-acetamide;
(S)-N-[3-(3-Furan-3-yl-5,6-dihydro-4H-1-oxa-2-aza-benzo[e]azulen-8-yl)-2-oxo-oxazolidin-5-ylmethyl]-acetamide;
(S)-N-{3-[3-(4-Hydroxy-furan-3-yl)-5,6-dihydro-4H-1-oxa-2-aza-benzo[e]azulen-8-yl]-2-oxo-oxazolidin-5-ylmethyl}-acetamide;
(S)-N-{3-[3-(4-Methoxy-furan-3-yl)-5,6-dihydro-4H-1-oxa-2-aza-benzo[e]azulen-8-yl]-2-oxo-oxazolidin-5-ylmethyl}-acetamide;
(S)-N-{2-Oxo-3-[3-(4-trifluoromethoxy-furan-3-yl)-5,6-dihydro-4H-1-oxa-2-aza-benzo[e]azulen-8-yl]-oxazolidin-5-ylmethyl}-acetamide;
(S)-N-{2-Oxo-3-[3-(4-trifluoromethyl-furan-3-yl)-5,6-dihydro-4H-1-oxa-2-aza-benzo[e]azulen-8-yl]-oxazolidin-5-ylmethyl}-acetamide;
(S)-N-{3-[3-(4-Fluoro-thiophen-3-yl)-5,6-dihydro-4H-1-oxa-2-aza-benzo[e]azulen-8-yl]-2-oxo-oxazolidin-5-ylmethyl}-acetamide;
(S)-N-{3-[3-(5-Hydroxy-furan-3-yl)-5,6-dihydro-4H-1-oxa-2-aza-benzo[e]azulen-8-yl]-2-oxo-oxazolidin-5-ylmethyl}-acetamide;
(S)-N-{3-[3-(5-Methoxy-furan-3-yl)-5,6-dihydro-4H-1-oxa-2-aza-benzo[e]azulen-8-yl]-2-oxo-oxazolidin-5-ylmethyl}-acetamide;
(S)-N-{2-Oxo-3-[3-(5-trifluoromethoxy-furan-3-yl)-5,6-dihydro-4H-1-oxa-2-aza-benzo[e]azulen-8-yl]-oxazolidin-5-ylmethyl}-acetamide;
(S)-N-{2-Oxo-3-[3-(5-trifluoromethyl-furan-3-yl)-5,6-dihydro-4H-1-oxa-2-aza-benzo[e]azulen-8-yl]-oxazolidin-5-ylmethyl}-acetamide;
(S)-N-{3-[3-(5-Fluoro-furan-3-yl)-5,6-dihydro-4H-1-oxa-2-aza-benzo[e]azulen-8-yl]-2-oxo-oxazolidin-5-ylmethyl}-acetamide;
(S)-N-[2-Oxo-3-(3-pyridin-4-yl-5,6-dihydro-4H-1-oxa-2-aza-benzo[e]azulen-8-yl)-oxazolidin-5-ylmethyl]-acetamide;
(S)-N-{3-[3-(3-Hydroxy-pyridin-4-yl)-5,6-dihydro-4H-1-oxa-2-aza-benzo[e]azulen-8-yl]-2-oxo-oxazolidin-5-ylmethyl}-acetamide;
(S)-N-{3-[3-(3-Methoxy-pyridin-4-yl)-5,6-dihydro-4H-1-oxa-2-aza-benzo[e]azulen-8-yl]-2-oxo-oxazolidin-5-ylmethyl}-acetamide;
(S)-N-{2-Oxo-3-[3-(3-trifluoromethoxy-pyridin-4-yl)-5,6-dihydro-4H-1-oxa-2-aza-benzo[e]azulen-8-yl]-oxazolidin-5-ylmethyl}-acetamide;
(S)-N-{2-Oxo-3-[3-(3-trifluoromethyl-pyridin-4-yl)-5,6-dihydro-4H-1-oxa-2-aza-benzo[e]azulen-8-yl]-oxazolidin-5-ylmethyl}-acetamide;
(S)-N-{3-[3-(3-Fluoro-pyridin-4-yl)-5,6-dihydro-4H-1-oxa-2-aza-benzo[e]azulen-8-yl]-2-oxo-oxazolidin-5-ylmethyl}-acetamide;
(S)-N-{3-[3-(2-Hydroxy-pyridin-4-yl)-5,6-dihydro-4H-1-oxa-2-aza-benzo[e]azulen-8-yl]-2-oxo-oxazolidin-5-ylmethyl}-acetamide;
(S)-N-{3-[3-(2-Methoxy-pyridin-4-yl)-5,6-dihydro-4H-1-oxa-2-aza-benzo[e]azulen-8-yl]-2-oxo-oxazolidin-5-ylmethyl}-acetamide;
(S)-N-{2-Oxo-3-[3-(2-trifluoromethoxy-pyridin-4-yl)-5,6-dihydro-4H-1-oxa-2-aza-benzo[e]azulen-8-yl]-oxazolidin-5-ylmethyl}-acetamide;
(S)-N-{2-Oxo-3-[3-(2-trifluoromethyl-pyridin-4-yl)-5,6-dihydro-4H-1-oxa-2-aza-benzo[e]azulen-8-yl]-oxazolidin-5-ylmethyl}-acetamide;
(S)-N-{3-[3-(2-Fluoro-pyridin-4-yl)-5,6-dihydro-4H-1-oxa-2-aza-benzo[e]azulen-8-yl]-2-oxo-oxazolidin-5-ylmethyl}-acetamide;
(S)-N-[2-Oxo-3-(1,4,5,6-tetrahydro-1,2-diaza-benzo[e]azulen-9-yl)-oxazolidin-5-ylmethyl]-acetamide;
(S)-N-[2-Oxo-3-(3-phenyl-1,4,5,6-tetrahydro-1,2-diaza-benzo[e]azulen-9-yl)-oxazolidin-5-ylmethyl]-acetamide;
(S)-N-{3-[3-(2-Hydroxy-phenyl)-1,4,5,6-tetrahydro-1,2-diaza-benzo[e]azulen-9-yl]-2-oxo-oxazolidin-5-ylmethyl}-acetamide;
(S)-N-{3-[3-(2-Methoxy-phenyl)-1,4,5,6-tetrahydro-1,2-diaza-benzo[e]azulen-9-yl]-2-oxo-oxazolidin-5-ylmethyl}-acetamide;
(S)-N-{2-Oxo-3-[3-(2-trifluoromethoxy-phenyl)-1,4,5,6-tetrahydro-1,2-diaza-benzo[e]azulen-9-yl]-oxazolidin-5-ylmethyl}-acetamide;
(S)-N-{2-Oxo-3-[3-(2-trifluoromethyl-phenyl)-1,4,5,6-tetrahydro-1,2-diaza-benzo[e]azulen-9-yl]-oxazolidin-5-ylmethyl}-acetamide;
(S)-N-{3-[3-(2-Fluoro-phenyl)-1,4,5,6-tetrahydro-1,2-diaza-benzo[e]azulen-9-yl]-2-oxo-oxazolidin-5-ylmethyl}-acetamide;
(S)-N-{3-[3-(3-Hydroxy-phenyl)-1,4,5,6-tetrahydro-1,2-diaza-benzo[e]azulen-9-yl]-2-oxo-oxazolidin-5-ylmethyl}-acetamide;
(S)-N-{3-[3-(3-Methoxy-phenyl)-1,4,5,6-tetrahydro-1,2-diaza-benzo[e]azulen-9-yl]-2-oxo-oxazolidin-5-ylmethyl}-acetamide;
(S)-N-{2-Oxo-3-[3-(3-trifluoromethoxy-phenyl)-1,4,5,6-tetrahydro-1,2-diaza-benzo[e]azulen-9-yl]-oxazolidin-5-ylmethyl}-acetamide;
(S)-N-{2-Oxo-3-[3-(3-trifluoromethyl-phenyl)-1,4,5,6-tetrahydro-1,2-diaza-benzo[e]azulen-9-yl]-oxazolidin-5-ylmethyl}-acetamide;
(S)-N-{3-[3-(3-Fluoro-phenyl)-1,4,5,6-tetrahydro-1,2-diaza-benzo[e]azulen-9-yl]-2-oxo-oxazolidin-5-ylmethyl 1-acetamide;
(S)-N-{3-[3-(4-Hydroxy-phenyl)-1,4,5,6-tetrahydro-1,2-diaza-benzo[e]azulen-9-yl]-2-oxo-oxazolidin-5-ylmethyl}-acetamide;
(S)-N-{3-[3-(4-Methoxy-phenyl)-1,4,5,6-tetrahydro-1,2-diaza-benzo[e]azulen-9-yl]-2-oxo-oxazolidin-5-ylmethyl}-acetamide;
(S)-N-{2-Oxo-3-[3-(4-trifluoromethoxy-phenyl)-1,4,5,6-tetrahydro-1,2-diaza-benzo[e]azulen-9-yl]-oxazolidin-5-ylmethyl}-acetamide;
(S)-N-{2-Oxo-3-[3-(4-trifluoromethyl-phenyl)-1,4,5,6-tetrahydro-1,2-diaza-benzo[e]azulen-9-yl]-oxazolidin-5-ylmethyl}-acetamide;
(S)-N-{3-[3-(4-Fluoro-phenyl)-1,4,5,6-tetrahydro-1,2-diaza-benzo[e]azulen-9-yl]-2-oxo-oxazolidin-5-ylmethyl}-acetamide;
(S)-N-[3-(5,6-Dihydro-4H-1-oxa-2-aza-benzo[e]azulen-9-yl)-2-oxo-oxazolidin-5-ylmethyl]-acetamide;
(S)-N-[2-Oxo-3-(3-phenyl-5,6-dihydro-4H-1-oxa-2-aza-benzo[e]azulen-9-yl)-oxazolidin-5-ylmethyl]-acetamide;
(S)-N-{3-[3-(2-Hydroxy-phenyl)-5,6-dihydro-4H-1-oxa-2-aza-benzo[e]azulen-9-yl]-2-oxo-oxazolidin-5-ylmethyl}-acetamide;
(S)-N-{3-[3-(2-Methoxy-phenyl)-5,6-dihydro-4H-1-oxa-2-aza-benzo[e]azulen-9-yl]-2-oxo-oxazolidin-5-ylmethyl}-acetamide;
(S)-N-{2-Oxo-3-[3-(2-trifluoromethoxy-phenyl)-5,6-dihydro-4H-1-oxa-2-aza-benzo[e]azulen-9-yl]-oxazolidin-5-ylmethyl}-acetamide;
(S)-N-{2-Oxo-3-[3-(2-trifluoromethyl-phenyl)-5,6-dihydro-4H-1-oxa-2-aza-benzo[e]azulen-9-yl]-oxazolidin-5-ylmethyl}-acetamide;
(S)-N-{3-[3-(2-Fluoro-phenyl)-5,6-dihydro-4H-1-oxa-2-aza-benzo[e]azulen-9-yl]-2-oxo-oxazolidin-5-ylmethyl}-acetamide;
(S)-N-{3-[3-(3-Hydroxy-phenyl)-5,6-dihydro-4H-1-oxa-2-aza-benzo[e]azulen-9-yl]-2-oxo-oxazolidin-5-ylmethyl}-acetamide;
(S)-N-{3-[3-(3-Methoxy-phenyl)-5,6-dihydro-4H-1-oxa-2-aza-benzo[e]azulen-9-yl]-2-oxo-oxazolidin-5-ylmethyl}-acetamide;
(S)-N-{2-Oxo-3-[3-(3-trifluoromethoxy-phenyl)-5,6-dihydro-4H-1-oxa-2-aza-benzo[e]azulen-9-yl]-oxazolidin-5-ylmethyl}-acetamide;
(S)-N-{2-Oxo-3-[3-(3-trifluoromethyl-phenyl)-5,6-dihydro-4H-1-oxa-2-aza-benzo[e]azulen-9-yl]-oxazolidin-5-ylmethyl}-acetamide;
(S)-N-{3-[3-(3-Fluoro-phenyl)-5,6-dihydro-4H-1-oxa-2-aza-benzo[e]azulen-9-yl]-2-oxo-oxazolidin-5-ylmethyl}-acetamide;
(S)-N-{3-[3-(4-Hydroxy-phenyl)-5,6-dihydro-4H-1-oxa-2-aza-benzo[e]azulen-9-yl]-2-oxo-oxazolidin-5-ylmethyl}-acetamide;
(S)-N-{3-[3-(4-Methoxy-phenyl)-5,6-dihydro-4H-1-oxa-2-aza-benzo[e]azulen-9-yl]-2-oxo-oxazolidin-5-ylmethyl}-acetamide;
(S)-N-{2-Oxo-3-[3-(4-trifluoromethoxy-phenyl)-5,6-dihydro-4H-1-oxa-2-aza-benzo[e]azulen-9-yl]-oxazolidin-5-ylmethyl}-acetamide;
(S)-N-{2-Oxo-3-[3-(4-trifluoromethyl-phenyl)-5,6-dihydro-4H-1-oxa-2-aza-benzo[e]azulen-9-yl]-oxazolidin-5-ylmethyl}-acetamide;
(S)-N-{3-[3-(4-Fluoro-phenyl)-5,6-dihydro-4H-1-oxa-2-aza-benzo[e]azulen-9-yl]-2-oxo-oxazolidin-5-ylmethyl}-acetamide;
(S)-N-[3-(2-Methyl-9,10-dihydro-4H-3-thia-1-aza-benzo[f]azulen-7-yl)-2-oxo-oxazolidin-5-ylmethyl]-acetamide;
(S)-N-[3-(2-Methyl-9,10-dihydro-4H-3-thia-1-aza-benzo[f]azulen-6-yl)-2-oxo-oxazolidin-5-ylmethyl]-acetamide;
(S)-N-[3-(2-Methyl-5,6-dihydro-4H-3-thia-1-aza-benzo[e]azulen-8-yl)-2-oxo-oxazolidin-5ylmethyl] acetamide;
(S)-N-[3-(2-Amino-5,6-dihydro-4H-3-thia-1-aza-benzo[e]azulen-8-yl)-2-oxo-oxazolidin-5-ylmethyl]-acetamide;
(S)-N-[3-(2-Methyl-3,4,5,6-tetrahydro-1,3-diaza-benzo[e]azulen-8-yl)-2-oxo-oxazolidin-5-ylmethyl]-acetamide;
(S)-N-[2-Oxo-3-(2-trifluoromethyl-3,4,5,6-tetrahydro-1,3-diaza-benzo[e]azulen-8-yl)-oxazolidin-5-ylmethyl]-acetamide;
(S)-N-[2-Oxo-3-(3,4,5,6-tetrahydro-2,3-diaza-benzo[e]azulen-9-yl)-oxazolidin-5-ylmethyl]-acetamide; or
(S)-N-[3-(5,6-Dihydro-4H-3-oxa-2-aza-benzo[e]azulen-9-yl)-2-oxo-oxazolidin-5-ylmethyl]-acetamide.

What is also provided is a pharmaceutical formulation comprising a compound of one of formulas I-V admixed with a pharmaceutically acceptable diluent, carrier, or excipient.

What is also provided is a method of treating a bacterial infection in a mammal, comprising administering to a mammal in need thereof an effective amount of a compound of one of formulas I-V.

### DETAILED DESCRIPTION OF THE INVENTION

Reference will now be made in detail to presently preferred compositions or embodiments and methods of the invention, which constitute the best modes of practicing the invention presently known to the inventors.

The term “alkyl” as used herein refers to a straight or branched hydrocarbon of from 1 to 11 carbon atoms and includes, for example, methyl, ethyl, n-propyl, isopropyl, n-butyl, sec-butyl, isobutyl, tert-butyl, n-pentyl, n-hexyl, and the like. The alkyl group can also be substituted with one or more of the substituents selected from lower alkoxy, lower thioalkoxy, halogen, nitro, cyano, oxo, thio, —OH, —SH, —F, —CF₃, —OCF₃, —NO₂, —CO₂H, —CO₂C₁-C₆ alkyl, —NH₂, —NHC₁-C₆ alkyl, 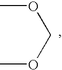
—CONR⁸R⁹, or —N(C₁-C₆alkyl)₂. Preferred alkyl groups have from 1 to 6 carbon atoms (C₁-C₆ alkyl).

The terms “(C₁-C₈)alkyl”, “(C₁-C₆)alkyl”, and “(C₁-C₄)alkyl” as used herein refer to subsets of alkyl which mean a straight or branched hydrocarbon radical having from 1 to 8, 1 to 6, or 1 to 4 carbon atoms respectivly, and include, for example, methyl, ethyl, n-propyl, isopropyl, n-butyl, sec-butyl, isobutyl, tert-butyl, n-pentyl, n-hexyl, n-heptyl, and n-octyl and the like.

The term “(C₃-C₆)cycloalkyl” means a hydrocarbon ring containing from 3 to 6 carbon atoms, for example, cyclopropyl, cyclobutyl, cyclopentyl, or cyclohexyl. Where possible, the cycloalkyl group may contain double bonds, for example, 3-cyclohexen-1-yl. The cycloalkyl ring may be unsubstituted or substituted by one or more substituents selected from alkyl, alkoxy, thioalkoxy, hydroxy, thiol, nitro, halogen, amino, alkyl and dialkylamino, formyl, carboxyl, CN, —NH—CO—R, —CO—NHR, —CO₂R, —COR, wherein R is defined as above, aryl, heteroaryl, wherein alkyl, aryl, and heteroaryl are as defined herein, or as indicated above for alkyl, alkenyl, and alkynyl substitutents. Examples of substituted cycloalkyl groups include fluorocyclopropyl, 2-iodocyclobutyl, 2,3-dimethylcyclopentyl, 2,2-dimethoxycyclohexyl, and 3-phenylcyclopentyl.

The term “halo” includes chlorine, fluorine, bromine, and iodine.

The term “aryl” means a cyclic or polycyclic aromatic ring having from 5 to 12 carbon atoms, and being unsubstituted or substituted with one or more of the substituent groups recited above for alkyl groups including, halogen, nitro, cyano —OH, —SH, —F, —CF₃, —OCF₃, —NO₂, 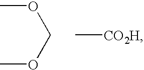
—CO₂H, —CO₂C₁-C₆ alkyl, —NH₂, —NHC₁-C₆ alkyl, —CONR^{a}R^{b}, wherein R^{a} and R^{b} are H or (C₁-C₆)alkyl or (C₃-C₆)cycloalkyl, SO₂alkyl, —SO₂NH₂, or —N(C₁-C₆alkyl)₂. Examples include, but are not limited to phenyl, 2-chlorophenyl, 3-chlorophenyl, 4-chlorophenyl, 2-methylphenyl, 3-methylphenyl, 4-methylphenyl, 2-methoxyphenyl, 3-methoxyphenyl, 4-methoxyphenyl, 2-chloro-3-methylphenyl, 2-chloro-4-methylphenyl, 2-chloro-5-methylphenyl, 3-chloro-2-methylphenyl, 3-chloro-4-methylphenyl, 4-chloro-2-methylphenyl, 4-chloro-3-methylphenyl, 5-chloro-2-methylphenyl, 2,3-dichlorophenyl, 2,5-dichlorophenyl, 3,4-dichlorophenyl, 2,3-dimethylphenyl, 3,4-dimethylphenyl, thienyl, naphthyl, 4-thionaphthyl, tetralinyl, anthracinyl, phenanthrenyl, benzonaphthenyl, fluorenyl, 2-acetamidofluoren-9-yl, and 4′-bromobiphenyl.

The term “heteroaryl” means an aromatic cyclic or polycyclic ring system having from 1 to 4 heteroatoms selected from N, O, and S. Typical heteroaryl groups include 2- or 3-thienyl, 2- or 3-furanyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridinyl, 3-, 4-, or 5-pyridazinyl, 2-pyrazinyl, 2-, 4-, or 5-pyrimidinyl, 2-, 3-, 4-, 5-, 6-, 7-, or 8-quinolinyl, 1-, 3-, 4-, 5-, 6-, 7-, or 8-isoquinolinyl, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl. The heteroaryl groups may be unsubstituted or substituted by 1 to 3 substituents selected from those described above for alkyl, alkenyl, and alkynyl, for example, cyanothienyl and formylpyrrolyl. Preferred aromatic fused heterocyclic rings of from 8 to 10 atoms include but are not limited to 2-, 3-, 4-, 5-, 6-, 7-, or 8-quinolinyl, 1-, 3-, 4-, 5-, 6-, 7-, or 8-isoquinolinyl-, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl. Heteroaryl also includes 2- and 3-aminomethylfuran, 2- and 3-aminomethylthiophene and the like.

The term “heterocyclic” means a monocyclic, fused, bridged, or spiro bicyclic heterocyclic ring systems. Monocyclic heterocyclic rings contain from about 3 to 12 ring atoms, with from 1 to 5 heteroatoms selected from N, O, and S, and preferably from 3 to 7 member atoms, in the ring. Bicyclic heterocyclics contain from about 5 to about 17 ring atoms, preferably from 5 to 12 ring atoms. Bicyclic heterocyclic rings may be fused, spiro, or bridged ring systems. Examples of heterocyclic groups include cyclic ethers (oxiranes) such as ethyleneoxide, tetrahydrofuran, dioxane, and substituted cyclic ethers, wherein the substituents are those described above for the alkyl and cycloalkyl groups. Typical substituted cyclic ethers include propyleneoxide, phenyloxirane (styrene oxide), cis-2-butene-oxide (2,3-dimethyloxirane), 3-chlorotetrahydrofuran, 2,6-dimethyl-1,4-dioxane, and the like. Heterocycles containing nitrogen are groups such as pyrrolidine, piperidine, piperazine, tetrahydrotriazine, tetrahydropyrazole, and substituted groups such as 3-aminopyrrolidine, 4-methylpiperazin-1-yl, and the like. Typical sulfur containing heterocycles include tetrahydrothiophene, dihydro-1,3-dithiol-2-yl, and hexahydrothiophen-4-yl and substituted groups such as aminomethyl thiophene. Other commonly employed heterocycles include dihydro-oxathiol-4-yl, dihydro-1H-isoindole, tetrahydro-oxazolyl, tetrahydro-oxadiazolyl, tetrahydrodioxazolyl, tetrahydrooxathiazolyl, hexahydrotriazinyl, tetrahydrooxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothiophene.

In the context of the present invention, the terms “5-membered heterocyclo” and “5-membered heteroaryl” refer to 5-membered heterocyclo- and heteroaryl groups that fall within the scope of the definitions provided above, or more particularly are summarized in Table 1.

TABLE 1

| | | |
|---|---|---|
| 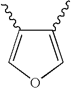 | 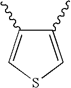 | 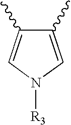 |
| 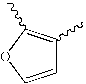 | 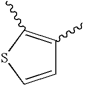 | 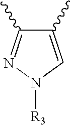 |
| 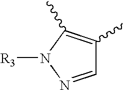 | 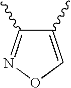 | 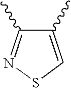 |
| 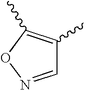 | 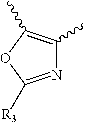 | 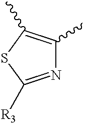 |
| 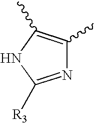 | 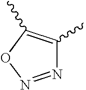 | 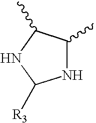 |
| 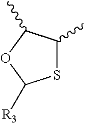 | 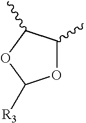 | 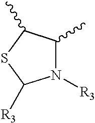 |
| 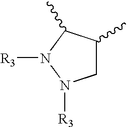 | 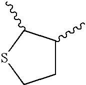 | 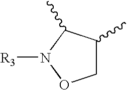 |
| 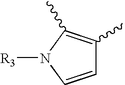 | 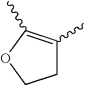 | 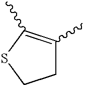 |
| 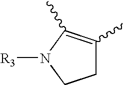 | 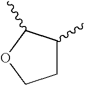 | 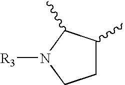 |
| 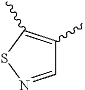 | 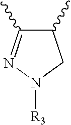 | 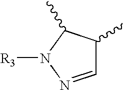 |
| 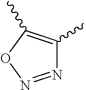 | 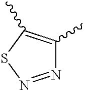 | 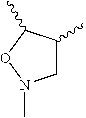 |
| 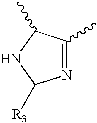 | 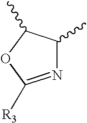 | 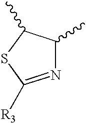 |
| 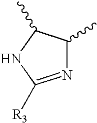 | 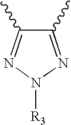 | |

When a bond is represented by a line such as “- - - - - -” this is meant to represent that the bond may be absent or present provided that the resultant compound is stable and of satisfactory valency.

The term “patient” means all mammals, including humans. Other examples of patients include cows, dogs, cats, goats, sheep, pigs, and rabbits.

A “therapeutically effective amount” is an amount of a compound of the present invention that, when administered to a patient, elicits the desired therapeutic outcome; i.e., inhibits bacterial infection.

It will be appreciated by those skilled in the art that compounds of the invention having one or more chiral centers may exist in and be isolated in optically active and racemic forms. Some compounds may exhibit polymorphism. It is to be understood that the present invention encompasses any racemic, optically-active, polymorphic, geometric, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine activity or cytotoxicity using the standard tests described herein, or using other similar tests which are well known in the art.

A “prodrug” is an inactive derivative of a drug molecule that requires a chemical or an enzymatic biotransformation in order to release the active parent drug in the body.

Specific and preferred values for compounds of Formula I are listed below for radicals, substituents, and ranges are for illustration purposes only, and they do not exclude other defined values or other values within defined ranges for the radicals and substituents.

Thus, in formula I, a specific value for 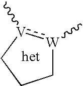
is any value disclosed in Table 1.

A specific value for A is NH, as designated in formula IA.

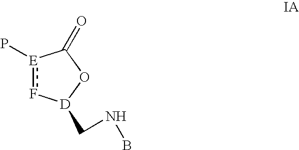

A specific value for B is acetyl as designated in formula IB.

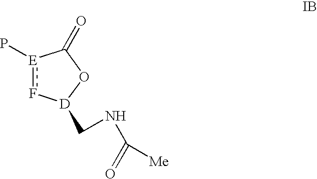

Specific values for D, E, and F, are CH, N, and CH₂, respectively, as designated in formula IC.

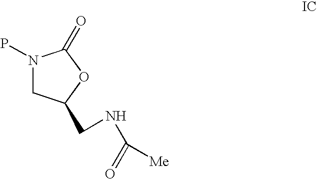

A secific value for P is 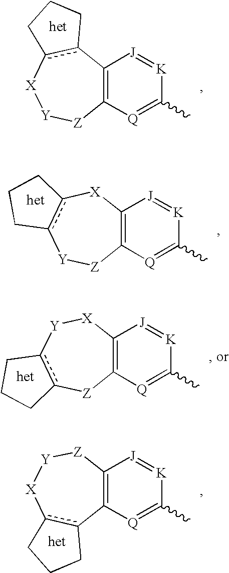
wherein J, K, and Q have any of the manings described herein.

A more specific value for P is 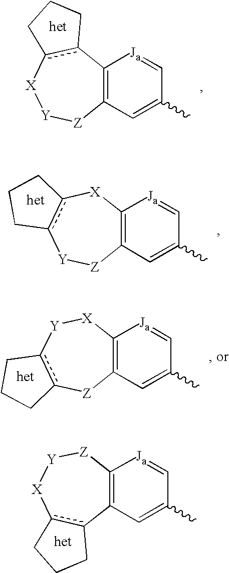
wherein Jₐ is N or CR₁₀, wherein R₁₀ is H or F, and wherein [symbol] indicates the point of attachment.

A still more specific value for P is 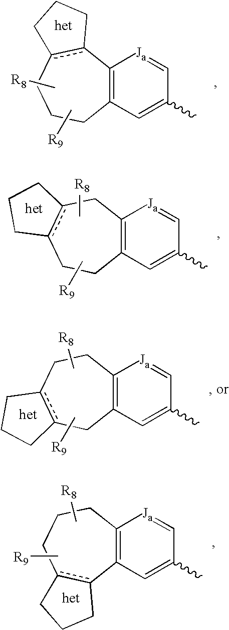
wherein [symbol] indicates the point of attachment; and wherein R₈ and R₉ are each independently H; halo, (C₁-C₈)alkyl, (C₃-C₆)cycloalkyl, O—(C₁-C₄) alkyl, S—(C₁-C₄) alkyl, aryl, (CH₂)ₙ-aryl, heterocyclo, (CH₂)ₙ-heterocyclo, heteroaryl, or (CH₂)ₙ-heteroaryl; wherein n is 0, 1, 2, or 3; or taken together R₈ and R₉ are bonded to the same C and form C═O.

Turning now to a compound of formula II, a specific value for 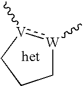
is as defined for 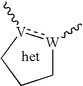
in compounds of formula I.

A specific value for A is NH, as designated in formula IIA.

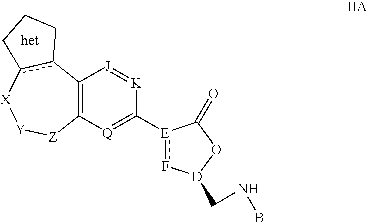

A specific value for B is acetyl, as designated in formula IIB.

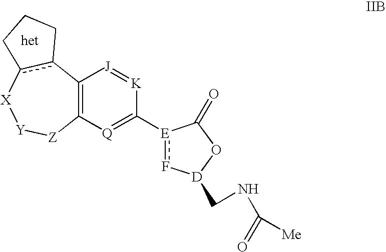

Specific values for D, E, and F, are CH, N, and CH₂, respectively, as designated in formula IIC.

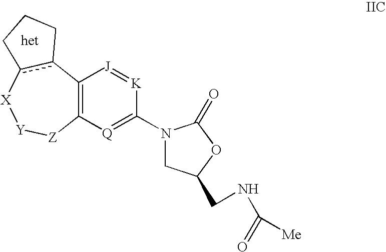

As designated in formula IID, a specific value for J is Ja, wherein Jₐ is N or CR₁₀, wherein R₁₀ is H or F. Specific calues for K and Q are CH, and CH, respectively.

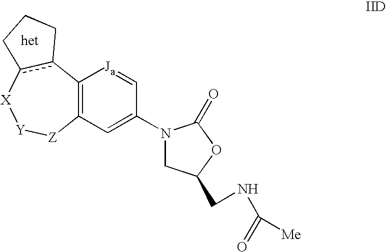

Specific values for X, Y, and Z, are as designated in formula IIE 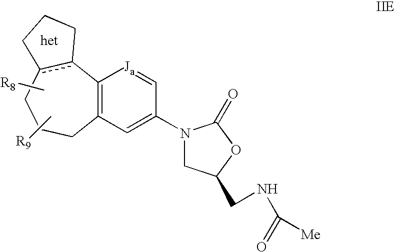
wherein R₈ and R₉ are each independently H; halo, (C₁-C₈)alkyl, (C₃-C₆)cycloalkyl, O—(C₁-C₄) alkyl, S—(C₁-C₄) alkyl, aryl, (CH₂)ₙ-aryl, heterocyclo, (CH₂)ₙ-heterocyclo, heteroaryl, or (CH₂)ₙ-heteroaryl, wherein n is 0, 1, 2, or 3; or taken together R₈ and R₉ are bonded to the same C and form C═O.

Turning now to compounds of formula IIII, a specific value for 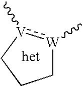
is as defined for 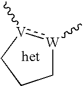
in formula I.

A specific value for A is NH as designated in formula IIIA.

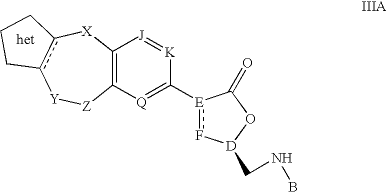

A specific value for B is acetyl as designated in formula IIIB.

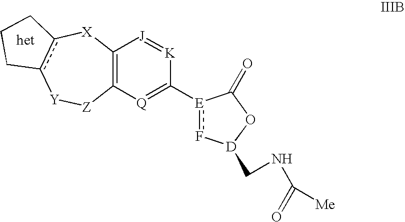

Specific values for D, E, and F, are CH, N, and CH2, respectively, as designated in formula IIIC.

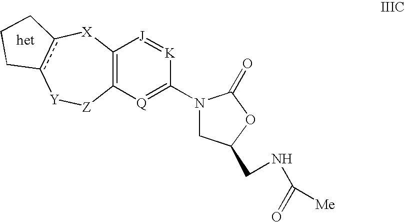

As designated in formula HID, a specific value for J is Ja, wherein Ja is N or CR₁₀, wherein R₁₀ is H or F. Specific calues for K and Q are CH, and CH, respectively.

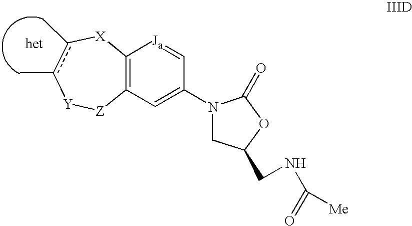

Specific values for X, Y, and Z, are as designated in formula IIIE 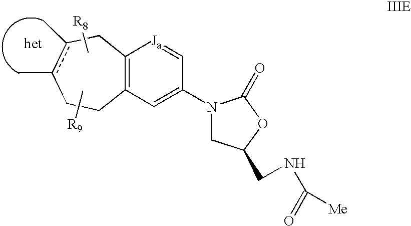
wherein R₈ and R₉ are each independently H; halo, (C₁-C₈)alkyl, (C₃-C₆)cycloalkyl, O—(C₁-C₄) alkyl, S—(C₁-C₄) alkyl, aryl, (CH₂)ₙ-aryl, heterocyclo, (CH₂)ₙ-heterocyclo, heteroaryl, or (CH₂)ₙ-heteroaryl, wherein n is 0, 1, 2, or 3; or taken together R₈ and R₉ are bonded to the same C and form C═O.

Turning now to a compound of formula IV, a specific value for 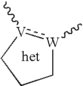
is as defined for 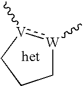
in formula I.

A specific value for A is NH as designated in formula IVA.

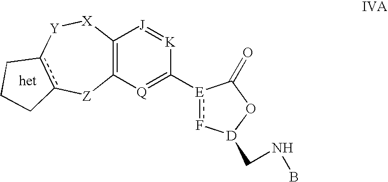

A specific value for B is acetyl as designated in formula IVB.

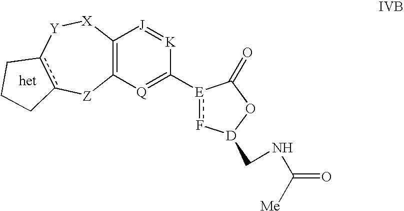

Specific values for D, E, and F, are CH, N, and CH₂, respectively, as designated in formula IVC.

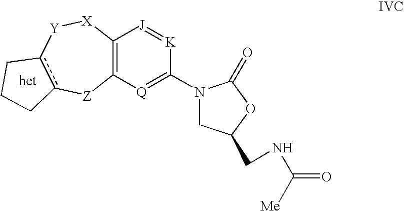

As designated in formula IVD, a specific value for J is Ja, wherein Jₐ is N or CR₁₀, wherein R₁₀ is H or F. Specific calues for K and Q are CH, and CH, respectively.

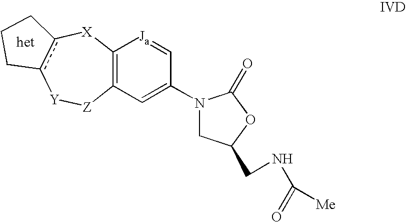

Specific values for X, Y, and Z, are as designated in formula IVE 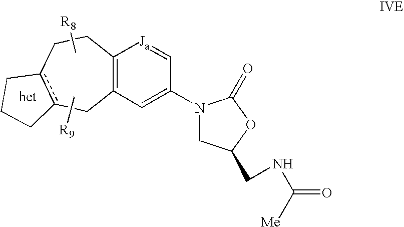
wherein R₈ and R₉ are each independently H; halo, (C₁-C₈)alkyl, (C₃-C₆)cycloalkyl, O—(C₁-C₄) alkyl, S—(C₁-C₄) alkyl, aryl, (CH₂)ₙ-aryl, heterocyclo, (CH₂)ₙ-heterocyclo, heteroaryl, or (CH₂)ₙ-heteroaryl, wherein n is 0, 1, 2, or 3; or taken together R₈ and R₉ are bonded to the same C and form C═O.

Turning now to a compound of formula V, a specific value for 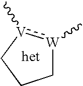
is as defined for 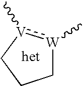
in formula I.

A specific value for A is NH as designated in formula VA.

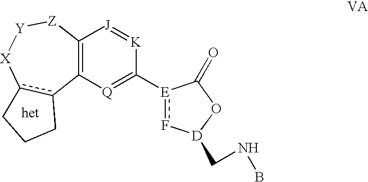

A specific value for B is acetyl as designated in formula VB.

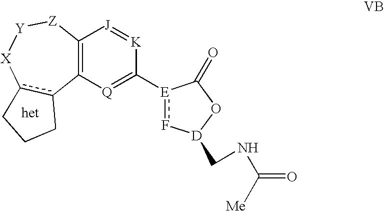

Specific values for D, E, and F, are CH, N, and CH₂, respectively, as designated in formula VC.

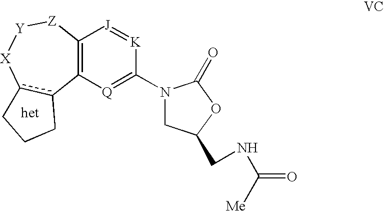

As designated in formula IVD, a specific value for J is Ja, wherein Jₐ is N or CR₁₀, wherein R₁₀ is H or F. Specific calues for K and Q are CH, and CH, respectively.

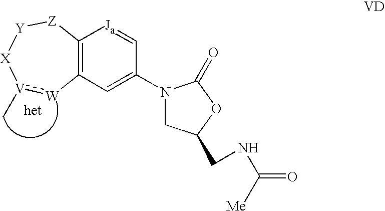

Specific values for X, Y, and Z, are as designated in formula VE 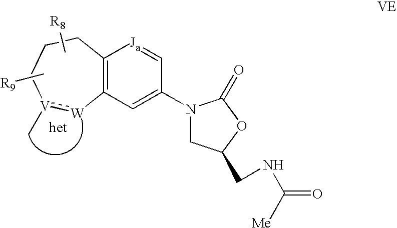
wherein R₉ and R₉ are each independently H; halo, (C₁-C₈)alkyl, (C₃-C₆)cycloalkyl, O—(C₁-C₄) alkyl, S—(C₁-C₄) alkyl, aryl, (CH₂)ₙ-aryl, heterocyclo, (CH₂)ₙ-heterocyclo, heteroaryl, or (CH₂)ₙ-heteroaryl, wherein n is 0, 1, 2, or 3; or taken together R₈ and R₉ are bonded to the same C and form C═O.

### Preparation of Invention Compounds

Strategies for the preparation of invention compounds are depicted generally in Schemes I and II, and more specifically in Schemes 1-##.

As is readily apparent from this disclosure, compounds of the present invention are characterized by a fused tricyclic subunit, covalently attached to a oxazolidinyl subunit. As depicted retrosynthetically in Scheme I, the invention compounds can be prepared from the corresponding bicyclo oxazolidinone intermediate via annelation procedures known to the skilled artisan. One useful platform for elaborating the third ring of the tricyclic subunit recognizable to the skilled artisan is thus the corresponding bicyclic ketone (e.g., V, W, X, Y, or Z is C═O). Many other platforms are available, depending on functional groups present in the cycloheptyl portion of the bicyclo subunit. The bicyclo oxazolidinone intermediates are prepared via covalent attachment of the bicyclo subunit under alkylation (X is NHR, wherein R is a protecting group) or coupling (X is halo, triflate, or another group known to the skilled artisan, that is susceptible to coupling) conditions, to an oxazolidinone core. Methods for the preparation of the requisite bicyclo and oxazolidinone subunits arer readily available to the skilled artisan.

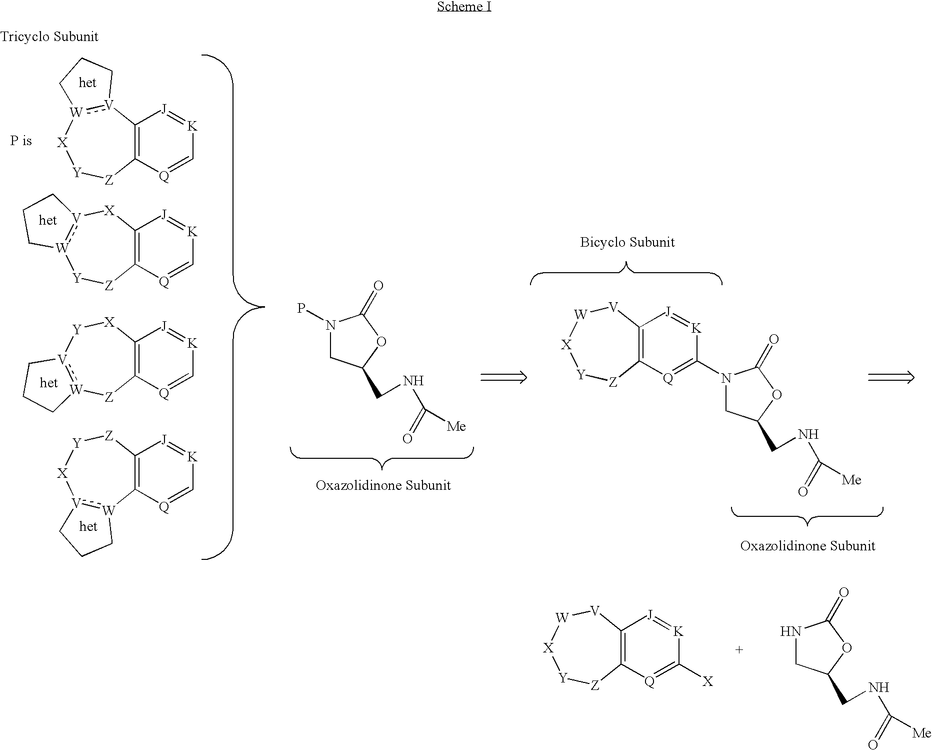

Alternatively, as depicted in Scheme II, the elaborated tricyclo subunit wherein X is halo, triflate, or another group known to the skilled artisan that is susceptible to coupling conditions may be directly appended to the oxazolidinone core.

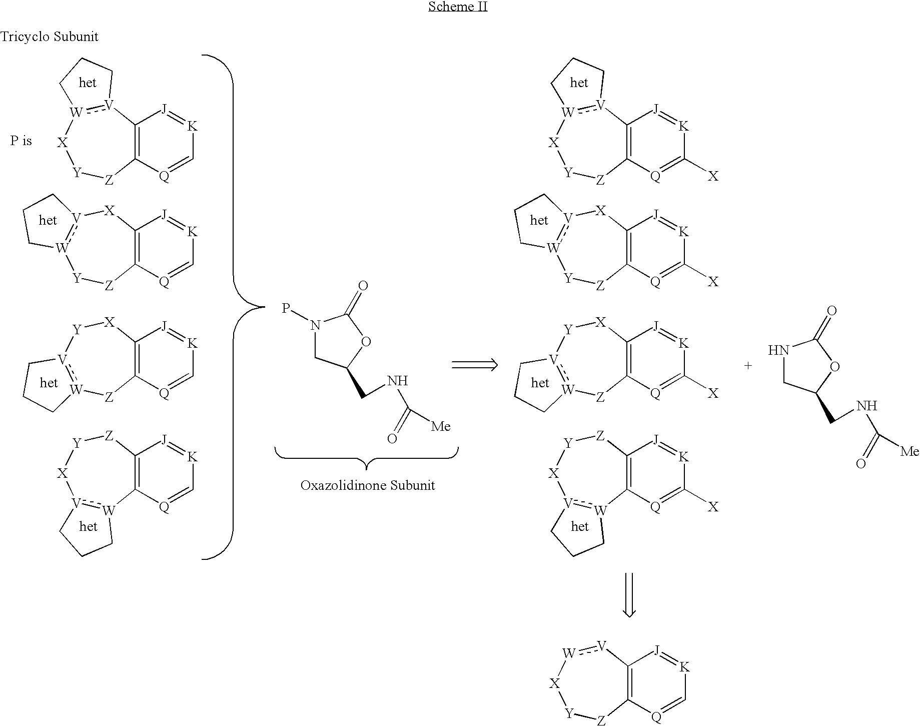

Again alternatively, as depicted in Scheme III, the oxazolidinyl subunit can be elaborated from the corresponding acetamides III-1 or III-2 via treatment with the epoxide or halo acetate as shown. The

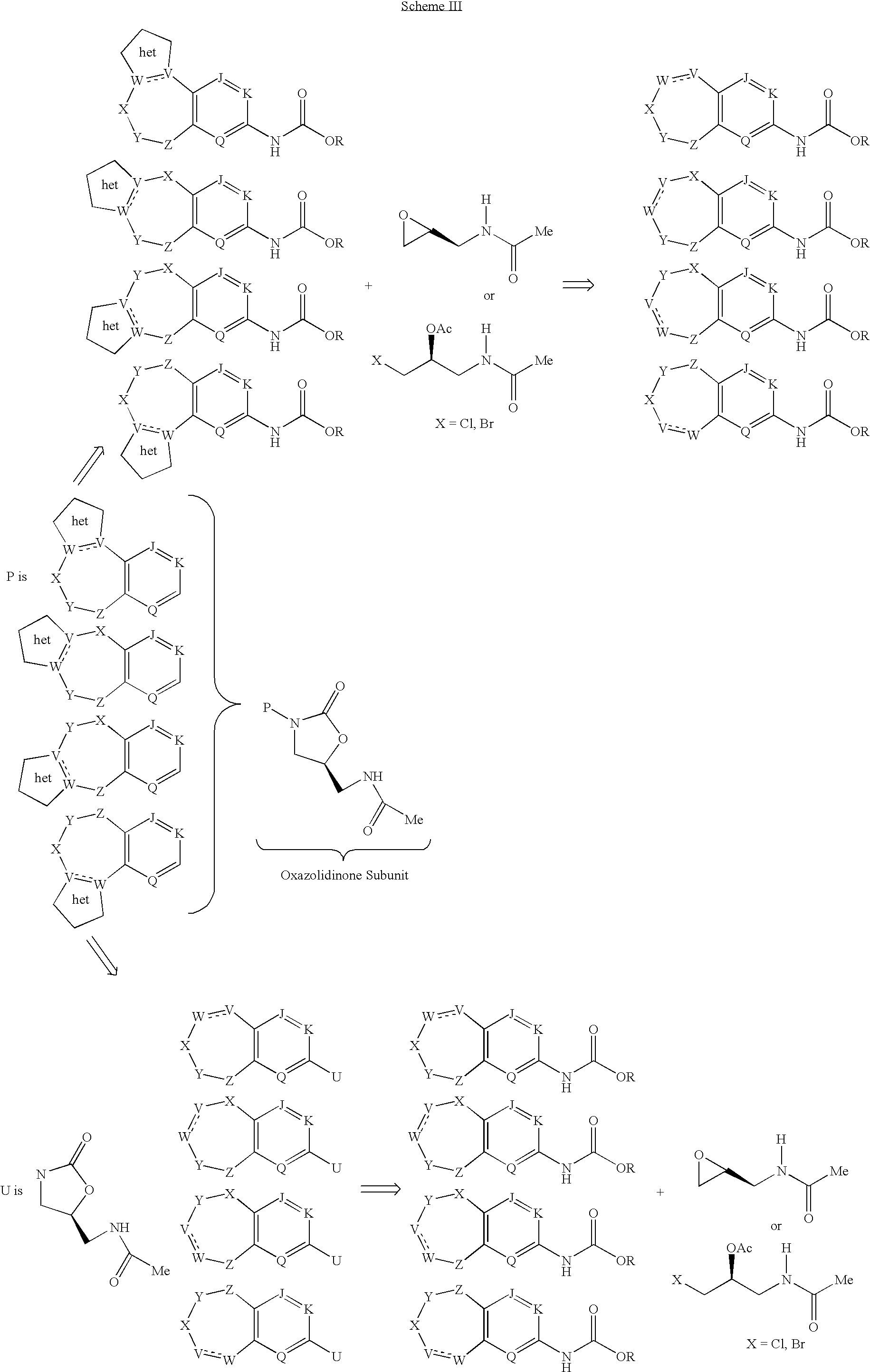

Reflecting the synthetic strategies summarized in Schemes I, II, and III, the following section describing the preparation of the invention compounds has three sections. The first section summarizes the preparation of common intermediates (for instance, the oxazolidinone core). The second section summarizes the preparation and attachment of bicyclo subunits to the oxazolidinyl core to from the bicyclo oxazolidinone intermediates. The third section summarizes the elaboration of the tricyclo subunit using either the bicyclo subunit or bicyclo oxazolidinone intermediate as a platform.

1. Preparation of Common Intermediates

The following compounds which were used in the synthesis of the compounds of the invention, were prepared as follows.

### (R)-5-Hydroxymethyl-oxazolidin-2-one

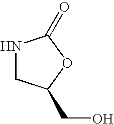

The title compound was prepared according to the procedure described by K. Danielmeier and E. Steckhan in Tetrahedron Assymetry 1995, 6(5), 1181-1190.

### N-(2,4-Dimethoxy-benzyl)-N-(2-oxo-oxazolidin-5-ylmethyl)-acetamide

The title compound was prepared as described in Tetrahedron Letters, 2001, 42, 3681.

### (S)-N-Oxiranylmethyl-acetamide

To a solution of (S)-N-acetyl-3-bromo-2-acetoxypropylamine (5 g, 0.021 mmol) in acetonitrile (20 mL) and methanol (20 mL) was added potassium carbonate (0.021 mmol) portion-wise. The reaction mixture was stirred at 0° C. for 1 hour and then warmed to room temperature slowly and stirred overnight. To it 50 mL of ethyl acetate was added and the precipitate was removed by filtration. Organic solvents were removed and the residue was dissolved in 60 mL of ethyl acetate and remaining precipitate was filtered and organic solution was concentration under reduced pressure to yield 1.6 g (90% yield) to obtain the title compound.

2. Preparation of Bicyclo-Containing Oxazolidinone Intermediates

Approaches to the preparation of the bicyclo-containing intermediates are depicted generally in Schemes 1-10. Schemes 1A-D summarize the preparation of ketone-containing bicyclo cores. Thus in Scheme 1A, nitration of bicyclo cycloheptanone 1A-1 (step I) provides nitro compound 1A-2, which is subsequently reduced to the amine 1A-3 (step II). Protection of the amine moiety in 1A-3 (step III), followed by treatment with (R)-gycidol butyrate provides oxazolidinone 1A-5 (step IV). Mesylation of the alcohol moiety in 1A-5 (step V), followed by treatment with sodium azide, provides azide 1A-7 (step VI). Hydrogenation (step VII) and acetylation (step VII) provides the target compound 1A-9

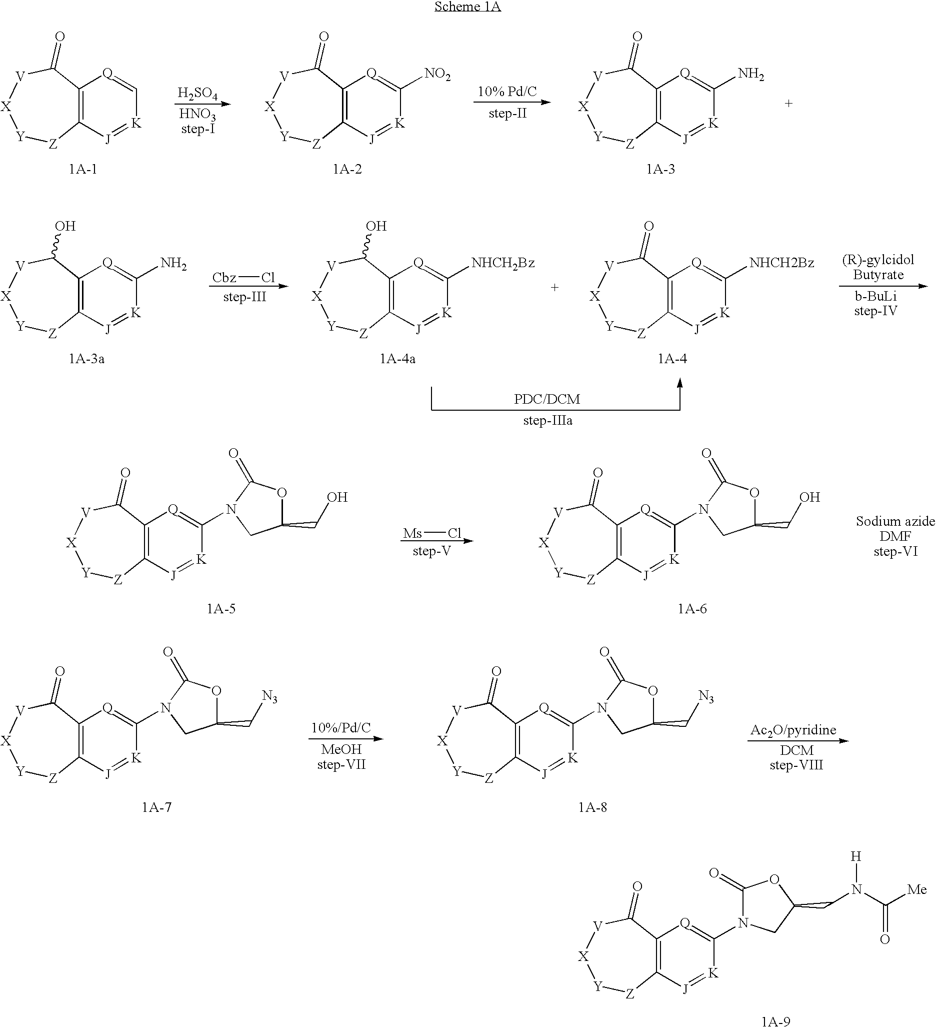

Scheme 1B provides a variant of the Scheme 1A approach wherein keto moiety is “walked” around the ring. Nitration of ketone 1B-1 (step I) provides nitro compound 1B-2, which is reduced to the corresponding amine 1B-3 (step II) under conditions known to the skilled artisan. Protection of the amine moiety (step III), followed by attachment of the oxazolidinone core using reagfents known to the skilled artisan provides 1B-5. Elaboration of the acetamide sidechain of the oxazolidinone subunit in 1B-5 commences with formation of the mesylate or an equivalent (step VI), followed by displacmement with azide, reduction (step VII) and acetylation (step VIII) to provide the target compound 1B-9.

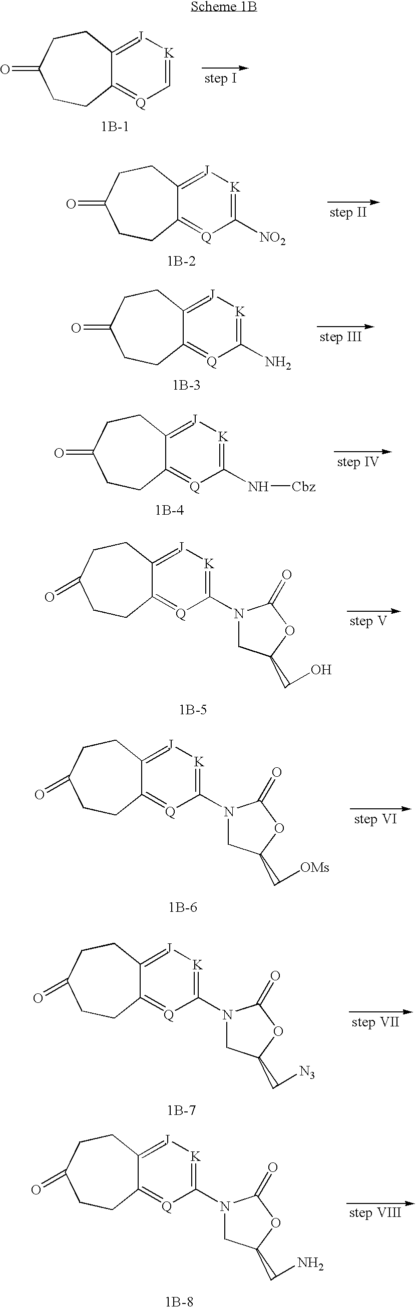

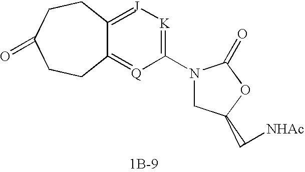

Scheme 1C provides another variant of the Scheme 1A approach wherein keto moiety is “walked” around the ring. Thus, the keto moiety in compound 1C 1 is converted to the exo methylene compound 1C-2 (step I). Epoxidation and ring enlargement of 1C-2 affords ketone 1C-3. Coupling of compound 1C-2 to the oxazolidinone subunit (step III) provides 1C-4. Elaboration of the acetamide sidechain of the oxazolidinone subunit is as provided in Scheme 1B.

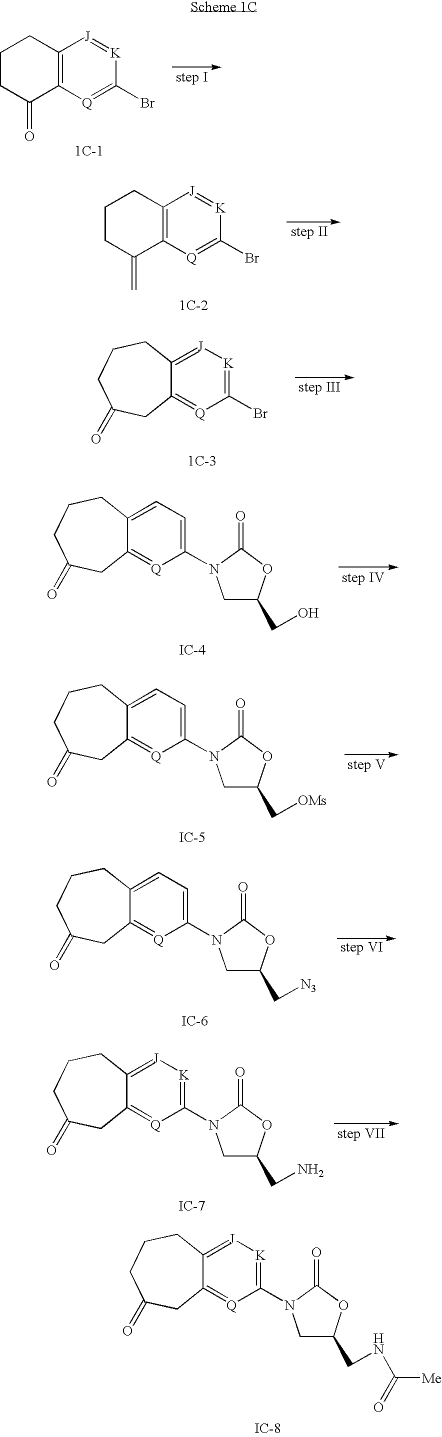

Scheme 1D provides a variant of the Scheme 1C approach. Thus, deprotection and bromination of 1D-1 (step I) provides compound 1D-2. Steps II and III are similar to steps II and III in Scheme IC. Coupling (step IVB) and deprotection (step V) provide the target compound 1D-6.

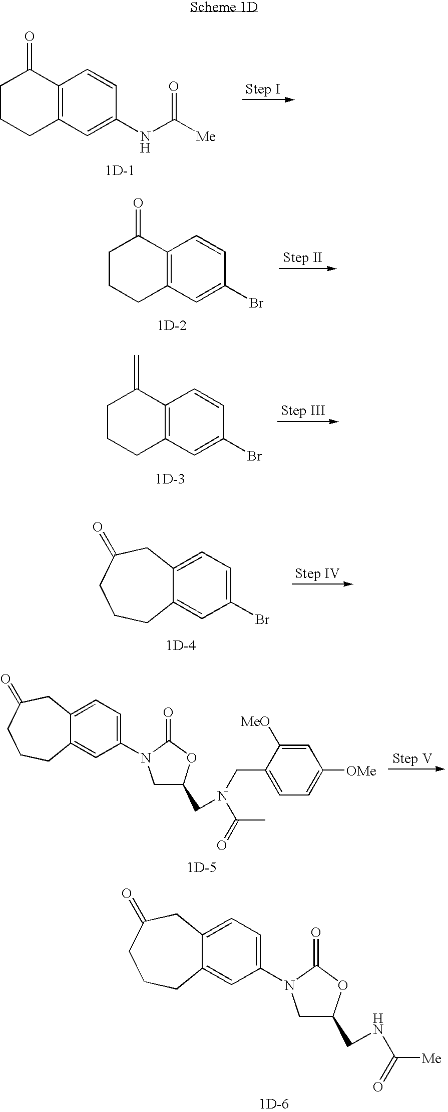

Schemes 2 A-C provide alternative approaches to the attachment of the oxazolidinone subunit of the invention compounds to the fused bicyclo ketone subunit. Method A commences with bromination of 2A-1 to provide 2A-2 (step I), followed by reduction of the ketone moiety (step II) to provide alocohol 2A-3. The alcohol moiety in 2A-3 is removed by techniques known to the skilled artisan (step III), for instance, via conversion to a leaving group such as a mesylate or tosylate, followed by reduction using a trialkyl tin hydride, to provide bromide 2A-4. A variety of coupling procedures may be used to couple bromide 2A-4 to the requisite N-protected acetamide 2-4a (step IV) to provide the protected core 2A-5. Deprotection and oxidation provides the target compound.

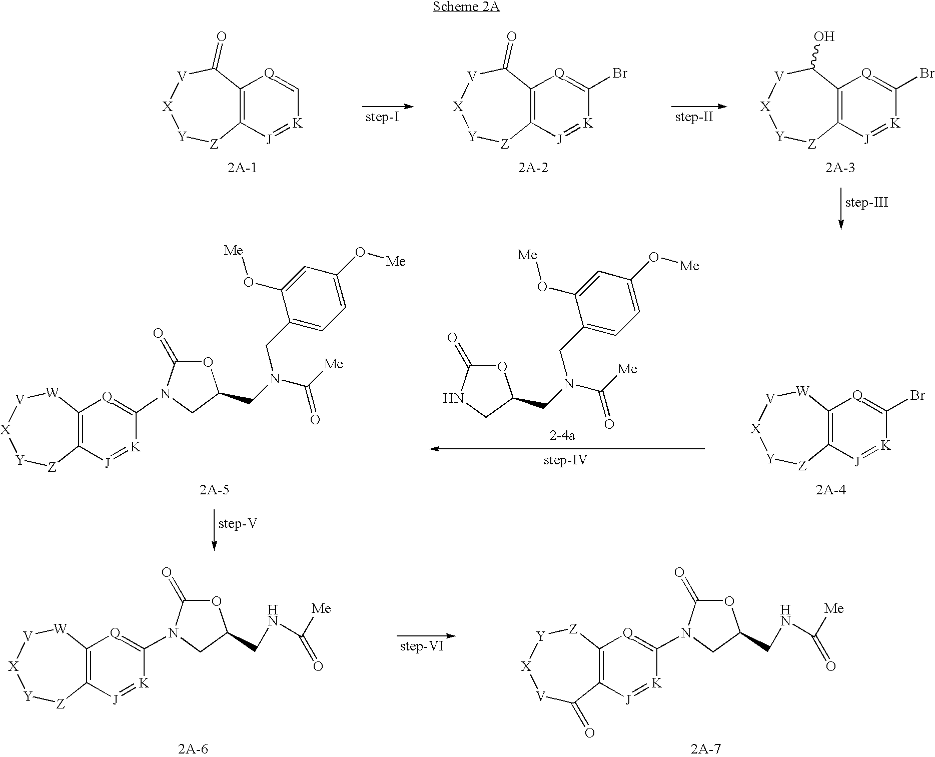

Method B of Scheme 2 provides another variant of the general approach. Thus, iodonitro compound 2B-1-1 is combined with methyl 4-pentynoate 2B-1-2 under conditions known to the skilled artisan (step 1) to provide the coupled product 2B-2. Reduction of the triple bond and nitro groups in 2B-2 (step II) provides methyl ester 2B-3. Acetylation of the amine moiety in 2B-3 (step III) and saponification of the methyl ester (step iv) yiels the acid 2B-5. Intramolecular cyclization of 2B-5 (step V), followed by elaboration of the oxazolidinone subunit (steps VI-X) provides the compound material 2B-11.

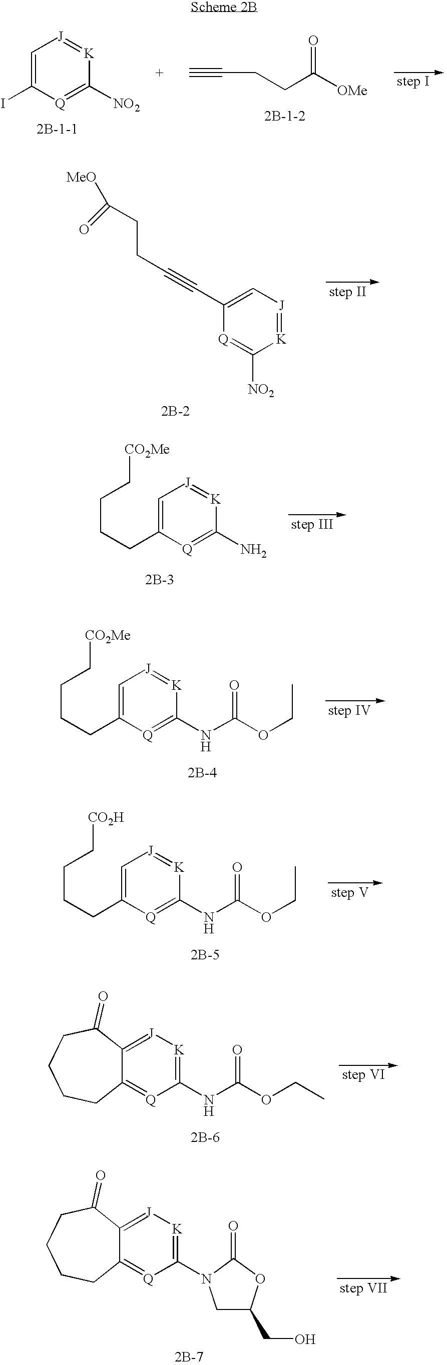

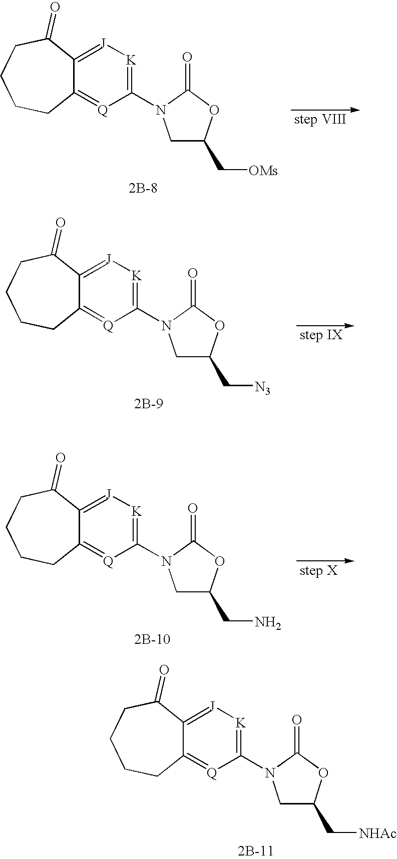

Scheme 2C provides an alternative strategy for the elaboration of the oxazolidinone subunit, compared to steps VI-X in Scheme 2B. Thus compound 2B-6 is treated with N-oxiranyl acetamide in the presence of base to provide 2-11.

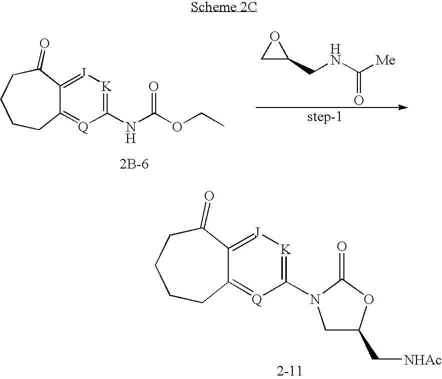

Schemes 3A and 3B provides an approach to unsaturated bicyclo saturated subunits. Thus, in Scheme 3A, reduction of the ketone moiety in 2B-B (step 1), followed by conversion of the resulting alcohol moiety to a leaving group, and base mediated elimination (step II), provides the target compound 3A-2.

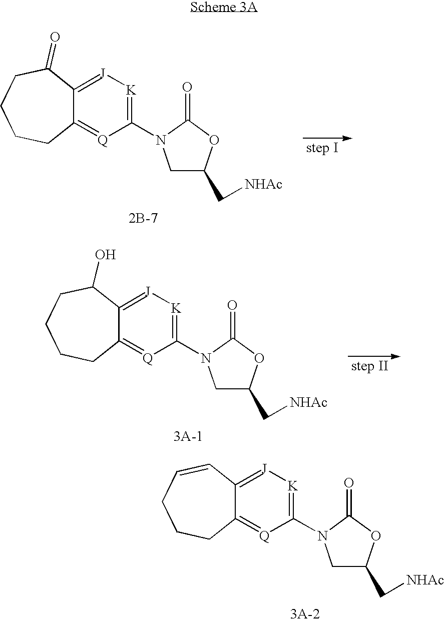

In Scheme 3B, ketone 1-9 is reduced (step I) to provide alcohol 3B-1. Conversion of the alcohol moiety in 3B-1 to leaving group such as a mesylate or tosylate, followed by base-mediated elimination (step II) provides the target compound 3B-2. 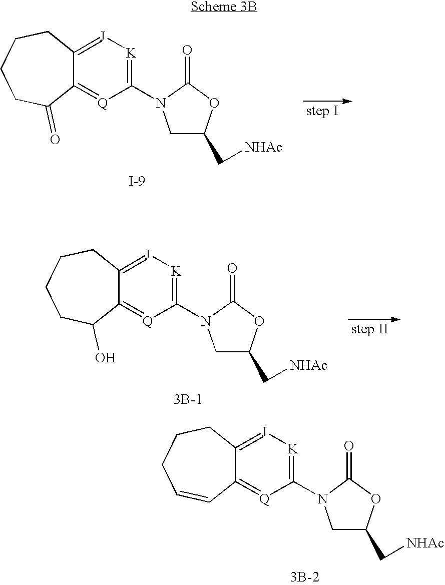
3. Preparation of Fused Bicyclo-Containing Oxazolidinones

Schemes 4-8 provide approaches to the preparation of various fused bicyclo-contiaining oxazolidinones employing intermediates, that were prepared according to Schemes 1-3. Thus, Schemes 4A-J depict the preparation of an invention compound incorporating a fused diazinyl ring. Treatment of compound 2B-11 (Scheme 2B) with DMF acetal in Scheme 4A provides enamine 4A-1. Enamine 4A-1 can be treated with hydrazine or an alkyl substituted Hydrazine to provide diazines 4A-2 and 4A-3, which can be separated using conventional techniques such as silica gel chromatography.

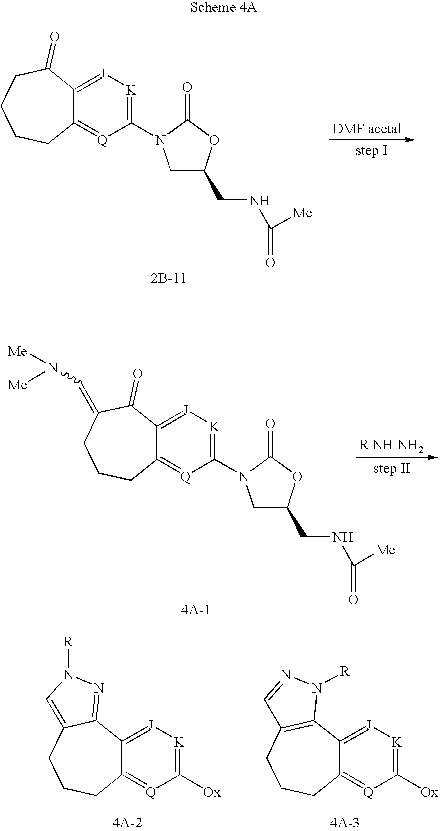

Scheme 4B provides an alternative strategy for the preparation of substituted fused diazines. Thus compound 2B-11 is treated with an acid chloride or anhydride to provide the β-diketo compound 4B-1 (step I). As in Scheme 4A, treatment of compound 4B-1 with hydrazine or an alkyl-substituted hydrazine (step II) provides diazines 4B-2 and 4B-3, which can be separated using conventional techniques such as silica gel chromatography. Alternatively, compound 2B-11 can be treated directly with hydrazine or an alkyl substituted hydrazine (step III) to provide the cycloheptylidene hydrazine derivative 4B-4. Treatment of compound 4B-4 with base and an ester (step IV) provides the fused diazinyl target compound 4B-5.

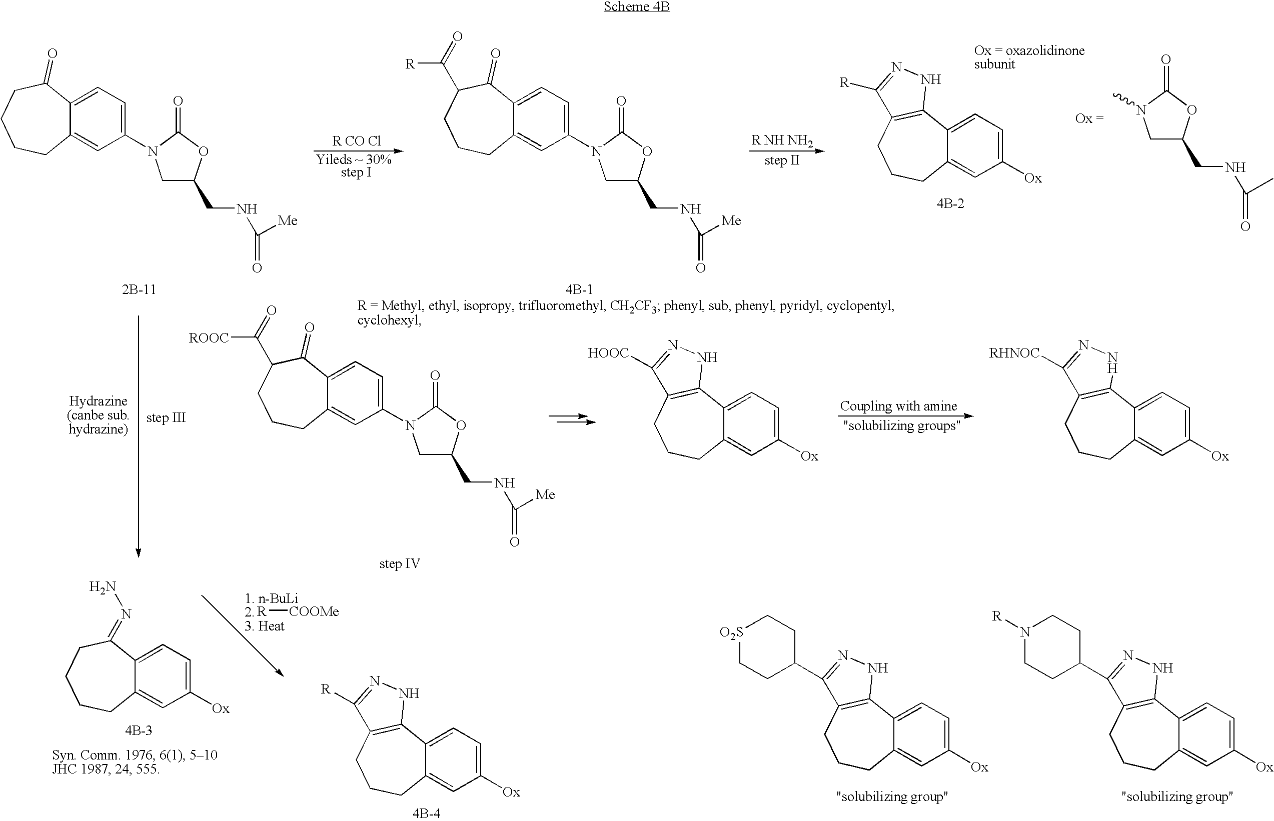

Scheme 4C provides an alternative approach to the synthesis of fused substituted diazinyl systems, that focuses on the preparation of invention compounds with enhanced solubilities. Thus compound 4C-1, which is readily prepared according to methods available to the skilled artisan, is converted to the diazinyl system 4C-2 (step I) as provided in Schemes 4A and 4B. The acid moiety in compound 4C-2 provides a platform for appending various solubilizing groups on the invention compound skeleton, such as depicted in compounds 4C-3,

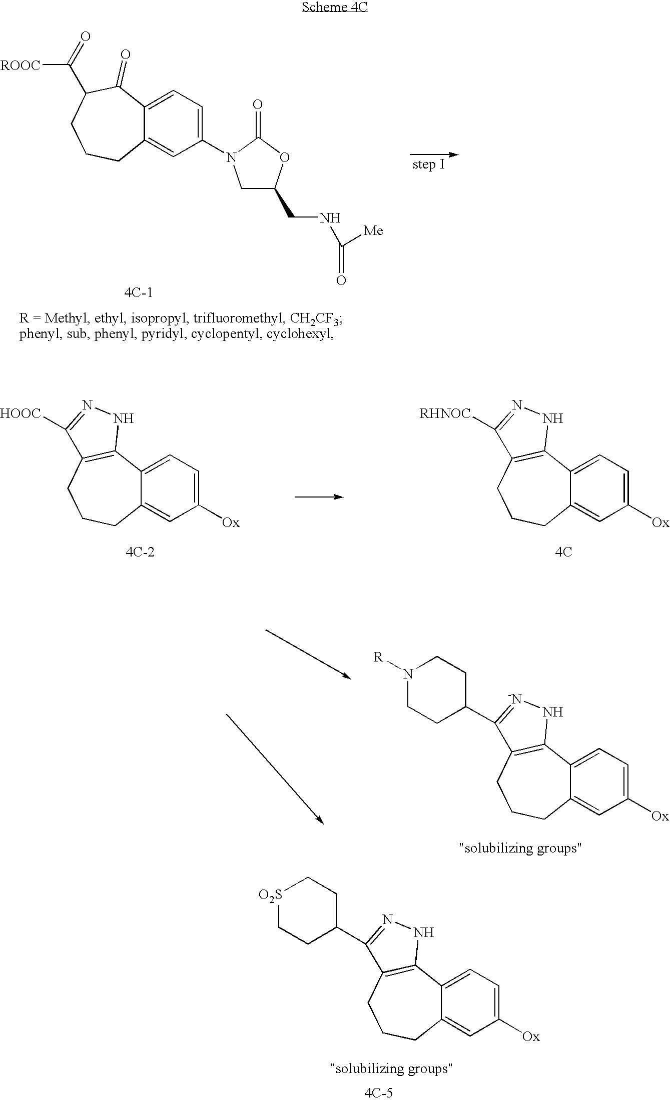

Scheme 4D summarizes an alternative strategy for the preparation of substituted diazinyl systemts. Thus, alkylation of 2B-11 using base and diethyloxalate, followed by treatment with hydrazine or substituted hydrazine provides the hydroxymethyl-substituted diazine 4D-1. Compound 4D-1 can be converted to the substituted amine 4D-2 via conversion of the alcohol moiety to a leaving group such as a tosylate, mesylate, or halide, followed by displacement with an alkyl amine. Alternatively, 1-carbon homolgues of 4D-2 suchs as 4D-5 can be constructed via the cyano compound 4D-4.

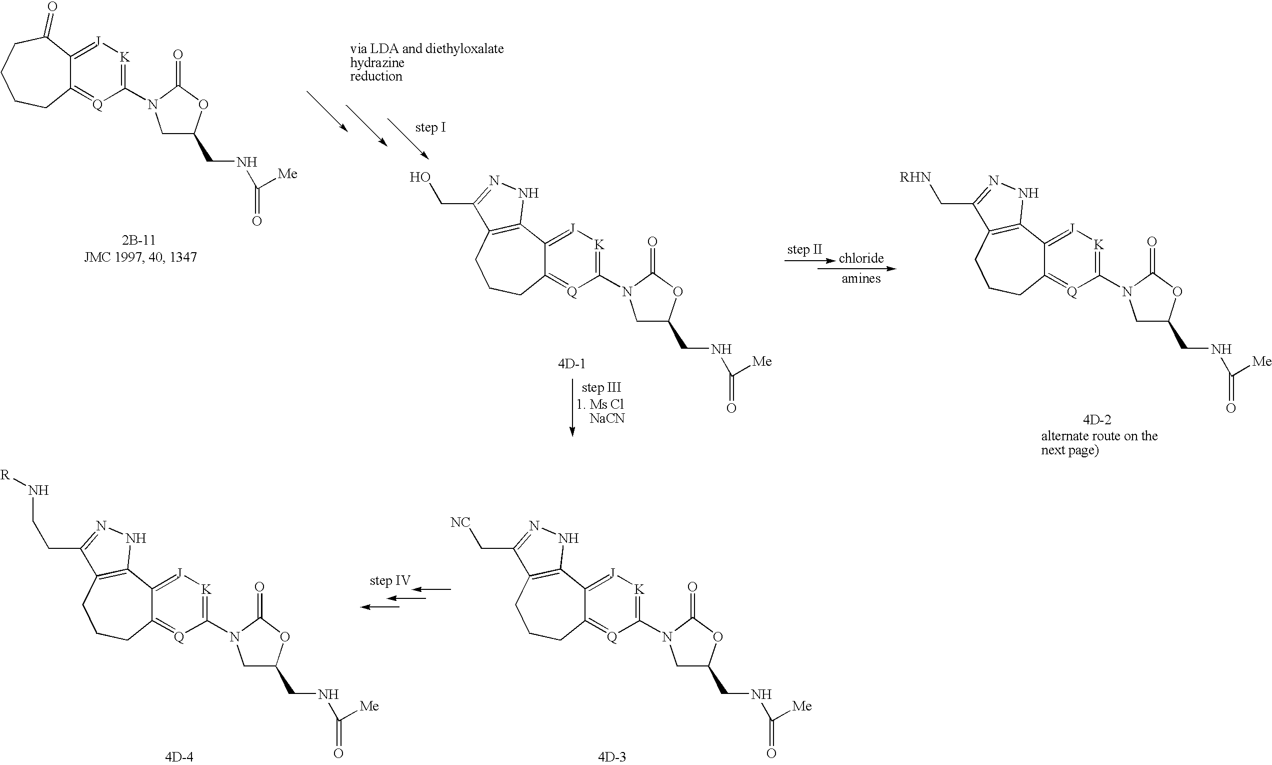

Scheme 4E summarizes another strategy for the preparation of substituted diazinyl containing invention compounds. Thus compound 2B-11 is treated with dimethylcarbonate or nitilo acetic acid methyl ester in the presence of base to afford the β-ketoester 4E-1. Treatment of β-ketoester 4E-1 with hydrazine or a substituted hydrazine provides the diazinyl system 4E-2. Compound 4E-2 can be used as an intermediate in the preparation of other compounds, such as various ethers (via alkylations; see, e.g., 4E-3), or other systems via coupling procedures (see, e.g., 4E-4). Alternatively, compound 2B-11 can be converted to the β-ketoester 4E-1 and alkylated in situ to provide 4E-5. Compound 4E-5 can be treated with hydrazine or a substituted hydrazine to give pyrazolone analogue 4E-6. Alternatively, 2B-11 can be converted to 4E-7 via esterification of the corresponding carboxylic acid (see Schemes 4B and 4C for the synthesis of the acid), converted to the diazine as provided above to give 4E-8, reduced to the hydroxymethyl compound 4E-9, and alkylated or coupled as provided for 4E-3 or 4E-4 to give 4E-10.

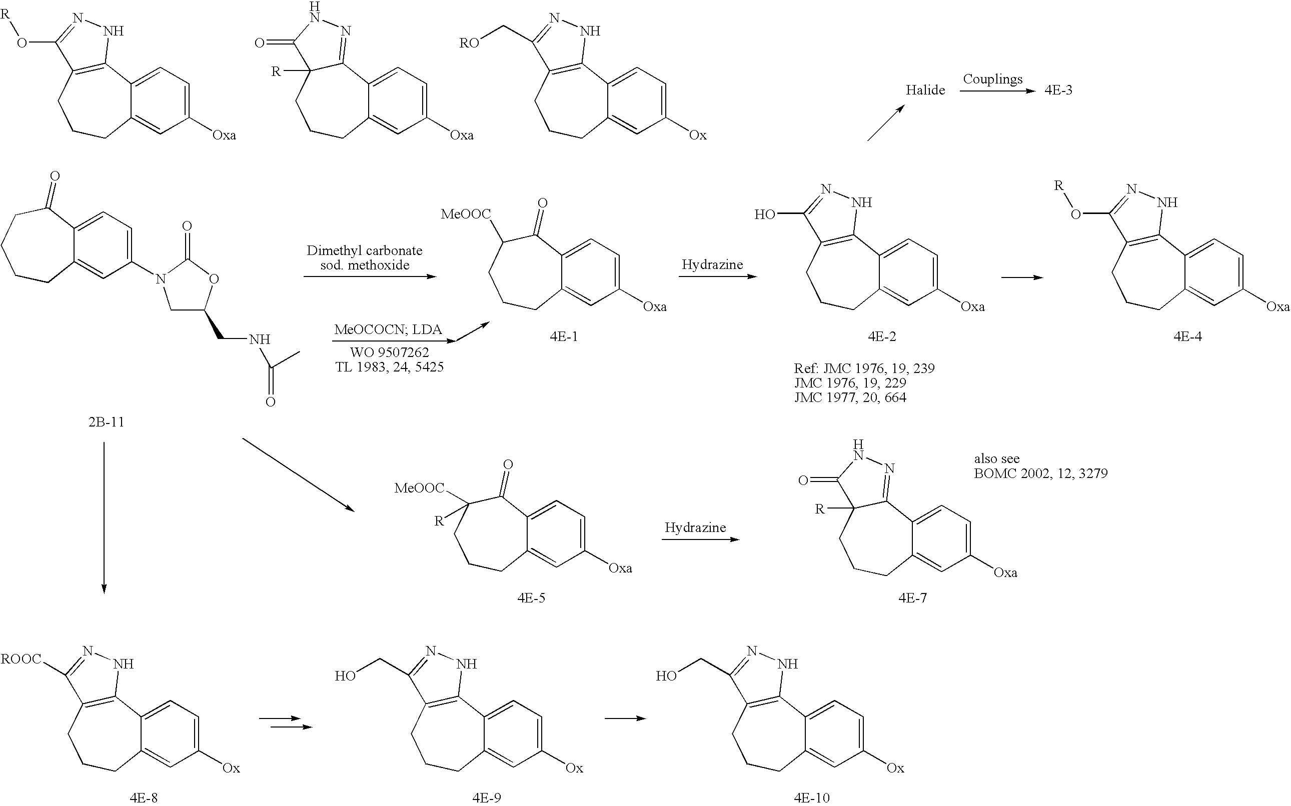

Scheme 4F highlights the synthesis of aminated diazinyl systems. Thus, compound 2B-11 is treated with carbon disulfide, and amine (such as piperizine, although the other, and methyl iodide in the presence of base to provide intermediate 4F-1. Compound 4F-1 is converted to diazinyl system 4F-2 via a series of reactions, including treatment with hydrazine or a substituted hydrazine; deprotection; acylation, followed by a carbon-nitogernt bond forming reaction such as sulfonylation, alkylation; or the like.

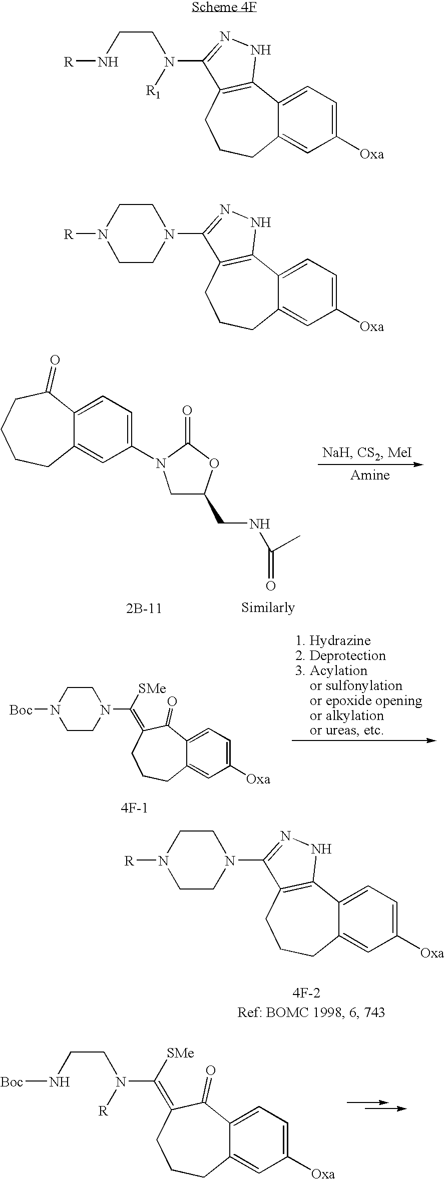

Scheme 4G provides an alternative approach to the synthesis of substituted diazinyl systems. Thus, compound 2B-11 is converted to the β-keto amide via treatment with a protected α, β, or γ-amino acid in the presence of carbonyl di-imidazole or the like to provide 4G-1. Treatment of 4G-1 with hydrazone or a substituted hydrazone as provided in earlier schemes gives rise to the target compound 4G-2, which may be derivatized further as provided in earlier schemes.

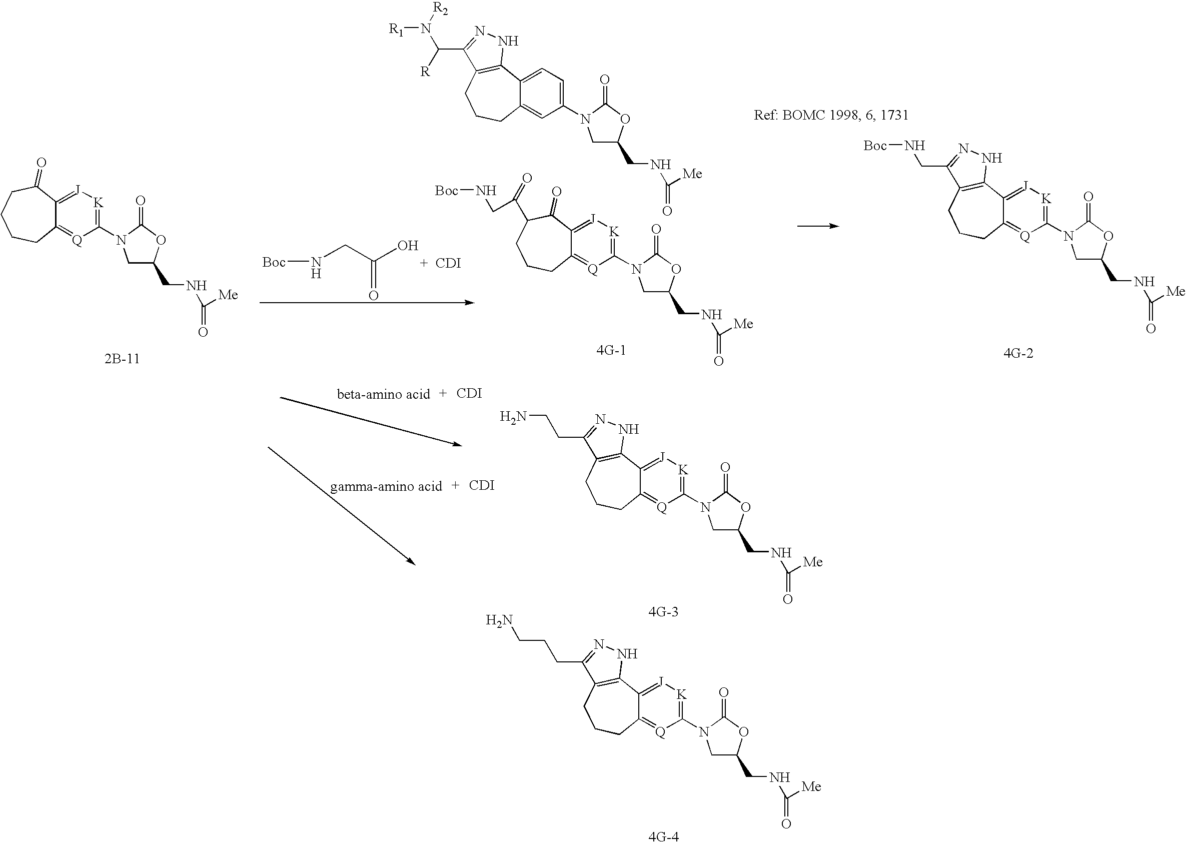

Scheme 4H provides another approach to the synthesis of substituted diazinyl systems. Thus, compound 2B-11 is converted to β-keto ester 4H-1 using methoxy acetic acid methyl ester. The diazinyl system 4H-2 is prepared as provided earlier using hydrazine or a substituted hydrazine. Conversion of 4H-2 to aldehyde 4H-3, followed by reductive amination, provides the target compound 4H-4. Alternatively, 4H-2 can be converted to the hydroxymethyl compound 4H-5, which may be alkylated or homolgated as indicated to give 4H-6 and 4H-8, respectively.

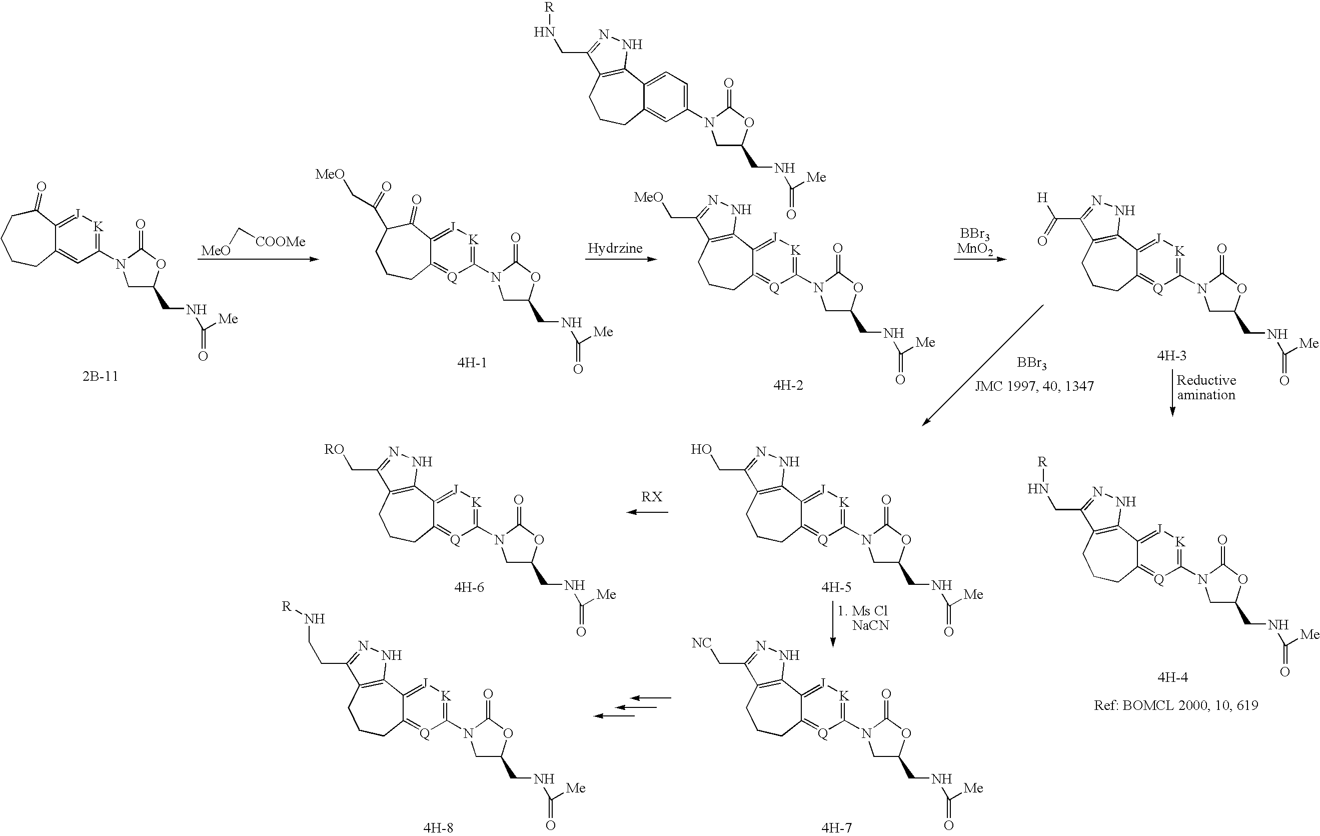

Scheme 4J provides an approach to other substituted diazinyl systems. Thus, compound 2B-11 is converted to the exo olefin 4J-1 via procedures well known to the skilled artisan. Epoxidation of 4J-1 provides 4J-2. Oxidative ring opening of the epoxide and treatment with hydrazine or a substituted hydrazine provides the target compound 4J-4.

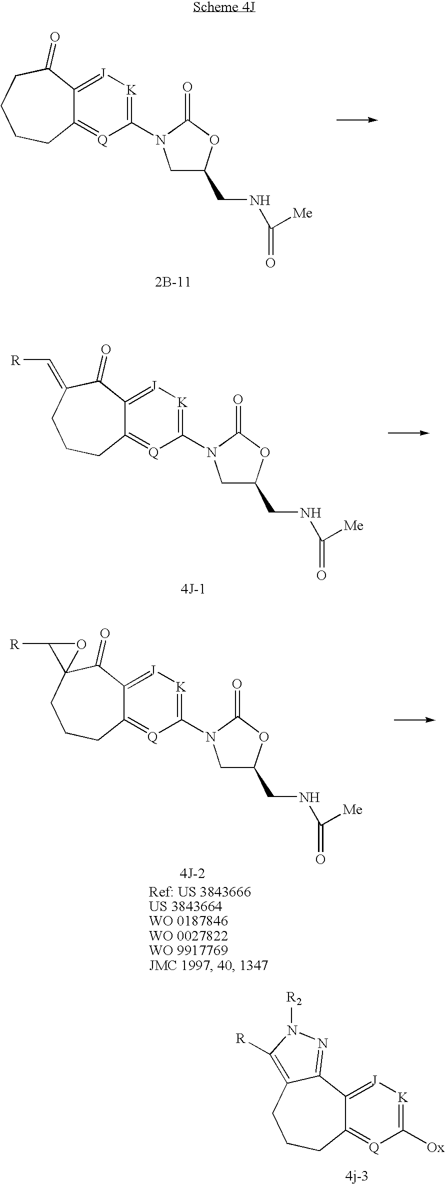

Scheme 5 provides an approach to diazines and isoxazoles via an α-cyano intermediate. Thus, compound 2B-11 undergoes bromination and subsequent cyanation to provide compound 5-1. Treatment of cyano compound 5-1 with hydrazine or hydroxylamine, or substituted variants thereof gives riste to diazine 5-2 or isoxazole 5-3.

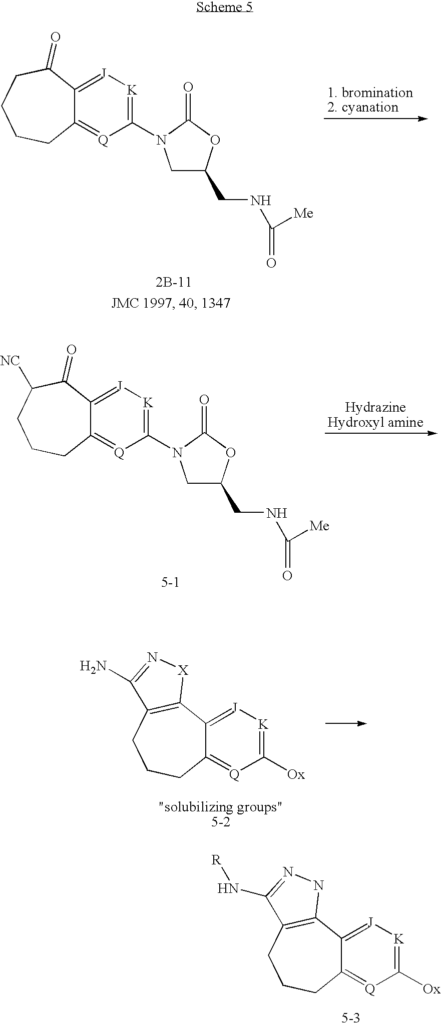

Scheme 6 provides an approach to pyrrole-containing systems, as well as furna-containing systems. The exo olefin 6-1 can be prepared as indicated in Scheme 4J. Conversion of 6-1 to a dicarbonyl compound 6-4, followed by base-mediated cyclization treatment, provides furan 6-5. Similarly, formation of the imine of 6-1, followed by cyclization, gives the corresponding pyrrole 6-6.

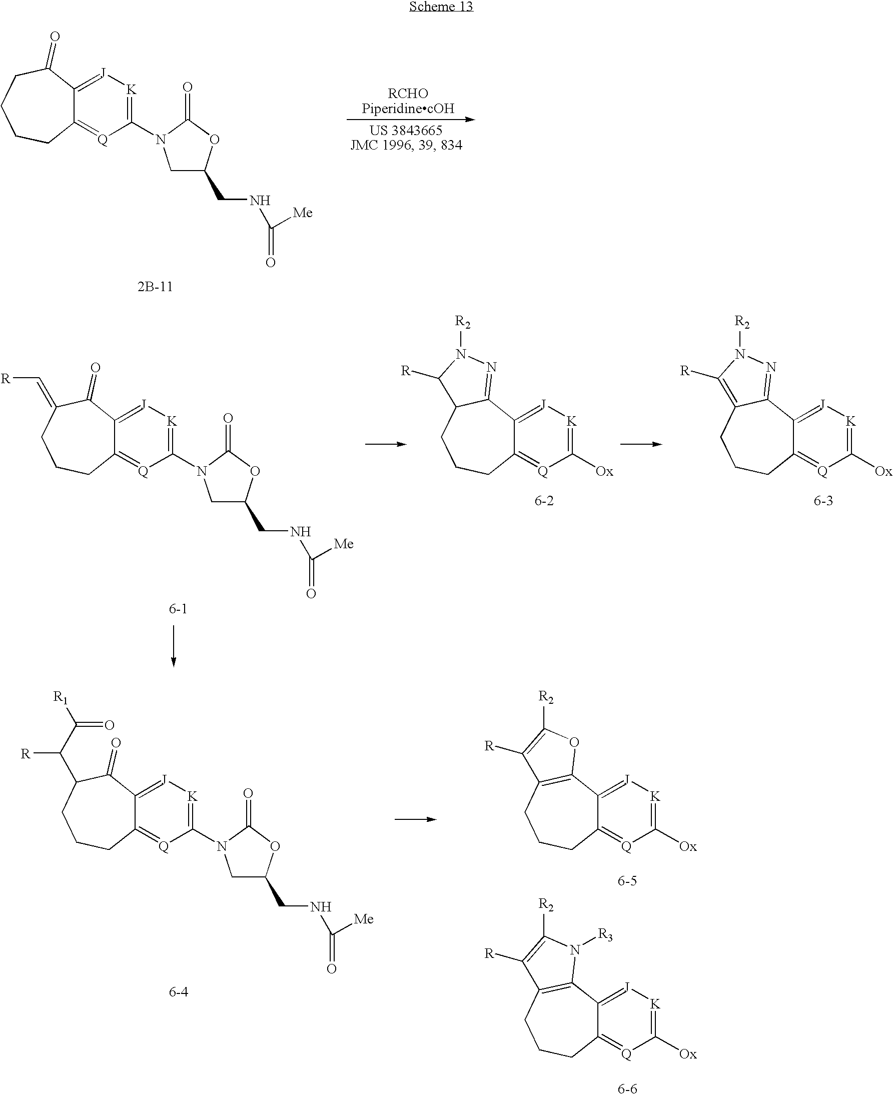

Scheme 7 provides approaches to thiazole- oxazole-, and imidazole-containing systems. Thus, bromination of compound 7-11 provides α-bromoketone 7-1. Treatment of 7-1 with a thiamide or thioacetic acid affords the requisite thiazole 7-2. Alternatively, treatment of 7-1 with a urea or an amine in the presence of hydroxylamine provides the corresponding imidazoles 7-3 and 7-4. The corresponding oxazole 7-5 can also be prepared via this general strategy,

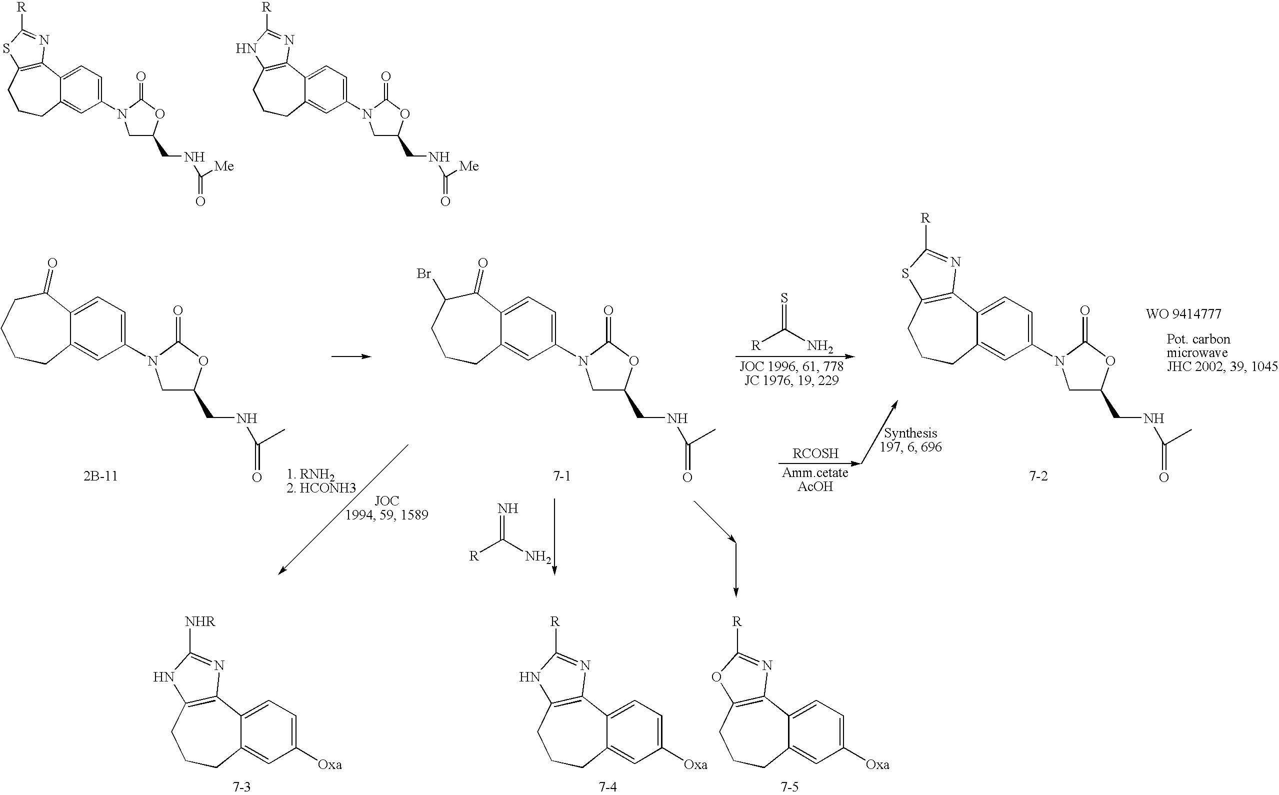

Scheme 8 summarzies an approach to isoazole-containing systems. Thus, compound 2B-11 is treated with hydydroxylamine to provide the oxime 8-1. Treatment of 8-1 with base in the presence of an ester, followed by heating, provides the target isoxazole 8-2.

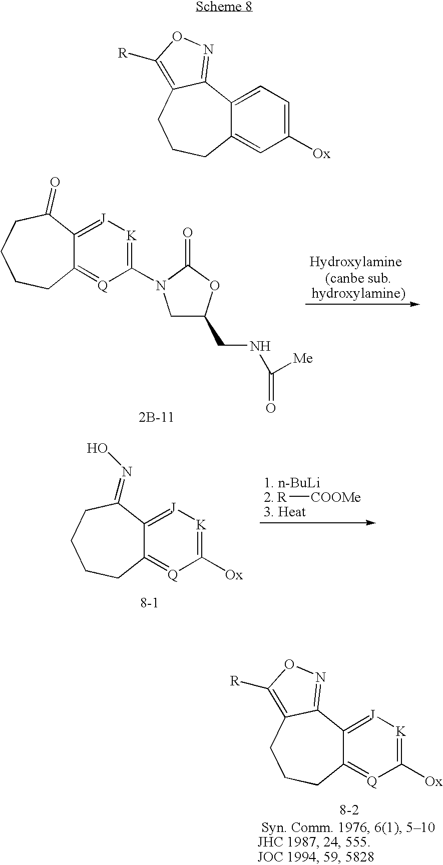

The invention compounds can be screened to identify bioactive molecules with different biological activities using methods available in the art. The bioactive molecules, for example, can possess activity against a cellular target, including but not limited to enzymes and receptors, or a microorganism. A target cellular ligand or microorganism is one that is known or believed to be of importance in the etiology or progression of a disease. Examples of disease states for which compounds can be screened for biological activity include, but are not limited to, inflammation, infection, hypertension, central nervous system disorders, and cardiovascular disorders.

Pharmaceutical Formulations

The present invention also provides pharmaceutical compositions which comprise a bioactive invention compound or a salt such as a pharmaceutically acceptable salt thereof and optionally a pharmaceutically acceptable carrier. The compositions include those in a form adapted for oral, topical or parenteral use and can be used for the treatment of bacterial infection in mammals including humans.

The compounds, such as antibiotic compounds, also referred to herein as antimicrobial compounds, according to the invention can be formulated for administration in any convenient way for use in human or veterinary medicine, by analogy with other bioactive agents such as antibiotics. Such methods are known in the art and are not described in detail herein.

The composition can be formulated for administration by any route known in the art, such as subdermal, by-inhalation, oral, topical or parenteral. The compositions may be in any form known in the art, including but not limited to tablets, capsules, powders, granules, lozenges, creams or liquid preparations, such as oral or sterile parenteral solutions or suspensions.

The topical formulations of the present invention can be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present, for example, from about 1% up to about 98% of the formulation. For example, they may form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods will known in normal pharmaceutical practice.

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

For parenteral administration, fluid unit dosage forms are prepared utilizing the compound and a sterile vehicle, water being preferred. The compound, depending on the vehicle and concentration used, can be either suspended or dissolved in the vehicle or other suitable solvent. In preparing solutions, the compound can be dissolved in water for injection and filter sterilized before filling into a suitable vial or ampoule and sealing. Advantageously, agents such as a local anesthetic preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

The compositions may contain, for example, from about 0.1% by weight, e.g., from about 10-60% by weight, of the active material, depending on the method of administration. Where the compositions comprise dosage units, each unit will contain, for example, from about 50-500 mg of the active ingredient. The dosage as employed for adult human treatment will range, for example, from about 100 to 3000 mg per day, for instance 1500 mg per day depending on the route and frequency of administration. Such a dosage corresponds to about 1.5 to 50 mg/kg per day. Suitably the dosage is, for example, from about 5 to 20 mg/kg per day.

The invention compounds disclosed herein can be used in a variety of pharmaceutical applications. In one embodiment, the compounds may be used as antimicrobial agents for the treatment of infectious disorders that are caused by microbial agents, such as bacteria.

In one embodiment, compositions, for treating or preventing infectious disorders are provided, comprising an oxazolidone compound as disclosed herein in combination with a pharmaceutically acceptable carrier.

In another embodiment, there is provided a dosage amount of an invention compound as disclosed herein in an effective amount for the treatment, prevention or alleviation of a disorder, such as an infectious disorder.

The invention compounds can be screened for activity against different microbial agents and appropriate dosages may be determined using methods available in the art.

The compounds may be used to treat a subject to treat, prevent, or reduce the severity of an infection. Subjects include animals, plants, blood products, cultures and surfaces such as those of medical or research equipment, such as glass, needles and tubing.

Antiinfective Activity

In one embodiment, methods of treating or preventing an infectious disorder in a subject, such as a human or other animal subject, are provided, by administering an effective amount of an invention compound as disclosed herein to the subject. In one embodiment, the compound is administered in a pharmaceutically acceptable form optionally in a pharmaceutically acceptable carrier. As used herein, an “infectious disorder” is any disorder characterized by the presence of a microbial infection, such as bacterial infections. Such infectious disorders include, for example central nervous system infections, external ear infections, infections of the middle ear, such as acute otitis media, infections of the cranial sinuses, eye infections, infections of the oral cavity, such as infections of the teeth, gums and mucosa, upper respiratory tract infections, lower respiratory tract infections, genitourinary infections, gastrointestinal infections, gynecological infections, septicemia, bone and joint infections, skin and skin structure infections, bacterial endocarditis, burns, antibacterial prophylaxis of surgery, and antibacterial prophylaxis in immunosuppressed patients, such as patients receiving cancer chemotherapy, or organ transplant patients. The compounds and compositions comprising the compounds can be administered by routes such as topically, locally or systemically. Systemic application includes any method of introducing the compound into the tissues of the body, e.g., intrathecal, epidural, intramuscular, transdermal, intravenous, intraperitoneal, subcutaneous, sublingual, rectal, and oral administration. The specific dosage of antimicrobial to be administered, as well as the duration of treatment, may be adjusted as needed.

The compounds of the invention may be used for the treatment or prevention of infectious disorders caused by a variety of bacterial organisms. Examples include Gram positive and Gram negative aerobic and anaerobic bacteria, including Staphylococci, for example S. aureus; Enterococci, for example E. faecalis; Streptococci, for example S. pneumoniae; Haemophilus, for example H. influenza; Moraxella, for example M. catarrhalis; and Escherichia, for example E. coli. Other examples include Mycobacteria, for example M. tuberculosis; intercellular microbes, for example Chlamydia and Rickettsiae; and Mycoplasma, for example M. pneumoniae.

The ability of a compound of the invention to inhibit bacterial growth, demonstrate in vivo activity, and enhanced pharmacokinetics are demonstrated using pharmacological models that are well known to the art, for example, using models such as the tests described below.

Test A—Antibacterial Assays

The compounds of the present invention were tested against an assortment of Gram-negative and Gram-positive organisms using standard microtitration techniques (Cohen et. al., Antimicrob., 1985; 28:766; Heifetz, et. al., Antimicrob., 1974; 6:124). The results of the evaluation are shown in Tables 2A and B.

TABLE 2A

| | Minimum Inhibitory Concentrations μg/mL | | |
|---|---|---|---|
| | Gram Negative Bacteria | | |
| Compound No. or | H. influenzae | M. catarrhalis | E. coli |
| Example No. | HI3542 | BC3534 | Tol C |
| 1 | >64 | >64 | >64 |
| 3 | 2 | 2 | >64 |
| 4 | 2 | 1 | >64 |
| 56 | >64 | 16 | >64 |
| 95 | 8 | 1 | >64 |
| 96 | 4 | 2 | >64 |
| 97 | >4 | 2 | >64 |
| 98 | 8 | 2 | >64 |
| 99 | 4 | 4 | >64 |
| 122 | >64 | >64 | >64 |
| 123 | >64 | >64 | >64 |
| 138 | No data | No data | No data |
| 158 | >64 | 8 | >64 |
| 164 | >64 | >64 | >64 |
| 165 | >64 | >64 | >64 |
| 186 | 4 | 8 | >64 |
| 209 | 32 | >64 | >64 |

TABLE 2B

| | Minimum Inhibitory Concentrations μg/mL | | |
|---|---|---|---|
| | Gram Positive Bacteria | | |
| Compound Structure | E. faecalis | S. aureus | S pyogenes |
| or Example No. | MGH-2 | UC-76 | C203 |
| 1 | >64 | >64 | >64 . . . |
| 3 | 0.25 | 0.5 | 0.125 |
| 4 | 0.25 | 0.5 | 0.125 |
| 56 | 4 | 1 | 4 |
| 95 | 0.5 | 4 | 0.5 |
| 96 | 1 | 2 | 0.25 |
| 97 | 1 | 1 | 0.5 |
| 98 | 0.5 | 0.5 | 0.5 |
| 99 | 1 | 1 | 0.5 |
| 122 | 1 | 1 | 0.5 |
| 138 | No data | No data | No data |
| 158 | 2 | 1 | 1 |
| 159 | 2 | 4 | 2 |
| 164 | 4 | 4 | 2 |
| 165 | 4 | 4 | 2 |
| 186 | 1 | 2 | 1 |
| 209 | 8 | 16 | 4 |

Enzymatic Assay

The compounds of the present invention were tested against E. coli transcription and translation (TnT) assay. The TnT assay is a cell free system that utilizes an E. coli S30 fraction and a “premix” to transcribe and translate the firefly luciferase gene from an exogenously supplied plasmid DNA. The amount of luciferase produced is measured by observing the luminescence produced after addition of a luciferase assay reagent. The TnT assay reagents, including the luciferase reporter plasmid pBESTluc, were purchased from Promega Corporation. The protocol was based upon the manufacturer's instructions (Promega Technical Bulletin number 92 “E. coli S30 Extract System for Circular DNA”). Luciferase assay reagent (LucLite Plus) was purchased from Packard Biosciences.

The assay was conducted in white, flat-bottomed, polystyrene 96-well plates. Each well contained S30, premix, amino acids, compound and DNA in a total volume of 35 microliters. The reactions were allowed to incubate at room temperature for 20 minutes, then quenched with 35 microliters of LucLite Plus. The plate was then sealed with an aluminum foil lid and allowed to mix on a plate shaker for five minutes. The plate was then uncovered and read on the LJL Analyst using the standard luminescence protocol. The assay can also be read with a Perkin-Elmer Microbeta Trilux using a 1450-105 96 well plate cassette utilizing a protocol with a 10 second counting time, no background correction, and upper PMT usage. The results of the evaluation are shown in Table 2C.

TABLE 2C

| Compound Structure or | Minimum Inhibitory Concentrations μg/mL |
|---|---|
| Example No. | E. coli TnT Assay |
| 1 | >163 |
| 3 | 1.3 |
| 4 | 1 |
| 56 | 2.9 |
| 95 | 1.5 |
| 96 | 1.1 |
| 97 | 1.3 |
| 98 | 2 |
| 99 | 1.2 |
| 122 | 13 |
| 123 | 27 |
| 138 | 2 |
| 158 | 2.1 |
| 159 | 4.5 |
| 164 | 3.5 |
| 165 | 3.5 |
| 186 | 2.7 |
| 209 | 8.9 |

Test B—In Vivo Activity (Mouse)

The in vivo activity was obtained when the compounds were tested according to the procedure of Miller, et al. (Proc. Soc. Exp. Biol. Med., 1944; 57:261). The median protective dose (PD₅₀) was determined in mice given lethal systemic infections, as depicted in Table 4.

TABLE 4
In Vivo Median Protective Dose (PD₅₀) in Mice (PO)

| Compound Number or | | |
|---|---|---|
| Structure | Organism | PD₅₀ (mg/kg) |
| 3 | S. pyogenes | 6.4 mg/kg |
| | C-203 | (oral) |
| | | 2.1 |
| | | (subcutaneous) |

Test C—Cross Resistance Antibacterial Assay

The compounds of the present invention were tested against an assortment of drug resistant organisms described below using standard microtitration techniques (Cohen, et. al., Antimicrob. Agents Chemother., 1985; 28:766; Heifetz, et. al., Antimicrob. Agents Chemother, 1974; 6:124). The results of the evaluation are shown in Tables 4A and B.
1. E. coli EC-1: Mouse virulent strain.
2. E. faecalis EF-13524: Vancomicin resitant strain.
3. E. faecalis EF-3838: Multiple drug resitant strain (vancomicin, erythromycin, gentamicin).
4. E. faeciaum EF4-3525: Vancomicin resitant strain.
5. E. faeciaum EF4-3836: Multiple drug resitant strain (lineozolid, AZD2563-R, vancomicin, ampicillin, macrolide-R, quinolone-R, trimeothprim/sulfamethoxazole).
6. S. aureus SA-2017: Multiple drug resitant strain (methicillin, ciprofloxacin)
7. S. aureus SA-3528: Multiple drug resitant strain (macrolide, lincosamide, and strptogramin (MLS)).
8. S. aureus SA-3839: Multiple drug resitant strain (linezolid and methicillin).
9. S. aureus SA-3840: Multiple drug resitant strain (linezolid and methicillin).
10. S. pneumoniae SP-3536: Multiple drug resitant strain (B-lactamase and macrolide-R).
11. S. pneumoniae SP-3561: Multiple drug resitant strain (penicillin, macrolide-R, tetracycline, trimeothprim/sulfamethoxazole, erythromycin, clindamicin, cephalosoprin).
12. S. pyogenes SP1-3541: Multiple drug resitant strain (MLS).

13. H. influenziae Hi-3113: B-lactamase

TABLE 4A
Antibacterial Activities Against Resistant Strains

| | Minimum Inhibitory Concentrations | | | | | | |
|---|---|---|---|---|---|---|---|
| CompoundNumber | μg/mL in Strain (by Number) | | | | | | |
| or Structure | 2 | 3 | 4 | 5 | 6 | 7 | 8 |
| 3 | 0.25 | 0.5 | 0.5 | 1 | 1 | 8 | 16 |
| 3A | 0.25 | 1 | 0.5 | 1 | 0.5 | 48 | 4 |

TABLE 4B
Antibacterial Activities Against Resistant Strains

| | Minimum Inhibitory Concentrations | | | | |
|---|---|---|---|---|---|
| CompoundNumber | μg/mL in Strain (by Number) | | | | |
| or Structure | 9 | 10 | 11 | 12 | 13 |
| 352351 | 8 | 16 | 0.125 | 0.25 | 1 |
| 3A | 4 | 4 | 0.25 | 0.5 | 2 |

The following examples are provided to illustrate but not limit the claimed invention.

### EXAMPLES

The following examples are provided to illustrate but not limit the claimed invention.

General Procedure AA: To a 0° C. stirred solution of the amine salt (see Examples 59-61, 64-66) (100 mg, 0.3 mmol) in dry methylene chloride (5 mL) and triethylamine (1-10 eq.) was added the respective acid chloride or anhydride (1-10 eq.). The mixture was stirred overnight while slowly warming to room temperature. The mixture was then diluted with ethyl acetate and washed with saturated sodium bicarbonate and brine. The organic layer was dried, filtered and concentrated in vacuo and the resultant residue was purified via flash chromatography with 0-10% MeOH/methylene chloride to give the desired amides.

General Procedure BB: To a stirred solution of the amine salt (for example, see Examples 62, 63, 68, and 72) (100 mg, 0.3 mmol), the corresponding acid (1.2 eq), t-butanol (1.1 eq) and 1-(3-dimethylaminopropyl)-3-ethylcarbodiimide hydrochloride (EDCI, 1.1 eq.) in DMF (5 mL) at room temperature under nitrogen was added diisopropylethylamine (3.6 eq) and the mixture was stirred for 24 hours. The mixture was then diluted with ethyl acetate and washed with saturated sodium bicarbonate and brine. The organic layer was dried, filtered and concentrated in vacuo and the residue purified via flash chromatography with 0-10% MeOH/methylene chloride to afford the desired amides.

General Procedure CC: To a stirring solution of the amine salt (for example, see Examples 69-71, 74, 75, and 77) (200 mg, 0.6 mmol) in dry methylene chloride (5 mL) and triethylamine (3.5 eq.) at 0° C. was added the respective acid chloride or anhydride (2.5 eq.). The mixture was stirred overnight while slowly warming to room temperature. The mixture was then diluted with saturated sodium bicarbonate and brine and extracted with methylene chloride. The combined organic layers were dried, filtered and concentrated in vacuo and the residue purified via flash chromatography with 0-10% MeOH/methylene chloride to give the desired amides.

General Procedure DD: To a stirring solution of the amine (for example, see Example 73, 76, and 78) (200 mg, 0.3 mmol), the corresponding acid (2.2 eq), t-butanol (2.2 eq) and EDCI (2.2 eq.) in DMF (5 mL) at 0° C. under nitrogen was added diisopropylethylamine (6 eq). The mixture was stirred for 24 hours while slowly warming to room temperature. The reaction mixture was then diluted with saturated sodium bicarbonate and brine and extracted with methylene chloride. The combined organic layers were dried, filtered and concentrated in vacuo and the residue purified via flash chromatography to afford the desired amides.

General procedure EE: To a stirring solution of the diacylated products (for example, see Examples 79-86) (1.0 eq.) was added benzylamine (1-3.5 eq.) and the mixture stirred at room temperature for 18 hours. It was then diluted with ethyl acetate and washed with saturated sodium bicarbonate and brine. The organic layer was dried, filtered and concentrated and the residue purified via flash chromatography to afford the desired amide.

General procedure FF: 1,3-Diketone formation #1: The starting ketone (for example, see Examples 95 and 97) was dissolved in dry THF under nitrogen atmosphere, and cooled to −78° C. in dry ice bath. Lithium diisopropylamide (LDA, 2M, 2.0-2.4 eq.) was added and the resulting mixture stirred at −78° C. for approximately 20 minutes. The corresponding acid chloride or ester (neat, 1.0-1.5 eq.) was added and the mixture was allowed to stir at −78° C. for 15-20 minutes followed by stirring at 0° C. and then allowing to warm to room temperature overnight. The reaction was quenched with saturated ammonium chloride or 0.5N HCl, followed by Ethyl acetate or dichloromethane extraction; the organic phase was washed with brine, dried over magnesium sulfate, filtered and concentrated. The isolated residue was subjected to silica gel flash chromatography to afford the desired compound unless otherwise noted.

General procedure GG: 1,3-Diketone formation #2: To the starting ketone (for example, see Example 96 and 102) dissolved in THF was added lithium t-butoxide (1 M in hexanes, 2.1-3.1 eq.) followed by addition of the corresponding acid chloride or ester (1.1-1.2 eq.) The resulting mixture was heated at reflux overnight. 0.5N HCl or saturated ammonium chloride was then added, and the mixture was extracted with ethyl acetate or dichloromethane. The organic phase was washed with brine, dried over magnesium sulfate, filtered, and concentrated. The resulting residue was subjected to flash silica gel chromatography to afford the desired product unless otherwise noted.

General procedure HH: 1,3-Diketone formation #3: The starting ketone (for example, see Examples 98-101 and 103-115) was dissolved in THF, cooled to 0° C., and lithium hexamethyldisilazide (LiHMDS, 1 M in T}F, 2.0-3.15 eq.) added dropwise via syringe. The reaction mixture was then stirred for approximately 30 minutes, after which time the corresponding acid chloride (1.0-1.2 eq.) was added as a solid or dissolved in THF added dropwise via syringe. The resulting mixture was stirred at 0° C. and then allowed to warm slowly to room temperature overnight. The solution was quenched with 0.5N HCl or saturated ammonium chloride and extracted with ethyl acetate or dichloromethane; the organic phase was washed with brine, dried over magnesium sulfate, filtered, and concentrated. The resulting residue was subjected to flash silica gel chromatography to afford the desired product unless otherwise noted.

General procedure II: Pyrazole formation: The starting 1,3-diketone (for example, see Examples 96, 98, 99-115, and 122) was placed in ethanol and to this was added hydrazine hydrate (2.5-5.0 eq.) or an appropriately substituted hydrazine (2.5-4.0 eq.). If a slurry resulted the reaction flask was sometimes heated with warm water (approximately 50-60° C.) until all solids dissolved. The slurry or solution was then stirred at room temperature 24-72 hours. The solvent was then removed in vacuo and the resulting residue was subjected to flash silica gel chromatography to afford the desired product unless otherwise noted.

General Procedure JJ: The requisite bromoketone (for example, see Examples 126-137, 139, 140-142, and 151) (1 mmol), the appropriate hydrazide XCSNHNH2 (X=NHR, SR) (1 mmol), and 10 mL of absolute ethanol were heated to 88° C. Upon completion of the reaction, the solution was cooled to room temperature, treated with 4 mL of saturated sodium bicarbonate, and concentrated in vacuo. The aqueous layer was extracted with several portions of dichloromethane or dichloromethane/MeOH. The combined organic layers were dried over sodium sulfate, filtered, concentrated in vacuo, and then purified by silica gel chromatography.

General procedure KK: α,β-unsaturated ketone formation: To 1 eq. of the ketone (for example, see Examples 193-196, 198, 200, 202, 204, 205, and 208) the appropriate aromatic aldehyde (4 eq.) was added, followed by acetic acid and piperidine. The reaction was kept at 80 to 100° C. for 4-12 hours. The reaction was cooled to room temperature and taken up in dichloromethane; the solution was washed with water, potassium carbonate solution, dilute hydrochloric acid and brine. The organic layers were dried over magnesium sulfate, filtered, concentrated in vacuo and then purified by silica gel chromatography.

General Procedure LL: Pyrazole formation from enones: To 1 eq. of the enone (for example, see Examples 197, 199, 201, and 203) taken in ethanol was added para-toluenesulfonyl hydrazide (2.2 eq.) and para-toluenesulfonic acid (2.0 eq.). The reaction was kept under reflux for 24 hours. The solvents were evaporated. The crude reaction mixture was diluted with dichloromethane, washed with sodium bicarbonate solution and dried over sodium sulfate. The organic layers were dried over magnesium sulfate, filtered, concentrated in vacuo and then purified by silica gel chromatography.

### Example 1

### (S)-N-[2-Oxo-3-(1,4,5,6-tetrahydro-1,2-diaza-benzo[e]azulen-9-yl)oxalidin-5-ylmethyl]acetamide 1-10

The title compound was prepared from (S)-N-[2-oxo-3-(9-oxo-6,7,8,9-tetrahydro-5H-benzocyclohepten-2-yl)-oxazolidin-5-yl-methyl]acetamide (I-9), the synthesis of which is depicted in the Scheme and described below.

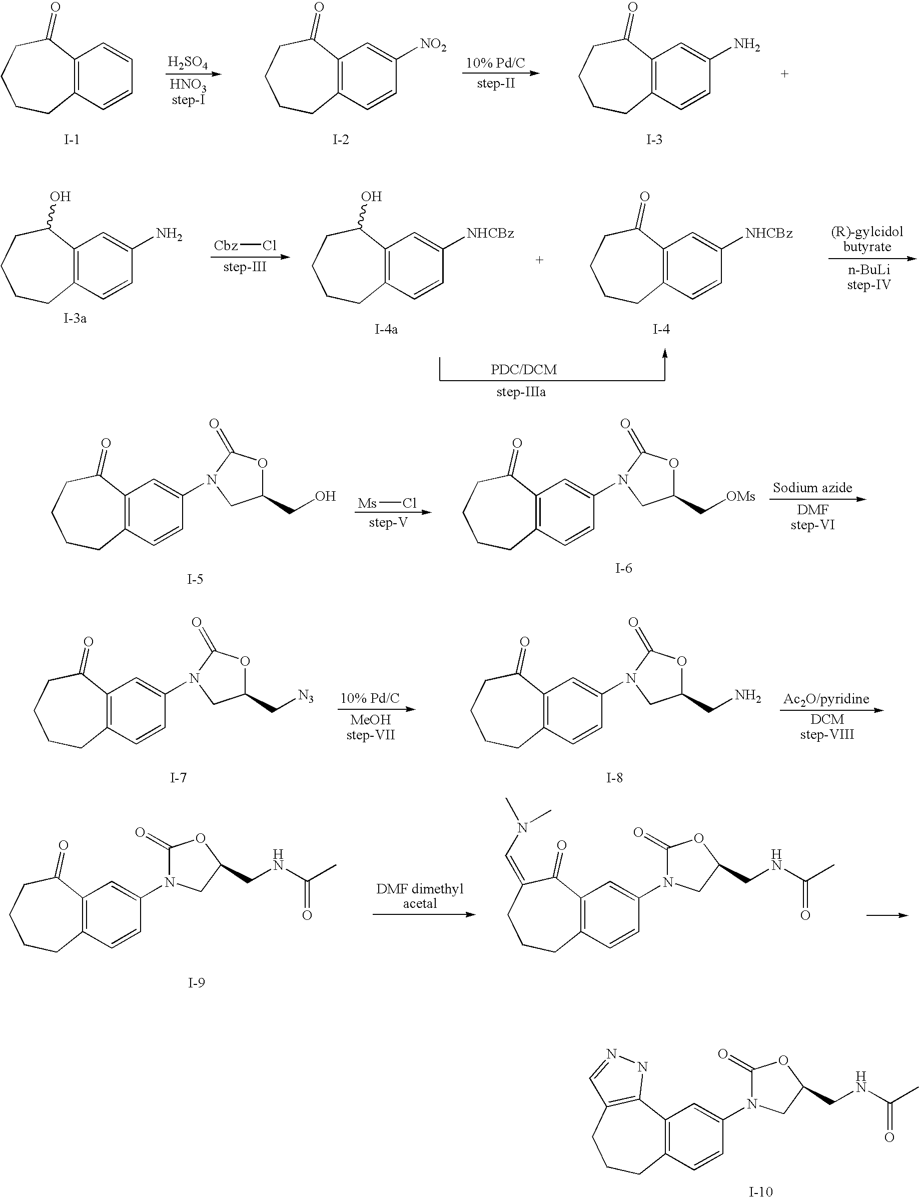

### 3-Nitro-6,7,8,9-tetrahydro-benzocyclohepten-5-one (Step I, I-2)

1-Benzosuberone (175.0 g, 1.09 mol) was dissolved in concentrated sulfuric acid (3.5 L) and cooled to 0° C. To this mixture was added drop wise, a solution of fuming nitric acid (65.03 mL, 1.13 mol) in sulfuric acid (425 mL) and after the addition was complete, the reaction mixture was stirred at 0° C. for 30 minutes. The reaction mixture was poured into ice water and extracted with diethyl ether (3×2.0 L). The organic extracts were pooled, washed with brine, dried over anhydrous Na₂SO₄, filtered and evaporated under vacuum. The residue obtained was triturated with hexane (3×1.0 L), filtered and dried to give the title compound. Yield: 125.0 g (55.8%), mp. 88-89° C.

### 3-Amino-6,7,8,9-tetrahydro-benzocycloheptene-5-one (Scheme I, Step II, I-3)

A solution of 3-nitro-6,7,8,9-tetrahydro-benzocyclohepten-5-one (I-2) in methanol (1.5 L) was hydrogenated in the presence of 10% Pd/C (15.2 g) at 50 psi for 1 hour and filtered through Celite. The filtrate was evaporated under vacuum to give a solid (105 g). The resulting mixture of desired amino compound and an over reduced product, (I-3a, 3-amino-5-hydroxy-6,7,8,9-tetrahydro-benzocycloheptane) was carried to the next step, without further purification. Yield: 105 g (99%).

### (9-Oxo-6,7,8,9-tetrahydro-5H-benzocyclohepten-2-yl)carbamic acid benzyl ester (Scheme I, Step III, I-4)

A solution of the mixture obtained from step-3 (3-amino-6,7,8,9-tetrahydro-benzocyclohepten-5-one (1-3) and 3-amino-5-hydroxy-6,7,8,9-tetrahydro-benzocycloheptane (I-3a, 105.0 g, 0.6 mol) in a mixture of acetone/water, 2:1/2 L:1 L) was treated with sodium bicarbonate (189.0 g, mol) and cooled to 0° C. The reaction mixture was treated with Cbz-chloride (189 mL, 1.32 mol), stirred over night at room temperature and the acetone removed in vacuo. The aqueous residue was extracted with ethyl acetate (3×1.0 L) and the organic extracts were pooled, washed with brine, dried over anhydrous Na₂SO₄ and concentrated. The residue was purified by silica gel column chromatography (TLC-Ethyl acetate:Hex/3:7) to give both the title compound along with [N-5-hydroxy(1,2,3,4,5-pentahydrobenzo[a][7]annulene-7-yl)(phenylmethoxy) carboxamide], I-4a. Yield of 4: 39.0 g. Yield of 4a: 105.0 g.

A solution of [N-5-hydroxy(1,2,3,4,5-pentahydrobenzo[a][7]annulene-7-yl)(phenylmethoxy)carboxamide] (I-4a) in methylene chloride (1.5 L) was treated with pyridinium dichromate (120 g) and stirred over night at room temperature. The reaction mixture was filtered through a bed of Celite and the filtrate was evaporated under vacuum to give the title compound. Total Yield: 114.0 g (62%), mp. 120-121° C.

### (R)-5-Hydroxymethyl-3-(9-oxo-6,7,8,9-tetrahydro-5H-benzocyclohepten-2-yl)-oxazolidin-2-one (Scheme I, Step IV, I-5)

To a flame-dried flask charged with diisopropyl amine (19.5 mL, 0.139 mol) and THF (400 mL) was added n-butyl lithium (68.55 mL, 2.5 M in hexanes) drop wise at −78° C. The reaction mixture was allowed to warm to 0° C. and then transferred by a canula in to a separate flask containing (9-oxo-6,7,8,9-tetrahydro-5H-benzocyclohepten-2-yl)carbamic acid benzyl ester (I-4, 39.0 g, 0.109 mol) in THF (800 mL) at −78° C. The reaction mixture was stirred at −78° C. for 30 minutes and was treated with R-glycidyl butyrate (19.5 g, 0.135 mol). The reaction mixture was warmed to room temperature, then heated at 70° C. for 12 hours and quenched by diluting with a saturated solution of ammonium chloride (500 mL). The aqueous mixture was extracted with ethyl acetate (3×1 L) and the combined organic fractions were washed with brine, dried over anhydrous Na₂SO₄, filtered and evaporated under vacuum. The residue obtained was triturated with ether to give the title compound, which was used in the next step without further purification. Yield: 34.2 g, mp. 144-146° C.

### (R)-Methanesulfonic acid-2-oxo-3-(9-oxo-6,7,8,9-tetrahydro-5H-benzocyclohepten-2-yl)oxazolidin-5-yl methyl ester (Scheme I, Step V, I-6)

To a solution of (R)-5-hydroxymethyl-3-(9-oxo-6, 7, 8, 9-tetrahydro-5H-benzocyclohepten-2-yl)-oxazolidin-2-one (1-S, 34.2 g, 0.130 mol) in methylene chloride (1.0 L) at 0° C. was added triethyl amine (36.8 mL, mol) followed by methanesulfonyl chloride (13.5 mL, 0.174 mol). The reaction mixture was warmed to room temperature, stirred for 2 hours, and diluted with ethyl acetate. The ethyl acetate solution was washed with brine (3×300 mL), dried over anhydrous Na₂SO₄, filtered and evaporated under vacuum to give the title compound. Yield: 42.0 g (93%).

### (R)-5-Azidomethyl-3-(9-oxo-6,7,8,9-tetrahydro-5H-benzocyclohepten-2-yl)oxazolidin-2-one (Scheme I, Step VI, I-7)

To a solution of (R)-methanesulfonic acid-2-oxo-3-(9-oxo-6,7,8,9-tetrahydro-5H-benzocyclohepten-2-yl)oxazolidin-5-yl methyl ester (I-6, 42.0 g, 0.123 mol) in DMF (300 mL) was added sodium azide (29.4 g, 0.452 mol), and the mixture was heated overnight at 70° C. The reaction mixture was diluted with ethyl acetate (1.0 L), washed with water (3×300 mL), brine (1×500 mL), dried over anhydrous Na₂SO₄, filtered and evaporated under vacuum to give the title compound, which was directly used in the next step. Yield: 34.5 g (93%).

### (S)-5-aminomethyl-3-(9-oxo-6,8,9-tetrahydro-5H-benzocyclohepten-2-yl)-oxazolidin-2-one (Scheme I, Step VII, I-8)

A solution of (R)-5-azidomethyl-3-(9-oxo-6,7,8,9-tetrahydro-5H-benzocyclo-hepten-2-yl)oxazolidin-2-one (I-7, 34.5 g, 0.115 mol) in methanol (1.0 L) was hydrogenated in the presence of 10% Pd/C (11.71 g) at 35 pounds per squeare inch for 1 hour and filtered through a short bed of celite. Evaporation of the filtrate under vacuum gave the title compound, which was used in the proceeding step without further purification. Yield: 27.4 g (85%)

### (S)-N-[2-oxo-3-(9-oxo-6,7,8,9-tetrahydro-5H-benzocyclohepten-2-yl)-oxazolidin-5-yl-methyl]acetamide (Scheme I, Step VIII, I-9)

To a flame dried flask was charged (S)-5-aminomethyl-3-(9-oxo-6,8,9-tetrahydro-5H-benzocyclohepten-2-yl)-oxazolidin-2-one (1-8, 27.4 g, 0.1 mol) and pyridine (25.0 mL, mol) in methylene chloride (1.0 L) at 0° C., followed by acetic anhydride (13.12 mL). The reaction mixture was allowed to come to room temperature, stirred overnight, and evaporated under vacuum. The residue obtained was purified by silica gel column chromatography (50%) ethyl acetate in hexanes to 100% Ethyl acetate) to give the title compound. Yield: 12.5 g (39.5%), mp. 122-123° C.

### Conversion of (S)-N-[2-oxo-3-(9-oxo-6,7,8,9-tetrahydro-5H-benzocyclohepten-2-yl)-oxazolidin-5-yl-methyl]acetamide (I-9) to the Title Compound

### (S)-N-[3-(8-Dimethyaminomethylene-9-oxo-6,7,8,9-tetrahydro-5H-benzocyclohepten-2-yl)-2-oxo-oxalidin-5-ylmethyl]acetamide (Step IX)

To (S)-N-[2-oxo-3-(9-oxo-6,7,8,9-tetrahydro-5H-benzocyclohepten-2-yl)oxalidin-5-ylmethyl]acetamide (1.0, 3.16 mmol) in n-propanol (25 mL) was added dimethylformamide dimethyl acetal (1.51 g, 12.644 mmol, 4.0 eq.) and the resulting mixture was heated at reflux overnight. Heat was then removed; the reaction mixture was allowed to cool, and the solvent was removed in vacuo. The isolated residue was triturated with diethyl ether/ethyl acetate mixture to afford the title compound as a solid that was filtered off and washed with diethyl ether. Isolated yield: 0.90 g (77%). MS-APCI (m/z+): 372 (M+H).

### (S)-N-[2-Oxo-3-(1,4,5,6-tetrahydro-1,2-diaza-benzo[e]azulen-9-yl)oxalidin-5-ylmethyl]acetamide (Step X)

To (S)-N-[3-(8-dimethyaminomethylene-9-oxo-6,7,8,9-tetrahydro-5H-benzocyclohepten-2-yl)-2-oxo-oxalidin-5-ylmethyl]acetamide (0.58 g, 1.56 mmol) in ethanol (16 mL) was added hydrazine hydrate (0.20 g, 6.25 mmol, 4.0 eq.). The reaction mixture was stirred at room temperature overnight. The isolated residue was subjected to chromatography using Combiflash system, eluting with MeOH/CH₂Cl₂ gradient (0-7% MeOH over 1 hour) to afford the title compound. Isolated yield: 0.30 g (57%). MS-APCI (m/z+): 297, 341 (M+H).

### Example 2

### (S)-N-[2-Oxo-3-(1,4,5,6-tetrahydro-1,2-diaza-benzo[e]azulen-8-yl)-oxazolidin-5-ylmethyl]-acetamide II-7

The compound was prepared from (S)-N-[2-oxo-3-(5-oxo-6,7,8,9-tetrahydro-5H-benzocyclohepten-2-yl)-oxazolidin-5-ylmethyl]-acetamide (II-7), which was prepared via three different methods, as described below.

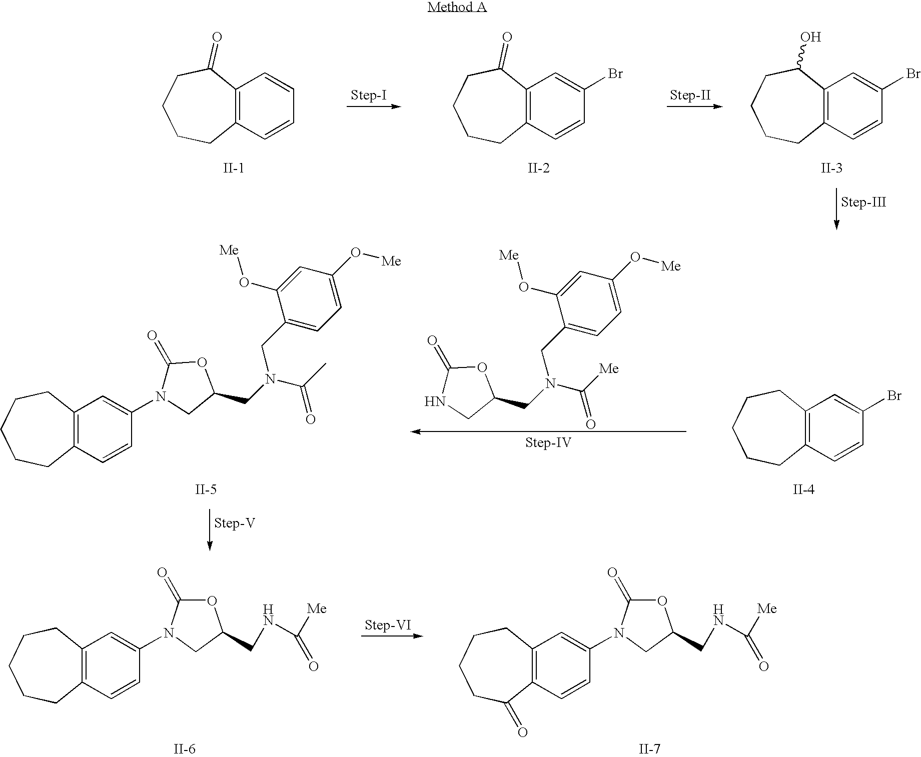

### 3-Bromo-6,7,8,9-tetrahydro-benzocyclohepten-5-one (Step-I)

The title compound was prepared from 1-benzosuberone as described in Synthetic Communications (1994), 24(19), 2777-88.

### 3-Bromo-6,7,8,9-tetrahydro-5H-benzocyclohepten-5-ol (Step-II)

To a solution of 3-bromo-6,7,8,9-tetrahydro-5H-benzocyclohepten-5-ol (II-2, 1.01 g, 4.22 mmol) in dichloromethane (21 mL) cooled to 0° C. was added sodium borohydride (175 mg, 4.64 mmol). The ice bath was removed and the reaction stirred one hour. DMF (5.0 mL) and methanol (5.0 mL) were added followed by an additional 1.5 equivalents of sodium borohydride (239 mg, 6.33 mmol). The reaction was stirred at room temperature overnight, was diluted with ethyl acetate, and washed with water and brine. The solution was dried over sodium sulfate and concentrated. Chromatography on an Isco 10 g column eluting with 0-20% ethyl acetate in hexanes over 30 minutes gave the title compound (880 mg, 86%). MS (CI) 223.1 (M-17 (loss of OH)).

### 2-Bromo-6,7,8,9-tetrahydro-5H-benzocycloheptene (Step III)

To a solution of 3-bromo-6,7,8,9-tetrahydro-5H-benzocyclohepten-5-ol (I-3, 520 mg, 2.16 mmol) in dichloromethane (7.3 mL) was added triethylsilane (0.63 mL, 3.94 mmol) followed by dropwise addition of trifluoroacetic acid (1.56 mL, 20.2 mmol). The reaction stirred at room temperature overnight. The solvent was evaporated and the resulting residue was dissolved in ethyl acetate and a saturated solution of sodium bicarbonate. The mixture was stirred vigorously for several minutes and then the layers were separated. The organic layer was washed twice with sat. sodium bicarbonate and brine, dried over sodium sulfate, and concentrated. Chromatography on a Biotage Flash 40S column eluting with 100% hexanes gave the title compound (309 mg, 63% Yield). ¹H NMR (400 MHz, CDCl₃): δ 1.61 (m, 4H), 1.81 (m, 2H), 2.71 (m, 4H), 6.94 (d, J=7.9 Hz, 1H), 7.17 (d,d, J=7.9, 2.1 Hz, 1H), 7.22 (d, J=2.1 Hz, 1H).

### (S)-N-(2,4-Dimethoxy-benzyl)-N-[2-oxo-3-(6,7,8,9-tetrahydro-5H-benzocyclohepten-2-yl)-oxazolidin-5-ylmethyl]-acetamide (Step-IV)

The title compound was prepared from 2-bromo-6,7,8,9-tetrahydro-5H-benzocycloheptene (II-4) and (R)-N-(2,4-dimethoxy-benzyl)-N-(2-oxo-oxazolidin-5-ylmethyl)-acetamide according to procedure as described in Tetrahedron Letters (2001), 42(22), 3681-3684. (Yield: 260 mg, 42%). MS (CI) m/z 453.4 (M+1), 497.4 (M−1+46).

### (S)-N-[2-Oxo-3-(6,7,8,9-tetrahydro-5H-benzocyclohepten-2-yl)-oxazolidin-5-ylmethyl]-acetamide (Step-V)

The title compound was prepared from (S)-N-(2,4-dimethoxy-benzyl)-N-[2-oxo-3-(6,7,8,9-tetrahydro-5H-benzocyclohepten-2-yl)-oxazolidin-5-ylmethyl]-acetamide (II-5), dissolved in 3 mL of trifluoroacetic acid, and stirred at room temperature for 1.5 hours (Yield: 124 mg, 71%). MS (CI) m/z 303.3 (M+1).

### (S)-N-[2-Oxo-3-(5-oxo-6,7,8,9-tetrahydro-5H-benzocyclohepten-2-yl)-oxazolidin-5-ylmethyl]-acetamide (Step-VI)

To a solution of (S)-N-[2-oxo-3-(6,7,8,9-tetrahydro-5H-benzocyclohepten-2-yl)-oxazolidin-5-ylmethyl]-acetamide (II-6, 50 mg, 0.165 mmol) in acetic acid (0.5 mL) and acetic anhydride (0.068 mL) was added a solution of chromium trioxide (67 mg, 0.23 mmol) in acetic acid (0.3 mL) and water (0.063 mL). The reaction was stirred at room temperature overnight followed by addition of more chromium trioxide (30 mg, 0.30 mmol). The reaction was stirred over night and additional water (0.06 mL) was added. The reaction was stirred open to the air for three hours and was then diluted with water and extracted with twice with Ethyl acetate. The combined organic layers were washed with brine, dried over sodium sulfate and concentrated in vacuo. Purification using silica gel chromatography gave the title compound (7.5 mg, 14% Yield). MS (CI) m/z 317.3 (M+1).

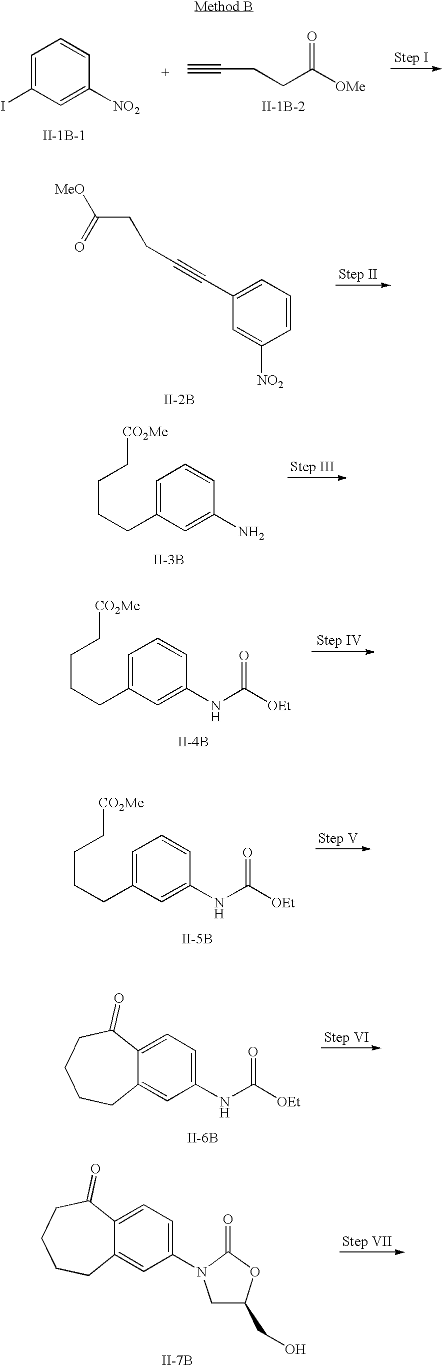

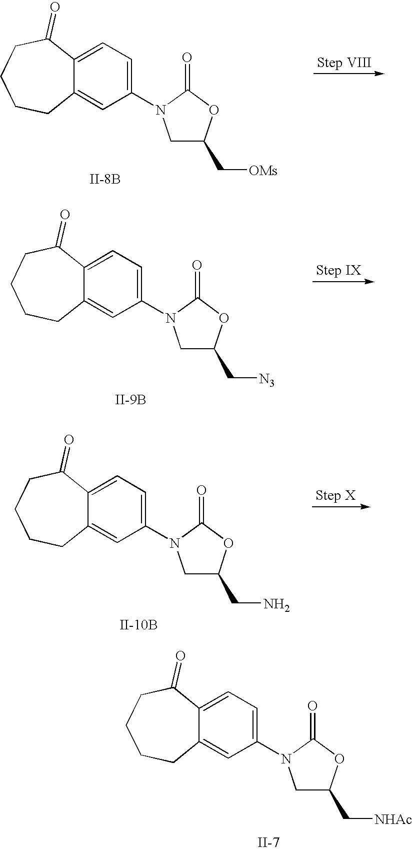

### Pent-4-ynoic acid methyl ester (II-1B-2)

Pent-4-ynoic acid (10 g. 96.8 mmol) was dissolved in 500 mL of anhydrous methanol, and the solution was cooled to 0° C. before thionyl chloride (8.9 mL, 119 mmol) was added dropwise. The resulting reaction solution was warmed to room temperature and stirred under nitrogen overnight. The solution was diluted with 1.5 L of dichloromethane and washed with 1 L of water. The organic solvents were removed using a rotary evaporator at 25° C. to afford the title compound (14.2 g, 100% crude yield). The crude product was taken into the next step without further purification.

### 5-(3-Nitro-phenyl)-pent-4-ynoic acid methyl ester (Step I)

1-Iodo-3-nitro-benzene (II-1B-1, 23.6 g, 94.8 mmol) and pent-4-ynoic acid methyl ester (II-1B-2, 14 g) were dissolved in 125 mL of anhydrous DMF. To this reaction solution were added triphenyl phosphine (1.99 g, 7.59 mmol), followed by palladium (II) acetate (0.85 g, 3.79 mmol), and copper (I) iodide (1.45 g, 7.61 mmol), and finally triethylamine (50 mL, 360 mmol) at 0° C. This resulting black reaction mixture was warmed to room temperature and stirred under nitrogen for 24 hours. The reaction was quenched by pouring it into 200 mL of ice water and 150 mL 3N HCl. The solids were filtered out and the mother liquid was extracted with 50 mL of ethyl acetate. The ethyl acetate solution was concentrated to dryness and the resulting solid was combined with the previously isolated solids and slurried with 450 mL of ethanol. The undissolved solid was removed via suction filtration, and the solution was concentrated; this residue was further purified by silica gel column chromatography using hexanes/ethyl acetate (15.8 g, 72.4% yield for two steps).

### 5-(3-Aminophenyl)-pentanoic acid methyl ester (Step II)

A reaction flask containing 5-(3-nitrophenyl)pent-4-ynoic acid methyl ester (II-2B, 15.2 g, 65.2 mmol) and Pd/C 10% wet (3.0 g) in 200 mL of methanol was shaken under hydrogen (45 psi) atmosphere at room temperature. After four hours, the reaction mixture was filtered through celite, and the methanol solution was concentrated to dryness. The resulting residue was purified using silica gel column chromatography to afford the title compound (6.27 g, 46.4% yield).

### 5-(3-Ethoxycarbonylamino-phenyl)-pentanoic acid methyl ester (Step III)

5-(3-Amino-phenyl)-pentanoic acid methyl ester (II-3B, 5.1 g, 24.6 mmol) was dissolved in 50 mL of anhydrous dichloromethane and the solution was cooled in an ice-water bath. To it was added ethyl diisopropyl amine (5.46 mL, 31.3 mmol), followed by ethyl chloroformate (2.77 mL, 29.0 mmol). This reaction solution was then warmed to room temperature and stirred under nitrogen overnight. The solvent was evaporated and the residue was purified using silica gel column chromatography to yield the title compound (6.02 g, 87.6% yield).

### 5-(3-Ethoxycarbonylamino-phenyl)-pentanoic acid (Step IV)

5-(3-Ethoxycarbonylamino-phenyl)-pentanoic acid methyl ester (II-4B, 5.47 g, 19.6 mmol) was dissolved in 65 mL of THF and 10 mL of water. To this solution was added lithium hydroxide, and the resulting reaction mixture was heated to 55° C. for three hours. Heating was removed and the mixture was carefully neutralized, then further acidified with 3N HCl to a pH of 4-5. The mixture was separated into two layers. The aqueous phase was separated and extracted with 30 mL of dichloromethane; the organic phases were combined and solvents were evaporated to afford the title compound (4.98 g, 95.8% yield).

### (5-Oxo-6,7,8,9-tetrahydro-5H-benzocyclohepten-2-yl)-carbamic acid ethyl ester (Step V)

Polyphosphoric acid (22 g) was taken in 75 mL of toluene and to it 25 g. of celite was added. While stirring, 5-(3-ethoxycarbonylamino-phenyl)-pentanoic acid (II-5B,m 3.65 g, 13.76 mmol) was added and the reaction was kept under reflux. After two hours, the reaction mixture was cooled to room temperature and water was added while stirring vigorously. The mixture was diluted with ethyl acetate and the celite was filtered off and washed with ethyl acetate. The organic layer was separated and the aqueous layer was re-extracted with ethyl acetate; the organic solution was dried over sodium sulfate and concentrated. The residue was purified by flash silica gel chromatography to afford the title compound (3 g, 89% yield). MS-CI m/z: 248 (M+H).

### (R)-5-Hydroxymethyl-3-(5-oxo-6,7,8,9-tetrahydro-5H-benzocyclohepten-2-yl)-oxazolidin-2-one (Step VI)

The title compound was prepared according to the procedure described in Example 1, step IV using (5-oxo-6,7,8,9-tetrahydro-5H-benzocyclohepten-2-yl)-carbamic acid ethyl ester. MS m/z: 276 (M+H).

### (R)-Methanesulfonic acid 2-oxo-3-(5-oxo-6,7,8,9-tetrahydro-5H-benzocyclohepten-2-yl)-oxazolidin-5-ylmethyl ester (Example 2, Method B, Step VII)

The title compound was prepared according to the procedure described in Example 1, step V using (R)-5-hydroxymethyl-3-(5-oxo-6,7,8,9-tetrahydro-5H-benzocyclohepten-2-yl)-oxazolidin-2-one. MS m/z: 354 (M+H).

### (R)-5-Azidomethyl-3-(5-oxo-6,7,8,9-tetrahydro-5H-benzocyclohepten-2-yl)-oxazolidin-2-one (Example 2, Method B, Step VIII)

The title compound was prepared according to the procedure described in Example 1, step VI using (R)-methanesulfonic acid 2-oxo-3-(5-oxo-6,7,8,9-tetrahydro-5H-benzocyclohepten-2-yl)-oxazolidin-5-ylmethyl ester. MS m/z: 301 (M+H).

### (S)-5-Aminomethyl-3-(5-oxo-6,7,8,9-tetrahydro-5H-benzocyclohepten-2-yl)-oxazolidin-2-one (Example 2, Method B, Step IX)

The title compound was prepared according to the procedure described in Example 1, step VII using (R)-5-azidomethyl-3-(5-oxo-6,7,8,9-tetrahydro-5H-benzocyclohepten-2-yl)-oxazolidin-2-one. MS m/z: 275 (M+H).

### (S)-N-[2-Oxo-3-(5-oxo-6,7,8,9-tetrahydro-5H-benzocyclohepten-2-yl)-oxazolidin-5-ylmethyl]-acetamide (Example 2, Method B, Step X)

The title compound was prepared according to the procedure described in Example 1, step VIII using (S)-5-aminomethyl-3-(5-oxo-6,7,8,9-tetrahydro-5H-benzocyclohepten-2-yl)-oxazolidin-2-one. MS m/z: 317 (M+H).

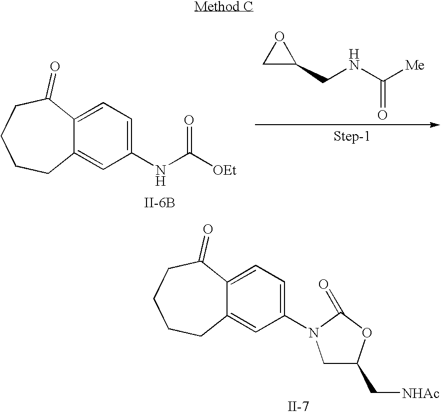

### (S)-N-[2-Oxo-3-(5-oxo-6,7,8,9-tetrahydro-5H-benzocyclohepten-2-yl)-oxazolidin-5-ylmethyl]-acetamide

(5-Oxo-6,7,8,9-tetrahydro-5H-benzocyclohepten-2-yl)-carbamic acid ethyl ester (II-6B, 0.4 g, 1.62 mmol) was taken in 12 mL of tetrahydrofuran. To it n-butyllithium (1.6M, 1.78 mmol) was added by dropwise at −78° C. After stirring the reaction mixture for 90 minutes at −78° C., (S)-N-oxiranylmethyl-acetamide (0.37 g, 3.24 mmol) in 2 mL of tetrahydrofuran was added. The reaction mixture was slowly allowed to warm to room temperature. After stirring at room temperature for 30 minutes, the solution was heated to 60° C. for 2 hours. The reaction was cooled to room temperature, quenched with saturated ammonium chloride, and diluted with ethyl acetate; the organic layer was washed with saturated sodium bicarbonate solution and brine and was dried over magnesium sulfate. The solvents were evaporated and the residue was purified by flash column silica gel chromatography to afford the title compound (0.25 g, 49% yield). MS-CI m/z: 317 (M+H).

### Modification of Method C: Preparation of (S)-N-[2-Oxo-3-(5-oxo-6,7,8,9-tetrahydro-5H-benzocyclohepten-2-yl)-oxazolidin-5-ylmethyl]-acetamide

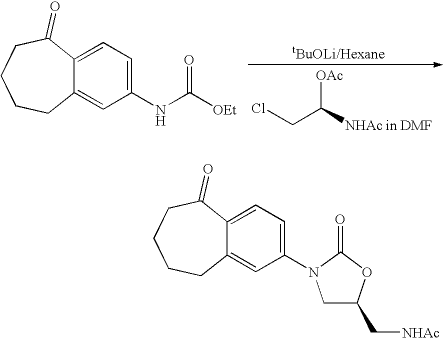

A stirred solution of (5-oxo-6,7,8,9-tetrahydro-5H-benzocyclohepten-2-yl)-carbamic acid ethyl ester (II-6B, 30 g, 0.121 mol) in a mixture of dry methanol (9.9 mL) and DMF (120 mL) was treated dropwise with a solution of lithium tert-butoxide in hexane (1 M in hexane, 370 mL, 0.37 mol) over the course of 2 hours 45 minutes at room temperature. The mixture was stirred at room temperature for 1.5 hours, cooled to 0° C. and was treated dropwise with a solution of (1S)-1-[(acetylamino)methyl]-2-chloroethyl acetate (46.97 g, 0.226 mol) in DMF (100 mL) of the course of 1 hour 10 minutes. The mixture was stirred at room temperature overnight, cooled in an ice-bath and treated with aqueous saturated ammonium chloride (300 mL) solution followed by ice water (500 mL). The mixture was extracted with ethyl acetate (4×500 mL). The organic extracts were combined, washed with brine (3×500 mL), dried over magnesium sulfate, filtered and evaporated under vacuum. The residue was further triturated with ether at ice-bath temperature, filtered, washed with ether followed by hexanes and vacuum dried to give the title compound. Yield: 23 g (approximately 60%).

### (S)-N-[3-(6-Dimethyaminomethylene-5-oxo-6,7,8,9-tetrahydro-5H-benzocyclohepten-2-yl)-2-oxo-oxazolidin-5-ylmethyl]acetamide

To (S)-N-[2-oxo-3-(5-oxo-6,7,8,9-tetrahydro-5H-benzocyclohepten-2-yl)oxalidin-5-ylmethyl]acetamide (II-7, 0.25 grams, 0.79 mmol) in n-propanol (8 mL) was added dimethylformamide dimethyl acetal (0.38 g, 3.16 mmol, 4.0 eq.) and the resulting mixture was refluxed overnight. The reaction mixture was allowed to cool and the solvent was removed in vacuo. The residue was triturated with diethyl ether/ethyl acetate mixture to afford the title compound which was filtered off and washed with diethyl ether. Isolated yield: 0.25 g (85%). MS-APCI (m/z+): 372 (M+H).

### Example 3

### (S)-N-[2-Oxo-3-(1,4,5,6-tetrahydro-1,2-diaza-benzo[e]azulen-8-yl)oxazolidin-5-ylmethyl]acetamide

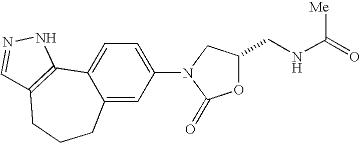

To (S)-N-[3-(6-dimethyaminomethylene-5-oxo-6,7,8,9-tetrahydro-5H-benzocyclohepten-2-yl)-2-oxo-oxazolidin-5-ylmethyl]acetamide (0.47 g, 1.27 mmol) in ethanol (15 mL) was added hydrazine hydrate (0.16 g, 5.06 mmol, 4.0 eq.). The slurry was heated gently with warm water (˜50° C.) until all solids dissolved, and the reaction mixture was stirred at room temperature overnight. The solids that formed were filtered and washed with ethanol and ethyl acetate to afford the title compound. Isolated yield: 0.29 g (67%). MS-APCI (m/z+): 297, 341 (M+H).

### Example 3A

### (S)-N-[3-(5,6-dihydro-4H-1-oxa-2-aza-benzo[e]azulen-8-yl)-2-oxo-oxalidin-5-ylmethyl]acetamide (PD 0353881)

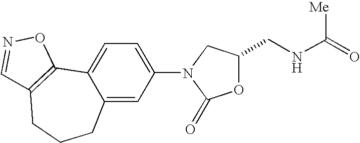

(S)-N-[3-(6-Dimethyaminomethylene-5-oxo-6,7,8,9-tetrahydro-5H-benzocyclohepten-2-yl)-2-oxo-oxalidin-5-ylmethyl]acetamide (0.12 g, 0.323 mmol) was dissolved in methanol (4 mL) and cooled to 0° C. in an ice bath. Hydroxylamine-O-sulfonic acid (0.040 g, 0.355 mmol. 1.1 eq.) in methanol (2 mL) was added dropwise to the reaction mixture over 2 minutes. The reaction mixture was then stirred at 0° C. for ten minutes and at room temperature for 30 minutes. The reaction mixture was then poured into saturated sodium bicarbonate (20 mL)/water (18 mL) mixture, transferred to separatory funnel, and extracted with ethyl acetate (2 times) and dichloromethane. The organics were washed with brine, combined, dried over sodium sulfate, filtered and concentrated. The isolated residue was subjected to chromatography using Combiflash system, eluting with MeOH/CH₂Cl₂ gradient (0-5% MeOH over 1 hour) to afford the title compound. Isolated yield: 0.10 g (91%). MS-APCI (m/z+): 298, 342 (M+H).

### Example 4

### (S)-N-[3-(5,6-dihydro-4H-1-oxa-2-aza-benzo[e]azulen-9-yl)-2-oxo-oxazolidin-5-ylmethyl]acetamide

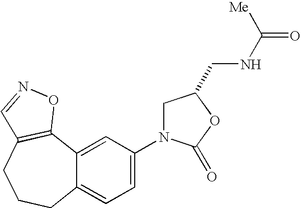

(S)-N-[3-(8-Dimethyaminomethylene-9-oxo-6,7,8,9-tetrahydro-5H-benzocyclohepten-2-yl)-2-oxo-oxalidin-5-ylmethyl]acetamide (0.20 g, 0.54 mmol) was dissolved in methanol (5 mL) and cooled to 0° C. in an ice bath. Hydroxylamine-O-sulfonic acid (0.067 g, 0.59 mmol, 1.1 eq.) in methanol (2 mL) was added dropwise to the reaction mixture over a 2 minute period. The reaction mixture was then stirred at 0° C. for ten minutes followed by 30 minutes stirring at room temperature. The reaction mixture was poured into a saturated sodium bicarbonate (20 mL)/water (18 mL) mixture, transferred to a separatory funnel, and extracted with ethyl acetate (twice) and dichloromethane. The organic layers were washed with brine, combined, dried over sodium sulfate, filtered and concentrated. The isolated residue was subjected to chromatography using Combiflash system, eluting with MeOH/CH₂CL₂ gradient (0-5% MeOH over 1 hour) to afford the title compound. Isolated yield: 0.110 g (60%). MS-APCI (m/z+): 298, 342 (M+H).

### Examples 5 and 6

### (S)-N-[3-(2-Methyl-9,10-dihydro-4H-3-thia-1-aza-benzo[f]azulen-7-yl)-2-oxo-oxazolidin-5-ylmethyl]-acetamide and (S)-N-[3-(2-Methyl-9,10-dihydro-4H-3-thia-1-aza-benzo[f]azulen-6-yl)-2-oxo-oxazolidin-5-ylmethyl]-acetamide

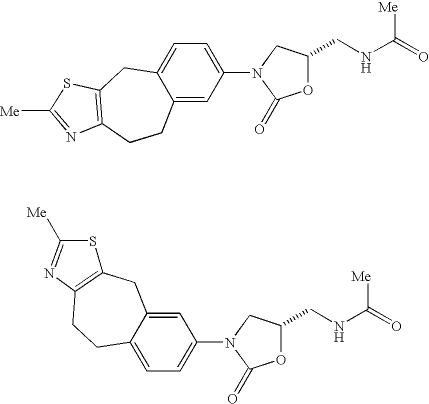

The title compounds were prepared from intermediate (S)-N-[2-Oxo-3-(7-oxo-6,7,8,9-tetrahydro-5H-benzocyclohepten-2-yl)-oxazolidin-5-ylmethyl]-acetamide, the synthesis of which is depicted in the Scheme and described below.

### Preparation of (S)-N-[2-Oxo-3-(7-oxo-6,7,8,9,-tetrahydro-5H-benzocyclohepten-2-yl)-oxazolidin-5(S)-ylmethyl]-acetamide

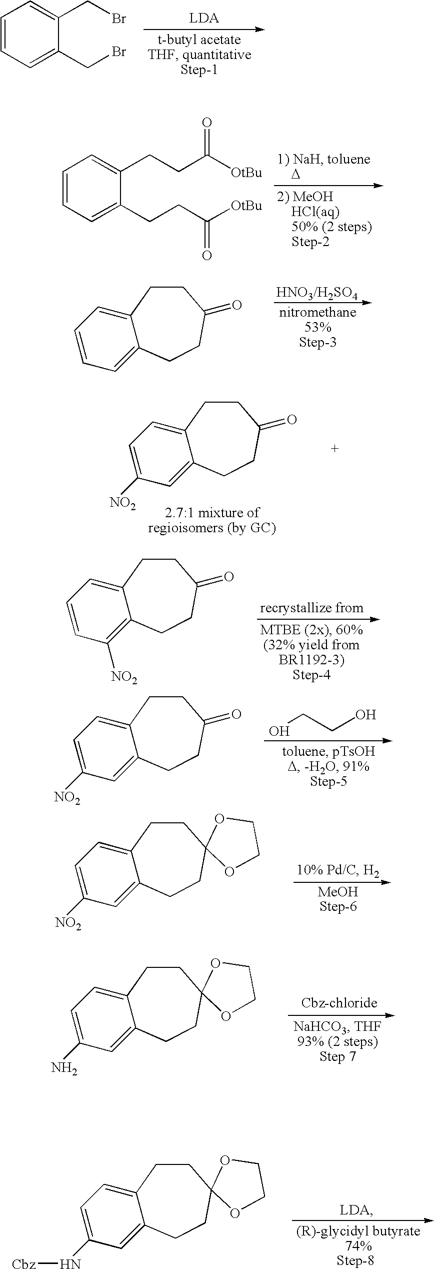

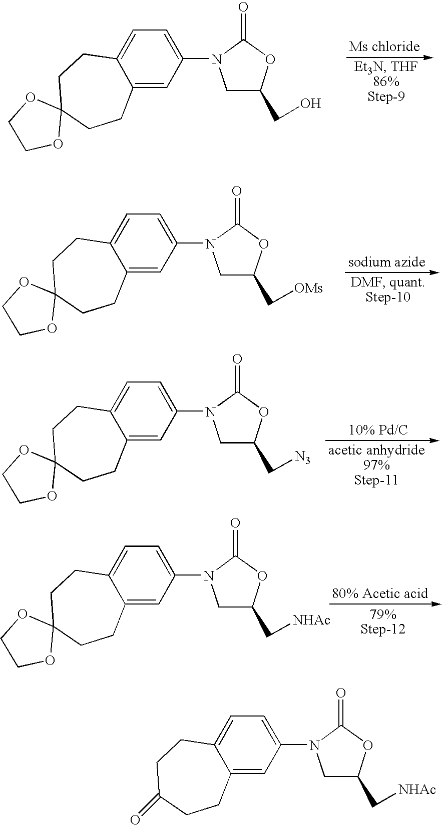

### 3-[2-(2-Tert-Butoxycarbonyl-ethyl)-phenyl]-propionic acid tert-butyl ester (Step 1)

To a solution of a diisopropylamine (578 mL, 4.13 mol) stirring in anhydrous THF (5 L) at −35° C. was added 1.60 L of a 2.5 M solution of n-BuLi in hexanes. The addition was controlled so as to maintain the internal reaction temperature at below −20° C. After the addition was complete the reaction was cooled to −78° C. and tert-butyl acetate (462 g, 3.98 mol) was added dropwise. The rate of addition was controlled so as to maintain the internal reaction temperature below 65° C. After the addition was complete the reaction was allowed to stir an additional 45 minutes as it recooled to −78° C. α,α′-Dibromo-o-xylene (436 g, 1.65 mol) in THF (1.5 L) was then added dropwise over a period of 90 minutes. After the addition was complete the reaction was allowed to slowly warm to room temperature while stirring overnight. The reaction was quenched by the slow addition of 4 L of cold aqueus 1M HCl. The resulting mixture was then transferred to a large separatory funnel, equipped with a mechanical stirrer, containing 4 L of brine. This mixture was stirred for several minutes and the layers were then separated. The aqueous layer was extracted with methyltert-butyl ether (MTBE) (2×2 L). The organic layers were combined and washed with cold 1M HCl (aq) (4 L) and brine (4 L). The solvent was evaporated, and the resulting crude oil was dissolved toluene (4 L) which was then removed under reduce pressure to yield 502 g (91% yield) of the title compound.

### 5,6,8,9-Tetrahydro-benzocyclohepten-7-one (Step 2)

To a suspension of NaH (69.0 g, 2.88 mol; used 115 g of unrinsed 60% NaH in mineral oil) stirring in anhydrous toluene (8.3 L) was added tert-butanol (17.8 mL). The suspension was then heated to 95° C. The diester 3-[2-(2-tert-butoxycarbonyl-ethyl)-phenyl]-propionic acid tert-butyl ester (481 g, 1.44 mol) in toluene (2 L) was then slowly added over a period of 48 hours. After the addition was complete the reaction was allowed to stir at 95° C. for an additional 24 hours. The reaction was allowed to cool to room temperature and was quenched by the slow addition of acetic acid (144 mL). The reaction mixture became very thick. 2 L of ice water was added and the biphasic mixture was allowed to stir for several minutes. The reaction mixture was then transferred to a large separatory funnel where the layers were separated and the aqueous layer was extracted with MTBE (2×2 L). The organic layers were combined and concentrated; the resulting residue was dissolved in methanol (2.8 L). To this solution was added 1.38 L of 6M aqueous HCl. This mixture was heated to reflux with stirring for 6 hours. The reaction was allowed to cool to room temperature. Most of the methanol was evaporated in vacuo, and the aqueous phase was extracted with MTBE (3×). The combined organic layers were washed with brine, dried over sodium sulfate, filtered and concentrated to yield a residue which was purified by bulb-to-bulb distillation (0.4 mm Hg at 90-110° C.) to yield 310 g of the title compound.

### 2-Nitro-5,6,8,9-tetrahydro-benzocyclohepten-7-one (Step 3 and 4)

To a solution of cold (0° C.) concentrated sulfuric acid (102 mL) was added 70% nitric acid (102 mL) dropwise. The addition was controlled to maintain the internal reaction temperature below 5° C. After the addition was complete the solution was added dropwise to a solution 5,6,8,9-tetrahydro-benzocyclohepten-7-one (120 g, 0.75 mol) in nitromethane (720 mL) at 15° C., over a period of 1 hour. The addition was controlled so as to maintain the internal reaction temperature at 15±3° C. The reaction was then poured in to 750 mL of ice water. The mixture was stirred for approximately 30 minutes and transferred to a separatory funnel where the layers were separated. The aqueous layer was extracted with ethyl acetate (3×300 mL). The organic layers were combined, washed with a solution of saturated NaHCO₃ (aqueous) followed by brine. The organic layer was then dried over sodium sulfate, filtered and concentrated to yield a residue which was purified by column chromatography on silica gel, eluting with 30 to 35% ethyl acetate in heptanes, to yield 81.1 g (52%) of a 2.7:1 (by gas chromatography) mixture of nitro regioisomers. This mixture was then purified by recrystallization from MTBE to yield 33.92 g of essentially pure title compound (82:1 by GC).

### Nitro ketal (Step 5)

To a suspension of 2-nitro-5,6,8,9-tetrahydro-benzocyclohepten-7-one (73.7 g, 0.36 mol) stirring in toluene (1.44 L) at room temperature was added ethylene glycol (33.3 g, 0.54 mol) and p-toluenesulfonic acid (3.42 g, 0.018 mol). The resulting mixture was heated to reflux and water was removed with a Dean-Stark trap. After 90 minutes, the reaction was allowed to cool to room temperature and concentrated. The crude mixture was dissolved in ethyl acetate (600 mL) and was washed with saturated aqueous NaHCO₃ (1×150 mL) and water (1×150 mL). The organic layer was then dried over sodium sulfate, filtered and concentrated. The resulting residue was triturated with 25% ethyl acetate in heptanes to yield a solid that was collected by vacuum filtration. The material was dried to yield 82 g (91% yield) of the title compound.

### Amino ketal (Step 6)

To a suspension of 10% Pd/C (5 g) in methanol (130 mL) was added a suspension of nitro ketal (100 g, 0.4 mol) in methanol (700 mL). The resulting mixture was shaken under an atmosphere of H₂ (40 psi) for 90 minutes. During that time, the pressure dropped below 10 psi twice. Each time the bottle was recharged with H₂ to 40 psi. The hydrogen was removed and replaced with an atmosphere of nitrogen. The palladium catalyst was then filtered, and the filtrate was concentrated under reduced pressure to yield 83 g of the title compound. This material was used directly without purification.

### Protected amino ketal (Step 7)

To a solution of the aniline (82.8 g, 0.38 mol) stirring in THF (1.66 liter) at 0° C. was added solid NaHCO₃ (51.2 g, 0.80 mol) followed by the drop-wise addition of benzyl chloroformate (74.8 g, 0.44 mol). The internal reaction temperature rose to 8° C. during the course of the addition. The reaction was allowed to slowly warm to room temperature while stirring overnight. Water (400 mL) was added to the reaction mixture and the solution was allowed to stir at room temperature for 30 minutes. The mixture was diluted with Ethyl acetate (500 mL) and transferred to a separatory funnel where the layers were separated. The aqueous layer was extracted with Ethyl acetate (2×300 mL). The combined organic layers were washed with brine, dried over sodium sulfate, filtered and concentrated. The resulting crude solid was triturated with MTBE to yield a solid that was collected by vacuum filtration. The collected solid was dried to yield 75 g (93% yield for 2 steps) of the title compound.

### Oxazolidinone (Step 8)

To a solution of the protected amino ketal (79.0 g, 0.224) in anhydrous THF at −78° C. was added 137 mL of a 1.8M solution of LDA in heptane/THF/ethylbenzene. After the addition was complete (approximately 30 minutes), the mixture was allowed to stir at −78° C. for an additional 45 minutes. (R)-Glycidyl butyrate (33.9 g, 0.235 mol) was then added dropwise. After the addition was complete, the reaction was allowed to slowly warm to room temperature overnight. The mixture was quenched by the slow addition of a solution of saturated NH₄Cl (aqueous) and diluted with ethyl acetate. The layers were separated, and the aqueous layer was extracted with ethyl acetate (3×200 mL). The combined organic layers were washed with brine, dried over sodium sulfate, filtered, and concentrated. The residue was triturated with ethyl acetate to yield 52.6 g (74% yield) of the title compound.

### Mesylate (Step 9)

To a solution of the alcohol (from step 8) (52.6 g, 0.165 mol) in anhydrous THF (1.65 L) was added Et₃N (23 mL, 0.165 mol) followed by methanesulfonyl chloride (18.8 g, 0.165 mol). After the addition was complete, the reaction mixture was allowed to stir at room temperature for 15 minutes and was then filtered through a plug of celite. The solution was concentrated under reduced pressure to yield a residue that was triturated with methanol to produce the title compound (58.6 g, 86% yield).

### Azide (Step 10)

To a solution of the mesylate (58.5 g, 0.147 mol) stirring in DMF at room temperature was added sodium azide (14.4 g, 0.22 mol). The resulting mixture was heated to 90° C. for 2 hours, then cooled to room temperature and poured into 1 liter of water. The resulting mixture was stirred for approximately 20 minutes and filtered. The product was dried to yield the title compound (53.2 g).

### Acetylamine (Step 11)

To a suspension of 10% Pd/C (3 g) in THF (50 mL) was added a solution of the azide (25 g, 0.073 mol) in THF (450 mL) followed by acetic anhydride (22.3 g, 0.22 mol). The resulting mixture was then shaken under an atmosphere of H₂ (15 psi) for 2 hours. The hydrogen atmosphere was replaced with nitrogen and the palladium catalyst was removed via filtration through a plug of celite. The solution was concentrated and the residue was purified by column chromatography using a Biotage Flash 75 (M) radial compression unit. The crude material was loaded in dichloromethane and the column was eluted with a gradient of 100% ethyl acetate to 1% MeOH to yield 25.1 g (97% yield) of the title compound.

### Deprotection (Step 12): (S)-N-[2-Oxo-3-(7-oxo-6,7,8,9-tetrahydro-5H-benzocyclohepten-2-yl)-oxazolidin-5-ylmethyl]-acetamide

A solution of the dioxolane (56 g, 0.156 mol) in 80% aqueous acetic acid was heated to 65° C. for 5 hours. After cooling the reaction was concentrated under reduced pressure. The residue was dissolved in dichloromethane and was washed with saturated aqueous NaHCO₃ (4 times). The aqueous layers were combined and back extracted with dichloromethane (2 times). The organic layers were combined, washed with brine, dried over sodium sulfate, filtered and concentrated. The residue was triturated with hot MTBE to afford the title compound (39.0 g, 79% yield).

The title compounds were prepared from (S)-N-[2-oxo-3-(7-oxo-6,7,8,9-tetrahydro-5H-benzocyclohepten-2-yl)-oxazolidin-5-ylmethyl]-acetamide as follows.

### N-[3-(6(R,S)-Bromo-7-oxo-6,7,8,9-tetrahydro-5H-benzocyclohepten-2-yl)-2-oxo-oxazolidin-5(S)-ylmethyl]-acetamide and N-[3-(8-Bromo-7-oxo-6,7,8,9-tetrahydro-5H-benzocyclohepten-2-yl)-2-oxo-oxazolidin-5-ylmethyl]-acetamide

To a solution of (S)-N-[2-oxo-3-(7-oxo-6,7,8,9-tetrahydro-5H-benzocyclohepten-2-yl)-oxazolidin-5-ylmethyl]-acetamide (0.050 g, 0.16 mmol) in 5 mL of chloroform at 0° C. was added 8.10 μL (0.16 mmol) of bromine. The mixture was warmed up slowly to room temperature and stirred overnight. Then 2 mL of saturated sodium bicarbonate solution and 2 mL of water were added to the reaction mixture and the layers separated. The aqueous layer was extracted with methylene chloride (3×5 mL). The organic portions were combined and dried (MgSO₄). The solvent was evaporated to give 0.07 g of crude material which was chromatographed in a prep silica gel plate in a system of ethyl acetate; both isomers were collected: 0.025 g (40% yield) of N-[3-(6(R,S)-Bromo-7-oxo-6,7,8,9-tetrahydro-5H-benzocyclohepten-2-yl)-2-oxo-oxazolidin-5(S)-ylmethyl]-acetamide and 0.010 g (16% yield) of N-[3-(8(R,S)-Bromo-7-oxo-6,7,8,9-tetrahydro-5H-benzocyclohepten-2-yl)-2-oxo-oxazolidin-5(S)-ylmethyl]-acetamide. Isomers were determined by NOE spectra.

N-[3-(6(R,S)-Bromo-7-oxo-6,7,8,9-tetrahydro-5H-benzocyclohepten-2-yl)-2-oxo-oxazolidin-5(S)-ylmethyl]-acetamide: LC-MS: m/z 396 (M+1); ¹H NMR (CDCl₃, 400 MHz): δ 7.55 (d, 1H), 7.30 (m, 1H), 7.20 (d, 1H), 6.20 (br t, 1H), 4.77 (m, 1H), 4.53 (m, 1H), 4.05 (dt, 1H), 3.79 (m, 1H), 3.70 (m, 1H), 3.60 (m, 1H), 3.37 (dd, 1H), 3.15 (m overlapping, 2H), 2.95 (m, 1H), 2.83 (m, 1H), 2.62 (m, 1H), 2.02 (s, 3H).

N-[3-(8(R,S)-Bromo-7-oxo-6,7,8,9-tetrahydro-5H-benzocyclohepten-2-yl)-2-oxo-oxazolidin-5(S)-ylmethyl]-acetamide: LC-MS: m/z 396 (M+1); ¹H NMR (CDCl₃, 400 MHz): δ 7.43 (dd, 1H), 7.35 (m, 1H), 7.20 (d, 1H), 6.20 (br t, 1H), 4.77 (m, 1H), 4.53 (m, 1H), 4.05 (dt, 1H), 3.79 (m, 1H), 3.70 (m, 1H), 3.60 (m, 1H), 3.41 (dd, 1H), 3.22 (ddd, 1H), 3.15 (m, 1H) 2.95 (m, 1H), 2.83 (m, 1H), 2.62 (m, 1H), 2.02 (s, 3H).

### Example 5

### IIA. (S)-N-[3-(2-Methyl-9,10-dihydro-4H-3-thia-1-aza-benzo[f]azulen-7-yl)-2-oxo-oxazolidin-5-ylmethyl]-acetamide

To a solution of N-[3-(6(R,S)-bromo-7-oxo-6,7,8,9-tetrahydro-5H-benzocyclohepten-2-yl)-2-oxo-oxazolidin-5(S)-ylmethyl]-acetamide (0.030 g, 0.076 mmol) in 1 mL of ethanol was added 0.006 g (0.076 mmol) of thioacetamide and 0.006 g of sodium bicarbonate. This reaction mixture was heated for 15 minutes at 160° C. in a microwave reactor. The solvent was evaporated and the residue was chromatographed on a prep TLC plate in a system of 5% MeOH/CH₂Cl₂ to provide 0.008 g of the title compound (28% yield). LC-MS: m/z 371 (M+1).

### Example 6

### IIB. (S)-N-[3-(2-Methyl-9,10-dihydro-4H-3-thia-1-aza-benzo[f]azulen-6-yl)-2-oxo-oxazolidin-5-ylmethyl]-acetamide

To a solution of N-[3-(8(R,S)-bromo-7-oxo-6,7,8,9-tetrahydro-5H-benzocyclohepten-2-yl)-2-oxo-oxazolidin-5(S)-ylmethyl]-acetamide (0.030 g, 0.076 mmol) in 1 mL of ethanol was added 0.006 g (0.076 mmol) of thioacetamide and 0.006 g of sodium bicarbonate. This mixture was heated for 15 minutes at 160° C. in a microwave reactor. The solvent was evaporated and the residue was chromatographed on a prep TLC plate in a system of 5% MeOH/CH₂Cl₂ to provide the title compound. LC-MS: m/z 371 (M+1).

### Example 7

### (S)-N-[3-(2-Methyl-5,6-dihydro-4H-3-thia-1-aza-benzo[e]azulen-8-yl)-2-oxo-oxazolidin-5ylmethyl] acetamide

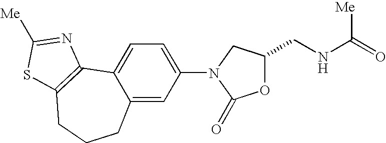

### N-[3-(6(R,S)-Bromo-5-oxo-6,7,8,9-tetrahydro-5H-benzocyclohepten-2-yl)-2-oxo-oxazolidin-5(S)-ylmethyl]-acetamide

To a solution of (S)-N-[2-oxo-3-(5-oxo-6,7,8,9-tetrahydro-5H-benzocyclohepten-2-yl)-oxazolidin-5-ylmethyl]-acetamide (II-7, 1.0 g, 31.65 mmol) in 30 mL of chloroform was added 0.162 mL (31.65 mmol) of bromine. The solution was stirred at room temperature overnight. Saturated aqueous sodium bicarbonate (15 mL) was added followed by 20 mL of methylene chloride. The layers were separated and the aqueous layer was extracted with methylene chloride (3×20 mL). The combined organic layers were dried (MgSO₄) and concentrated under reduced pressure to provide 1.3 g of the title compound.

### (S)-N-[3-(2-Methyl-5,6-dihydro-4H-3-thia-1-aza-benzo[e]azulen-8-yl)-2-oxo-oxazolidin-5ylmethyl] acetamide

To a solution of N-[3-(6(R,S)-bromo-5-oxo-6,7,8,9-tetrahydro-5H-benzocyclohepten-2-yl)-2-oxo-oxazolidin-5(S)-ylmethyl]-acetamide (0.035 g, 0.089 mmol) in 1 mL of ethanol was added 0.007 g (0.089 mmol) of thioacetamide and 0.007 g of sodium bicarbonate. This mixture was heated for 15 minutes at 160° C. in a microwave reactor. The solvent was then evaporated and the residue was chromatographed on a prep TLC plate in a system of 5% MeOH/CH₂Cl₂ to provide 0.008 g of the title compound (24% yield). LC-MS: m/z 371 (M+1).

### Example 8

### (S)-N-[3-(2-Amino-5,6-dihydro-4H-3-thia-1-aza-benzo[e]azulen-8-yl)-2-oxo-oxazolidin-5-ylmethyl]-acetamide

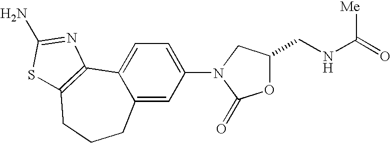

To a solution of 0.035 g (0.089 mmol) of N-[3-(6(R,S)-bromo-5-oxo-6,7,8,9-tetrahydro-5H-benzocyclohepten-2-yl)-2-oxo-oxazolidin-5(S)-ylmethyl]-acetamide in 1 mL of ethanol was added 0.007 g (0.089 mmol) of thiourea and 0.007 g of sodium bicarbonate. This solution was heated for 15 minutes at 160° C. in a microwave reactor. The solvent was evaporated and the residue was chromatographed on a prep TLC plate in a system of 5% MeOH/CH₂Cl₂ to provide 0.007 g of the title compound (21% yield). LC-MS: m/z 408 (M+1)

### Example 9

### (S)-N-[3-(2-Methyl-3,4,5,6-tetrahydro-1,3-diaza-benzo[e]azulen-8-yl)-2-oxo-oxazolidin-5-ylmethyl]-acetamide

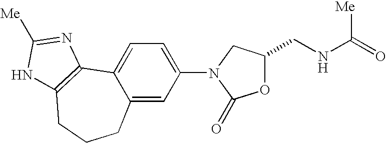

To a solution of N-[3-(6(R,S)-bromo-5-oxo-6,7,8,9-tetrahydro-5H-benzocyclohepten-2-yl)-2-oxo-oxazolidin-5(S)-ylmethyl]-acetamide (0.035 g, 0.088 mmol) in 1 mL of chloroform and 1 mL of dimethylformamide was added 0.021 g (0.356 mmol) of acetamidine. This mixture was heated for 15 minutes at 140° C. in a microwave reactor. The solvent was evaporated and the residue was chromatographed on a prep TLC plate in a system of 5% MeOH/CH₂Cl₂ to provide 0.006 g of the title compound (20% yield). LC-MS: m/z 354 (M+1).

### Example 10

### (S)-N-[2-Oxo-3-(2-trifluoromethyl-3,4,5,6-tetrahydro-1,3-diaza-benzo[e]azulen-8-yl)-oxazolidin-5-ylmethyl]-acetamide

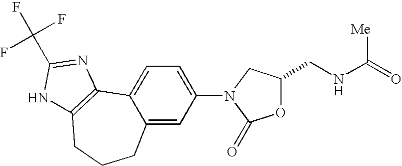

To a solution of N-[3-(6(R,S)-bromo-5-oxo-6,7,8,9-tetrahydro-5H-benzocyclohepten-2-yl)-2-oxo-oxazolidin-5(S)-ylmethyl]-acetamide (0.050 g, 1.26 mmol) in 1 mL of DMF was added 0.057 g (5.04 mmol) of 2,2,2-trifluoromethylacetamidine. The solution was heated at 140° C. in a microwave reactor for 20 minutes. The solvent was removed under reduced pressure, and the crude residue was purified by prep TLC plate in a system of 5% MeOH/CH₂Cl₂ to provide 0.010 g of the title compound (19% yield). LC-MS: m/z 408 (M+1).

### Example 11

### (S)-N-[2-Oxo-3-(3,4,5,6-tetrahydro-2,3-diaza-benzo[e]azulen-9-yl)-oxazolidin-5-ylmethyl]-acetamide

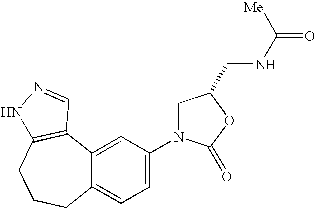

The title compound was prepared from (S)-N-[2-oxo-3-(8-oxo-6,7,8,9-tetrahydro-5H-benzocyclohepten-2-yl)-oxazolidin-5-ylmethyl]-acetamide (XI-8), the synthesis of which is schematically depicted and described below.

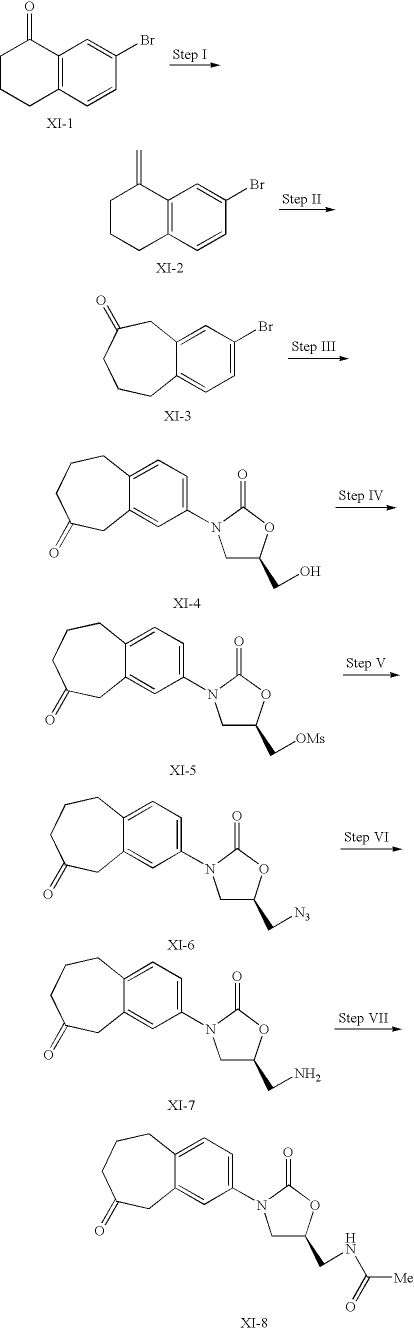

### 7-Bromo-1-methylene-1,2,3,4-tetrahydro-naphthalene (XI-2, Step I)

7-Bromo-3,4-dihydro-2H-naphthalen-1-one (1.62 g, 7.2 mmol) was dissolved in tetrahydrofuran (18 mL) and cooled to 0° C. To this solution was slowly added Tebbe's reagent (0.5 M in toluene, 18 mL, 9 mmol), and the reaction mixture was stirred for 3 hours at 0° C. The reaction was quenched with 0.1N sodium hydroxide by drop wise addition. Ethyl acetate was added, and the organic layer was separated, dried over sodium sulfate, and concentrated. The crude material was purified by flash silica gel chromatography to afford the title compound (Yield: 54%). LC-MS m/z: 225 (M+H).

### 3-Bromo-5,7,8,9-tetrahydro-benzocyclohepten-6-one (XI-3, Step II)

Silver nitrate was dissolved in methanol (36 mL) and refluxed for 1 hour until all the material had dissolved. A solution of 7-bromo-1-methylene-1,2,3,4-tetrahydro-naphthalene (0.87 g, 3.91 mmol) in methanol (24 mL) and iodine (0.992 g, 3.91 mmol) was added to the hot silver nitrate solution, and the resulting reaction mixture was kept under reflux for 2 hours. The reaction mixture was cooled to room temperature, filtered through celite, and treated with 1N hydrochloric acid. The methanol was evaporated; the residue was dissolved in ether, washed with 10% sodium thiosulfate and brine, dried over sodium sulfate, and concentrated. The residue was purified by flash silica gel chromatography to obtain the title compound (0.48 g, 52% yield). LC-MS m/z: 239 (M+H).

### (R)-5-Hydroxymethyl-3-(8-oxo-6,7,8,9-tetrahydro-5H-benzocyclohepten-2-yl)-oxazolidin-2-one (XI-4, Step III)

3-Bromo-5,7,8,9-tetrahydro-benzocyclohepten-6-one (0.070 g, 0.29 mmol), 5-hydroxymethyl-oxazolidin-2-one (0.034 g, 0.29 mmol), copper (I) iodide (0.011 g, 0.059 mmol), trans-1,2-diaminocyclohexane (7 μL, 0.059 mmol), potassium carbonate (0.084 g, 0.61 mmol) and dimethylformamide (0.5 mL) were combined and purged of oxygen. The mixture was purged with stirring another four times and heated to 105° C. overnight under nitrogen. The reaction mixture was diluted with ethyl acetate and water; the organic layer was washed with 1M hydrochloric acid (2×), brine, dried over sodium sulfate and concentrated in vacuo to give the title compound. Yield: 51%; LC-MS m/z: 276 (M+H).

### (R)-Methanesulfonic acid 2-oxo-3-(8-oxo-6,7,8,9-tetrahydro-5H-benzocyclohepten-2-yl)-oxazolidin-5-ylmethyl ester (XI-5, Step IV)

(R)-5-Hydroxymethyl-3-(8-oxo-6,7,8,9-tetrahydro-5H-benzocyclohepten-2-yl)-oxazolidin-2-one (1.72 mmol), methanesulfonyl chloride (200 μL, 2.58 mmol), triethylamine (0.44 mL, 3.44 mmol) and dichloromethane (8.6 mL) were combined and stirred at room temperature overnight. The solution was diluted with ethyl acetate, washed with 1M hydrochloric acid (2 times) and brine, dried over sodium sulfate and concentrated. The residue was triturated with diethyl ether (2×) and dried in vacuo to give the title compound. Yield: 72% over two steps. LC-MS m/z: 354 (M+H).

### (R)-5-Azidomethyl-3-(8-oxo-6,7,8,9-tetrahydro-5H-benzocyclohepten-2-yl)-oxazolidin-2-one (XI-6, Step V)

(R)-Methanesulfonic acid 2-oxo-3-(8-oxo-6,7,8,9-tetrahydro-5H-benzocyclohepten-2-yl)-oxazolidin-5-ylmethyl ester (0.44 g, 1.25 mmol), sodium azide (0.31 g, 4.7 mmol) and dimethyl formamide (6.25 mL) were combined and heated at 80° C. for 2 hours. The solution was cooled to room temperature and diluted with ethyl acetate. The organic phase was washed with water (2 times) and brine and dried over sodium sulfate, and concentrated to give the title compound. LC-MS: 301 (M+H).

### (S)-5-Aminomethyl-3-(8-oxo-6,7,8,9-tetrahydro-5H-benzocyclohepten-2-yl)-oxazolidin-2-one (XI-7, Step VI)

(R)-5-Azidomethyl-3-(8-oxo-6,7,8,9-tetrahydro-5H-benzocyclohepten-2-yl)-oxazolidin-2-one (0.12 g, 0.4 mmol), Pd/C (50 mg), and methanol (5 mL) were combined in a hydrogenation bottle. The mixture was hydrogenated at 50 psi for 2.5 hours. The solution was diluted with methanol, filtered through a pad of celite and concentrated in vacuo to give the title compound. Yield: 71%; LC-MS m/z: 275 (M+H).

### (S)-N-[2-Oxo-3-(8-oxo-6,7,8,9-tetrahydro-5H-benzocyclohepten-2-yl)-oxazolidin-5-ylmethyl]-acetamide (XI-8, Step VII)

(S)-5-Aminomethyl-3-(8-oxo-6,7,8,9-tetrahydro-5H-benzocyclohepten-2-yl)-oxazolidin-2-one (0.088 g, 0.28 mmol), acetic anhydride (0.37 μL, 0.39 mmol), pyridine (66 μL, 0.84 mmol) and dichloromethane (2 mL) were combined at room temperature and stirred for 20 minutes. The solution was diluted with ethyl acetate, washed with 1M hydrochloric acid (2×) and brine, dried over sodium sulfate and concentrated. The residue was dissolved in methanol and filtered through a DOWEX 1×4-100 ion exchange resin plug (strongly basic anion, 4% cross-linking) and concentrated in vacuo to give the acetylated compound. A solution of the acetylated compound in 4N hydrochloric acid in dioxane was then stirred at room temperature under nitrogen for 20 minutes. The solvent was decanted and the residue was dried under high vacuum to give the title compound. Yield: 71%; LC-MS m/a: 317 (M+H).

### (S)-N-[3-(9-Dimethylaminomethylene-8-oxo-6,7,8,9-tetrahydro-5H-benzocyclohepten-2-yl)-2-oxo-oxazolidin-5-ylmethyl]-acetamide

A solution of (S)-N-[2-oxo-3-(8-oxo-6,7,8,9-tetrahydro-5H-benzocyclohepten-2-yl)-oxazolidin-5-ylmethyl]-acetamide (0.8 g, 2.53 mmol) in 1-propanol (20 mL) was treated with dimethylformamide-dimethyl acetal (1.21 g, 10.12 mmol). The resulting mixture was refluxed for 25 hours. The mixture was cooled to room temperature and concentrated to give the title compound. Yield: 0.98 g, still wet. MS AP+=372.2.

### (S)-N-[2-Oxo-3-(3,4,5,6-tetrahydro-2,3-diaza-benzo[e]azulen-9-yl)-oxazolidin-5-ylmethyl]-acetamide

A solution of (S)-N-[3-(9-dimethylaminomethylene-8-oxo-6,7,8,9-tetrahydro-5H-benzocyclohepten-2-yl)-2-oxo-oxazolidin-5-ylmethyl]-acetamide in ethanol was treated dropwise with hydrazine hydrate and stirred at room temperature over 4-8 hours. The solution was concentrated, and the residue was chromatographed over silica gel, eluting with 5% MeOH in ethyl acetate (with CH₂Cl₂ to solublize). NMR CDCl₃ MS (APCI): AP+, 341.1; AP−, 339.1. Elemental Analysis: calc'd. for C₁₈H₂₀N₄O₃.0.60H₂O: C, 61.56; H, 6.08; N, 15.95. Found: C, 61.80; H, 5.87; N, 15.39

### Example 12

### (S)-N-[3-(5,6-Dihydro-4H-3-oxa-2-aza-benzo[e]azulen-9-yl)-2-oxo-oxazolidin-5-ylmethyl]-acetamide

A solution of the (S)-N-[3-(9-dimethylaminomethylene-8-oxo-6,7,8,9-tetrahydro-5H-benzocyclohepten-2-yl)-2-oxo-oxazolidin-5-ylmethyl]-acetamide (0.47 g, 1.27 mmol) was cooled in an ice bath, then treated dropwise with a solution of hydroxylamine-O-sulfonic acid (0.16 g, 1.4 mmol) in 2 m/L of MeOH. The mixture was allowed to warm to room temperature over 4-8 hours, then treated with ice and saturated sodium bicarbonate, and extracted with dichloromethane (2×50 mL) and ethyl acetate (1×50 mL). The extracts were combined, washed with brine, dried over magnesium sulfate, and concentrated. The product was chromatographed over silica gel, eluting with 5% MeOH in ethyl acetate, to give the title compound. Yield: 0.11 g. MS (APCI): AP+, 342.1; AP−, 340.1.

### Example 13

### (S)-N-[2-Oxo-3-(6-oxo-6,7,8,9-tetrahydro-5H-benzocyclohepten-2-yl)-oxazolidin-5-ylmethyl]-acetamide (Method A)

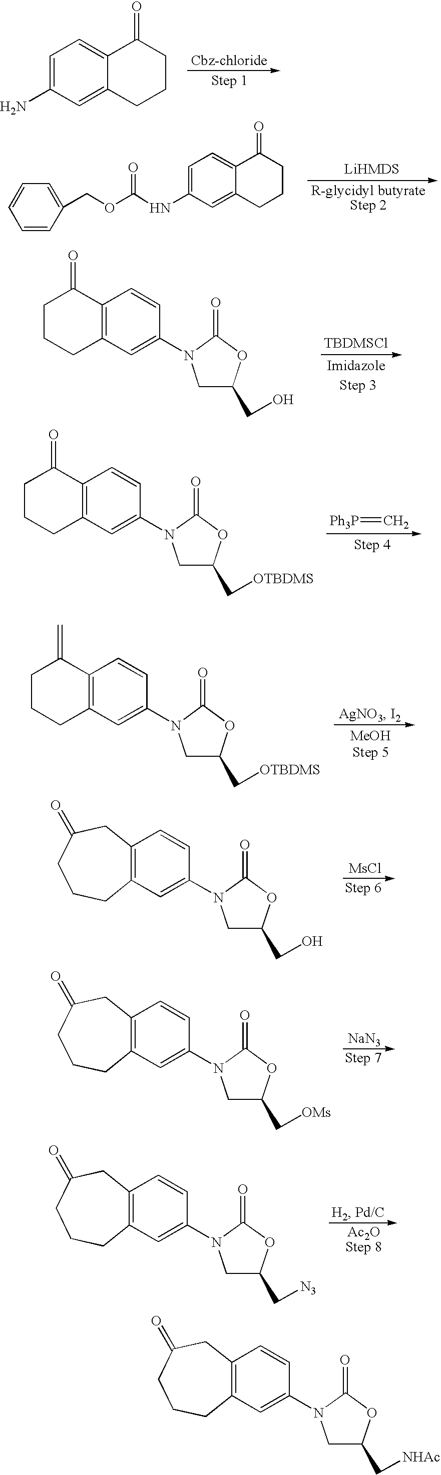

### (5-Oxo-5,6,7,8-tetrahydro-naphthalen-2-yl)-carbamic acid benzyl ester (Step 1)

To a mixture of 6-amino-3,4-dihydro-2H-naphthalen-1-one (145.0 g, 0.9 mol), NaHCO₃ (151.2 g, 1.8 mol) and 1.8 L of THF at 0±5° C. was added benzyl chloroformate (156.7 mL of 97%, 1.14 mol). The mixture was stirred cold for 2 hours and allowed to stir overnight at room temperature. The solids were filtered off, washed with THF, and the solvent evaporated. The residue was taken up in 1.5 L of ethyl acetate, washed with saturated NaHCO₃ and brine, dried over sodium sulfate, filtered, concentrated, and then triturated with heptane to give 248.4 g (93% yield) of the title compound.

### (R)-5-Hydroxymethyl-3-(5-oxo-5,6,7,8-tetrahydro-naphthalen-2-yl)-oxazolidin-2-one (Step 2)

To a solution of (5-oxo-5,6,7,8-tetrahydro-naphthalen-2-yl)-carbamic acid benzyl ester (153.0 g, 0.518 mol) in THF (1.7 L) at >−70° C. was added lithium (trimethylsilyl) amide (1.0 M in THF, 544 mL, 0.54 mol) at −70° C., and the mixture was stirred for 1 hour. (R)-(−) Glycidyl butyrate (77.0 mL, 0.54 mol) was added over 40 minutes. The solution was stirred for 2 hours at −70° C. and allowed to warm to room temperature overnight. The reaction was quenched with 300 mL of saturated NH₄Cl; the layers were separated and the organic layer was concentrated. The residue was triturated with 25% ethyl acetate in heptane several times to give 250 g (92% yield) of the title compound.

### (R)-5-(tert-Butyl-dimethyl-silanyloxymethyl)-3-(5-oxo-5,6,7,8-tetrahydro-naphthalen-2-yl)-oxazolidin-2-one (Step 3)

(R)-5-Hydroxymethyl-3-(5-oxo-5,6,7,8-tetrahydro-naphthalen-2-yl)-oxazolidin-2-one (340.82 g, 1.3 mol) was treated with dimethyl-tert-butyl silyl chloride (235.9 g, 1.57 mol) and imidazole (221.9 g, 3.26 mol) in dimethylformamide 900 mL at room temperature. The reaction was stirred overnight, and then water (6.0 L) was added and stirred for 20 minutes. The reaction mixture was filtered and concentrated. The residue was dissolved in ethyl acetate (6.0 L), washed with water and brine, dried over sodium sulfate, and concentrated to give 452.2 g (92% yield) of the title compound.

### (R)-5-(tert-Butyl-dimethyl-silanyloxymethyl)-3-(5-methylene-5,6,7,8-tetrahydro-naphthalen-2-yl)-oxazolidin-2-one (Step 4)

To a suspension of methyltriphosphonium iodide (177.6 g, 0.44 mol) in dry THF (0.8 L) was added dropwise potassium hexamethyldisilazide (0.5 M in toluene, 878.8 mL, 0.44 mol) at 0° C. under nitrogen. The reaction mixture was allowed to warm to room temperature over 2 hours, then recooled to 0° C. A solution of (R)-5-(tert-butyl-dimethyl-silanyloxymethyl)-3-(5-oxo-5,6,7,8-tetrahydro-naphthalen-2-yl)-oxazolidin-2-one (150.0 g, 0.40 mol) in dry THF (1 L) was added. The reaction mixture was allowed to warm slowly to room temperature over 4 hours and stirred overnight. The reaction was quenched with saturated NH₄Cl solution and extracted with ethyl acetate. The organic layer was washed with brine and dried over sodium sulfate. Evaporation of solvent and purification of the residue by column chromatography (silica gel: ethyl acetate/heptane 1:3) gave 138.0 g (92%) of the title compound. [α] D22: −42.7° (c 0.690 g/100 mL-chloroform).

### (R)-5-Hydroxymethyl-3-(6-oxo-6,7,8,9-tetrahydro-5H-benzocyclohepten-2-yl)-oxazolidin-2-one (Step 5)

Silver nitrate (125.5 g, 0.74 mol) was added to dry methanol (3 L). The reaction mixture was refluxed over 1.5 h until all solids dissolved. Iodine (93.7 g, 0.40 mol) and (R)-5-(tert-butyl-dimethyl-silanyloxymethyl)-3-(5-methylene-5,6,7,8-tetrahydro-naphthalen-2-yl)-oxazolidin-2-one (138.0 g, 0.37 mol) were added simultaneously in dry methanol (3 L) and the reaction mixture refluxed for 3 hours. The reaction mixture was then cooled in an ice bath, after which time the silver iodide was filtered off and the filtrate concentrated. The residue was diluted with water (4.0 L) and extracted with ethyl acetate, and the organic layer was separated, washed with saturated sodium bicarbonate and saturated sodium hydrogensulfite. The precipitates in the aqueous layer were filtered and then 6 L of dichloromethane was added with stirring. 1N Aqueous HCl was added slowly until the solution turns to acidic (pH>>3) and the precipitates redissolved to organic layer. The organic layer was separated and washed with water and brine and was dried over sodium sulfate to give 47.5 g of the title compound.

### (R)-5-Methanesulfonic acid 2-oxo-3-(6-oxo-6,7,8,9-tetrahydro-5H-benzocyclohepten-2-yl)-oxazolidin-5-ylmethyl ester (Step 6)

To a cooled solution (0° C.) of (R)-5-hydroxymethyl-3-(6-oxo-6,7,8,9-tetrahydro-5H-benzocyclohepten-2-yl)-oxazolidin-2-one (47.15 g, 171 mmol) in methylene chloride (1 L) was added triethylamine (34.65 g, 343 mmol), followed by methanesulfonyl chloride (29.38 g, 257 mmol). The reaction mixture was stirred for 30 minutes at 0° C. and was allowed to warm up to room temperature and stirred overnight. The mixture was diluted with ethyl acetate, washed with water and brine, and dried over sodium sulfate. Evaporation of the solvent gave 59.0 g (98% yield) of the title compound, which was used to next step without further purification.

### (R)-5-Azidomethyl-3-(6-oxo-6,7,8,9-tetrahydro-5H-benzocyclohepten-2-yl)-oxazolidin-2-one (Step 7)

A mixture of (R)-5-methanesulfonic acid 2-oxo-3-(6-oxo-6,7,8,9-tetrahydro-5H-benzocyclohepten-2-yl)-oxazolidin-5-ylmethyl ester (118.0 g, 334 mmol), NaN₃ (435 g, 668 mmol) and DMF (1 L) was stirred at 60° C. for 5 hours. The reaction mixture was then poured into cold water rapid stirring, The solids that formed were filtered, washed with water, and dried to give 86.7 g (86%) of the title compound.

### (S)-N-[2-Oxo-3-(6-oxo-6,7,8,9-tetrahydro-5H-benzocyclohepten-2yl)-oxazolidin-5-ylmethyl]-acetamide (Step 8)

A mixture of (R)-5-azidomethyl-3-(6-oxo-6,7,8,9-tetrahydro-5H-benzocyclohepten-2-yl)-oxazolidin-2-one (86.7 g, 288.7 mmol), acetic anhydride (88.34 g, 866 mmol) and 5% Pd/C (20 g) in THF (1 L) was hydrogenated at 25 psi of hydrogen for 8 hours at room temperature. The catalyst was filtered through celite and washed with THF, dichloromethane, and then MeOH. The filtrate was concentrated. The residue was redissolved in dichloromethane and treated with saturated NaHCO₃ solution to neutralize the acetic acid. The organic layer was washed with brine, dried over sodium sulfate and concentrated to a small volume. The precipitates that formed were filtered and triturated with 80% ethyl acetate in heptane to give 44.0 g the title compound. [α] D22: −6.8° (c 0.487 g/100 mL-chloroform).

### Alternate Route for the Preparation of (S)-N-[2-Oxo-3-(6-oxo-6,7,8,9-tetrahydro-5H-benzocyclohepten-2yl)-oxazolidin-5-ylmethyl]-acetamide (Method B)

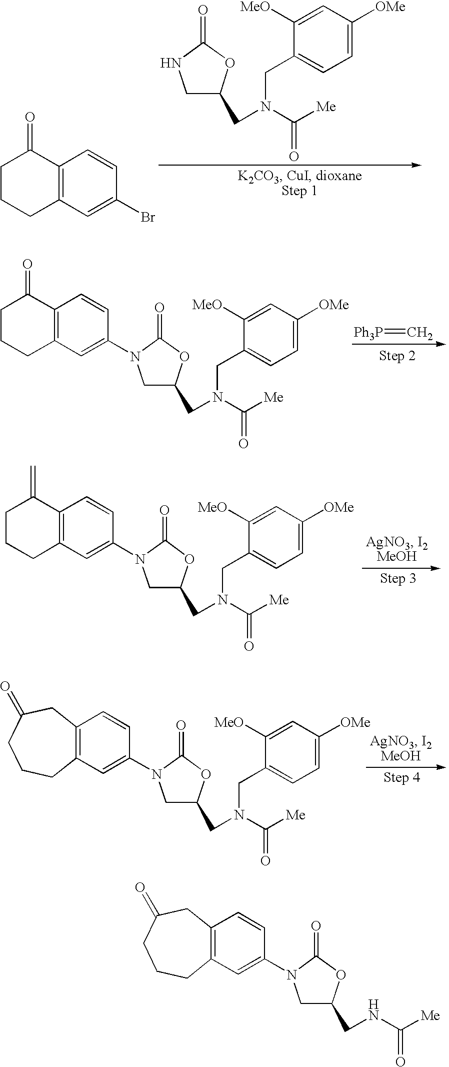

### (S)-N-(2,4-Dimethoxy-benzyl)-N-[2-oxo-3-(5-oxo-5,6,7,8-tetrahydro-naphthalen-2-yl)-oxazolidin-5-ylmethyl]-acetamide (Step 1)

A solution of 1.02 g (4.53 mmol) of 6-bromotetralone in dioxane (8 mL) was treated with (R)-N-(2,4-dimethoxy-benzyl)-N-(2-oxo-oxazolidin-5-ylmethyl)-acetamide (1.40 g, 4.54 mmol), powdered potassium carbonate (1.25 g, 9.04 mmol), and trans-1,2-diaminocyclohexane (0.11 mL, 0.91 mmol). The mixture was purged of oxygen by bubbling nitrogen through the solution and then evacuating the system, a process that was repeated 5 times. Copper (I) iodide (0.17 g, 0.89 mmol) was added, and the purging sequence was repeated another 6 times. The resulting solution was stirred at 110° C. under nitrogen for 19 hours. The suspension was cooled to room temperature and diluted with ethyl acetate (50 mL), methanol (30 mL), and 0.5 N HCl (40 mL). The aqueous phase was extracted with ethyl acetate; the combined organic solutions were washed with water, dried (magnesium sulfate), and concentrated. The residue was chromatographed over silica gel, eluting with 5% methanol in ethyl acetate, to give the title compound. MS (APCI) AP+, 453.2

### (S)-N-2,4-Dimethoxy-benzyl)-N-[3-(5-methylene-5,6,7,8-tetrahydro-naphthalen-2-yl)-2-oxo-oxazolidin-5-ylmethyl]-acetamide (Step 2)

A suspension of 1.27 g (0.62 mmol) of methyl triphenylphosphonium iodide in tetrahydrofuran (10 mL) was cooled to 0° C. under nitrogen and treated dropwise with 6.3 mL of 0.5 M potassium hexamethyldisilazide. The mixture was stirred at 0° C. for 30 minutes, and (S)-N-(2,4-dimethoxy-benzyl)-N-[2-oxo-3-(5-oxo-5,6,7,8-tetrahydro-naphthalen-2-yl)-oxazolidin-5-ylmethyl]-acetamide (0.43 g, 0.95 mmol) was added all at once. The reaction mixture was allowed to warm to room temperature over 4 hours. The mixture was cooled to −30° C. and treated with saturated ammonium chloride. Ethyl acetate was added, and the organic layer was separated, washed with brine and dried over magnesium sulfate. Concentration at reduce pressure gave a residue which was chromatographed on silica gel, eluting with 5% methanol in ethyl acetate, to give the title compound. MS (APCI) AP+, 451.2.

### (S)-N-(2,4-Dimethoxy-benzyl)-N-[3-(6-methylene-6,7,8,9-tetrahydro-5H-benzocyclohepten-2-yl)-2-oxo-oxazolidin-5-ylmethyl]-acetamide (Step 3)

A mixture of 0.20 g (1.15 mmol) of silver nitrate in MeOH (20 mL) was refluxed for 1 hour, until all solids had dissolved. A solution of (S)-N-(2,4-dimethoxy-benzyl)-N-[3-(5-methylene-5,6,7,8-tetrahydro-naphthalen-2-yl)-2-oxo-oxazolidin-5-ylmethyl]-acetamide (0.26 g, 0.58 mmol), iodine (0.15 g, 0.58 mmol), and MeOH (20 mL) was added all at once to the hot silver nitrate solution; a suspension formed immediately which rapidly turned dark. This suspension was refluxed for 4.5 hours and cooled. The solids were filtered and washed with ethyl acetate and the filtrate was concentrated. The residue was partitioned between ethyl acetate and saturated ammonium chloride. The organic layer was washed with 5% sodium bisulfite and brine and was dried over magnesium sulfate. Concentration gave a residue which was chromatographed over silica gel, eluting with 5% MeOH in ethyl acetate. MS (APCI) AP+, 467.2.

### (S)-N-[2-Oxo-3-(6-oxo-6,7,8,9-tetrahydro-5H-benzocyclohepten-2yl)-oxazolidin-5-ylmethyl]-acetamide (Step 4)

The (S)-N-(2,4-dimethoxy-benzyl)-N-[3-(6-methylene-6,7,8,9-tetrahydro-5H-benzocyclohepten-2-yl)-2-oxo-oxazolidin-5-ylmethyl]-acetamide was dissolved in 4 mL of trifluoroacetic acid (TFA) and stirred at room temperature for 1.5 hours. The solvent was evaporated, and the residue was chromatographed over silica gel, eluting with 5% MeOH in ethyl acetate, to give the title compound. MS (APCI) AP+, 317.1

### Example 14

### N-[3-(5(R,S)-hydroxy-6,7,8,9-tetrahydro-5H-benzocyclohepten-2-yl)-2-oxo-oxazolidin-5(S)-ylmethyl]-acetamide

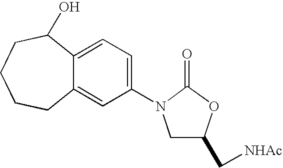

A solution of (S)-N-[2-oxo-3-(5-oxo-6,7,8,9-tetrahydro-5H-benzocyclohepten-2-yl)-oxazolidin-5-ylmethyl]-acetamide (54 g, 0.711 mol) in a mixture of ethanol (98%, 1.2 L) and THF (120 mL) was cooled to 3° C. and treated with sodium borohydride (12.91 g, 37.13 mmol) in portions and stirred at 3-4° C. for 75 hours. The mixture was carefully quenched with a solution of aqueous saturated sodium bicarbonate (1.2 L) and extracted with ethyl acetate (4×1.5 L). The emulsion was filtered, washed with brine (2×600 mL), dried over magnesium sulfate, filtered and evaporated under vacuum to give the title compound. Yield: quantitative.

### Example 15

### (S)-N-[3-(8,9-Dihydro-7H-benzocyclohepten-2-yl)-2-oxo-oxazolidin-5-ylmethyl]-acetamide

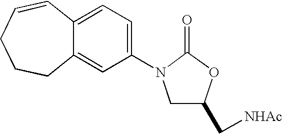

To a solution of N-[3-(5(R,S)-hydroxy-6,7,8,9-tetrahydro-5H-benzocyclohepten-2-yl)-2-oxo-oxazolidin-5(S)-ylmethyl]-acetamide (54.3 g, 0.171 mol) in a mixture of toluene (1.13 L) and DMF (345 mL) was added p-toluene sulfonic acid (114 g, 0.6 mol) and the mixture was refluxed in a Dean Stark condenser for 16 hours with the removal of water. The mixture was stirred over night at room temperature and poured over water (3 L). The mixture was extracted with ethyl acetate, washed with brine (6×800 mL) then with sodium bicarbonate solution, dried over magnesium sulfate, filtered, and concentrated. The residue was purified by column chromatography over silica gel (ethyl acetate). The product was further purified by trituration with ether, filtered, then dried to give the title compound. Yield: 29 g (57.6%), Melting point. 145-147° C.

### Example 16

### (S)-N-[3-(4-Fluoro-5-oxo-6,7,8,9-tetrahydro-5H-benzocyclohepten-2-yl)-2-oxo-oxazolidin-5-ylmethyl]-acetamide

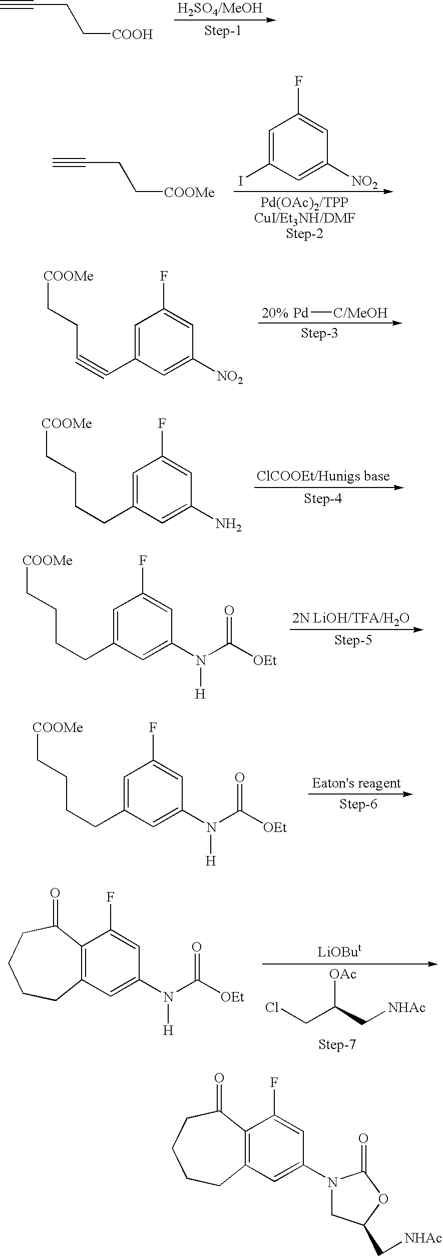

### Methyl pent-4-ynoate (Step-1)

To a stirred solution of the pent-4-ynoic acid (225.0 g, 2.29 mol) and NaOH (9.18 g) in water (800 mL) was added dimethyl sulfate (240 mL) dropwise over 2 hours. During the addition, the temperature changed from 34 to 38° C. The mixture was heated at 60° C. for 1 hour 45 minutes, treated with potassium carbonate (60.6 g) and heated at 50-60° C. for 1 hour. The mixture was cooled to room temperature, extracted with dichloromethane (4×500 mL) and the combined organic extracts were washed with water, dried over MgSO₄, filtered and concentrated under reduced pressure to give the title compound. Yield: 200 g (77.77%).

### 5-(3-Fluoro-5-nitro-phenyl)-pent-4-ynoic acid methyl ester (Step-2)

A mixture of 1-fluoro-3-iodo-5-nitrobenzene (376.19 g, 1.40 mol), methyl pent-4-ynoate (158.0 g, 1.40 mol), Pd(OAc)₂ (12.79 g, 56.42 mmol), triphenyl phospine (29.71 g, 112.8 mmol), CuI (21.28 mmol) and diethylamine (564.28 mL) in DMF (300 mL) was stirred at room temperature for 15 hours. The mixture was filtered through a short bed of silica gel and the solvent was removed under reduced pressure. The residue was taken up in water (1.0 L) and extracted with ethyl acetate (3×500 mL); the combined organic layers were washed with brine solution (1×500 mL), dried over anhydrous MgSO₄, filtered concentrated. The residue was purified by flash chromatography over silica gel (Hex:ethyl acetate 0-30%) to give the title compound. Yield: 150 g (44.51%). Melting point: 111-112° C.

### Methyl 5-(3-amino-5-fluorophenyl)pentanoate (Step-3)

A solution of methyl 5-(3-fluoro-5-nitrophenyl)pentanoate (150.0 g, 1.59 mol) and 10% Pd—C (75.0 g) in methanol (1.5 L) was hydrogenated at 50 psi for about 15 hours at 24° C. The solution was then filtered through a pad of celite and concentrated to give the title compound. Yield: 134 g (100%).

### Methyl-5-[5-(ethoxycarbonylamino)3-fluorophenyl]pentanoate (Step-4)

A solution of methyl 5-(3-amino-5-fluorophenyl)pentanoate (134.0 g, 0.629 mol) in dichloromethane (2.0 L) at 0° C. was treated with ethyl chloroformate (108.54 mL, 0.919 mol) followed by Hunigs base (134.84 mL). The mixture was stirred at 0° C. for 3 hours, then quenched by the addition of water (200 mL) and extracted with dichloromethane (3×200 mL). The combined organic layers were washed with brine (1×200 mL), dried over anhydrous MgSO₄ and concentrated to give the title compound. Yield: 155 g (86%), Melting point: 61-62° C.

### 5-[5-(ethoxycarbonylamino)-3-fluorophenyl]pentanoic acid (Step-5)

A solution of methyl-5-[5-(ethoxycarbonylamino)₃-fluorophenyl]pentanoate (155.0 g, 0.543 mol) in tetrahydrofuran (1.5 L) at 0° C. was treated with LiOH (52.01 g, 2.78 L, 0.78 N, 2.172 mol). The mixture was warmed to 23° C. and stirred for 15 hours. The solution was cooled to 0° C. and acidified to pH 2 with HCl (6.0 M), followed by extraction with ethyl acetate (3×500 mL). The combined organic layers were washed with brine (1×300 mL), dried over anhydrous MgSO₄, and the solvent was removed in vacuo to give 132.0 g of the title compound. Yield: 132 g (89.56%), Melting point: 136-137° C.

### Ethoxy-N-(9-fluoro-1-oxo(2,3,4,5-tetrahydrobenzo[3,4-a][7]annulen-7yl)carboxamide (Step 6)

A flask charged with 5-[5-(ethoxycarbonylamino)-3-fluorophenyl]pentanoic acid (82.0 g, 0.30 mol) was treated with 1.3 kg of Eaton's reagent (phosphorus pentoxide-methanesulfonic acid, 1:10) and stirred at 23° C. for 8 hours. The solution was quenched by the addition of water (2.0 L), stirred for 30 minutes, and extracted with ethyl acetate (3×1.0 L). The combined organic layers were washed with saturated aqueous NaHCO₃ (500 mL) and brine (300 mL), dried over anhydrous MgSO₄ and concentrated. The residue was purified by flash column chromatography (Hex-ethyl acetate 50:50) to afford 60.0 g of the title compound. Yield: 60 g (78.38%).

### (S)-N-[3-(4-Fluoro-5-oxo-6,7,8,9-tetrahydro-5H-benzocyclohepten-2-yl)-2-oxo-oxazolidin-5-ylmethyl]acetamide (Step-7)

A solution of ethoxy-N-(9-fluoro-1-oxo(2,3,4,5-tetrahydrobenzo[3,4-a][7]annulen-7yl)carboxamide (25.0 g, 0.098 mol) in a mixture of DMF (100 mL) and methanol (7.4 mL) at 23° C. was treated with lithium-t-butoxide (1M in hexane, 276 mL) drop wise over 1.5 hours. The mixture was cooled to 0° C. and (1S)-1-[(acetylamino)methyl]-2-chloroethyl acetate (35.6 g, 184 mmol) in DMF (50 mL) was added dropwise. The solution was warmed to room temperature, stirred for 17 hours, and then cooled to 0° C. Saturated aqueous ammonium chloride (200 mL) was added, followed by water (200 mL). The resulting solution was extracted with ethyl acetate (3×300 mL) and the combined organic layers were washed with brine (500 mL), dried over MgSO₄, and concentrated under vacuum to give 17.0 g of the title compound. Yield: 17 g (52.3%), Melting point: 151-152° C.

### Example 17

### (S)-N-[3-(1-Fluoro-5-oxo-6,7,8,9-tetrahydro-5H-benzocyclohepten-2-yl)-2-oxo-oxazolidin-5-ylmethyl]-acetamide

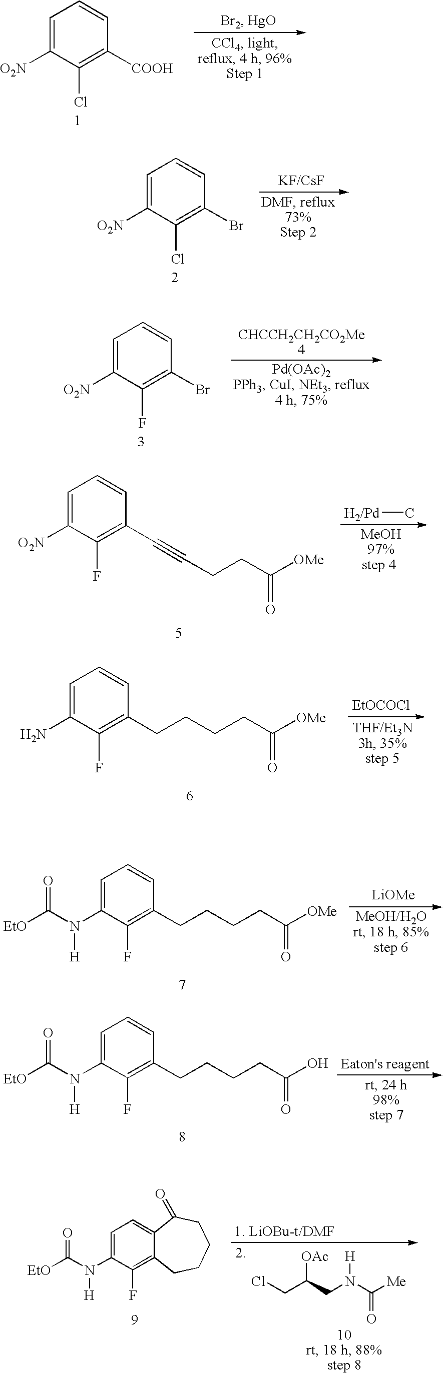

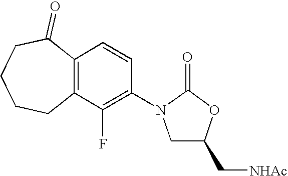

### 1-Bromo-2-chloro-3-nitro-benzene (Step 1)

To a solution of 2-chloro-3-nitro-benzoic acid (15.0 g, 74.4 mmol) in carbon tetrachloride (350 mL) was added mercury (II) oxide (24.2 g, 112 mmol) at room temperature. The mixture was heated to reflux and irradiated with light. Bromine (5.75 mL, 112 mmol) was added dropwise into the mixture in 0.5 hour. The reaction was kept at refluxing temperature for 4 hours. After cooling, the reaction mixture was quenched with 100 mL of aqueous sodium bicarbonate and stirred for 0.5 hours. After removal of the solid by filtration, the filtrate was washed with saturated sodium bicarbonate and water. Concentration gave 16.65 g of the title compound in 95% yield. ¹H NMR (400 MHz, CDCl₃): δ 7.86 (dd, 1H), 7.73 (dd, 1H), 7.30 (t, 1H).

### 1-Bromo-2-fluoro-3-nitro-benzene (Step 2)

To a solution of 1-bromo-2-chloro-3-nitro-benzene (9.47 g, 40 mmol) in N,N-dimethylformamide (20 mL) was added potassium fluoride (4.65 g, 80 mmol) and cesium fluoride (6.08 g, 40 mmol). The mixture was heated at reflux for 5 hours. After cooling, the reaction mixture was diluted with 200 mL of chloroform. The solid was collected by filtration. The filtrate was washed with saturated sodium bicarbonate and water. After removal of the solvent, the residue was purified by chromatography using chloroform as eluent to give the title compound which also contained 20% of 1-bromo-2-chloro-3-nitro-benzene. Yield 8.0 g. ¹H NMR (400 MHz, CDCl₃): δ 8.05 (m, 1H), 7.80 (m, 1H), 7.20 (m, 1H).

### 5-(2-Fluoro-3-nitro-phenyl)-pent-4-ynoic acid methyl ester (Step 3)

To a mixture of 1-bromo-2-fluoro-3-nitro-benzene (7.7 g, 35 mmol) and pent-4-ynoic acid methyl ester (5.9 g, 53 mmol) was added palladium (II) acetate (0.393 g, 1.75 mmol), triphenylphosphine (0.918 g, 3.5 mmol) and copper (I) iodide (0.667 g, 3.5 mmol). The mixture was stirred at room temperature under nitrogen atmosphere for 20 minutes then triethylamine (50 mL) was added. The mixture was heated to reflux for 4 hours. After removal of triethylamine, the residue was diluted with ethyl acetate/hexane (150 mL, 1:1). The solid was removed by filtration. The filtrate was concentrated and purified by chromatography using hexane/ethyl acetate (8:1 to :1) as eluent to give the title compound. Yield 6.56 g (75%). ¹H NMR (400 MHz, CDCl₃): δ 7.95 (m, 1H), 7.65 (m, 1H), 7.21 (m, 1H), 3.75 (s, 3H), 2.81 (m, 2H), 2.67 (m, 2H).

### 5-(2-Fluoro-3-nitro-phenyl)-pentanoic acid methyl ester (Step 4)

To a solution of 5-(2-fluoro-3-nitro-phenyl)-pent-4-ynoic acid methyl ester (6.2 g, 25 mmol) in methanol (100 mL) was added palladium on charcoal (3.0 g, 10%, wet). This mixture was hydrogenated under 45 psi at room temperature for 20 hours. Removal of the catalyst and the solvent gave the title compound. Yield 5.0 g (97%). ¹H NMR (400 MHz, CDCl₃): δ 6.85 (t, 1H), 6.67 (t, 4H), 6.57 (t, 1H), 3.65 (s, 3H), 2.62 (t, 2H), 2.35 (t, 2H), 1.65 (m, 4H).

### 5-(3-ethoxycarbonylamino-2-fluoro-phenyl)-pentanoic acid methyl ester (Step 5)

To a solution of 5-(2-fluoro-3-nitro-phenyl)-pentanoic acid methyl ester (5.0 g, 24 mmol) in tetrahydrofuran (120 mL) was added triethylamine (18 mL, 130 mmol) and ethyl chloroformate (4.8 mL, 48 mmol) dropwise at room temperature. After addition, the mixture was stirred at room temperature for 3 hours. After removal of the solvent and triethylamine, the residue was diluted with 100 mL of hexane/ethyl acetate (2:1) and stirred for 20 minutes. The solid was removed by filtration. The filtrate was concentrated and purified by chromatography using hexane/ethyl acetate (8:1 to 4:1) as eluent to give the title compound. Yield 2.0 g (28%). ¹H NMR (400 MHz, CDCl₃): δ 7.92 (br, 1H), 7.03 (t, 1H), 6.84 (m, 2H), 4.24 (q, 2H), 3.67 (s, 3H), 2.65 (t, 2H), 2.34 (t, 2H), 1.65 (m, 4H), 1.33 (t, 3H).

### 5-(3-Ethoxycarbonylamino-2-fluoro-phenyl)-pentanoic acid (Step 6)

To a solution of 5-(3-ethoxycarbonylamino-2-fluoro-phenyl)-pentanoic acid methyl ester (2.0 g, 6.7 mmol) in a mixture of methanol (50 mL) and water (10 mL) was added lithium methoxide (0.51 g, 13.4 mmol). The mixture was stirred at room temperature for 18 hours. After removal of the methanol, the aqueous solution was treated with 15 mL of 1M of hydrochloric acid (15 mmol) at 0° C. and stirred for 1 hour. The solid was collected by filtration, washed with a small amount of water, and dried to give the title compound. Yield 1.63 g (86%). ¹H NMR (400 MHz, CDCl₃): δ 7.91 (br, 1H), 7.03 (t, 1H), 6.84 (m, 2H), 4.24 (q, 2H), 2.66 (t, 2H), 2.39 (t, 2H), 1.67 (m, 4H), 1.33 (t, 3H).

### (1-Fluoro-5-oxo-6,7,8,9-tetrahydro-5H-benzocyclohepten-2-yl)-carbamic acid ethyl ester (Step 7)

5-(3-Ethoxycarbonylamino-2-fluoro-phenyl)-pentanoic acid (1.63 g, 5.76 mmol) was suspended in Eaton's reagent (30 g) and stirred at room temperature under nitrogen atmosphere for 24 hours After cooling in an ice bath, the reaction mixture was diluted with ice water (80 mL) and stirred for 1 hour. The solid was collected by filtration, washed with water, and dried to give the title compound. Yield 1.5 g (98%). ¹H NMR (400 MHz, CDCl₃): δ 8.08 (t, 1H), 7.58 (d, 1H), 7.00 (br, 1H), 4.25 (q, 2H), 2.95 (m, 2H), 2.75 (m, 2H), 1.85 (m, 4H), 1.35 (t, 3H).

### (S)-N-[3-(1-fluoro-5-oxo-6,7,8,9-tetrahydro-5H-benzocyclohepten-2-yl)-2-oxo-oxazolidin-5-ylmethyl]-acetamide (Step 8)

To a solution of (1-fluoro-5-oxo-6,7,8,9-tetrahydro-5H-benzocyclohepten-2-yl)-carbamic acid ethyl ester (1.5 g, 5.66 mmol) in a mixture of N,N-dimethylformamide (10 mL) and methanol (0.45 mL) was added lithium-tert-butoxide (1M in hexane, 17 mL, 17 mmol) dropwise at room temperature over 0.5 hours. The mixture was stirred at room temperature for 3 hours and then treated with (1S)-1-[(acetylamino)methyl]-2-chloroethyl acetate (2.2 g, 11.3 mmol) portion wise over 0.5 hour. The mixture was stirred at room temperature for 18 hours, quenched with 40 mL of saturated aqueous ammonium chloride solution, and extracted with ethyl acetate (5×50 mL). After removal of the solvent, the residue was purified by chromatography using 2.5% of methanol in chloroform as eluent to give the title compound (1.7 g, 88% yield). ¹H NMR (400 MHz, CDCl₃): δ 7.53 (d, 1H), 7.42 (t, 1H), 6.18 (t, 1H), 4.84 (m, 1H), 4.11 (dd, 1H), 3.85 (dd, 1H), 3.69 (m, 2H), 2.99 (m, 2H), 2.74 (m, 2H), 2.05 (s, 3H), 1.86 (m, 4H).

### Example 18

### (S)-N-[3-(3-Fluoro-5-oxo-6,7,8,9-tetrahydro-5H-benzocyclohepten-2-yl)-2-oxo-oxazolidin-5-ylmethyl]-acetamide

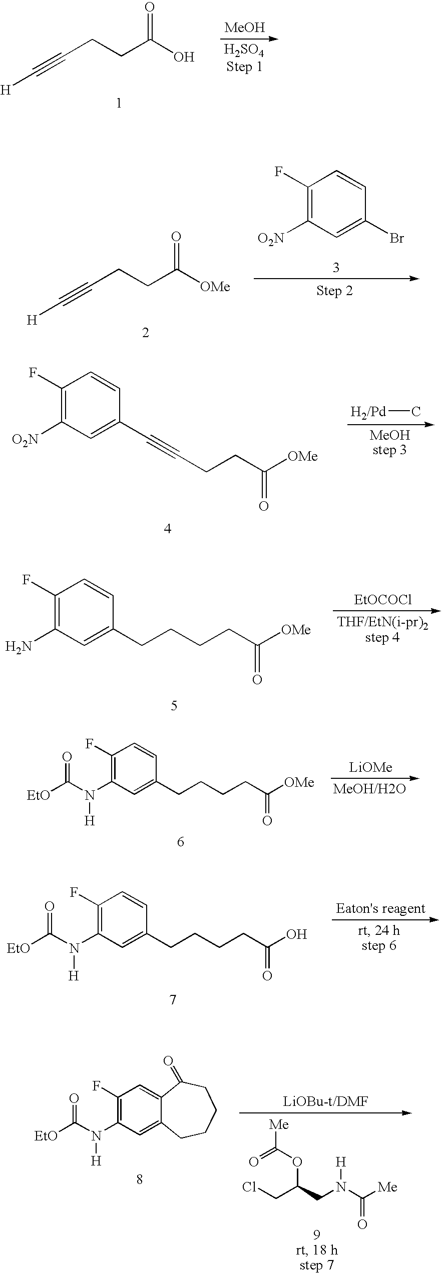

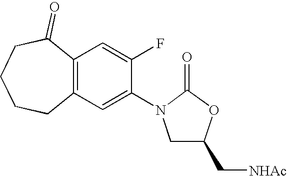

### Pent-4-ynoic acid methyl ester (Step 1)

To a solution of pent-4-ynoic acid (5.0 g, 53 mmol) in methanol (100 mL) was added concentrated sulfuric acid (3.0 g, 30 mmol) dropwise at room temperature. The resulting solution was refluxed for 20 hours. After removal of most of the methanol, the residue was dissolved in dichloromethane (100 mL) and washed with water. The dichloromethane solution was dried over sodium sulfate and concentrated under vacuum to the title compound. Yield 3.92 g (66%). ¹H NMR (400 MHz, CDCl₃): δ 3.70 (s, 3H), 2.55 (m, 4H), 2.00 (t, 1H).

### 5-(4-Fluoro-3-nitro-phenyl)-pent-4-ynoic acid methyl ester (Step 2)

To a mixture of pent-4-ynoic acid methyl ester (3.92 g, 35 mmol) and 4-bromo-1-fluoro-2-nitro-benzene (7.70 g, 35 mmol) was added palladium (II) acetate (0.393 g, 1.75 mmol), triphenylphosphine (0.918 g, 3.5 mmol) and copper (I) iodide (0.256 g, 3.5 mmol). The mixture was stirred at room temperature under a nitrogen atmosphere and triethylamine (50 mL) was added. The mixture was then heated at 100° C. for 18 hours. After removal of the triethylamine, the residue was diluted with ethyl acetate (100 mL). The solid was removed by filtration; the filtrate was concentrated and purified by chromatography using hexane-ethyl acetate (4:1) as eluent to give the title compound. Yield 4.7 g (54%). ¹H NMR (400 MHz, CDCl₃): δ 8.07 (dd, 1H), 7.61 (ddd, 1H), 7.21 (dd, 1H), 3.75 (s, 3H), 2.75 (m, 2H), 2.65 (m, 2H).

### 5-(3-Amino-4-fluoro-phenyl)-pentanoic acid methyl ester (Step 3)

To a solution of 5-(4-fluoro-3-nitro-phenyl)-pent-4-ynoic acid methyl ester (2.8 g, 11 mmol) in methanol (100 mL) was added palladium on charcoal (1.0 g, 10%, wet). This mixture was hydrogenated at 45 psi of hydrogen at room temperature for 20 hours. Removal of the catalyst and the solvent gave the title compound. Yield 2.42 g (98%). MS-ES: m/z 226 (MH⁺).

### 5-(3-Ethoxycarbonylamino-4-fluoro-phenyl)-pentanoic acid methyl ester (Step 4)

To a solution of 5-(3-amino-4-fluoro-phenyl)-pentanoic acid methyl ester (2.42 g, 11 mmol) in tetrahydrofuran (50 mL) was added diisopropylethylamine (7.7 mL, 44 mmol) and ethyl chloroformate (2.2 mL, 22 mmol) dropwise at room temperature. After addition, the mixture was stirred at room temperature for 3 hours. Removal of the solvent and diisopropylethylamine gave a residue that was diluted with 100 mL of hexane/ethyl acetate (2:1) and stirred for 20 minutes. The solid was removed by filtration. The filtrate was concentrated and purified by chromatography using hexane/ethyl acetate (8:1 to 4:1) as eluent to give the title compound. Yield 2.65 g (81%). MS-ES: m/z 298 (MH⁺).

### 5-(3-Ethoxycarbonylamino-4-fluoro-phenyl)-pentanoic acid (Step 5)

To a solution of 5-(3-ethoxycarbonylamino-4-fluoro-phenyl)-pentanoic acid methyl ester (2.58 g, 8.68 mmol) in a mixture of methanol (50 mL) and water (10 mL) was added lithium methoxide (0.66 g, 17.36 mmol). The mixture was stirred at room temperature for 18 hours. After removal of the methanol, the aqueous solution was treated with 25 mL of 1M of hydrochloric acid (25 mmol) at 0° C. and stirred for 1 hour. The solid was collected by filtration, washed with a small amount of water, and dried to give the title compound. Yield 2.31 g (94%). MS-ES: m/z 284 (MH⁺).

### (3-Fluoro-5-oxo-6,7,8,9-tetrahydro-5H-benzocyclohepten-2-yl)-carbamic acid ethyl ester (Step 6)

5-(3-Ethoxycarbonylamino-4-fluoro-phenyl)-pentanoic acid (2.23 g, 7.88 mmol) was suspended in Eaton's reagent (40 g) and stirred at room temperature under nitrogen atmosphere for 24 hours After cooling in an ice bath, the reaction mixture was diluted with water (150 mL) and stirred for 1 hour. The solid was collected by filtration, washed with water, and dried to give the title compound. Yield 2.0 g (96%). MS-ES: m/z 266 (MH⁺).

### (S)-N-[3-(3-Fluoro-5-oxo-6,7,8,9-tetrahydro-5H-benzocyclohepten-2-yl)-2-oxo-oxazolidin-5-ylmethyl]-acetamide (Step 7)

To a solution of (3-fluoro-5-oxo-6,7,8,9-tetrahydro-5H-benzocyclohepten-2-yl)-carbamic acid ethyl ester (2.0 g, 7.55 mmol) in a mixture of N,N-dimethylformamide (10 mL) and methanol (0.6 mL) was added lithium-tert-butoxide (1M in hexane, 22.7 mL, 22.7 mmol) dropwise at room temperature over 0.5 hours. The mixture was stirred at room temperature for another 3 hours and was then treated with (1S)-1-[(acetylamino)methyl]-2-chloroethyl acetate (2.92 g, 15.1 mmol) in one portion. This mixture was stirred at room temperature for 18 hours, quenched with 40 mL of saturated aqueous ammonium chloride solution and extracted with ethyl acetate (5×50 mL). After removal of the solvent, the residue was purified by chromatography using 2.5% of methanol in chloroform as eluent to give the title compound. Yield 0.83 g (53%). MS-ES: m/z 335 (MH⁺).

### Example 19

### (S)-N-[3-(1,4-Difluoro-5-oxo-6,7,8,9-tetrahydro-5H-benzocyclohepten-2-yl)-2-oxo-oxazolidin-5-ylmethyl]-acetamide

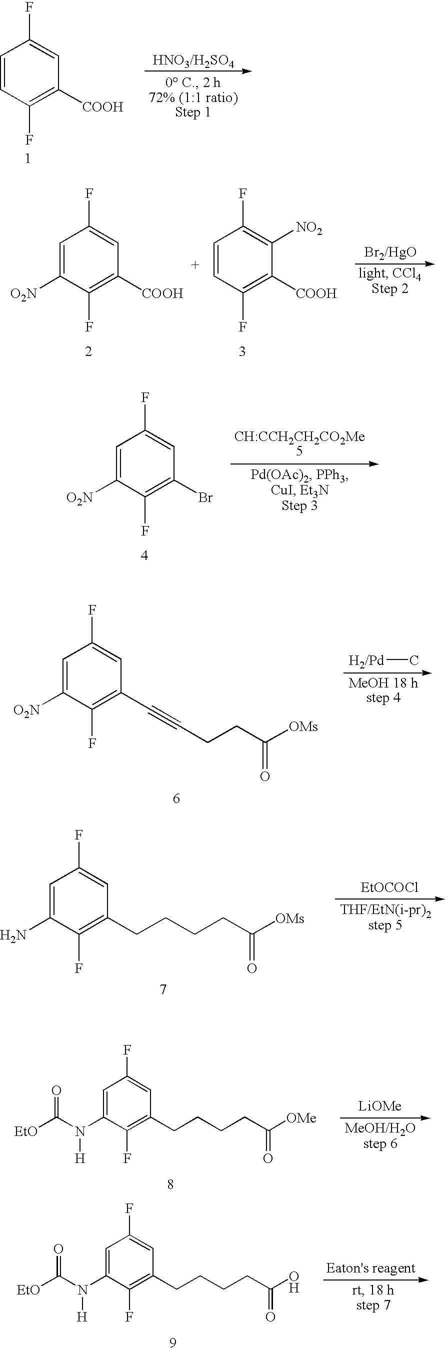

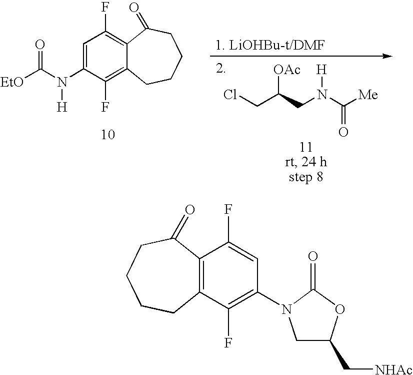

### 2,5-Difluoro-3-nitro-benzoic acid (Step 1)

To a solution of 2,5-difluoro-benzoic acid (3.66 g, 23 mmol) in concentrated sulfuric acid (10 mL) was added a mixture of 3 mL of nitric acid (90%, fuming) and 3 mL of concentrated sulfuric acid dropwise at 0° C. The reaction mixture was stirred at 0° C. for 2 hours. The resulting mixture was then poured onto ice and extracted with ethyl acetate (3×30 mL). After removal of the solvent, the residue was purified by chromatography using 5% of methanol in chloroform as eluent. A mixture of 2,5-difluoro-3-nitro-benzoic acid and 3,6-difluoro-2-nitro-benzoic acid was obtained. Yield 2.48 g. ¹H NMR (400 MHz, CDCl₃): 2,5-difluoro-3-nitro-benzoic acid: δ 8.36 (ddd, 1H), 8.07 (ddd, 1H).

### 1-Bromo-2,5-difluoro-3-nitro-benzene (Step 2)

To a solution of the mixture of 2,5-difluoro-3-nitro-benzoic acid and 3,6-difluoro-2-nitro-benzoic acid (2.48 g, 12.2 mmol, ratio 2:1) in carbon tetrachloride (60 mL) was added mercury (II) oxide (red) (5.2 g, 24 mmol) at room temperature. The mixture was heated to reflux and irradiated with light. Bromine (3.8 g, 24 mmol) was added dropwise to the mixture in 10 minutes. The reaction mixture was heated at reflux for 6 hours. After cooling, the solid was removed by filtration and the filtrate was washed with saturated sodium bicarbonate and water. After removal of the solvent, the residue was purified by chromatography using hexane/ethyl acetate (8:1) to give the title compound. Yield 1.07 g (38%). ¹H NMR (400 MHz, CDCl₃): δ 7.76 (ddd, 1H), 7.64 (ddd, 1H).

### 5-(2,5-Difluoro-3-nitro-phenyl)-pent-4-ynoic acid methyl ester (Step 3)

To a mixture of 1-bromo-2,5-difluoro-3-nitro-benzene (1.0 g, 4.2 mmol) and pent-4-ynoic acid methyl ester (0.94 g, 8.4 mmol) were added palladium (II) acetate (0.094 g, 0.42 mmol), triphenylphosphine (0.22 g, 0.84 mmol) and copper (I) iodide (0.08 g, 0.42 mmol). The mixture was stirred at room temperature under nitrogen atmosphere for 20 minutes and then treated with triethylamine (8 mL). The mixture was heated to reflux for 4 hours. After removal of the triethylamine, the residue was diluted with ethyl acetate (50 mL). The solid was removed by filtration. The filtrate was concentrated and purified by chromatography using hexane/ethyl acetate (8:1 to 4:1) as eluent to give the title compound in 72% yield (0.81 g). ¹H NMR (400 MHz, CDCl₃): δ 7.68 (ddd, 1H), 7.38 (ddd, 1H), 3.74 (s, 3H), 2.81 (t, 2H), 2.67 (t, 2H).

### 5-(3-Amino-2,5-difluoro-phenyl)-pentanoic acid methyl ester (Step 4)

To a solution of 5-(2,5-difluoro-3-nitro-phenyl)-pent-4-ynoic acid methyl ester (0.8 g, 3 mmol) in methanol (40 mL) was added palladium on charcoal (0.8 g, 10%, wet). The mixture was hydrogenated under 45 psi of hydrogen at room temperature for 20 hours. After removal of the catalyst and the solvent, the title compound was obtained. Yield 0.65 g (91%). ¹H NMR (400 MHz, CDCl₃): δ 6.39 (ddd, 1H), 6.28 (ddd, 1H), 3.65 (s, 3H), 3.45 (br, 2H), 2.59 (t, 2H), 2.34 (t, 2H), 1.65 (m, 4H).

### 5-(3-Ethoxycarbonylamino-2,5-difluoro-phenyl)-pentanoic acid methyl ester (Step 5)

To a solution of 5-(3-amino-2,5-difluoro-phenyl)-pentanoic acid methyl ester (0.65 g, 2.7 mmol) in tetrahydrofuran (50 mL) were added triethylamine (18 mL, 130 mmol) and ethyl chloroformate (0.9 mL, 9 mmol) dropwise at 0° C., followed by diisopropylethylamine (3 mL). The mixture was stirred at 0° C. for 1 hour and at room temperature for 48 hours. After removal of the solvent and diisopropylethylamine, the residue was purified by chromatography using hexane/ethyl acetate (4:1) as eluent to give the title compound. Yield 0.76 g (89%). ¹H NMR (400 MHz, CDCl₃): δ 7.78 (br, 1H), 6.87 (br, 1H), 6.54 (ddd, 1H), 4.25 (q, 2H), 3.67 (s, 3H), 2.63 (t, 2H), 2.34 (t, 2H), 1.65 (m, 4H), 1.33 (t, 3H).

### 5-(3-Ethoxycarbonylamino-2,5-difluoro-phenyl)-pentanoic acid (Step 6)

To a solution of 5-(3-ethoxycarbonylamino-2,5-difluoro-phenyl)-pentanoic acid methyl ester (0.75 g, 2.38 mmol) in a mixture of methanol (20 mL) and water (4 mL) was added lithium methoxide (0.18 g, 4.8 mmol). The mixture was stirred at room temperature for 18 hours. After removal of methanol, the aqueous solution was cooled to 0° C. and treated with 6 mL of 1M hydrochloric acid (6 mmol) and stirred for 0.5 hour. The solid was collected by filtration, washed with a small amount of water, and dried to give the title compound. Yield 0.588 g (82%). ¹H NMR (400 MHz, CDCl₃): δ 7.75 (br, 1H), 6.88 (br, 1H), 6.55 (m, 1H), 4.25 (q, 2H), 2.64 (t, 2H), 2.39 (t, 2H), 1.67 (m, 4H), 1.33 (t, 3H).

### (1,4-Difluoro-5-oxo-6,7,8,9-tetrahydro-5H-benzocyclohepten-2-yl)-carbamic acid ethyl ester (Step 7)

5-(3-Ethoxycarbonylamino-2,5-difluoro-phenyl)-pentanoic acid (0.58 g, 1.93 mmol) was added portion wise into Eaton's reagent (10 g) at room temperature and stirred at room temperature under nitrogen atmosphere for 18 hours. After cooling in an ice bath, the reaction mixture was diluted with ice water (50 mL) and extracted with ethyl acetate (2×30 mL). Removal of the solvent gave a the residue which was purified by chromatography using hexane/ethyl acetate (8:1 to 4:1) as eluent. Yield 0.28 g (51%). ¹H NMR (400 MHz, CDCl₃): δ 7.88 (dd, 1H), 7.01 (br, 1H), 4.27 (q, 2H), 2.88 (m, 2H), 2.67 (m, 2H), 1.83 (m, 4H), 1.34 (t, 3H).

### (S)-N-[3-(1,4-Difluoro-5-oxo-6,7,8,9-tetrahydro-5H-benzocyclohepten-2-yl)-2-oxo-oxazolidin-5-ylmethyl]-acetamide (Step 8)

To a solution of (1,4-difluoro-5-oxo-6,7,8,9-tetrahydro-5H-benzocyclohepten-2-yl)-carbamic acid ethyl ester (0.28 g, 1.0 mmol) in a mixture of N,N-dimethylformamide (2 mL) and methanol (0.1 mL) was added lithium-tert-butoxide (1M in hexane, 3 mL, 3 mmol) dropwise at room temperature over 0.5 hour. The mixture was stirred at room temperature for 3 hours. To the mixture compound was added (1S)-1-[(acetylamino)methyl]-2-chloroethyl acetate (0.39 g, 2 mmol) portion wise over 0.5 hour. The mixture was stirred at room temperature for 24 hours, quenched with 40 mL of saturated aqueous ammonium chloride solution and extracted with ethyl acetate (3×30 mL). After removal of the solvent, the residue was purified by chromatography using 2.5% to 5% of methanol in chloroform as eluent to give the title compound. Yield 0.205 g (58%). ¹H NMR (400 MHz, CDCl₃): δ 7.25 (dd, 1H), 6.53 (br, 1H), 4.84 (m, 1H), 4.13 (t, 1H), 3.88 (t, 1H), 3.68 (m, 2H), 2.88 (m, 2H), 2.70 (m, 2H), 2.04 (s, 3H), 1.86 (m, 4H).

### Example 20

### (S)-N-[2-Oxo-3-(9-oxo-6,7,8,9-tetrahydro-5H-cyclohepta[b]pyridin-3-yl)-oxazolidin-5-ylmethyl]-acetamide

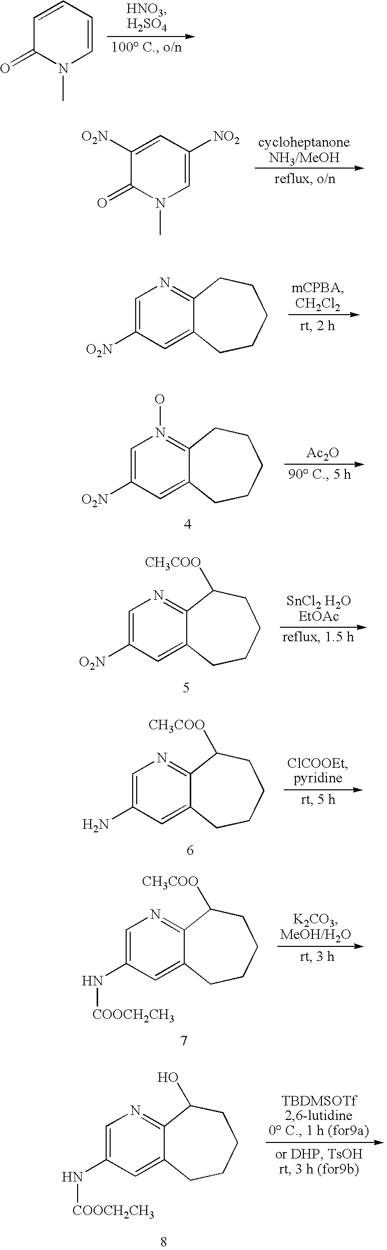

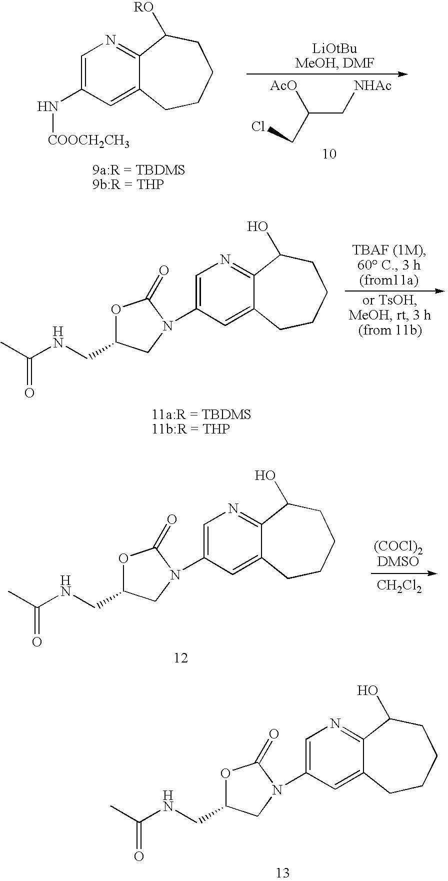

### 1-Methyl-3,5-dinitro-1H-pyridin-2-one (Step 1)

To a stirred mixture of 1-methyl-1H-pyridin-2-one (33.47 g, 0.306 mol) and concentrated H₂SO₄ (300 mL) at 100° C. was added concentrated HNO₃ (120 mL) in portions. The reaction mixture was heated at this temperature overnight, and then poured into ice (1400 mL). The precipitate was filtered off and washed with water to give the title compound (Yield: 18.66 g, 30%). ¹H NMR (400 MHz, CDCl₃): δ 9.05 (d, 1H), 8.90 (d, 1H), 3.80 (s, 3H).

### 3-Nitro-6,7,8,9-tetrahydro-5H-cyclohepta[b]pyridine (Step 2)

A mixture of 1-methyl-3,5-dinitro-1H-pyridin-2-one (16.00 g, 80 mmol), cycloheptanone (9.95 mL, 84 mmol) and 20% ammonia solution in methanol (300 mL) was refluxed overnight. The solvent was evaporated, and the residue was dissolved in ethyl acetate. The soluble portion was concentrated and purified by flash chromatography (Hexane/Ethyl acetate 3:1, silica gel) to yield the title compound (Yield: 14.00 g, 91%). ¹H NMR (400 MHz, CDCl₃): δ 9.05 (dd, 1H), 8.08 (dd, 1H), 3.10 (m, 2H), 2.90 (m, 2H), 1.90 (m, 2H), 1.70 (m, 4H).

### 3-Nitro-6,7,8,9-tetrahydro-5H-cyclohepta[b]pyridin-1-ol (Step 3)

To a solution of 3-nitro-6,7,8,9-tetrahydro-5H-cyclohepta[b]pyridine (1.20 g, 6.25 mmol) in dichloromethane (14 mL) was added m-CPBA (56-80%, 2.00 g). The resulting reaction mixture was stirred at room temperature for 2 hours. Di-isopropyl ether was then added and the solids that formed were collected by filtration (Yield: 1.00 g, 77%). ¹H NMR (400 MHz, CDCl₃): δ 8.95 (d, 1H), 7.75 (d, 1H), 3.42 (m, 2H), 2.92 (m, 2H), 1.90 (m, 2H), 1.72 (m, 4H).

### Carbonic acid methyl ester 3-nitro-6,7,8,9-tetrahydro-5H-cyclohepta[b]pyridin-9-yl ester (Step 4)

A mixture of 3-nitro-6,7,8,9-tetrahydro-5H-cyclohepta[b]pyridin-1-ol (0.90 g, 4.32 mmol) and acetic anhydride (6.00 mL) was heated at 90° C. for 5 hours, and the excess acetic anhydride was removed under reduced pressure. The residue thus obtained was purified by chromatography (4% MeOH/CH₂Cl₂) to give the title compound (Yield: 0.93 g, 86%). MS, m/Z (C₁₂H₁₄NO₄): 250.81 (MH⁺, 100%).

### Carbonic acid 3-amino-6,7,8,9-tetrahydro-5H-cyclohepta[b]pyridin-9-yl ester methyl ester (Step 5)

A mixture of carbonic acid methyl ester 3-nitro-6,7,8,9-tetrahydro-5H-cyclohepta[b]pyridin-9-yl ester (0.93 g, 3.72 mmol) and SnCl₂.2H₂O (4.19 g, 18.60 mmol) in ethyl acetate (30 mL) was refluxed for 1.5 hours. To the cooled reaction mixture was added a saturated solution of Na₂CO₃ to adjust the pH to 8, and then water was added. The aqueous layer was extracted with ethyl acetate (3×30 mL). The combined organic layers were evaporated to give the title compound (0.80 g), which was used in the next step without further purification. ¹H NMR (400 MHz, CDCl₃): δ 7.80 (d, 1H), 6.70 (d, 1H), 5.90 (m, 1H), 3.60 (br s, 2H), 2.10 (s, 3H), 2.95-1.60 (m, 9H).

### Carbonic acid 3-ethoxycarbonylamino-6,7,8,9-tetrahydro-5H-cyclohepta[b]pyridin-9-yl ester methyl ester (Step 6)

To a solution of carbonic acid 3-amino-6,7,8,9-tetrahydro-5H-cyclohepta[b]pyridin-9-yl ester methyl ester (0.80 g) in dichloromethane (20 mL) was added pyridine (0.58 mL) and ethyl chloroformate (0.52 mL, 5.45 mmol), and the resulting mixture was stirred at room temperature for 5 hours. The mixture was washed with brine (2×15 mL), dried over sodium sulfate and concentrated to give a crude product, which was purified by chromatography to yield the title compound (Yield: 0.70 g, 66% over two steps). ¹H NMR (400 MHz, CDCl₃): δ 8.08 (d, 1H), 7.80 (br s, 1H), 6.68 (br s, 1H), 5.90 (m, 1H), 4.12 (q, 2H), 2.20 (s, 3H), 2.95-1.60 (m, 9H), 1.30 (t, 3H).

### (9-Hydroxy-6,7,8,9-tetrahydro-5H-cyclohepta[b]pyridin-3-yl)-carbamic acid ethyl ester (Step 7)

To a solution of carbonic acid 3-ethoxycarbonylamino-6,7,8,9-tetrahydro-5H-cyclohepta[b]pyridin-9-yl ester methyl ester (0.70 g, 2.40 mmol) in a mixture of methanol (8 mL) and water (2 mL) was added K₂CO₃ (0.50 g, 3.60 mmol). The reaction mixture was stirred at room temperature for 2.5 hours and then the methanol was evaporated. The residue was treated with ethyl acetate (15 mL) and water (15 mL), and the aqueous layer was extracted with ethyl acetate (3×10 mL). The combined organic layers were dried over sodium sulfate and evaporated to give a crude product, which was purified by chromatography to yield the title compound (Yield: 0.37 g, 62%). ¹H NMR (400 MHz, CDCl₃): δ 8.10 (d, 1H), 7.70 (br s, 1H), 6.60 (br s, 1H), 5.68 (d, 1H), 4.70 (m, 1H), 2.80-1.20 (m, 9H).

### [9-(tert-Butyl-dimethyl-silanyloxy)-6,7,8,9-tetrahydro-5H-cyclohepta[b]pyridin-3-yl]-carbamic acid ethyl ester (Step 8)

To a solution of (9-hydroxy-6,7,8,9-tetrahydro-5H-cyclohepta[b]pyridin-3-yl)-carbamic acid ethyl ester (0.214 g, 0.856 mmol) in dichloromethane (6 mL) at 0° C. were added 2,6-lutidine (0.15 mL, 1.28 mmol) and t-butyl dimethylsilyl trifluoromethane sulfonate (0.30 mL, 1.28 mmol). The resulting reaction mixture was stirred at this temperature for 1 hour and then quenched with water. The aqueous layer was extracted with dichloromethane (2×10 mL), dried with sodium sulfate and concentrated to give a crude product, which was purified by chromatography to give the title compound (Yield: 0.284 g, 99%). ¹H NMR (400 MHz, CDCl₃): δ 8.38 (d, 1H), 7.86 (br s, 1H), 6.75 (br s, 1H), 5.20 (d, 1H), 4.45 (q, 2H), 3.55 (t, 1H), 2.76 (m, 1H), 2.50 (m, 1H), 2.20 (m, 2H), 1.90 (m, 1H), 1.84 (m, 1H), 1.60 (m, 1H), 1.50 (t, 3H), 1.05 (s, 9H), 0.20 (s, 6H).

### [9-(Tetrahydro-pyran-2-yloxy)-6,7,8,9-tetrahydro-5H-cyclohepta[b]pyridin-3-yl]-carbamic acid ethyl ester (Step 8)

To a solution of (9-hydroxy-6,7,8,9-tetrahydro-5H-cyclohepta[b]pyridin-3-yl)-carbamic acid ethyl ester (0.774 g, 3.096 mmol) in dichloromethane (15 mL) were added dihydropyran (0.537 mL, 6.200 mmol) and p-toluenesulfonic acid (0.66 g, 3.40 mmol). The reaction mixture was stirred at room temperature for 3 hours, then diluted with dichloromethane (15 mL) and washed with saturated NaHCO₃ solution. The organic layer was dried (NaSO₄) and evaporated. The crude product was purified by chromatography to afford two isomers of the title compound. (Yield: 0.934 g, 90%). ¹H NMR (400 MHz, CDCl₃): δ Isomer 1: 8.20 (d, 1H), 7.76 (br s, 1H), 6.60 (br s, 1H), 4.90 (d, 1H), 4.82 (m, 1H), 4.25 (q, 2H), 3.55 (t, 1H), 3.30 (m, 2H), 2.60 (m, 1H), 2.20-1.40 (m, 12H), 1.30 (t, 3H). Isomer 2: 8.18 (d, 1H), 7.76 (br s, 1H), 6.60 (br s, 1H), 5.05 (d, 1H), 4.45 (m, 1H), 4.25 (q, 2H), 3.95 (m, 1H), 3.50 (m, 1H), 3.10 (m, 1H), 2.58 (m, 1H), 2.20-1.40 (m, 12H), 1.30 (t, 3H).

### N-{3-[9(R,S)-(tert-Butyl-dimethyl-silanyloxy)-6,7,8,9-tetrahydro-5H-cyclohepta[b]pyridin-3-yl]-2-oxo-oxazolidin-5(S)-ylmethyl}-acetamide (Step 9)

A solution of [9-(tert-butyl-dimethyl-silanyloxy)-6,7,8,9-tetrahydro-5H-cyclohepta[b]pyridin-3-yl]-carbamic acid ethyl ester (0.270 g, 0.808 mmol) in a mixture of DMF (1.50 mL) and methanol (0.065 mL, 1.62 mmol) at room temperature was treated dropwise with lithium t-butoxide (1M solution in hexane, 2.42 mL, 2.42 mmol) and stirred for 1 hour. The mixture was cooled to 0° C. and (1S)-1-[(acetylamino)methyl]-2-chloroethyl acetate (0.312 g, 1.62 mmol) was added in one portion. The resulting mixture was warmed to room temperature, stirred overnight, and then quenched with saturated ammonium chloride solution. The aqueous layer was extracted with ethyl acetate. The combined organic layers were washed with brine, dried over sodium sulfate and evaporated. The crude product was purified by chromatography to give title compound as a mixture of isomers (Yield: 0.214 g, 61%). The product was directly used in the next step without further isomer separation.

### N-{2-Oxo-3-[9-(tetrahydro-pyran-2-yloxy)-6,7,8,9-tetrahydro-5H-cyclohepta[b]pyridin-3-yl]-oxazolidin-5(S)-ylmethyl}acetamide (Step 9)

A solution of [9-(tetrahydro-pyran-2-yloxy)-6,7,8,9-tetrahydro-5H-cyclohepta[b]pyridin-3-yl]-carbamic acid ethyl ester (0.934 g, 2.796 mmol) in a mixture of N,N-dimethylformamide (5.0 mL) and methanol (0.226 mL, 5.59 mmol) at room temperature was treated dropwise with lithium t-butoxide (1M solution in hexane, 8.39 m/L, 8.39 mmol) and stirred for 1 hour. The mixture was cooled to 0° C. and (1S)-1-[(acetylamino)methyl]-2-chloroethyl acetate (1.08 g, 5.59 mmol) was added in one portion. The resulting mixture was warmed to room temperature, stirred overnight, and then quenched with saturated ammonium chloride solution. The aqueous layer was extracted with ethyl acetate. The combined organic layers were washed with brine, dried over sodium sulfate and evaporated. The crude product was quickly purified by chromatography to give a mixture of two isomers of the title compound (Yield: 0.80 g, 71%), which was directly used in the next step without further isomer separation.

### N-[3-(9(R,S)-Hydroxy-6,7,8,9-tetrahydro-5H-cyclohepta[b]pyridin-3-yl)-2-oxo-oxazolidin-5(S)-ylmethyl]-acetamide (Step 10)

### Method A: Synthesis from N-[3-[9(R,S)-(tert-Butyl-dimethyl-silanyloxy)-6,7,8,9-tetrahydro-5H-cyclohepta[b] pyridin-3-yl]-2-oxo-oxazolidin-5(S)-ylmethyl]-acetamide

N-{3-[9(R,S)-(tert-butyl-dimethyl-silanyloxy)-6,7,8,9-tetrahydro-5H-cyclohepta[b]pyridin-3-yl]-2-oxo-oxazolidin-5(S)-ylmethyl}-acetamide (0.220 g, 0.50 mmol) was treated with 1 M tetrabutylammonium fluoride solution in THF at 60° C. for 3 hours. The reaction mixture was cooled to room temperature, treated with saturated NH₄Cl solution, and extracted with ethyl acetate (3×10 mL). The combined organic layers were washed with brine, dried over sodium sulfate and evaporated to give the crude product, which was purified by chromatography to give the title compound (Yield: 0.064 g, 40%). m/z (C₁₆H₂₁O₄N₃): 320.06 (MH⁺, 100%).

### Method B: Synthesis from N-{2-Oxo-3-[9-(tetrahydro-pyran-2-yloxy)-6,7,8,9-tetrahydro-5H-cyclohepta[b]pyridin-3-yl]-oxazolidin-5(S)-ylmethyl}acetamide

To a solution of N-{2-oxo-3-[9-(tetrahydro-pyran-2-yloxy)-6,7,8,9-tetrahydro-5H-cyclohepta[b]pyridin-3-yl]-oxazolidin-5(S)-ylmethyl}-acetamide (0.746 g, 1.85 mmol) in methanol (8 mL) was added p-toluenesulfonic acid (0.46 g, 2.40 mmol) and the reaction mixture was stirred at room temperature for 3 hours. The solvent was evaporated under vacuum; the residue was treated with ethyl acetate, washed with saturated NaHCO₃ solution until the aqueous layer was basic (pH 8.5), and finally washed with brine. The organic layer was dried and evaporated to give residue, which was purified by chromatography to give the title compound. (Yield: 0.564 g, 91%).

### (S)-N-[2-Oxo-3-(9-oxo-6,7,8,9-tetrahydro-5H-cyclohepta[b]pyridin-3-yl)-oxazolidin-5-ylmethyl]-acetamide (Step 11)

To a solution of oxalyl chloride (0.164 mL, 1.886 mmol) in anhydrous dichloromethane (4 mL) at −78° C. was added DMSO (0.134 mL, 1.886 mmol) in dichloromethane (0.50 mL) and stirring was continued for 2 minutes. N-[3-(9(R,S)-Hydroxy-6,7,8,9-tetrahydro-5H-cyclohepta[b]pyridin-3-yl)-2-oxo-oxazolidin-5(S)-ylmethyl]-acetamide (0.463 g, 1.45 mmol) in anhydrous dichloromethane (2 mL) was added and the reaction mixture was stirred at this temperature for 30 minutes. Triethylamine (1.01 mL, 7.25 mmol) was added and stirring was continued at −78° C. for 5 minutes and then allowed to warm to room temperature. Water (8 mL) was added and the aqueous layer was extracted with dichloromethane. The combined organic layers were washed with brine, dried over sodium sulfate, and evaporated. The residue was purified by chromatography to give the title compound (Yield: 0.306 g, 66%). M/Z (C₁₆H₁₉O₄N₃): 318.10 (MH⁺, 100%), 274.07 (MH⁺—CO₂, 40%).

### Example 21

### (S)-N-[3-(6-Dimethylaminomethylene-1-fluoro-5-oxo-6,7,8,9-tetrahydro-5H-benzocyclohepten-2-yl)-2-oxo-oxazolidin-5-ylmethyl]-acetamide

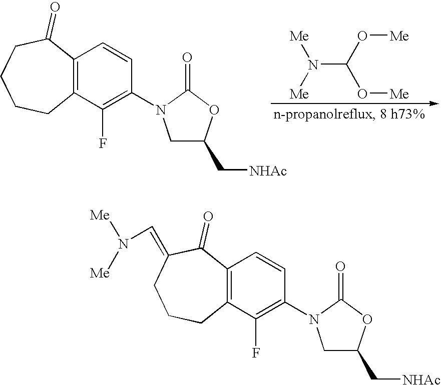

To a solution of (S)-N-[3-(1-fluoro-5-oxo-6,7,8,9-tetrahydro-5H-benzocyclohepten-2-yl)-2-oxo-oxazolidin-5-ylmethyl]-acetamide (0.48 g, 1.44 mmol) in n-propanol (15 mL) was added dimethylformamide dimethyl acetal (0.68 g, 5.75 mmol). The solution was heated to reflux for 8 hours. After removal of the solvent, the residue was purified by chromatography using 5% methanol in chloroform as eluent to give the title compound. Yield 0.41 g (59%). ¹H NMR (400 MHz, CDCl₃): δ 7.75 (s, 1H), 7.42 (d, 1H), 7.25 (t, 1H), 6.20 (t, 1H), 4.80 (m, 1H), 4.10 (m, 1H), 3.80 (m, 1H), 3.60 (m, 2H), 3.15 (s, 6H), 2.80 (m, 2H), 2.35 (m, 2H), 2.00 (s, 3H), 1.80 (m, 2H).

### Example 22

### (S)-N-[3-(7-Fluoro-5,6-dihydro-4H-1-oxa-2-aza-benzo[e]azulen-8-yl)-2-oxo-oxazolidin-5-ylmethyl]-acetamide

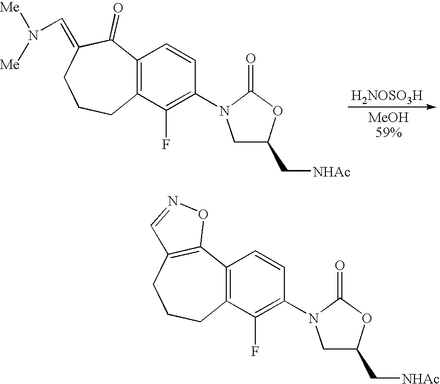

To a solution of (S)-N-[3-(6-dimethylaminomethylene-1-fluoro-5-oxo-6,7,8,9-tetrahydro-5H-benzocyclohepten-2-yl)-2-oxo-oxazolidin-5-ylmethyl]-acetamide (0.40 g, 1.03 mmol) in methanol (15 mL) was added hydroxylamine-O-sulfonic acid (0.14 g, 1.24 mmol) at 0° C. The solution was stirred at 0° C. for 0.5 hour, then at room temperature for 8 hours. After removal of the solvent, the residue was purified by chromatography using 5% methanol in chloroform as eluent to give the target compound. Yield 0.218 g (59%). ¹H NMR (400 MHz, CDCl₃): δ 8.18 (s, 1H), 7.82 (d, 1H), 7.42 (t, 1H), 6.08 (t, 1H), 4.40 (m, 1H), 4.10 (dd, 1H), 3.87 (dd, 1H), 3.70 (m, 2H), 2.98 (m, 2H), 2.82 (m, 2H), 2.05 (s, 3H), 2.00 (m, 2H).

### Example 23

### (S)-N-[3-(2-Amino-8-fluoro-6,7-dihydro-5H-benzo[6,7]cyclohepta[1,2-d]pyrimidin-9-yl)-2-oxo-oxazolidin-5-ylmethyl]-acetamide

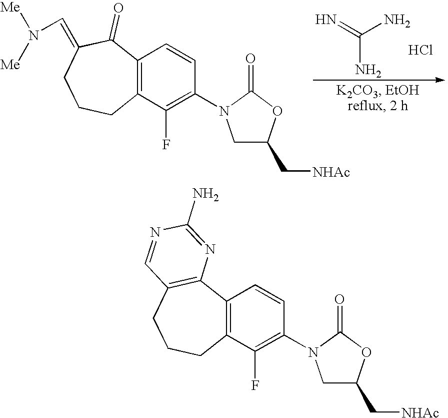

To a solution of (S)-N-[3-(6-dimethylaminomethylene-1-fluoro-5-oxo-6,7,8,9-tetrahydro-5H-benzocyclohepten-2-yl)-2-oxo-oxazolidin-5-ylmethyl]-acetamide (0.20 g, 0.514 mmol) in ethanol (15 mL) were added guanidine hydrochloride (0.49 g, 0.514 mmol) and potassium carbonate (0.71 g, 0.514 mmol). The mixture was refluxed for 3 hours. After cooling, the reaction mixture was diluted with chloroform (30 mL) and stirred at room temperature for 10 minutes. After filtration, the filtrate was concentrated. The residue was purified by chromatography using 5% to 10% methanol in chloroform as eluent to give the title compound. Yield 0.185 g (93%). ¹H NMR (400 MHz, CDCl₃): δ 8.16 (s, 1H), 7.51 (d, 1H), 7.46 (t, 1H), 6.34 (t, 1H), 5.11 (s, 2H), 4.85 (m, 1H), 4.11 (dd, 1H), 3.86 (dd, 1H), 3.85 (dd, 1H), 3.69 (m, 2H), 2.64 (m, 2H), 2.38 (t, 2H), 2.18 (m, 2H), 2.05 (s, 3H).

### Example 24

### (S)-N-[3-(7-Fluoro-1,4,5,6-tetrahydro-1,2-diaza-benzo[e]azulen-8-yl)-2-oxo-oxazolidin-5-ylmethyl]-acetamide

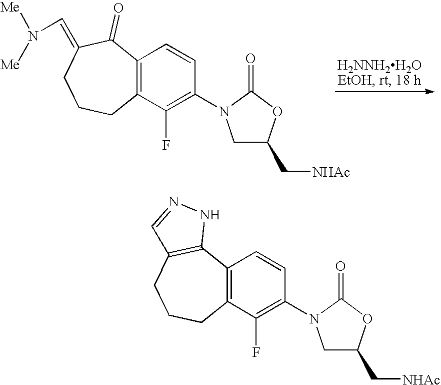

To a solution of (S)-N-[3-(6-dimethylaminomethylene-1-fluoro-5-oxo-6,7,8,9-tetrahydro-5H-benzocyclohepten-2-yl)-2-oxo-oxazolidin-5-ylmethyl]-acetamide (0.375 g, 0.96 mmol) in ethanol (10 mL) was added hydrazine hydrate (0.193 g, 3.86 mmol). The mixture was stirred at room temperature for 18 hours. After removal of the solvent, the residue was purified by chromatography using 5% to 10% methanol in chloroform as eluent to give the title compound. Yield 0.258 g (76%). ¹H NMR (400 MHz, CDCl₃): δ 7.50 (m, 2H), 7.26 (m, 1H), 6.80 (br, 1H), 4.88 (m, 1H), 4.12 (t, 1H), 3.85 (m, 1H), 3.75 (dd, 1H), 3.58 (m, 1H), 3.02 (m, 1H), 2.84 (m, 2H), 2.72 (m, 1H), 2.00 (m, 5H).

### Example 25

### (S)-N-[3-(6-Dimethylaminomethylene-3-fluoro-5-oxo-6,7,8,9-tetrahydro-5H-benzocyclohepten-2-yl)-2-oxo-oxazolidin-5-ylmethyl]-acetamide

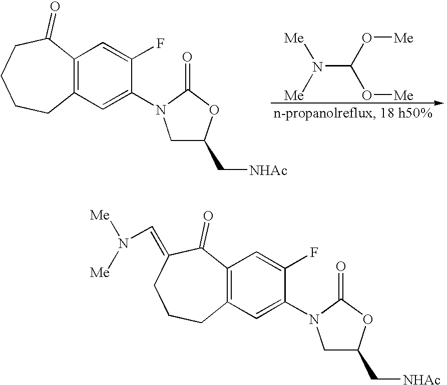

To a solution of (S)-N-[3-(3-fluoro-5-oxo-6,7,8,9-tetrahydro-5H-benzocyclohepten-2-yl)-2-oxo-oxazolidin-5-ylmethyl]-acetamide (0.5 g, 1.5 mmol) in n-propanol (10 mL) was added dimethylformamide dimethylacetal (0.71 g, 6.0 mmol). The solution was heated at 110° C. for 18 hours. After removal of the solvent, the residue was purified by chromatography using 5% methanol in chloroform as eluent to give the title compound. Yield 0.27 g (48%). MS-ES: m/z 390 (MH⁺).

### Example 26

### (S)-N-[3-(9-Fluoro-5,6-dihydro-4H-1-oxa-2-aza-benzo[e]azulen-8-yl)-2-oxo-oxazolidin-5-ylmethyl]-acetamide

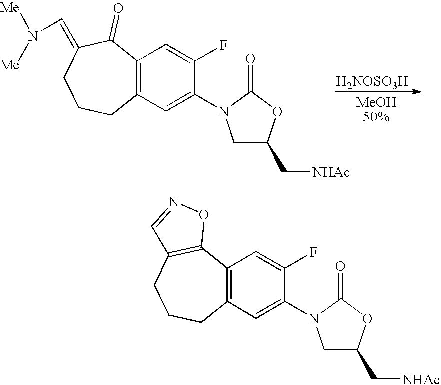

To a solution of (S)-N-[3-(6-dimethylaminomethylene-3-fluoro-5-oxo-6,7,8,9-tetrahydro-5H-benzocyclohepten-2-yl)-2-oxo-oxazolidin-5-ylmethyl]-acetamide (0.25 g, 0.65 mmol) in methanol (8 mL) was added hydroxylamine-O-sulfonic acid (0.088 g, 0.78 mmol) at 0° C. The solution was stirred at 0° C. for 1 hour, then at room temperature for 4 hours. After removal of the solvent, the residue was purified by chromatography using 5% methanol in chloroform as eluent to give the target compound. Yield 0.118 g (50%). MSES: m/z 360 (MH⁺).

### Example 27

### (S)-N-[3-(2-Amino-10-fluoro-6,7-dihydro-5H-benzo[6,7]cyclohepta[1,2-d]pyrimidin-9-yl)-2-oxo-oxazolidin-5-ylmethyl]-acetamide

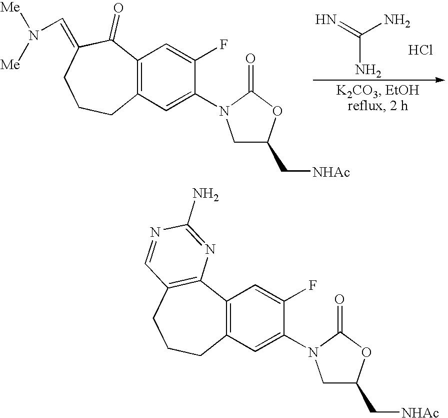

To a solution of (S)-N-[3-(6-dimethylaminomethylene-3-fluoro-5-oxo-6,7,8,9-tetrahydro-5H-benzocyclohepten-2-yl)-2-oxo-oxazolidin-5-ylmethyl]-acetamide (0.68 g, 1.75 mmol) in ethanol (30 mL) was added guanidine hydrochloride (1.67 g, 17.5 mmol) and potassium carbonate (2.42 g, 17.5 mmol). The mixture was refluxed for 2 hours. After cooling, the reaction mixture was diluted with chloroform (50 mL) and stirred at room temperature for 10 minutes. The solids were filtered and the filtrate was concentrated. The residue was purified by chromatography using 5% methanol in chloroform as eluent to give the title compound. Yield 0.44 g (65%). MS-ES: m/z 386 (MH⁺).

### Example 28

### (S)-N-[3-(9-Fluoro-1,4,5,6-tetrahydro-1,2-diaza-benzo[e]azulen-8-yl)-2-oxo-oxazolidin-5-ylmethyl]-acetamide

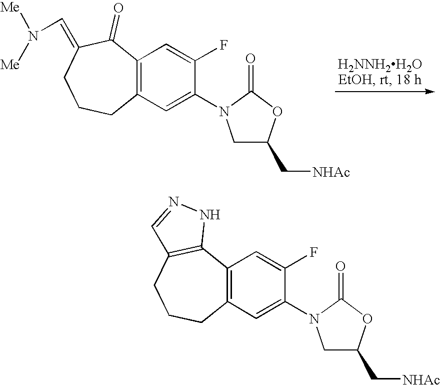

To a solution of (S)-N-[3-(6-dimethylaminomethylene-3-fluoro-5-oxo-6,7,8,9-tetrahydro-5H-benzocyclohepten-2-yl)-2-oxo-oxazolidin-5-ylmethyl]-acetamide (0.66 g, 1.7 mmol) in ethanol (20 mL) was added hydrazine hydrate (0.34 g, 6.8 mmol). The mixture was stirred at room temperature for 18 hours. After removal of the solvent, the residue was purified by chromatography using 5% methanol in chloroform as eluent to give the title compound. Yield 0.408 g (67%). MS-ES: m/z 359 (MH⁺).

### Example 29

### (S)-N-[3-(6-Dimethylaminomethylene-1,4-difluoro-5-oxo-6,7,8,9-tetrahydro-5H-benzocyclohepten-2-yl)-2-oxo-oxazolidin-5-ylmethyl]-acetamide

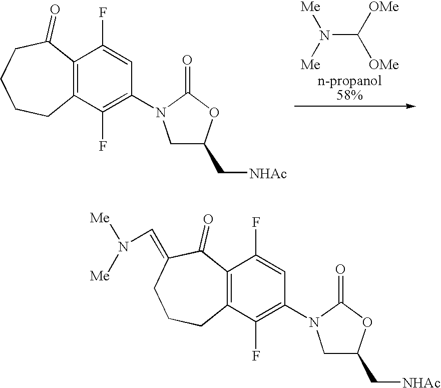

To a solution of (S)-N-[3-(1,4-difluoro-5-oxo-6,7,8,9-tetrahydro-5H-benzocyclo hepten-2-yl)-2-oxo-oxazolidin-5-ylmethyl]-acetamide (0.75 g, 2.13 mmol) in n-propanol (15 mL) was added dimethylformamide dimethyl acetal (1.0 g, 8.5 mmol). The solution was heated to reflux for 18 hours. After removal of the solvent, the residue was purified by chromatography using 5% methanol in chloroform as eluent to give the title compound. Yield 0.5 g (58%). ¹H NMR (400 MHz, CDCl₃): δ 7.85 (s, 1H), 7.18 (dd, 1H), 6.15 (t, 1H), 4.80 (m, 1H), 4.10 (m, 1H), 3.80 (m, 1H), 3.65 (m, 2H), 3.15 (s, 6H), 2.80 (m, 2H), 2.35 (m, 2H), 2.05 (s, 3H), 1.80 (m, 2H).

### Example 30

### (S)-N-[3-(2-Amino-8,11-difluoro-6,7-dihydro-5H-benzo[6,7]cyclohepta [1,2-d]pyrimidin-9-yl)-2-oxo-oxazolidin-5-ylmethyl]-acetamide

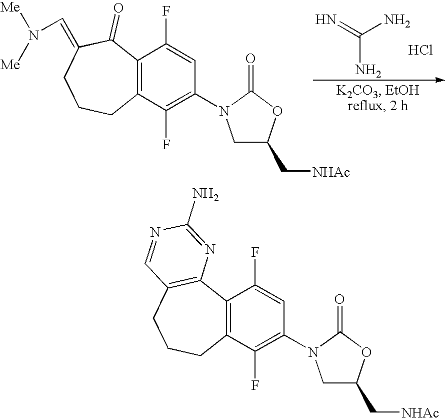

To a solution of (S)-N-[3-(6-dimethylaminomethylene-1,4-difluoro-5-oxo-6,7,8,9-tetrahydro-H-benzocyclohepten-2-yl)-2-oxo-oxazolidin-5-ylmethyl]-acetamide (0.23 g, 0.57 mmol) in ethanol (15 mL) were added guanidine hydrochloride (0.55 g, 5.7 mmol) and potassium carbonate (0.78 g, 5.7 mmol). The mixture was refluxed for 2 hours. After cooling, the reaction mixture was diluted with chloroform (30 mL) and stirred at room temperature for 10 minutes. The solids were filtered and the filtrate was concentrated. The residue was purified by chromatography using 5% to 10% methanol in chloroform as eluent to give the target compound. Yield 0.11 g (48%). ¹H NMR (400 MHz, CDCl₃): δ 8.21 (s, 1H), 7.33 (dd, 1H), 6.22 (t, 1H), 5.12 (s, 2H), 4.85 (m, 1H), 4.14 (dd, 1H), 3.88 (dd, 1H), 3.70 (m, 2H), 2.40 (m, 2H), 2.10 (m, 2H), 2.06 (s, 3H), 1.73 (m, 2H).

### Example 31

### (S)-N-[3-(3-Fluoro-5-oxo-6,7,8,9-tetrahydro-5H-benzocyclohepten-2-yl)-2-oxo-oxazolidin-5-ylmethyl] acetamide

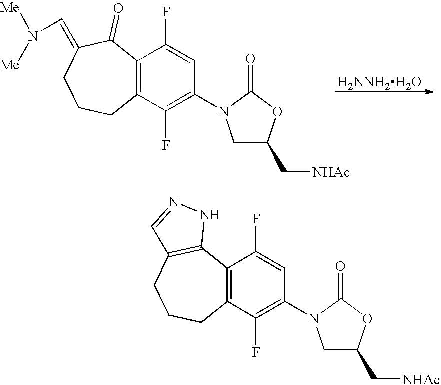

To a solution of (S)-N-[3-(6-dimethylaminomethylene-1,4-difluoro-5-oxo-6,7,8,9-tetrahydro-5H-benzocyclohepten-2-yl)-2-oxo-oxazolidin-5-ylmethyl]-acetamide (0.23 g, 0.57 mmol) in ethanol (15 mL) was added hydrazine hydrate (0.114 g, 2.28 mmol). The mixture was stirred at room temperature for 18 hours. After removal of the solvent, the residue was purified by chromatography using 5% to 10% methanol in chloroform as eluent to give the target compound. Yield 0.16 g (75%). ¹H NMR (400 MHz, CDCl₃): δ 7.53 (s, 1H), 7.23 (dd, 1H), 6.72 (t, 1H), 4.87 (m, 1H), 4.13 (t, 1H), 3.80 (m, 2H), 3.63 (ddd, 1H), 2.80 (m, 4H), 3.05 (m, 5H).

### Example 32

### (S)-N-[3-(8-Dimethylaminomethylene-9-oxo-6,7,8,9-tetrahydro-5H-cyclohepta[b]pyridin-3-yl)-2-oxo-oxazolidin-5-ylmethyl]-acetamide

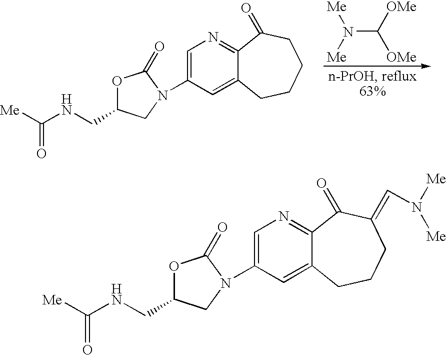

To (S)-N-[2-oxo-3-(9-oxo-6,7,8,9-tetrahydro-5H-cyclohepta[b]pyridin-3-yl)-oxazolidin-5-ylmethyl]-acetamide (2.70 g, 8.51 mmol) in n-propanol (100 mL) was added dimethyformamide dimethyl acetal (4.52 mL, 34.07 mmol). The resulting mixture was heated under reflux overnight, allowed to cool to room temperature and concentrated. The residue was purified by chromatography over silica gel (1:5:94, Et₃N:MeOH:CH₂Cl₂) to give the title compound (2.00 g, 63% yield). m/z (C₁₉H₂₄O₄N₄): 373.10 (MH⁺, 100%).

### Example 33

### (S)-N-[3-(5,6-Dihydro-4H-1-oxa-2,10-diaza-benzo[e]azulen-8-yl)-2-oxo-oxazolidin-5-ylmethyl]-acetamide

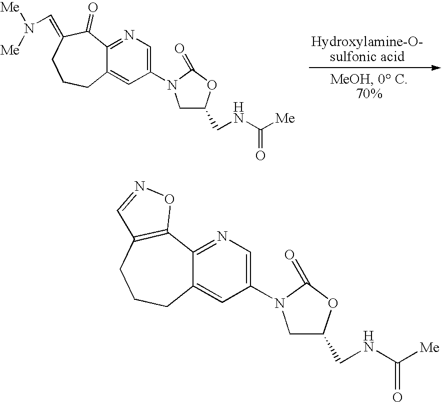

To a solution of (S)-N-[3-(8-dimethylaminomethylene-9-oxo-6,7,8,9-tetrahydro-5H-cyclohepta[b]pyridin-3-yl)-2-oxo-oxazolidin-5-ylmethyl]-acetamide (0.620 g, 1.66 mmol) in methanol (20 mL) at 0° C. was added dropwise hydroxylamine-O-sulfonic acid (0.226 g, 2.00 mmol) in methanol (4 mL). The reaction mixture was then stirred at 0° C. for 10 minutes followed by 30 minutes at room temperature. The reaction mixture was poured into a mixture of saturated sodium bicarbonate solution (50 mL) and water (40 mL) and extracted with ethyl acetate (4×50 mL). The combined organic layers were dried (sodium sulfate), filtered and evaporated to give the crude product, which was purified by chromatography over silica gel (1:5:94, Et₃N:MeOH:CH₂Cl₂) to afford the title compound (0.40 g, 70%). m/z (C₁₇H₁₈O₄N₄): 343.05 (MH⁺, 100%).

### Example 34

### (S)-N-[2-Oxo-3-(1,4,5,6-tetrahydro-1,2,10-triaza-benzo[e]azulen-8-yl)-oxazolidin-5-ylmethyl]-acetamide

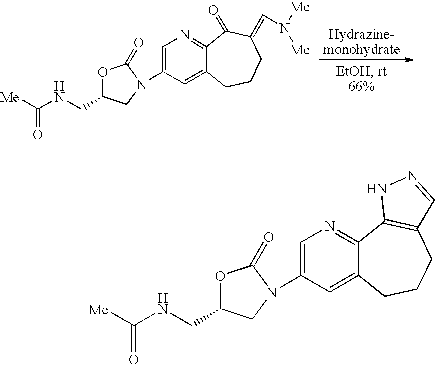

To a solution of (S)-N-[3-(8-dimethylaminomethylene-9-oxo-6,7,8,9-tetrahydro-5H-cyclohepta[b]pyridin-3-yl)-2-oxo-oxazolidin-5-ylmethyl]-acetamide (0.550 g, 1.47 mmol) in ethanol (15 mL) at room temperature was added hydrazine monohydrate (0.30 mL, 5.91 mmol). The reaction mixture was stirred for 1 hour. Water (20 mL) and ethyl acetate (20 mL) were added; the aqueous layer was extracted with ethyl acetate (4×20 mL). The combined organic layers were dried (sodium sulfate), filtered and evaporated to give the crude product, which was purified by chromatography over silica gel (1:5:94, Et₃N:MeOH:CH₂Cl₂) to afford the title compound (0.33 g, 66%). m/z (C₁₇H₁₉O₃N₅): 342.04 (MH⁺, 100%).

### Example 35

### (S)-N-[3-(2-Amino-6,7-dihydro-5H-1,3,11-triaza-dibenzo[a,c]cyclohepten-9-yl)-2-oxo-oxazolidin-5-ylmethyl]-acetamide

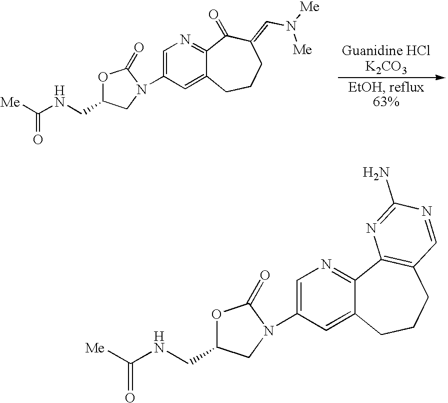

To a mixture of (S)-N-[3-(8-dimethylaminomethylene-9-oxo-6,7,8,9-tetrahydro-5H-cyclohepta[b]pyridin-3-yl)-2-oxo-oxazolidin-5-ylmethyl]-acetamide (0.640 g, 1.72 mmol) in ethanol (25 mL) was added guanidine hydrochloride (1.64 g, 17.20 mmol) and K₂CO₃ (2.37 g, 17.20 mmol). The reaction mixture was heated under reflux for 2 hours. Water (20 mL) and ethyl acetate (20 mL) were added; the aqueous layer was extracted with ethyl acetate (4×20 mL). The combined organic layers were dried (sodium sulfate), filtered and evaporated to give the crude product, which was purified by chromatography over silica gel (1:5:94, Et₃N:MeOH:CH₂Cl₂) to afford the title compound (0.40 g, 63%). m/z (C₁₈H₂₀O₃N₆): 369.07 (MH⁺, 100%).

### Example 36

### (S)-N-[3-(6-Dimethylaminomethylene-4-fluoro-5-oxo-6,7,8,9-tetrahydro-5H-benzocyclohepten-2-yl)-2-oxo-oxazolidin-5-ylmethyl]-acetamide

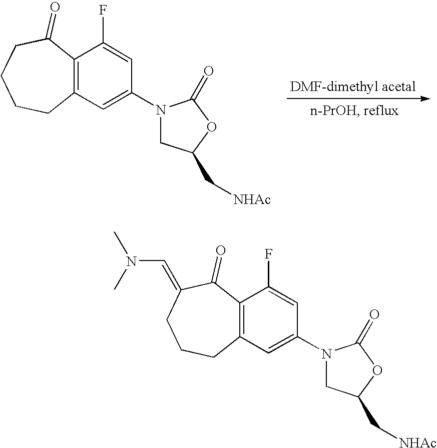

A solution of (S)-N-[3-(4-fluoro-5-oxo-6,7,8,9-tetrahydro-5H-benzocyclohepten-2-yl)-2-oxo-oxazolidin-5-ylmethyl]-acetamide (0.234 g, 0.70 mmol) in n-propanol (7 mL) was treated dropwise with dimethylformamide-dimethyl acetal (0.37 mL, 2.80 mmol) at 23° C. The mixture was heated to 100° C. and stirred for 6 hours. The solution was cooled to room temperature and the solvent removed in vacuo. The residue was placed under high vacuum over night to remove residual solvent, which provided crude material (0.27 g, 90%), which was used without further purification. MS-APCI (m/z+) 390 (M+H).

### Example 37

### (S)-N-[3-(10-Fluoro-1,4,5,6-tetrahydro-1,2-diaza-benzo[e]azulen-8-yl)-2-oxo-oxazolidin-5-ylmethyl]-acetamide

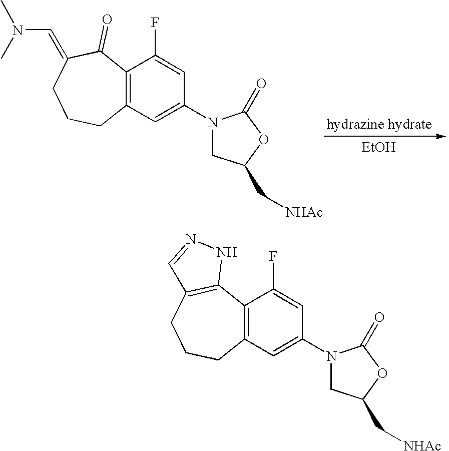

A solution of (S)-N-[3-(6-dimethylaminomethylene-4-fluoro-5-oxo-6,7,8,9-tetrahydro-5H-benzocyclohepten-2-yl)-2-oxo-oxazolidin-5-ylmethyl]-acetamide 2 (0.27 g, 0.70 mmol) in ethanol (8.0 mL) was treated dropwise with hydrazine hydrate (0.14 mL, 2.80 mmol) at 23° C. The mixture was stirred at 23° C. for 4 hours and then the solvent was removed in vacuo. The residue was placed under high vacuum over night to remove residual solvent. The sample was purified by flash chromatography (SiO₂, ethyl acetate/MeOH 1-7%), to afford the title compound (0.18 g, 72%): MS-APCI (m/z+) 359 (M+H).

### Example 38

### (S)-N-[3-(10-Fluoro-5,6-dihydro-4H-1-oxa-2-aza-benzo[e]azulen-8-yl)-2-oxo-oxazolidin-5-ylmethyl]-acetamide

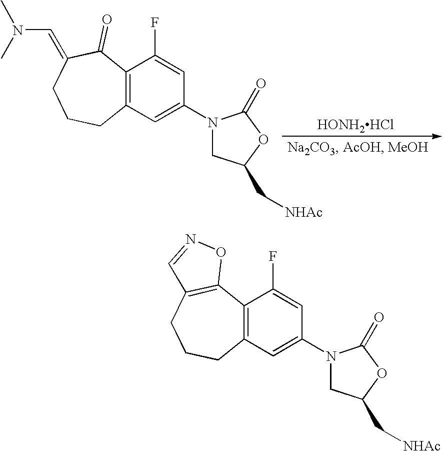

A solution of (S)-N-[3-(6-dimethylaminomethylene-4-fluoro-5-oxo-6,7,8,9-tetrahydro-5H-benzocyclohepten-2-yl)-2-oxo-oxazolidin-5-ylmethyl]-acetamide 2 (0.135 g, 0.346 mmol) in a mixture of MeOH (4 mL)/H₂O (2 mL) was treated with hydroxylamine hydrochloride (0.027 g, 0.385 mmol) and Na₂CO₃ (0.021 g, 0.193 mmol) at 23° C. The reaction was acidified with glacial acetic acid to pH 4-5 and the mixture was stirred at room temperature for 1 hour. The solution was cooled to 0° C. and then quenched with the addition of saturated aqueous sodium bicarbonate (5 mL), and extracted with ethyl acetate (3×10 mL). The combined organic extracts were washed with brine (20 mL), dried over magnesium sulfate and the solvent was removed in vacuo. The residue was purified by flash chromatography (SiO₂, MeOH/MeOH 0-7%), to afford the title compound (0.089 g, 71%): MS-APCI (m/z+) 360 (M+H).

### Example 39

### N-[3-(4-Fluoro-5(R,S)-hydroxy-6,7,8,9-tetrahydro-5H-benzocyclohepten-2-yl)-2-oxo-oxazolidin-5(S)-ylmethyl]-acetamide

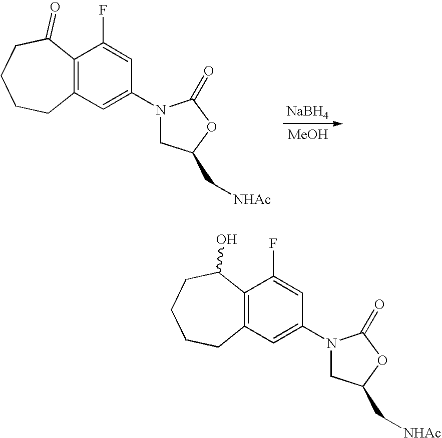

A solution of (S)-N-[3-(4-fluoro-5-oxo-6,7,8,9-tetrahydro-5H-benzocyclohepten-2-yl)-2-oxo-oxazolidin-5-ylmethyl]-acetamide (0.28 g, 0.84 mmol) in methanol (35 mL) was treated with sodium borohydride (0.038 g, 1.0 mmol) at 23° C. The mixture was stirred for 1 h at 23° C. and then was quenched by the addition of saturated aqueous sodium bicarbonate (5 mL). The solvent was removed in vacuo and the crude material was taken up in water (10 mL) and extracted with ethyl acetate (3×10 mL). The combined organic layers were washed with brine (10 mL), dried over magnesium sulfate and the solvent was removed in vacuo to give the title compound (0.22 g, 78%). The crude material was used in the next reaction without further purification: MS-APCI (m/z+) 336 (M+).

### Example 40

### (S)-N-[3-(4-Fluoro-8,9-dihydro-7H-benzocyclohepten-2-yl)-2-oxo-oxazolidin-5-ylmethyl]-acetamide

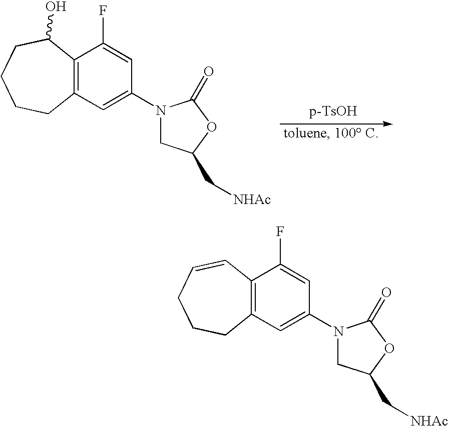

A solution of N-[3-(4-fluoro-5(R,S)-hydroxy-6,7,8,9-tetrahydro-5H-benzocyclohepten-2-yl)-2-oxo-oxazolidin-5(S)-ylmethyl]-acetamide 2 (0.22 g, 0.65 mmol) in toluene (25 mL) was treated with p-toluene sulfonic acid (0.041 g, 0.22 mmol) at 23° C. The mixture was stirred for 1 hour at 100° C. and then cooled to room temperature. The reaction mixture was quenched by the addition of saturated aqueous sodium bicarbonate (5 mL) and extracted with dichloromethane (3×10 mL). The combined organic layers were washed with brine (10 mL), dried over magnesium sulfate and the solvent was removed in vacuo. The crude material was purified by flash chromatography (SiO₂, ethyl acetate-MeOH 0-7%), to afford the title compound (0.195 g, 94%): MS-APCI (m/z+) 319 (M+).

### Example 41

### (S)-N-[3-(9-Methylene-6,7,8,9-tetrahydro-5H-benzocyclohepten-2-yl)-2-oxo-oxazolidin-5-ylmethyl]-acetamide

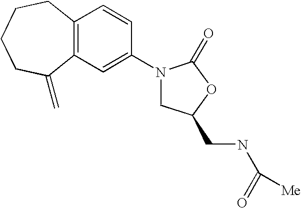

A suspension of 5.25 g (13.0 mmol) of methyl triphenylphosphonium iodide in tetrahydrofuran (100 mL) was cooled to 0° C. under nitrogen and treated drop-wise with 26 mL of 0.5 M potassium hexamethyldisilazide. The mixture was stirred at 0° C. for 30 minutes, and (S)-N-[2-oxo-3-(9-oxo-6,7,8,9-tetrahydro-5H-benzocyclohepten-2-yl)-oxazolidin-5-ylmethyl]-acetamide (1.9 g, 6.0 mmol) was added all at once. The reaction mixture was allowed to warm to room temperature and stirred overnight. Water and ethyl acetate were added; the organic layer was separated, washed with brine and dried over magnesium sulfate. Concentration gave a residue which was chromatographed on silica gel, eluting with 5% MeOH in ethyl acetate to give the title compound. MS (APCI) AP+, 315.2.

### Example 42

### N-[3-(8(R,S)-Fluoro-9-oxo-6,7,8,9-tetrahydro-5H-benzocyclohepten-2-yl)-2-oxo-oxazolidin-5(S)-ylmethyl]-acetamide

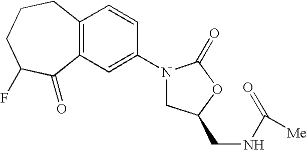

A mixture of 0.13 g (0.41 mmol) of (S)-N-[2-oxo-3-(9-oxo-6,7,8,9-tetrahydro-5H-benzocyclohepten-2-yl)-oxazolidin-5-ylmethyl]-acetamide, 0.28 g (0.43 mmol) of Accufluor (50% w/w on alumina) and MeOH (5 mL) was refluxed for 4 hours, and then stirred for 18 hours at room temperature. Dichloromethane was added, and the mixture was filtered. The filtrate was washed with dilute HCl and brine and was dried over magnesium sulfate. Concentration gave a residue which was chromatographed over silica gel, eluting with 3% MeOH in Ethyl acetate. The product obtained (the dimethyl acetal of the title compound) was dissolved in acetonitrile and treated with 2 mL of 3 N HCl. The reaction mixture was stirred overnight at room temperature. The solution was concentrated, and the residue was triturated with ether and reconcentrated to give the title compound. MS (APCI) AP+, 335.1.

### Example 43

### (S)-N-[3-(3-Methylsulfanyl-1,4,5,6-tetrahydro-1,2-diaza-benzo[e]azulen-8-yl)-2-oxo-oxazolidin-5-ylmethyl]-acetamide

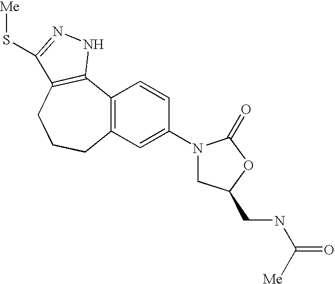

A solution of 0.20 g (0.63 mmol) of (S)-N-[2-oxo-3-(5-oxo-6,7,8,9-tetrahydro-5H-benzocyclohepten-2-yl)-oxazolidin-5-ylmethyl]-acetamide, 0.045 mL (0.058 g, 0.76 mmol) of carbon disulfide, 0.087 mL (0.20 g, 1.40 mmol) of iodomethane, and THF (10 mL) was cooled in an ice bath under nitrogen and then treated dropwise with 1.8 mL of 1.0 M lithium hexamethyl disilazide in THF (addition was complete in 5 minutes). The mixture was allowed to warm to room temperature and stirred overnight; the suspension was then treated with saturated ammonium chloride and extracted with ethyl acetate. The organic layer was washed with brine, dried (magnesium sulfate), and concentrated. The residue was chromatographed on silica gel, eluting with 5% MeOH in ethyl acetate. MS (APCI) AP+, 421.0.

A solution of this intermediate in 6 mL of ethanol was treated with 0.076° mL (0.077 g, 1.5 mmol) of hydrazine hydrate. The mixture was refluxed for 6 hours and stirred at room temperature for 18 hours. The suspension was cooled in an ice bath, and the solids were filtered, washed with cold ethanol, and dried to give the title compound. MS (APCI) AP+, 387.1.

### Example 44

### General Procedure for the Preparation of 3-Amino-Substituted Pyrazoles

### (S)-4-{8-[5-(Acetylamino-methyl)-2-oxo-oxazolidin-3-yl]-1,4,5,6-tetrahydro-1,2-diaza-benzo[e]azulen-3-yl}-piperazine-1-carboxylic acid tert-butyl ester

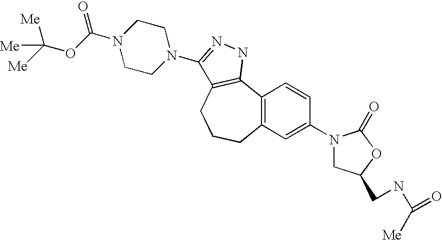

### Bromination of the oxazolidinone ketone (Step 1)

A solution of 0.32 g (1.0 mmol) of (S)-N-[2-oxo-3-(5-oxo-6,7,8,9-tetrahydro-5H-benzocyclohepten-2-yl)-oxazolidin-5-ylmethyl]-acetamide in dichloromethane (30 mL) and acetic acid (5 mL) was treated with 0.35 g (1.1 mmol) of pyridinium tribromide and stirred at room temperature for 18 hours. The solution was poured into a large separatory funnel and then treated (cautiously) with saturated aqueous sodium bicarbonate. When all gas evolution ceased, the organic solution was washed with brine and dried over magnesium sulfate. Concentration gave the bromoketone intermediate.

### Thiosemicarbazide (Step 2)

A solution of 0.37 g (2.0 mmol) of 4-methyl-4-phenyl-3-thiosemicarbazide (Rutter et. al., U.S. Pat. No. 4,282,031), 0.38 g (2.0 mmol) of N-t-butoxycarbonyl piperazine, and acetonitrile (15 mL) was refluxed for 5.0 hours, then stirred at room temperature overnight. The solution was cooled to −40° C. and stirred for 1.5 hours. The solids that formed were filtered and washed with acetonitrile and cold ether; air-drying of the solid gave the title compound. (J. Med. Chem. 1997, 40, 2374-2385) MS (APCI): AP+, 261.1.

### Reaction of bromoketone and thiosemicarbazide (Step 3)

A suspension of 0.41 g (1.0 mmol) of the bromo compound, 0.27 g (1.0 mmol) of the thiosemicarbazide, and ethanol (7 mL) was refluxed for 7 hours. The solution was cooled to room temperature and concentrated. The residue was dissolved in ethyl acetate, washed with aqueous sodium bicarbonate and brine, and dried (magnesium sulfate). Concentration gave the crude product which was chromatographed on silica gel, eluting with 95:5 dichloromethane: MeOH. The material was re-purified via prep TLC to give the title compound. MS (APCI) AP+, 525.2.

### Example 45

### (S)-N-[3-(3-Morpholin-4-yl-1,4,5,6-tetrahydro-1,2-diaza-benzo[e]azulen-8-yl)-2-oxo-oxazolidin-5-ylmethyl]-acetamide

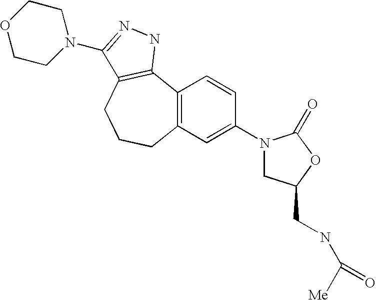

The bromoketone (0.25 g, 0.63 mmol) was reacted with the thiosemicarbazide (prepared from 4-methyl-4-phenyl-3-thiosemicarbazide and morpholine; 0.12 g, 0.73 mmol) in the same fashion as that reported for Example 44. The final product was purified via silica gel chromatography, eluting with 5% MeOH in dichloromethane. The material was re-purified via prep TLC to give the title compound. MS (APCI) AP+, 426.1.

### Example 46

### (S)-N-[3-(3-Dimethylamino-1,4,5,6-tetrahydro-1,2-diaza-benzo[e]azulen-8-yl)-2-oxo-oxazolidin-5-ylmethyl]-acetamide

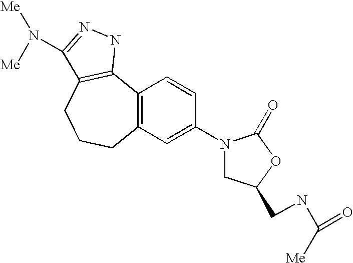

The title compound was prepared from the bromoketone (Example 44; step 1; 0.35 g, 0.88 mmol) and N,N-dimethyl thiosemicarbazide (0.10 g, 0.88 mmol) as described in Example 44. The final product was purified via silica gel chromatography, eluting with 5% MeOH in dichloromethane, followed by prep TLC. MS (APCI) AP+, 384.1.

### Example 47

### N-{3-[3-(3(R,S)-Diethylamino-pyrrolidin-1-yl)-1,4,5,6-tetrahydro-1,2-diaza-benzo[e]azulen-8-yl]-2-oxo-oxazolidin-5(S)-ylmethyl}-acetamide

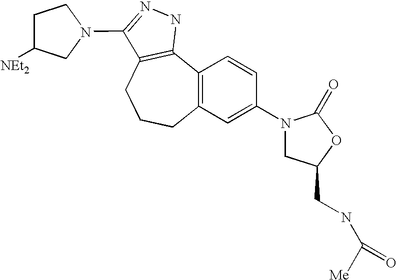

The title compound was prepared from the bromoketone (Example 44, step 1; 0.95 g, 0.88 mmol) and the thiosemicarbazide (prepared from 4-methyl-4-phenyl-3-thiosemicarbazide and diethyl-pyrrolidin-3-yl amine as in Example 44, step 2; 0.52 g, 0.88 mmol) as described in Example 44. The final product was purified via silica gel chromatography, eluting with 5% MeOH in dichloromethane, followed by prep TLC. MS (APCI) AP+, 481.1

### Example 48

### (S)-N-[2-Oxo-3-(3-piperazin-1-yl-1,4,5,6-tetrahydro-1,2-diaza-benzo[e]azulen-8-yl)-oxazolidin-5-ylmethyl]-acetamide

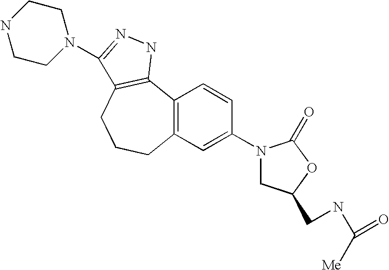

(S)-4-{8-[5-(Acetylamino-methyl)-2-oxo-oxazolidin-3-yl]-1,4,5,6-tetrahydro-1,2-diaza-benzo[e]azulen-3-yl}-piperazine-1-carboxylic acid tert-butyl ester prepared in Example 44 (0.77 mg, 0.15 mmol) was dissolved in ethyl acetate and treated with methanol (0.06 mL) and acetyl chloride (0.052 mL), in that order. The suspension was stirred at room temperature for 2 hours and then concentrated; the residue was dissolved in methanol and treated with 3 drops of ammonium hydroxide. The solution was stirred overnight at room temperature and concentrated. The crude product was purified via prep TLC, eluting with 10% MeOH in dichloromethane with 2% NH₄OH, to give the title compound. MS (APCI): AP+, 425.2

### Example 49

### (S)-N-[2-Oxo-3-(3,4,5,6-tetrahydro-2,3-diaza-benzo[e]azulen-8-yl)-oxazolidin-5-ylmethyl]-acetamide

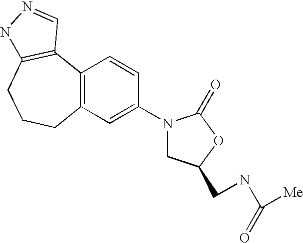

A solution of (S)-N-[2-oxo-3-(6-oxo-6,7,8,9-tetrahydro-5H-benzocyclohepten-2-yl)-oxazolidin-5-ylmethyl]-acetamide (0.40, 1.26 mmol), dimethylformamide-dimethyl acetal (0.60 g, 5.0 mmol), and 1-propanol (10 mL) was refluxed for 7 hours. The mixture was concentrated, and the residue was triturated with ether and re-concentrated. MS (APCI): AP+, 372.1. The crude imine prepared above (0.46 g, 1.24 mmol) was dissolved in ethanol (5 mL) and treated dropwise with 0.24 m/L (0.25 g, 4.9 mmol) of hydrazine hydrate. The reaction mixture was stirred at room temperature for 48 hours. The solution was concentrated to give a residue which was chromatographed over silica gel, eluting with 5% MeOH in ethyl acetate, to give the title compound. MS (APCI), AP+341.1.

### Example 50

### PF-00258246

### (S)-N-[3-(3-Amino-6,7-dihydro-5H-benzo[3,4]cyclohepta[1,2-d]pyrimidin-9-yl)-2-oxo-oxazolidin-5-ylmethyl]-acetamide

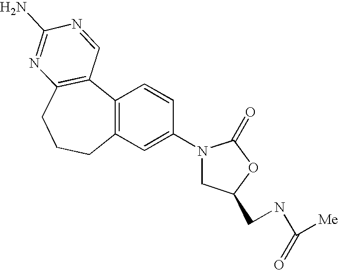

The intermediate imine was prepared as in Example 49 from 0.4 g (1.26 mmol) of the ketone and 0.61 g (5.05 mmol) of DMF dimethyl acetal. The resulting compound was dissolved in MeOH (20 mL) and treated with 1.21 g (12.7 mmol) of guanidine hydrochloride and 1.75 g (12.5 mmol) of potassium carbonate. The suspension was refluxed for 5 hours, then stirred at room temperature over the weekend. The suspension was concentrated, and the residue was partitioned between dichloromethane and saturated ammonium chloride. The aqueous phase was extracted with ethyl acetate. The organic extracts were washed with brine, dried (magnesium sulfate) and concentrated. The product was chromatographed over silica gel, eluting with 10% MeOH in dichloromethane, to give the title compound. MS (APCI): AP+, 368.1.

### Example 51

### (S)-N-[3-(5,6-Dihydro-4H-1-oxa-2-aza-benzo[e]azulen-8-yl)-2-oxo-oxazolidin-5-ylmethyl]-thioacetamide

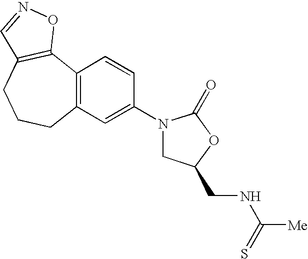

A solution of (S)-N-[3-(5,6-dihydro-4H-1-oxa-2-aza-benzo[e]azulen-8-yl)-2-oxo-oxazolidin-5-ylmethyl]-acetamide (0.150 g, 0.43 mmol) and Lawesson's reagent (0.280 g, 1.6 mmol) was stirred in dry 1,4-dioxane (10 mL) at room temperature for 18 hours. The mixture was then concentrated in vacuo and the residue diluted with ethyl acetate and washed with brine. The organic layer was dried, filtered, concentrated and purified via flash chromatography to give the desired compound. Yield: 0.132 g, 84%. Melting point: 185-195° C. Mass. Spec.: AP+: 358.1. AP−: 356.1. HRMS: 358.1225.

### Example 52

### PF-00183542

### (S)-N-[2-Oxo-3-(1,4,5,6-tetrahydro-1,2-diaza-benzo[e]azulen-8-yl)-oxazolidin-5-ylmethyl]-thioacetamide

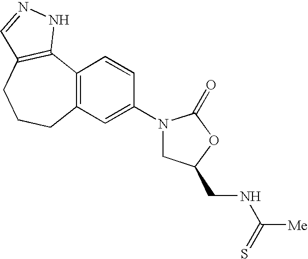

A mixture of (S)-N-[2-oxo-3-(1,4,5,6-tetrahydro-1,2-diaza-benzo[e]azulen-8-yl)-oxazolidin-5-ylmethyl]-acetamide (0.120 g, 0.35 mmol) and Lawesson's reagent (0.285 g, 0.7 mmol) in dry 1,4-dioxane (5 mL) was stirred at 95° C. for 5 hours and then permitted to cool to room temperature and stirred for 18 hours. The mixture was then diluted with ethyl acetate and washed with brine. The organic layer was dried, filtered, concentrated in vacuo and the resulting oil purified via flash chromatography with ether to afford the title compound, 0.066 g, 53%. Melting point: 190-195° C. Mass Spec: AP+: 357.1. AP−: 355.1. Anal Calc for C18H20N4O2S1: % Calc: C, 60.09; H, 5.88, N, 15.07. Found: C, 60.01; H: 5.69, N, 14.68.

### Example 53

### (S)-[2-Oxo-3-(5-oxo-6,7,8,9-tetrahydro-5H-benzocyclohepten-2-yl)-oxazolidin-5-ylmethyl]-carbamic acid tert-butyl ester

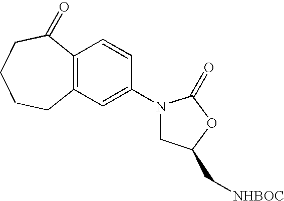

To a stirring suspension of the amine salt ((S)-5-aminomethyl-3-(5-oxo-6,7,8,9-tetrahydro-5H-benzocyclohepten-2-yl)-oxazolidin-2-one hydro chloride (18.8 g, 60 mmol) in THF (375 mL) at 0° C. was added triethylamine (10.2 mL, 73 mmol) and di-t-butyl-dicarbonate (15.8 g, 73 mmol), followed by methylene chloride (300 mL). The slightly cloudy mixture was then stirred for 6 hours while slowly warming to room temperature, whereupon it was concentrated in vacuo and the residue diluted with methylene chloride and washed with water. The organic layer was dried, filtered and concentrated to a solid that was purified via flash chromatography with ethyl acetate/hexane (1:1) to give the title compound. Yield: 14.5 g, 65%. Melting point: 155-157° C. Anal Calc for C20H26N2O3: % Calc: C, 64.16; H, 7.00, N, 7.48. Found: C, 63.85; H, 7.03, N, 7.38.

### Example 54

### (S)-[3-(6-Dimethylaminomethylene-5-oxo-6,7,8,9-tetrahydro-5H-benzocyclohepten-2-yl)-2-oxo-oxazolidin-5-ylmethyl]-carbamic acid tert-butyl ester

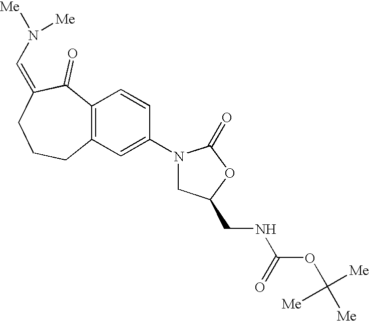

A mixture of (S)-[2-oxo-3-(5-oxo-6,7,8,9-tetrahydro-5H-benzocyclohepten-2-yl)-oxazolidin-5-ylmethyl]-carbamic acid tert-butyl ester (4 g, 10.6 mmol) and Brederick's reagent (21 g, 120 mmol) was stirred at 55° C. for 4 hours. The solution was then cooled, diluted with ethyl acetate and washed with water. The organic layer was dried, filtered and concentrated in vacuo to give a solid that was purified via flash chromatography with ethyl acetate to give the title compound. Yield: 3.4 g, 74%. Melting point: 157-160° C. Anal. Calc. for C23H31N3O5: % Calc: C=64.32, H=7.27, N=9.78. % Found: C=63.46, H=7.4, N=9.69.

### Example 55

### PF-00271681

### (S)-[3-(5,6-Dihydro-4H-1-oxa-2-aza-benzo[e]azulen-8-yl)-2-oxo-oxazolidin-5-ylmethyl]-carbamic acid tert-butyl ester

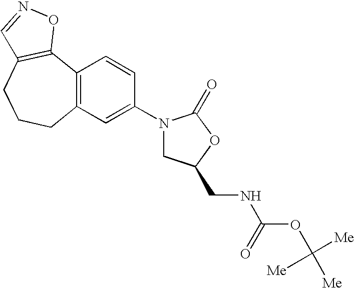

To a suspension of hydroxylamine-O-sulfonic acid (2.57 g, 22.7 mmol) and sodium acetate (1.86 g, 22.7 mmol) in 300 mL of a 3:1 mixture of MeOH/water was added a methanolic solution of (S)-[3-(6-dimethylamino methylene-5-oxo-6,7,8,9-tetrahydro-5H-benzocyclohepten-2-yl)-2-oxo-oxazolidin-5-ylmethyl]-carbamic acid tert-butyl ester (3.9 g, 9.08 mmol) and the mixture stirred at 48° C. for 24 hours. The mixture was then cooled and allowed to stir at room temperature for 2 hours and then concentrated in vacuo. The residue was diluted with ethyl acetate and washed with saturated sodium bicarbonate and brine. The organic layer was dried, filtered and concentrated in vacuo and purified via flash chromatography with 50:50, ethyl acetate:hexane to afford the title compound. Yield: 2.8 g, 77%. Melting point: 180-184° C. MS: AP+: 385.0. AP−: 398.1, 298.1 Anal. Calc for C21H25N3O5: % Calc: C, 63.15; H, 6.31, N, 10.52. % Found: C, 63.00; H, 6.26, N, 10.39.

### Example 56

### (S)-[2-Oxo-3-(1,4,5,6-tetrahydro-1,2-diaza-benzo[e]azulen-8-yl)-oxazolidin-5-ylmethyl]-carbamic acid tert-butyl ester

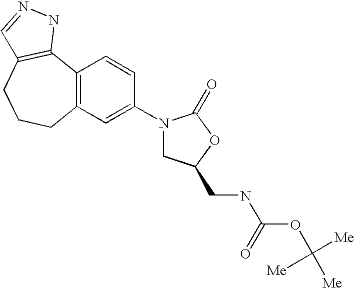

To a stirring solution of (S)-[3-(6-dimethylaminomethylene-5-oxo-6,7,8,9-tetrahydro-5H-benzocyclohepten-2-yl)-2-oxo-oxazolidin-5-ylmethyl]-carbamic acid tert-butyl ester (3.33 g, 7.8 mmol) in absolute ethanol (150 mL) at room temperature was added an ethanolic solution of hydrazine hydrate (1.55 g, 31 mmol) and the resulting mixture stirred for 24 hours. It was then concentrated in vacuo and the residue diluted with ethyl acetate and washed with water. The organic layer was dried, filtered, concentrated and dried under high vacuum to give the desired product, 3.1 g, 100%. Melting point: 85-100° C. MS: AP+: N/A. AP−: 397.1. Anal Calc. For C21H26N4O4/0.31C4H10O2: % Calc: C, 62.65; H: 6.88, N, 13.14. % Found: C, 62.26; H, 6.91, N, 12.77.

### Example 57

### (S)-5-Aminomethyl-3-(5,6-dihydro-4H-1-oxa-2-aza-benzo[e]azulen-8-yl)-oxazolidin-2-one hydrochloride

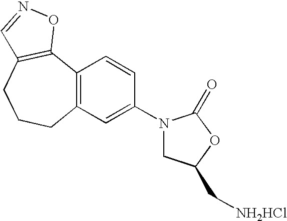

To a stirring solution of (S)-[3-(5,6-dihydro-4H-1-oxa-2-aza-benzo[e]azulen-8-yl)-2-oxo-oxazolidin-5-ylmethyl]-carbamic acid tert-butyl ester (0.5 g, 1.25 mmol) in dry 1,4-dioxane (5 mL) was added HCl (4.0 M/dioxane, 4.7 mL, 18.8 mmol) and the mixture stirred at room temperature for 24 hours. It was then concentrated in vacuo and the residue triturated with ether and filtered to give the title compound, 0.41 g (97%). Melting point: 245-258° C. MS: AP+: N/A. AP−: 397.1. Anal Calc. For C16H17N3O3-HCl/0.45H2O: % Calc: C, 55.88; H: 5.54, N, 12.22% Found: C, 56.25; H, 5.53, N, 11.82.

### Example 58

### (S)-5-Aminomethyl-3-(1,4,5,6-tetrahydro-1,2-diaza-benzo[e]azulen-8-yl)-oxazolidin-2-one hydrochloride

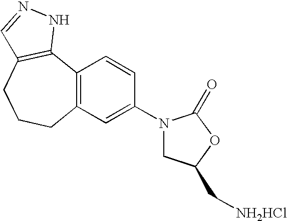

To a stirring solution of (S)-[2-oxo-3-(1,4,5,6-tetrahydro-1,2-diaza-benzo[e]azulen-8-yl)-oxazolidin-5-ylmethyl]-carbamic acid tert-butyl ester (3.1 g, 7.8 mmol) in dry 1,4-dioxane (25 mL) was added HCl (4.0 M/dioxane, 10 mL, 29 mmol) and the mixture stirred at ambient temperature for 1 hour. The mixture was then concentrated in vacuo and then triturated with ether, filtered, and washed with ether and ethyl acetate. The filtrate was placed on high vacuum for 18 hours to afford the title compound, 2.55 g (98% yield). Melting point: 201-223° C. MS: AP+: N/A. AP−: 297.1. Anal Calc. For C16H18N4O2-HCl/1.35HCl/0.05H₂O: % Calc: C, 49.92; H, 5.53, N, 14.56% Found: C, 50.09; H, 5.75, N, 14.21.

### Example 59

### (S)-N-[3-(5,6-Dihydro-4H-1-oxa-2-aza-benzo[e]azulen-8-yl)-2-oxo-oxazolidin-5-ylmethyl]-propionamide

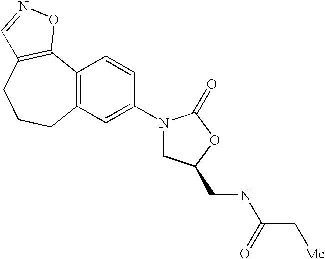

The title compound was prepared using (S)-5-aminomethyl-3-(5,6-dihydro-4H-1-oxa-2-aza-benzo[e]azulen-8-yl)-oxazolidin-2-one hydrochloride, 10 eq of triethylamine and propionyl chloride according to the general procedure AA. Yield: 0.09 g (86%). Melting point: 185-186° C. MS: AP+: 312.1. AP−: 310.1. Anal Calc. For C19H21N3O4/0.62H2O: % Calc: C, 62.26; H, 6.12, N, 11.46% Found: C, 61.87; H, 6.09, N, 11.09.

### Example 60

### (S)-N-[3-(5,6-Dihydro-4H-1-oxa-2-aza-benzo[e]azulen-8-yl)-2-oxo-oxazolidin-5-ylmethyl]-isobutyramide

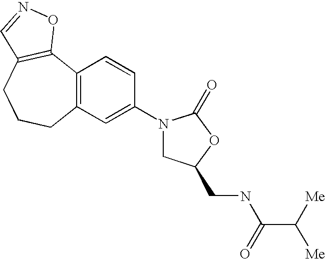

The title compound was prepared using (S)-5-aminomethyl-3-(5,6-dihydro-4H-1-oxa-2-aza-benzo[e]azulen-8-yl)-oxazolidin-2-one, 3.5 eq. of triethylamine and 1.2 eq of isobutyryl chloride as described in the general procedure AA. Yield: 0.097 g, 88%. Melting point: 187-193° C. MS: AP+: 370.1. AP−: 324.2. Anal Calc. For C₂₀H₂₃N₃O₄: % Calc: C, 65.03; H, 6.28, N, 11.37. % Found: C, 64.71; H, 6.30, N, 11.14.

### Example 61

### (S)-Cyclopropanecarboxylic acid [3-(5,6-dihydro-4H-1-oxa-2-aza-benzo[e]azulen-8-yl)-2-oxo-oxazolidin-5-ylmethyl]-amide

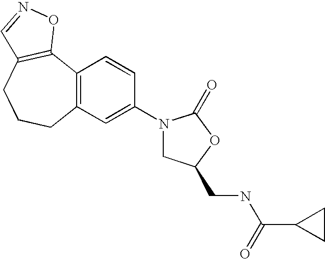

The title compound was prepared as described in general procedure AA using (S)-5-aminomethyl-3-(5,6-dihydro-4H-1-oxa-2-aza-benzo[e]azulen-8-yl)-oxazolidin-2-one, 10 eq of triethylamine and 10 eq. of cyclopropanecarbonyl chloride. Melting point: 211-215° C. HRMS: AP+: 368.1606.

### Example 62

### (S)-2-Cyclopropyl-N-[3-(5,6-dihydro-4H-1-oxa-2-aza-benzo[e]azulen-8-yl)-2-oxo-oxazolidin-5-ylmethyl]-acetamide

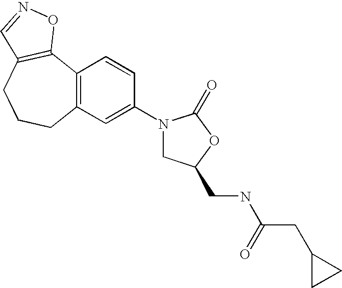

The title compound was prepared using (S)-5-aminomethyl-3-(5,6-dihydro-4H-1-oxa-2-aza-benzo[e]azulen-8-yl)-oxazolidin-2-one and cyclopropylacetic acid as described in general procedure BB. Yield: 0.080 g (70%). Melting point: 187-191° C. MS: AP+: 382.1. AP−: 336.2. Anal Calc. For C21H23N3O4: % Calc: C, 66.13; H, 6.08, N, 11.02. % Found: C, 66.24; H, 6.16, N, 10.93.

### Example 63

### (S)-2-Cyclopentyl-N-[3-(5,6-dihydro-4H-1-oxa-2-aza-benzo[e]azulen-8-yl)-2-oxo-oxazolidin-5-ylmethyl]-acetamide

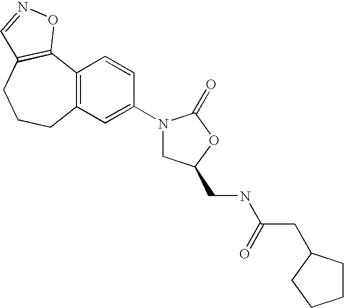

The title compound was prepared using (S)-5-aminomethyl-3-(5,6-dihydro-4H-1-oxa-2-aza-benzo[e]azulen-8-yl)-oxazolidin-2-one one and cyclopentylacetic acid as described in the general procedure BB. Yield: 0.095 g (78%). Melting point: 193-195° C. MS: AP+: 410.1. AP−: 364.2. Anal Calc. For C23H27N3O4/0.09H2O. % Calc: C, 67.20; H, 6.66, N, 10.22. % Found: C, 66.80; H, 6.67, N, 10.30.

### Example 64

### (S)-N-[3-(5,6-Dihydro-4H-1-oxa-2-aza-benzo[e]azulen-8-yl)-2-oxo-oxazolidin-5-ylmethyl]-malonamic acid methyl ester

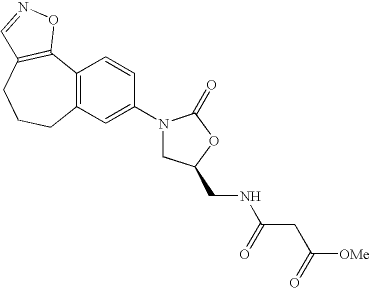

The title compound was prepared using (S)-5-aminomethyl-3-(5,6-dihydro-4H-1-oxa-2-aza-benzo[e]azulen-8-yl)-oxazolidin-2-one as described in general procedure AA with 1.2 eq. of methylmalonyl chloride and 3.5 eq. of triethylamine. Yield: 0.053 g (45%). Melting point: 205-217° C. MS: AP+: 400.1. AP−: 398.2. Anal Calc. For C20H21N3O6/0.16H2O. % Calc: C, 59.71; H, 5.34, N, 10.45. % Found: C, 59.71; H, 5.12, N, 10.05.

### Example 65

### (S)-N-[3-(5,6-Dihydro-4H-1-oxa-2-aza-benzo[e]azulen-8-yl)-2-oxo-oxazolidin-5-ylmethyl]-2,2,2-trifluoro-acetamide

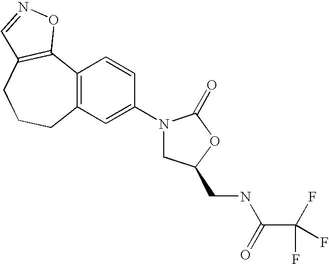

The title compound was prepared as described in general procedure AA using (S)-5-aminomethyl-3-(5,6-dihydro-4H-1-oxa-2-aza-benzo[e]azulen-8-yl)-oxazolidin-2-one, 2.5 eq. of triethylamine and 1.2 eq of trifluoroacetic anhydride. Yield: 0.093 g (79%). Melting point: 203-205° C. MS: AP+: 396.0.1. AP−: 394.0. Anal Calc. For C₁₈H₁₆N₃O₄: % Calc: C, 54.69; H, 4.08, N, 10.63. % Found: C, 54.70; H, 3.94; N, 10.45.

### Example 66

### (S)-N-[3-(5,6-Dihydro-4H-1-oxa-2-aza-benzo[e]azulen-8-yl)-2-oxo-oxazolidin-5-ylmethyl]-benzamide

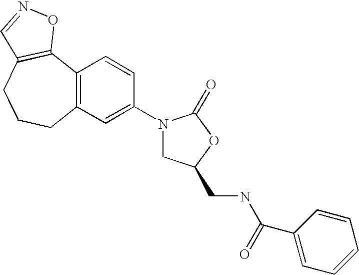

The title compound was prepared using (S)-5-aminomethyl-3-(5,6-dihydro-4H-1-oxa-2-aza-benzo[e]azulen-8-yl)-oxazolidin-2-one, 10 eq. of triethylamine and 10 eq of benzoyl chloride as described in the general procedure AA. Yield: 0.056 g (47%). Melting point: 240-245° C. MS: AP+: 404.1. AP−: N/A. Anal Calc. For C23H21N3O4/0.2CH₂Cl₂; % Calc: C, 67.87; H, 5.3, N: 10.32. % Found: C, 67.51; H, 5.17; N, 9.94.

### Example 67

### (S)-N-[3-(5,6-Dihydro-4H-1-oxa-2-aza-benzo[e]azulen-8-yl)-2-oxo-oxazolidin-5-ylmethyl]-3,3,3-trifluoro-propionamide

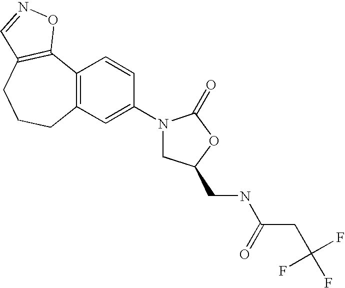

The title compound was prepared using (S)-5-aminomethyl-3-(5,6-dihydro-4H-1-oxa-2-aza-benzo[e]azulen-8-yl)-oxazolidin-2-one and 3,3,3-trifluoropropionic acid as described in general procedure BB. Yield: 0.097 g (80%). Melting point: 150-186° C. MS: AP+: 410.0. AP−: 408.1. Anal Calc. for C19H18F3N3O4/0.29H₂O. % Calc: C, 55.04; H, 4.52, N, 10.14. % Found: C, 54.65; H, 4.61, N, 10.09.

### Example 68

### (S)-N-[3-(5,6-Dihydro-4H-1-oxa-2-aza-benzo[e]azulen-8-yl)-2-oxo-oxazolidin-5-ylmethyl]-2,2-difluoro-acetamide

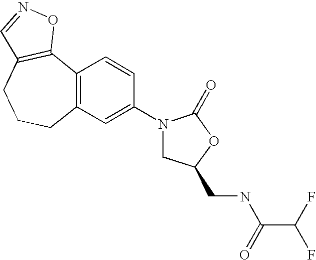

The title compound was prepared using (S)-5-aminomethyl-3-(5,6-dihydro-4H-1-oxa-2-aza-benzo[e]azulen-8-yl)-oxazolidin-2-one and difluoroacetic acid as described in general procedure BB. Yield: 0.069 g (61%). Melting point: 145-180° C. MS: AP+: 378.0. AP−: 376.1. Anal Calc. for C18H17F2N3O4. % Calc: C, 57.29; H, 4.54, N, 11.14. % Found: C, 57.67; H: 4.66, N, 10.99.

### Example 69

### (S)-N-[2-Oxo-3-(1-propionyl-1,4,5,6-tetrahydro-1,2-diaza-benzo[e]azulen-8-yl)-oxazolidin-5-ylmethyl]-propionamide

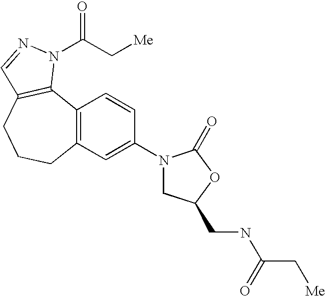

The title compound was prepared using (S)-5-aminomethyl-3-(1,4,5,6-tetrahydro-1,2-diaza-benzo[e]azulen-8-yl)-oxazolidin-2-one hydrochloride and propionyl chloride as described in procedure CC. Yield: 0.160 g (65%). MS: AP+: 411.1. AP−: 309.1. Anal Calc. For C22H26N4O4-0.34H₂O. % Calc: C, 63.43; H: 6.46, N, 13.45. % Found: C, 63.35; H, 6.35, N, 13.05

### Example 70

### (S)-N-[3-(1-Isobutyryl-1,4,5,6-tetrahydro-1,2-diaza-benzo[e]azulen-8-yl)-2-oxo-oxazolidin-5-ylmethyl]-isobutyramide

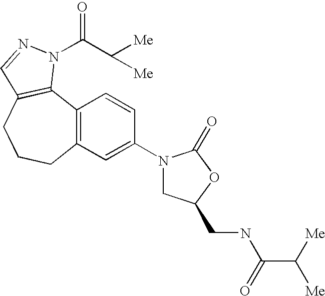

The title compound was prepared using (S)-5-aminomethyl-3-(1,4,5,6-tetrahydro-1,2-diaza-benzo[e]azulen-8-yl)-oxazolidin-2-one hydrochloride and isobutyryl chloride as described in the general procedure CC. Yield: 0.175 g (67%). Melting point: 75-80° C. MS: AP+: 439.2. AP−: 323.2. Anal Calc. for C24H30N4O4-0.04H2O. % Calc: C, 65.63; H, 6.90, N, 12.76. % Found: C, 65.48; H, 6.88, N, 12.36.

### Example 71

### (S)-Cyclopropanecarboxylic acid [3-(1-cyclopropanecarbonyl-1,4,5,6-tetrahydro-1,2-diaza-benzo[e]azulen-8-yl)-2-oxo-oxazolidin-5-ylmethyl]-amide

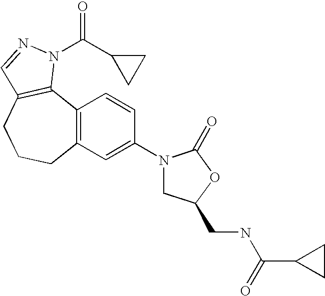

The title compound was prepared using (S)-5-aminomethyl-3-(1,4,5,6-tetrahydro-1,2-diaza-benzo[e]azulen-8-yl)-oxazolidin-2-one hydrochloride and cyclopropanecarbonyl chloride as described in the general procedure CC and was isolated as a 70% pure mixture of mono and di-acylated components as analyzed by LCMS. (70%, AP+: 435.3.)

### Example 72

### (S)-2-Cyclopropyl-N-{3-[1-(2-cyclopropyl-acetyl)-1,4,5,6-tetrahydro-1,2-diaza-benzo[e]azulen-8-yl]-2-oxo-oxazolidin-5-ylmethyl}-acetamide

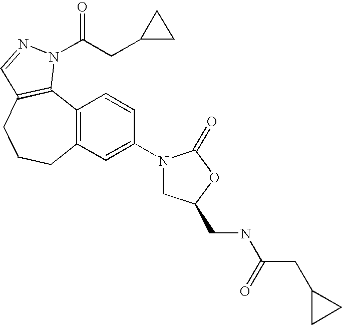

The title compound was prepared using as described in general procedure BB (S)-5-aminomethyl-3-(1,4,5,6-tetrahydro-1,2-diaza-benzo[e]azulen-8-yl)-oxazolidin-2-one hydrochloride (0.120 g, 0.36 mmol) and 1.1 eq of t-butanol, triethylamine, cyclopropylacetic acid and 2.2 eq EDCI. Yield: 0.055 g (33%). MS: AP+: 463.1. Anal Calc. For C26H30N4O4-0.69H2O. % Calc: C, 65.75; H: 6.66, N, 11.80. % Found: C, 66.12; H, 6.53, N, 11.40.

### Example 73

### (S)-2-Cyclopentyl-N-{3-[1-(2-cyclopentyl-acetyl)-1,4,5,6-tetrahydro-1,2-diaza-benzo[e]azulen-8-yl]-2-oxo-oxazolidin-5-ylmethyl}-acetamide

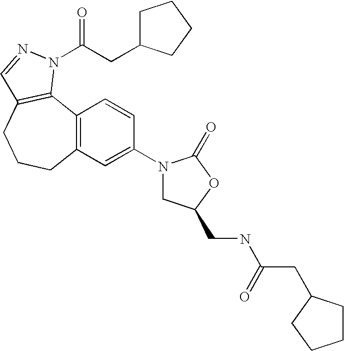

The title compound was prepared using (S)-5-aminomethyl-3-(1,4,5,6-tetrahydro-1,2-diaza-benzo[e]azulen-8-yl)-oxazolidin-2-one hydrochloride, 2.2 eq. t-butanol, 2.2 eq. cyclopentyl acetic acid, 2.2 eq EDCI and 6 eq. of triethylamine as described in general procedure DD. Yield: 0.175 g (56%). Melting point: 165-167° C. MS: AP+: 519.3.

### Example 74

### (S)-N-{3-[1-(2-Methoxycarbonyl-acetyl)-1,4,5,6-tetrahydro-1,2-diaza-benzo[e]azulen-8-yl]-2-oxo-oxazolidin-5-ylmethyl}-malonamic acid methyl ester

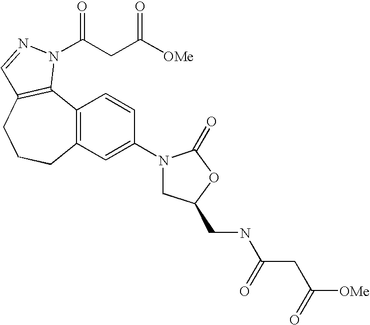

The title compound was prepared using using (S)-5-aminomethyl-3-(1,4,5,6-tetrahydro-1,2-diaza-benzo[e]azulen-8-yl)-oxazolidin-2-one hydrochloride and methylmalonyl chloride as described in general procedure CC and was isolated as a 2:1 mixture of diacylated to monoacylated products as analyzed by LCMS. AP+: 499.2 (diacylated), 399.3 (monoacylated).

### Example 75

### (S)-N-[3-(1-Benzoyl-1,4,5,6-tetrahydro-1,2-diaza-benzo[e]azulen-8-yl)-2-oxo-oxazolidin-5-ylmethyl]-benzamide

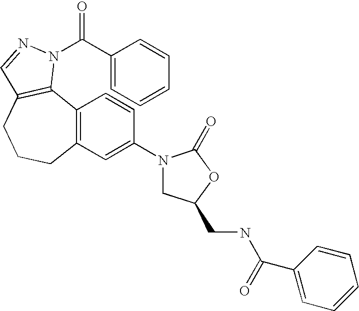

The title compound was prepared using (S)-5-aminomethyl-3-(1,4,5,6-tetrahydro-1,2-diaza-benzo[e]azulen-8-yl)-oxazolidin-2-one hydrochloride and benzoyl chloride as described in general procedure CC. Yield: 0.236 g (84%). Melting point: 75-95° C. MS: AP+: 507.1. AP−: 357.1. Anal Calc. for C30H26N4O4-0.35C4H10O1. % Calc: C, 70.82; H, 5.58, N, 10.52. % Found: C, 70.45; H, 5.31, N, 10.35.

### Example 76

### (S)-3,3,3-Trifluoro-N-{2-oxo-3-[1-(3,3,3-trifluoro-propionyl)-1,4,5,6-tetrahydro-1,2-diaza-benzo[e]azulen-8-yl]-oxazolidin-5-ylmethyl}-propionamide

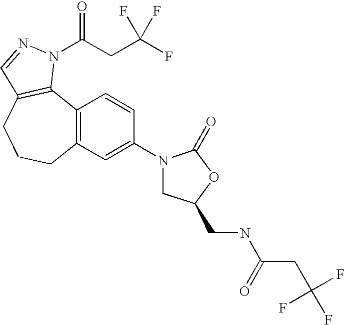

The title compound was prepared using (S)-5-aminomethyl-3-(1,4,5,6-tetrahydro-1,2-diaza-benzo[e]azulen-8-yl)-oxazolidin-2-one hydrochloride, 2.2 eq. t-butanol, 2.5 eq. 3,3,3-trifluoropropionyl chloride, 2.2 eq EDCI and 6 eq. of triethylamine as described in general procedure DD and was isolated as a 2.1:1 mixture of mono:diacylated products as analyzed by LCMS. AP+: 409.2, 519.2.

### Example 77

### (S)-2,2,2-Trifluoro-N-[2-oxo-3-(1,4,5,6-tetrahydro-1,2-diaza-benzo[e]azulen-8-yl)-oxazolidin-5-ylmethyl]-acetamide

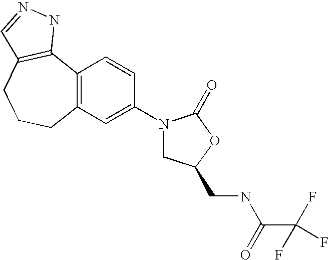

The title compound was prepared using (S)-5-aminomethyl-3-(1,4,5,6-tetrahydro-1,2-diaza-benzo[e]azulen-8-yl)-oxazolidin-2-one hydrochloride as described in general procedure CC with 2.5 eq. of trifluoroacetic anhydride. Yield: 0.120 g (51%). LCMS: AP+: 395.2. Anal Calc. For C18H17F3N4O3-0.17 dichloromethane. % Calc: C, 53.39; H, 4.28, N, 13.71. % Found: C, 53.00; H: 3.94, N, 13.58.

### Example 78

### (S)-2,2-Difluoro-N-[2-oxo-3-(1,4,5,6-tetrahydro-1,2-diaza-benzo[e]azulen-8-yl)-oxazolidin-5-ylmethyl]-acetamide

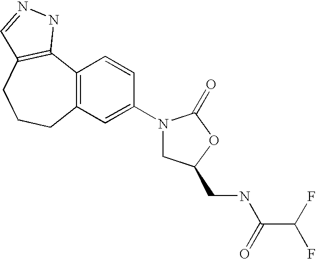

The title compound was prepared using (S)-5-aminomethyl-3-(1,4,5,6-tetrahydro-1,2-diaza-benzo[e]azulen-8-yl)-oxazolidin-2-one hydrochloride, 2.2 eq. t-butanol, 2.2 eq. difluoroactetic acid, 2.2 eq EDCI and 6 eq. of triethylamine as described in general procedure DD. Melting point: 147-150° C. LCMS: AP+: 377.2. Anal Calc. For C18H18F2N4O3-0.30H2O: % Calc: C, 56.63; H, 4.91, N: 14.68. % Found: C, 56.29; H, 4.81, N, 14.64.

### Example 79

### (S)-N-[2-Oxo-3-(1,4,5,6-tetrahydro-1,2-diaza-benzo[e]azulen-8-yl)-oxazolidin-5-ylmethyl]-propionamide

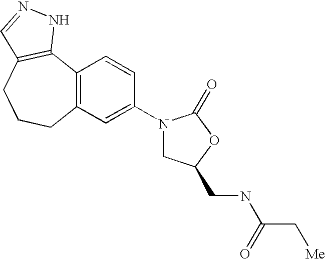

The title compound was prepared from (S)-N-[2-oxo-3-(1-propionyl-1,4,5,6-tetrahydro-1,2-diaza-benzo[e]azulen-8-yl)-oxazolidin-5-ylmethyl]-propionamide (0.145 g, 0.35 mmol) with 3.5 eq benzylamine as described in general procedure EE. Yield; 0.104 g, (83%). Melting point: 233-235° C. MS: AP+: 355.1. AP−: 353.1. Anal Calc. For C19H22N4O3-0.10H2O: % Calc: C, 64.07; H: 6.28, N, 15.73. % Found: C, 63.68; H, 5.93, N, 15.53.

### Example 80

### (S)-N-[2-Oxo-3-(1,4,5,6-tetrahydro-1,2-diaza-benzo[e]azulen-8-yl)-oxazolidin-5-ylmethyl]-isobutyramide

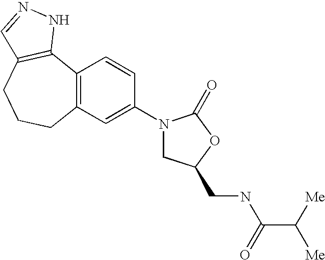

The title compound was prepared from (S)-N-[3-(1-isobutyryl-1,4,5,6-tetrahydro-1,2-diaza-benzo[e]azulen-8-yl)-2-oxo-oxazolidin-5-ylmethyl]-isobutyramide (0.164 g, 0.37 mmol) with 3.5 eq benzylamine as described in general procedure EE. Yield: 0.120 g, (87%). Melting point: 238-240° C. HRMS: AP+: 369.1931.

### Example 81

### (S)-Cyclopropanecarboxylic acid [2-oxo-3-(1,4,5,6-tetrahydro-1,2-diaza-benzo[e]azulen-8-yl)-oxazolidin-5-ylmethyl]-amide

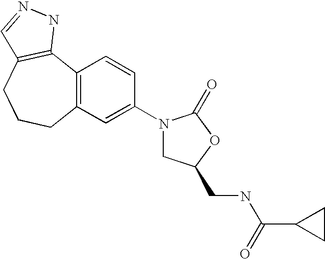

The title compound was prepared from (S)-cyclopropanecarboxylic acid [3-(1-cyclopropanecarbonyl-1,4,5,6-tetrahydro-1,2-diaza-benzo[e]azulen-8-yl)-2-oxo-oxazolidin-5-ylmethyl]-amide (0.170 g, 0.39 mmol) with 3.5 eq benzylamine as described in general procedure EE. Yield: 0.110 g, (77%). Melting point: 224-227° C. MS: AP+: 325.1 AP−: 323.2. Anal Calc. For C20H22N4O3-0.04H2O: % Calc: C, 65.43; H, 6.06, N, 15.26. % Found: C, 65.06; H, 6.04, N, 15.07.

### Example 82

### (S)-2-Cyclopropyl-N-[2-oxo-3-(1,4,5,6-tetrahydro-1,2-diaza-benzo[e]azulen-8-yl)-oxazolidin-5-ylmethyl]-acetamide

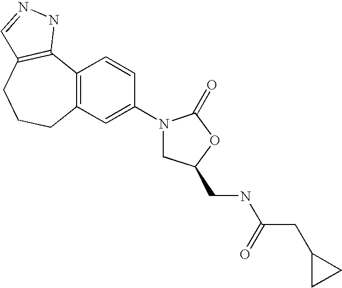

The title compound was prepared from (S)-2-cyclopropyl-N-{3-[1-(2-cyclopropyl-acetyl)-1,4,5,6-tetrahydro-1,2-diaza-benzo[e]azulen-8-yl]-2-oxo-oxazolidin-5-ylmethyl}-acetamide (0.045 g, 0.09 mmol) with 2 eq benzylamine as described in general procedure EE. Yield: 0.027 g, (73%). Melting point: 212-215° C. MS: AP+: 381.1 AP−: 225.2. Anal Calc. For C21H24N4O3-0.20H2O1: % Calc: C, 65.68; H, 6.40, N, 14.59. % Found: C, 65.88; H, 6.48, N, 14.19.

### Example 83

### (S)-2-Cyclopentyl-N-[2-oxo-3-(1,4,5,6-tetrahydro-1,2-diaza-benzo[e]azulen-8-yl)-oxazolidin-5-ylmethyl]-acetamide

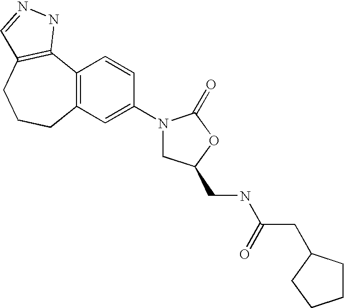

The title compound was prepared from (S)-2-cyclopentyl-N-{3-[1-(2-cyclopentyl-acetyl)-1,4,5,6-tetrahydro-1,2-diaza-benzo[e]azulen-8-yl]-2-oxo-oxazolidin-5-ylmethyl}-acetamide (0.168 g, 0.32 mmol) with 3.5 eq benzylamine as described in general procedure EE. Yield: 0.113 g (85%). Melting point: 205-208° C. MS: AP+: 409.1 AP−: 363.2. Anal Calc. For C23H28N4O3-0.03H2O1: % Calc: C, 67.54; H, 6.91, N, 13.70. % Found: C, 67.16; H, 6.81, N, 13.35.

### Example 84

### (S)-N-[2-Oxo-3-(1,4,5,6-tetrahydro-1,2-diaza-benzo[e]azulen-8-yl)-oxazolidin-5-ylmethyl]-malonamic acid methyl ester

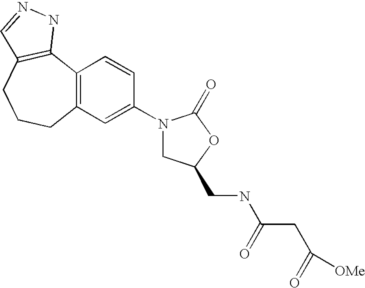

The title compound was prepared from (S)-N-{3-[1-(2-methoxycarbonyl-acetyl)-1,4,5,6-tetrahydro-1,2-diaza-benzo[e]azulen-8-yl]-2-oxo-oxazolidin-5-ylmethyl}-malonamic acid methyl ester (0.148 g, 0.29 mmol) with 3.5 eq benzylamine as described in general procedure EE. Yield: 0.097 g, 82%. HRMS: AP+: 399.1665.

### Example 85

### (S)-N-[2-Oxo-3-(1,4,5,6-tetrahydro-1,2-diaza-benzo[e]azulen-8-yl)-oxazolidin-5-ylmethyl]-benzamide

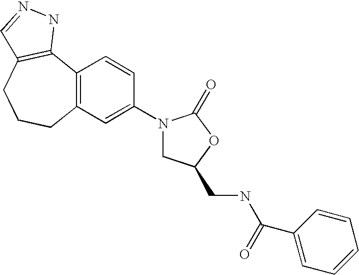

The title compound was prepared from (S)-N-[3-(1-benzoyl-1,4,5,6-tetrahydro-1,2-diaza-benzo[e]azulen-8-yl)-2-oxo-oxazolidin-5-ylmethyl]-benzamide (0.226 g, 0.44 mmol) with 3.5 eq benzylamine as described in general procedure EE. Yield: 0.162 g (90%). Melting point: 105-125° C. MS: AP+: 403.1 AP−: 401.1 Anal Calc. For C23H22N4O3-0.30H2O1: % Calc: C, 67.73; H, 5.59, N, 13.74. % Found: C, 67.42; H, 5.52, N, 13.34.

### Example 86

### (S)-3,3,3-Trifluoro-N-[2-oxo-3-(1,4,5,6-tetrahydro-1,2-diaza-benzo[e]azulen-8-yl)-oxazolidin-5-ylmethyl]-propionamide

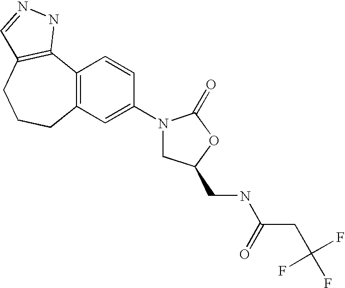

The title compound was prepared from (S)-3,3,3-trifluoro-N-{2-oxo-3-[1-(3,3,3-trifluoro-propionyl)-1,4,5,6-tetrahydro-1,2-diaza-benzo[e]azulen-8-yl]-oxazolidin-5-ylmethyl}-propionamide (0.200 g, 0.39 mmol) with 2.0 eq benzylamine as described in general procedure EE. Yield: 0.073 g (46%). Melting point: 243-245° C. MS: AP+: 409.2 AP−: 407.1 Anal Calc. For C19H19F3N4O3: % Calc: C, 55.88; H, 4.69, N, 13.72. % Found: C, 55.73; H: 4.44, N, 13.68.

### Example 87

### (S)-[3-(5,6-Dihydro-4H-1-oxa-2-aza-benzo[e]azulen-8-yl)-2-oxo-oxazolidin-5-ylmethyl]-carbamic acid methyl ester

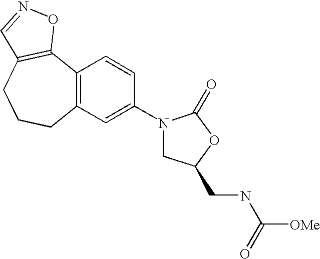

To a stirring solution of (S)-5-aminomethyl-3-(5,6-dihydro-4H-1-oxa-2-aza-benzo[e]azulen-8-yl)-oxazolidin-2-one (0.10 g, 0.3 mmol) and triethylamine (145 uL, 1.0 mmol) in dry methylene chloride (5.0 mL) at room temperature was added methyl chloroformate (0.034 g, 0.36 mmol) and the mixture stirred for 18 hours. The mixture was diluted with ethyl acetate and washed with saturated sodium bicarbonate and brine. The organic layer was dried, filtered and concentrated in vacuo to give a solid that was purified via flash chromatography with ethyl acetate to give the title compound, 0.100 g (93%). Melting point: 166-168° C. MS: AP+: 399.1. AP−: 356.1 Anal Calc. For C18H19N3O5: % Calc: C, 60.50; H, 5.36, N, 11.76. % Found: C, 60.26; H, 5.17, N, 11.59.

### Example 88

### (S)-[3-(5,6-Dihydro-4H-1-oxa-2-aza-benzo[e]azulen-8-yl)-2-oxo-oxazolidin-5-ylmethyl]-carbamic acid ethyl ester

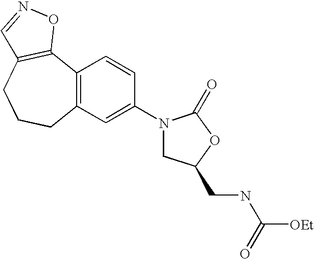

To a stirring solution of (S)-5-aminomethyl-3-(5,6-dihydro-4H-1-oxa-2-aza-benzo[e]azulen-8-yl)-oxazolidin-2-one (0.10 g, 0.3 mmol) and triethylamine (145 uL, 1.0 mmol) in dry methylene chloride (5.0 mL) at room temperature was added ethyl chloroformate (0.039 g, 0.36 mmol) and the mixture stirred for 18 hours. The mixture was diluted with ethyl acetate and washed with saturated sodium bicarbonate and brine. The organic layer was dried, filtered and concentrated in vacuo to give a solid that was purified via flash chromatography with ethyl acetate to give the title compound, 0.109 g (97%). Melting point: 181-183° C. MS: AP+: 372.1. AP−: 370.1 Anal Calc. For C19H21N3O5-0.02H2O1: % Calc: C, 61.39; H, 5.70, N, 11.30. % Found: C, 61.00; H, 5.63, N, 11.01.

### Example 89

### (S)-1-[3-(5,6-Dihydro-4H-1-oxa-2-aza-benzo[e]azulen-8-yl)-2-oxo-oxazolidin-5-ylmethyl]-3-ethyl-urea

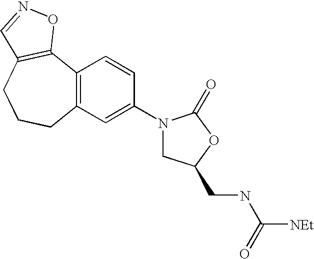

To a stirring solution of (S)-5-aminomethyl-3-(5,6-dihydro-4H-1-oxa-2-aza-benzo[e]azulen-8-yl)-oxazolidin-2-one (0.10 g, 0.3 mmol) and triethylamine (104 uL, 0.77 mmol) in dry methylene chloride (5.0 mL) at room temperature was added ethyl isocyanate (26 uL, 0.33 mmol) and the mixture stirred for 18 hours. The mixture was diluted with ethyl acetate and washed with saturated sodium bicarbonate and brine. The organic layer was dried, filtered and concentrated in vacuo to give a solid that was triturated with ether and filtered to give the title compound, 0.078 g (70%). Melting point: 193-196° C. MS: AP+: 371.1 AP−: 325.2. Anal Calc. For C₁₉H₂₂N₄O₄.0.25C₄H₁₀₀₁: % Calc: C, 61.13; H, 6.28, N: 14.26. % Found: C, 60.79; H, 6.06; N, 14.17.

### Example 90

### (S)-8-[5-(Methoxycarbonylamino-methyl)-2-oxo-oxazolidin-3-yl]-5,6-dihydro-4H-1,2-diaza-benzo[e]azulene-1-carboxylic acid methyl ester

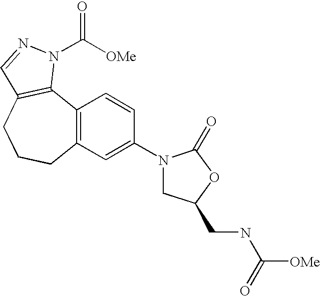

To a stirring solution of (S)-5-aminomethyl-3-(1,4,5,6-tetrahydro-1,2-diaza-benzo[e]azulen-8-yl)-oxazolidin-2-one hydrochloride (0.2 g, 0.6 mmol) and triethylamine (0.29 mL, 2.0 mmol) in dry methylene chloride at 0° C. was added methyl chloroformate (0.141 g, 1.49 mmol) at the mixture stirred for 18 hours while slowly warming to room temperature. It was then diluted with ethyl acetate and washed with saturated sodium bicarbonate and brine. The organic layer was dried, filtered and concentrated in vacuo and the residue purified via flash chromatography to give the title compound as a 6:1 mixture of mono and diacylated products as analyzed by LCMS, 0.109 g. MS: AP+: 415.2.

### Example 91

### (S)-8-[5-(Ethoxycarbonylamino-methyl)-2-oxo-oxazolidin-3-yl]-5,6-dihydro-4H-1,2-diaza-benzo[e]azulene-1-carboxylic acid ethyl ester

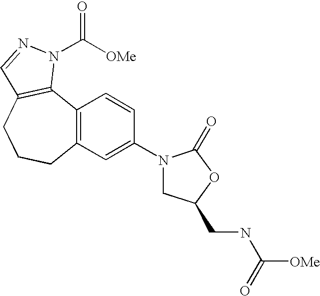

To a stirring solution of (S)-5-aminomethyl-3-(1,4,5,6-tetrahydro-1,2-diaza-benzo[e]azulen-8-yl)-oxazolidin-2-one hydrochloride (0.2 g, 0.6 mmol) and triethylamine (0.4 mL, 3 mmol) in dry methylene chloride (5 mL) at 0° C. was added ethyl chloroformate (0.26 g, 2.39 mmol) and the mixture stirred for 18 hours while slowly warming to room temperature. It was then diluted with ethyl acetate and washed with saturated sodium bicarbonate and brine. The organic layer was dried, filtered and concentrated in vacuo and the residue purified via flash chromatography (0-5% MeOH/dichloromethane) to give the title compound as a 6:1 mixture of mono and diacylated products as analyzed by LCMS, 0.109 g. MS: AP+: 433.3.

### Example 92

### (S)-1-Ethyl-3-[2-oxo-3-(1,4,5,6-tetrahydro-1,2-diaza-benzo[e]azulen-8-yl)-oxazolidin-5-ylmethyl]-urea

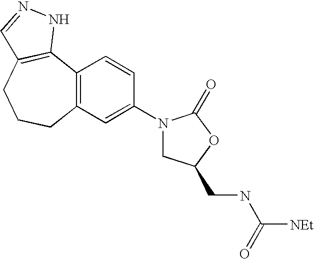

To a solution of (S)-5-aminomethyl-3-(1,4,5,6-tetrahydro-1,2-diaza-benzo[e]azulen-8-yl)-oxazolidin-2-one hydrochloride (0.1 g, 0.3 mmol) in dry methylene chloride (5 mL) and triethylamine (104 uL, 0.75 mmol) at 0° C. was added ethyl isocyanate (0.023 g, 0.33 mmol) in dry methylene chloride (2 mL) and the mixture stirred for 18 hours while slowly warming to room temperature. It was then diluted with ethyl acetate and washed with saturated sodium bicarbonate and brine. The organic layer was dried, filtered and concentrated in vacuo and the residue purified via flash chromatography to give the title compound, 0.052 g (47%). MS: AP+: 370.1. AP−: 297.1. Anal Calc. For C₁₉H₂₃N₅O₅.0.5H201: % Calc: C, 60.30; H, 6.39, N, 18.51. % Found: C, 60.11; H, 6.38; N, 18.18.

### Example 93

### (S)-2-Oxo-3-(1,4,5,6-tetrahydro-1,2-diaza-benzo[e]azulen-8-yl)-oxazolidin-5-ylmethyl]-carbamic acid methyl ester

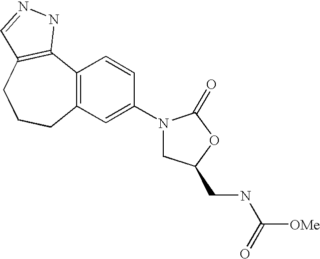

To a stirring solution of (S)-8-[5-(methoxycarbonylamino-methyl)-2-oxo-oxazolidin-3-yl]-5,6-dihydro-4H-1,2-diaza-benzo[e]azulene-1-carboxylic acid methyl ester (0.090 g, 0.22 mmol) in dry methanol (2 mL) at room temperature was added 25% methanolic sodium methoxide (0.25 mL, 1.1 mmol) and the mixture stirred by hand for 5 minutes. It was then diluted with ethyl acetate and washed with saturated sodium bicarbonate. The organic layer was dried, filtered and concentrated in vacuo and the residue purified via flash chromatography with 0-15% MeOH/dichloromethane to give a residue that was triturated with ether and filtered to give the title compound, 0.043 g (56%). Melting point: 87-93° C. MS: AP+: N/A. AP−: 355.1. Anal Calc. For C18H20N4O4-0.35H2O1: % Calc: C, 56.95; H, 5.42, N, 14.48. % Found: C, 57.29; H, 5.47; N, 14.08.

### Example 94

### (S)-[2-Oxo-3-(1,4,5,6-tetrahydro-1,2-diaza-benzo[e]azulen-8-yl)-oxazolidin-5-ylmethyl]-carbamic acid ethyl ester

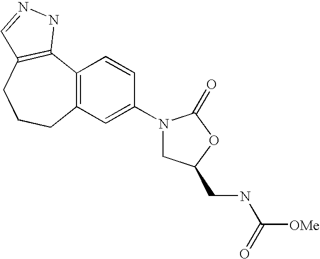

To a stirring solution of (S)-8-[5-(ethoxycarbonylamino-methyl)-2-oxo-oxazolidin-3-yl]-5,6-dihydro-4H-1,2-diaza-benzo[e]azulene-1-carboxylic acid ethyl ester (0.110 g, 0.25 mmol) in dry methanol (2 mL) at room temperature was added 25% methanolic sodium methoxide (0.28 mL, 1.24 mmol) and the mixture stirred by hand for 5 minutes. It was then diluted with ethyl acetate and washed with saturated sodium bicarbonate. The organic layer was dried, filtered and concentrated in vacuo and the residue purified via flash chromatography to give the title compound, 0.081 g (89%). Melting point: 122-126° C. MS: AP+: 371.1. AP−: 369.1. Anal Calc. For C19H22N4O4-0.01H2O1: % Calc: C, 61.58; H, 5.99, N, 15.12. % Found: C, 61.18; H, 5.96; N, 14.93.

### Example 95

### (S)-N-{3-[3-(4-Fluoro-phenyl)-1,4,5,6-tetrahydro-1,2-diaza-benzo[e]azulen-8-yl]-2-oxo-oxazolidin-5-ylmethyl}-acetamide

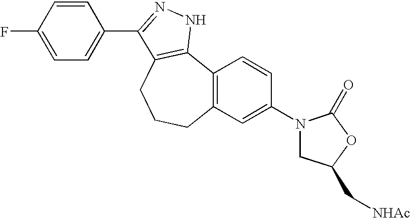

### N-[3-[6(R,S)-(4-Fluoro-benzoyl)-5-oxo-6,7,8,9-tetrahydro-5H-benzocyclohepten-2-yl]-2-oxo-oxazolidin-5(S)-Ylmethyl]-acetamide (84249x15) (Step 1)

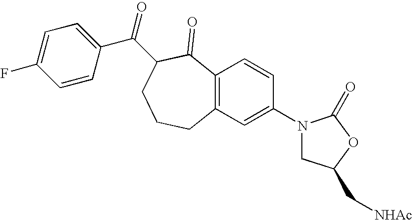

The title compound was prepared according to general method FF using (S)-N-[2-oxo-3-(5-oxo-6,7,8,9-tetrahydro-5H-benzocyclohepten-2-yl)-oxazolidin-5-ylmethyl]-acetamide (0.25 g, 0.79 mmol) was dissolved in THF (10 mL), LDA, (2M, 2.4 eq., 0.95 mL), and 4-fluorobenzoyl chloride (0.15 g, 1.2 eq., 0.95 mmol.). The isolated residue was subjected to silica gel flash chromatography, eluting with MeOH/CH₂Cl₂ gradient (0-6% MeOH over 1½ hours to afford the title compound. Isolated yield: 20%. MS-APCI (m/z+): 395, 439.

### (S)-N-[3-[3-(4-Fluoro-phenyl)-1,4,5,6-tetrahydro-1,2-diaza-benzo[e]azulen-8-yl]-2-oxo-oxazolidin-5-ylmethyl]-acetamide (PF-00111696-00-0001: 84249x20) (Step 2)

The title compound was prepared according to general method II using N-{3-[6(R,S)-(4-fluoro-benzoyl)-5-oxo-6,7,8,9-tetrahydro-5H-benzocyclohepten-2-yl]-2-oxo-oxazolidin-5(S)-ylmethyl}-acetamide (0.072 g, 0.16 mmol) and hydrazine hydrate (0.026 g, 0.80 mmol, 5.0 eq.) in ethanol (4 mL). The isolated residue was subjected to silica gel flash chromatography, eluting with MeOH/CH₂Cl₂ gradient (1-7% MeOH over 1 hour, then 7-9% MeOH over 30 minutes.) to afford the title compound. Isolated yield: 0.030 g (43%). MS-APCI (m/z+): 391, 435.

### Example 96

### (S)-N-[2-Oxo-3-(3-pyridin-4-yl-1,4,5,6-tetrahydro-1,2-diaza-benzo[e]azulen-8-yl)-oxazolidin-5-ylmethyl]-acetamide

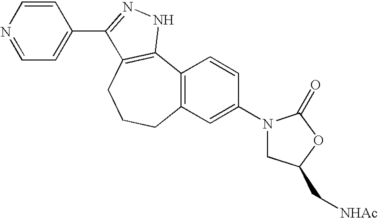

### N-{2-Oxo-3-[5-oxo-6(R,S)-(pyridine-4-carbonyl)-6,7,8,9-tetrahydro-5H-benzocyclo hepten-2-yl]-oxazolidin-5(S)-ylmethyl}-acetamide (80474x55A) (Step 1)

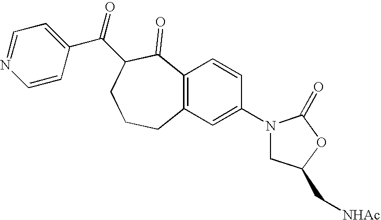

The title compound was prepared according to general procedure GG using (S)-N-[2-oxo-3-(5-oxo-6,7,8,9-tetrahydro-5H-benzocyclohepten-2-yl)-oxazolidin-5-ylmethyl]-acetamide (0.30 g, 0.95 mmol) dissolved in THF (10 mL), lithium t-butoxide (3.1 eq., 2.94 mL) and isonicotinoyl chloride hydrochloride (1.2 eq., 0.20 g, 1.13 mmol). The isolated residue was subjected to silica gel flash chromatography, eluting with MeOH/dichloromethane gradient (0-7% MeOH over 1 hour and 10 minutes) to afford the title compound. Isolated yield: 30%. MS-APCI (m/z+): 378, 422.

### (S)-N-[2-Oxo-3-(3-pyridin-4-yl-1,4,5,6-tetrahydro-1,2-diaza-benzo[e]azulen-8-yl)-oxazolidin-5-ylmethyl]-acetamide (Step 2)

The title compound was prepared according to general procedure II using N-{2-oxo-3-[5-oxo-6(R,S)-(pyridine-4-carbonyl)-6,7,8,9-tetrahydro-5H-benzocyclohepten-2-yl]-oxazolidin-5(S)-ylmethyl}-acetamide (0.12 g, 0.29 mmol) and hydrazine hydrate (0.041 g, 1.28 mmol, 4.5 eq.) in ethanol (6 mL). The isolated residue was subjected to silica gel flash chromatography, eluting with MeOH/dichloromethane gradient (2-8% MeOH over 1 hour, then 8-10% MeOH over 30 minutes.) to afford the title compound. Isolated yield: 0.055 g (46%). MS-APCI (m/z+): 374, 418.

### Example 97

### (S)-N-[2-Oxo-3-(3-trifluoromethyl-1,4,5,6-tetrahydro-1,2-diaza-benzo[e]azulen-8-yl)-oxazolidin-5-ylmethyl]-acetamide

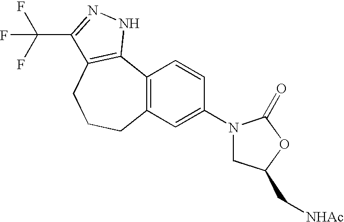

### N-{2-Oxo-3-[5-oxo-6(R,S)-(2,2,2-trifluoro-acetyl)-6,7,8,9-tetrahydro-5H-benzocyclohepten-2-yl]-oxazolidin-5(S)-ylmethyl}-acetamide (Step 1)

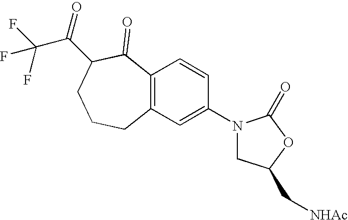

The title compound was prepared according to general procedure FF using (S)-N-[2-oxo-3-(5-oxo-6,7,8,9-tetrahydro-5H-benzocyclohepten-2-yl)-oxazolidin-5-ylmethyl]-acetamide (0.30 g, 0.95 mmol) dissolved in THF (12 mL), LDA, (2M, 2.5 eq., 1.19 L), and ethyl trifluoroacetate (0.149 g, 1.5 eq., 1.42 mmol.). The isolated residue was subjected to silica gel flash chromatography, eluting with MeOH/dichloromethane gradient (0-6% MeOH over 1½ hours to afford the title compound. Isolated yield: 38%. MS-APCI (m/z+): 369, 413.

### (S)-N-[2-Oxo-3-(3-trifluoromethyl-1,4,5,6-tetrahydro-1,2-diaza-benzo[e]azulen-8-yl)-oxazolidin-5-ylmethyl]-acetamide (Step 2)

N-{2-Oxo-3-[5-oxo-6(R,S)-(2,2,2-trifluoro-acetyl)-6,7,8,9-tetrahydro-5H-benzocyclohepten-2-yl]-oxazolidin-5(S)-ylmethyl}-acetamide (0.15 g, 0.36 mmol) and hydrazine hydrate (0.052 g, 1.64 mmol, 4.5 eq.) in ethanol (9 mL) were stirred at room temperature overnight. The reaction mixture, containing mostly uncylcized hydrazone, was concentrated in vacuo. After stirring, by the isolated residue was placed in acetic acid (5 mL) and heated to 100° C. After 1½ hours heating was stopped and solvent was removed in vacuo. The isolated residue was triturated with ethyl acetate. The resulting solids were filtered off and washed with ethyl acetate and ether to afford the title compound. Isolated yield: 0.085 g (57MS-APCI (m/z+): 409.

### Example 98

### PF-00184178

### (S)-N-[2-Oxo-3-(3-phenyl-1,4,5,6-tetrahydro-1,2-diaza-benzo[e]azulen-8-yl)-oxazolidin-5-ylmethyl]-acetamide

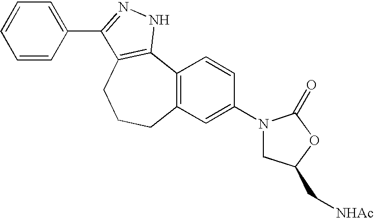

### N-[3-(6(R,S)-Benzoyl-5-oxo-6,7,8,9-tetrahydro-5H-benzocyclohepten-2-yl)-2-oxo-oxazolidin-5(S)-ylmethyl]-acetamide (Step 1)

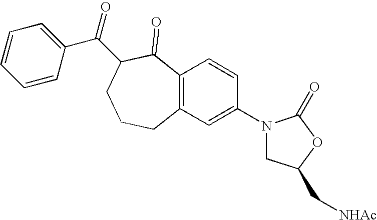

The title compound was prepared according to general procedure HH using (S)-N-[2-oxo-3-(5-oxo-6,7,8,9-tetrahydro-5H-benzocyclohepten-2-yl)-oxazolidin-5-ylmethyl]-acetamide (0.30 g, 0.95 mmol) dissolved in THF (8 mL), LiHMDS (1M, 2.0 eq., 1.90 mL) and benzoyl chloride (1.05 eq., 0.14 g, 0.10 mmol) in THF (2 mL). The isolated residue was subjected to silica gel flash chromatography, eluting with MeOH/dichloromethane gradient (0-6% MeOH over 1 hour and 10 minutes) to afford the title compound. Isolated yield: 21%. MS-APCI (m/z+): 377, 421.

### (S)-N-[2-Oxo-3-(3-phenyl-1,4,5,6-tetrahydro-1,2-diaza-benzo[e]azulen-8-yl)-oxazolidin-5-ylmethyl]-acetamide (Step 2)

The title compound was prepared according to general procedure II using N-[3-(6(R,S)-benzoyl-5-oxo-6,7,8,9-tetrahydro-5H-benzocyclohepten-2-yl)-2-oxo-oxazolidin-5(S)-ylmethyl]-acetamide (0.085 g, 0.20 mmol) and hydrazine hydrate (0.032 g, 1.01 mmol, 5.0 eq.) in ethanol (8 mL). The isolated residue was triturated with ethyl acetate. The resulting solids were filtered off and washed with ethyl acetate and ether to afford the title compound. Isolated yield: 0.065 g (77%). MS-APCI (m/z+): 373, 417.

### Example 99

### (S)-N-[3-(3-Methyl-1,4,5,6-tetrahydro-1,2-diaza-benzo[e]azulen-8-yl)-2-oxo-oxazolidin-5-ylmethyl]-acetamide

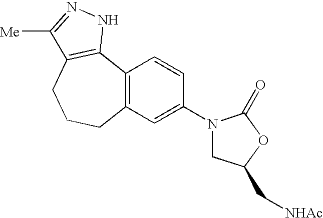

### N-[3-(6(R,S)-Acetyl-5-oxo-6,7,8,9-tetrahydro-5H-benzocyclohepten-2-yl)-2-oxo-oxazolidin-5(S)-ylmethyl]-acetamide (Step 1)

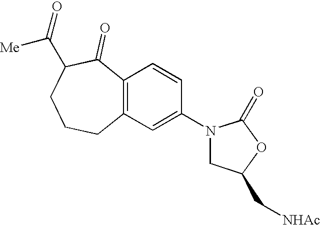

The title compound was prepared according to general procedure HH using (S)-N-[2-oxo-3-(5-oxo-6,7,8,9-tetrahydro-5H-benzocyclohepten-2-yl)-oxazolidin-5-ylmethyl]-acetamide (0.40 g, 1.26 mmol) dissolved in THF (10 mL), LiHMDS (1M, 2.0 eq., 2.53 mL) and acetyl chloride (1.05 eq., 0.104 g, 1.33 mmol) in THF (2 mL). The isolated residue was subjected to silica gel flash chromatography, eluting with MeOH/dichloromethane gradient (0-6% MeOH over 1 hour and 10 minutes) to afford the title compound. Isolated yield: 62%. MS-APCI (m/z+): 315, 359.

### (S)-N-[3-(3-Methyl-1,4,5,6-tetrahydro-1,2-diaza-benzo[e]azulen-8-Yl)-2-oxo-oxazolidin-5-ylmethyl]-acetamide (Step 2)

The title compound was prepared according to general procedure II using N-[3-(6(R,S)-acetyl-5-oxo-6,7,8,9-tetrahydro-5H-benzocyclohepten-2-yl)-2-oxo-oxazolidin-5(S)-ylmethyl]-acetamide (0.280 g, 0.78 mmol) and hydrazine hydrate (0.125 g, 3.91 mmol, 5.0 eq.) in ethanol (12 mL). The isolated residue was subjected to silica gel flash chromatography, eluting with MeOH/dichloromethane gradient (0-7% MeOH over 1 hour) to afford the title compound. Isolated yield: 0.067 g (24%). MS-APCI (m/z+): 311, 355.

### Example 100

### (S)-N-{2-Oxo-3-[3-(4-trifluoromethoxy-phenyl)-1,4,5,6-tetrahydro-1,2-diaza-benzo[e]azulen-8-yl]-oxazolidin-5-ylmethyl}-acetamide

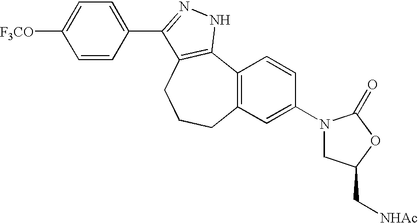

### N-{2-Oxo-3-[5-oxo-6(R,S)-(4-trifluoromethoxy-benzoyl)-6,7,8,9-tetrahydro-5H-benzocyclohepten-2-yl]-oxazolidin-5(S)-ylmethyl}-acetamide (Step 1)

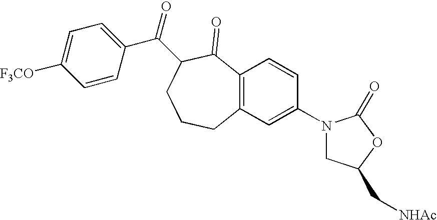

The title compound was prepared according to general procedure HH using (S)-N-[2-oxo-3-(5-oxo-6,7,8,9-tetrahydro-5H-benzocyclohepten-2-yl)-oxazolidin-5-ylmethyl]-acetamide (0.30 g, 0.95 mmol) dissolved in THF (8 mL), LiHMDS (1M, 2.1 eq., 1.992 mL) and 4-triflouormethoxybenzoyl chloride (1.2 eq., 0.256 g, 1.14 mmol) in THF (2 mL). The isolated residue was subjected to silica gel flash chromatography, eluting with MeOH/dichloromethane gradient (0-5% MeOH over 1 hour and 10 minutes) to afford the title compound. Isolated yield: 50%. MS-APCI (m/z+): 461, 505.

### (S)-N-{2-Oxo-3-[3-(4-trifluoromethoxy-phenyl)-1,4,5,6-tetrahydro-1,2-diaza-benzo[e]azulen-8-yl]-oxazolidin-5-ylmethyl}-acetamide (Step 2)

The title compound was prepared according to general procedure II using N-{2-Oxo-3-[5-oxo-6(R,S)-(4-trifluoromethoxy-benzoyl)-6,7,8,9-tetrahydro-5H-benzocyclohepten-2-yl]-oxazolidin-5(S)-ylmethyl}-acetamide (0.240 g, 0.48 mmol) and hydrazine hydrate (0.076 g, 2.40 mmol, 5.0 eq.) in Ethanol (10 mL). The isolated residue was triturated with ethanol/ethyl acetate. The resulting solids were filtered and washed with cold ethanol and ether to afford the title compound. Isolated yield: 0.165 g (69%). MS-APCI (m/z+): 457, 501.

### Example 101

### (S)-N-{2-Oxo-3-[3-(4-cyano-phenyl)-1,4,5,6-tetrahydro-1,2-diaza-benzo[e]azulen-8-yl]-oxazolidin-5-ylmethyl}-acetamide

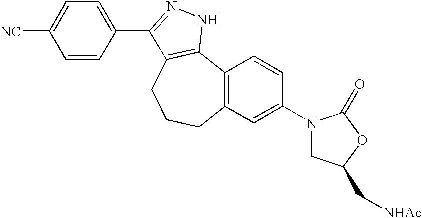

### N-[3-[6(R,S)-(4-Cyano-benzoyl)-5-oxo-6,7,8,9-tetrahydro-5H-benzocyclohepten-2-yl]-2-oxo-oxazolidin-5(S)-ylmethyl]-acetamide (Step 1)

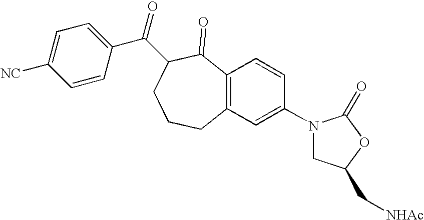

The title compound was prepared according to general procedure HH using (S)-N-[2-oxo-3-(5-oxo-6,7,8,9-tetrahydro-5H-benzocyclohepten-2-yl)-oxazolidin-5-ylmethyl]-acetamide (0.30 g, 0.95 mmol) dissolved in THF (8 mL), LiHMDS (1M, 2.1 eq., 1.99 mL) and 4-cyanobenzoyl chloride (1.2 eq., 0.188 g, 1.14 mmol) in THF (2 mL). The isolated residue was subjected to silica gel flash chromatography, eluting with MeOH/dichloromethane gradient (0-5% MeOH over 1 hour and 10 minutes) to afford the title compound. Isolated yield: 36%. MS-APCI (m/z+): 402, 546.

### (S)-N-{2-Oxo-3-[3-(4-cyano-phenyl)-1,4,5,6-tetrahydro-1,2-diaza-benzo[e]azulen-8-yl]-oxazolidin-5-ylmethyl}-acetamide (Step 2)

The title compound was prepared according to general procedure II using N-{3-[6(R,S)-(4-cyano-benzoyl)-5-oxo-6,7,8,9-tetrahydro-5H-benzocyclohepten-2-yl]-2-oxo-oxazolidin-5(S)-ylmethyl}-acetamide (0.150 g, 0.34 mmol) and hydrazine hydrate (0.054 g, 1.68 mmol, 5.0 eq.) in Ethanol (10 mL). The isolated residue was subjected to silica gel flash chromatography, eluting with MeOH/dichloromethane gradient (0-6% MeOH over 1 hour, 10 minutes) to afford the title compound. Isolated yield: 0.065 g (44%). MS-APCI (m/z+): 398, 442.

### Example 102

### (S)-N-[2-Oxo-3-(3-thiazol-4-yl-1,4,5,6-tetrahydro-1,2-diaza-benzo[e]azulen-8-yl)-oxazolidin-5-ylmethyl]-acetamide

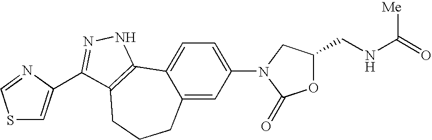

### N-{2-Oxo-3-[5-oxo-6(R,S)-(thiazole-4-carbonyl)-6,7,8,9-tetrahydro-5H-benzocyclohepten-2-yl]-oxazolidin-5(S)-ylmethyl}-acetamide (Step 1)

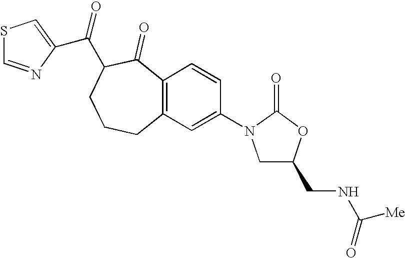

The title compound was prepared according to general procedure GG using (S)-N-[2-oxo-3-(5-oxo-6,7,8,9-tetrahydro-5H-benzocyclohepten-2-yl)-oxazolidin-5-ylmethyl]-acetamide (0.35 g, 1.11 mmol) dissolved in THF (12 mL), lithium t-butoxide (2.5 eq., 2.77 mL) and ethyl thiazole-4-carboxylate (1.2 eq., 0.21 g, 1.33 mmol). The isolated residue was subjected to silica gel flash chromatography, eluting with MeOH/dichloromethane gradient (0-6% MeOH over 1 hour and 10 minutes) to afford the title compound. Isolated yield: 36%. MS-APCI (m/z+): 384, 428.

### (S)-N-[2-Oxo-3-(3-thiazol-4-yl-1,4,5,6-tetrahydro-1,2-diaza-benzo[e]azulen-8-yl)-oxazolidin-5-ylmethyl]-acetamide (Step 2)

The title compound was prepared according to general procedure II using N-{2-oxo-3-[5-oxo-6(R,S)-(thiazole-4-carbonyl)-6,7,8,9-tetrahydro-5H-benzocyclohepten-2-yl]-oxazolidin-5(S)-ylmethyl}-acetamide (0.170 g, 0.40 mmol) and hydrazine hydrate (0.064 g, 1.99 mmol, 5.0 eq.) in ethanol (8 mL). The isolated residue was subjected to silica gel flash chromatography, eluting with MeOH/dichloromethane gradient (1-8% MeOH over 1 hour, 10 minutes.) to afford the title compound. Isolated yield: 0.008 g (5%). MS-APCI (m/z+): 380, 424.

### Example 103

### (S)-N-[3-(3-Isoxazol-5-yl-1,4,5,6-tetrahydro-1,2-diaza-benzo[e]azulen-8-yl)-2-oxo-oxazolidin-5-ylmethyl]-acetamide

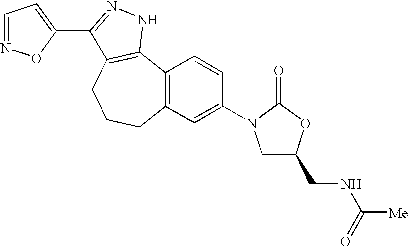

### N-{3-[6(R,S)-(Isoxazole-5-carbonyl)-5-oxo-6,7,8,9-tetrahydro-5H-benzocyclohepten-2-yl]-2-oxo-oxazolidin-5(S)-ylmethyl}-acetamide (Step 1)

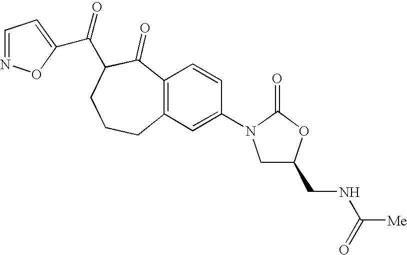

The title compound was prepared according to general procedure HH using (S)-N-[2-oxo-3-(5-oxo-6,7,8,9-tetrahydro-5H-benzocyclohepten-2-yl)-oxazolidin-5-ylmethyl]-acetamide (0.40 g, 1.26 mmol) dissolved in THF (11 mL), LiHMDS (1M, 2.1 eq., 2.66 mL) and isoxazole-5-carbonyl chloride (1.2 eq., 0.20 g, 1.52 mmol) in THF (3 mL). The isolated residue was subjected to silica gel flash chromatography, eluting with MeOH/dichloromethane gradient (0-6% MeOH over 1 hour and 10 minutes) to afford the title compound. Isolated yield: 38%. MS-APCI (m/z+): 412.

### (S)-N-[3-(3-Isoxazol-5-yl-1,4,5,6-tetrahydro-1,2-diaza-benzo[e]azulen-8-yl)-2-oxo-oxazolidin-5-ylmethyl]-acetamide (Step 2)

The title compound was prepared according to general procedure II using N-{3-[6(R,S)-(isoxazole-5-carbonyl)-5-oxo-6,7,8,9-tetrahydro-5H-benzocyclohepten-2-yl]-2-oxo-oxazolidin-5(S)-ylmethyl}-acetamide (0.210 g, 0.51 mmol) and hydrazine hydrate (0.041 g, 1.28 mmol, 2.5 eq.) in ethanol (10 mL). The isolated residue was subjected to silica gel flash chromatography, eluting with MeOH/dichloromethane gradient (1-7% MeOH over 1 hour, 10 minutes.) to afford the title compound. Isolated yield: 0.100 g (48%). MS-APCI (m/z+): 364, 408.

### Example 104

### (S)-N-[3-(3-Furan-3-yl-1,4,5,6-tetrahydro-1,2-diaza-benzo[e]azulen-8-yl)-2-oxo-oxazolidin-5-ylmethyl]-acetamide and (S)-Furan-3-carboxylic acid [3-(3-furan-3-yl-1,4,5,6-tetrahydro-1,2-diaza-benzo[e]azulen-8-yl)-2-oxo-oxazolidin-5-ylmethyl]-amide

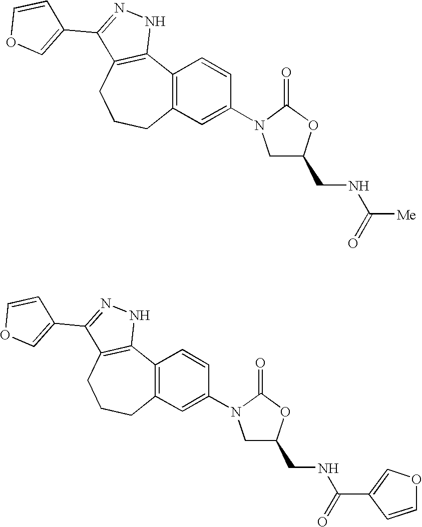

### N-{3-[6(R,S)-(Furan-3-carbonyl)-5-oxo-6,7,8,9-tetrahydro-5H-benzocyclohepten-2-yl]-2-oxo-oxazolidin-5(S)-ylmethyl}-acetamide and (S)-Furan-3-carboxylic acid {3-[6(R,S)-(furan-3-carbonyl)-5-oxo-6,7,8,9-tetrahydro-5H-benzocyclohepten-2-yl]-2-oxo-oxazolidin-5(S)-ylmethyl}-amide (Step 1)

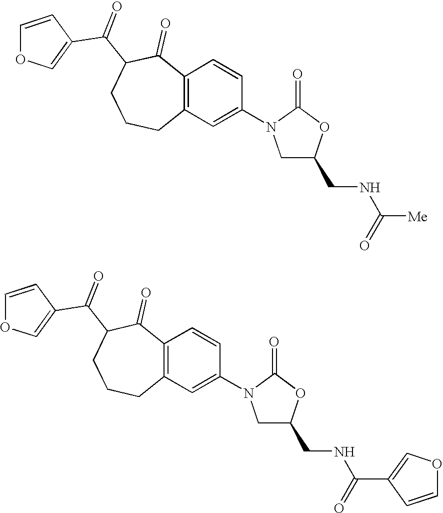

The title compounds were prepared according to general procedure HH using (S)-N-[2-oxo-3-(5-oxo-6,7,8,9-tetrahydro-5H-benzocyclohepten-2-yl)-oxazolidin-5-ylmethyl]-acetamide (0.50 g, 1.58 mmol) dissolved in THF (14 mL), LiHMDS (1 M, 2.1 eq., 3.32 mL) and furan-3-carbonyl chloride (1.1 eq., 0.227 g, 1.74 mmol) in THF (4 mL). The isolated residue was subjected to silica gel flash chromatography, eluting with MeOH/dichloromethane gradient (0-6% MeOH over 1 hour and 10 minutes) to afford the title compounds.

For N-{3-[6(R,S)-(Furan-3-carbonyl)-5-oxo-6,7,8,9-tetrahydro-5H-benzocyclohepten-2-yl]-2-oxo-oxazolidin-5(S)-ylmethyl}-acetamide: Isolated yield: 31%. MS-APCI (m/z+): 367, 411;

For Furan-3-carboxylic acid {3-[6(R,S)-(furan-3-carbonyl)-5-oxo-6,7,8,9-tetrahydro-5H-benzocyclohepten-2-yl]-2-oxo-oxazolidin-5(S)-ylmethyl}-amide: Isolated yield: 27%. MS-APCI (m/z+): 419, 463.

### (S)-N-[3-(3-Furan-3-yl-1,4,5,6-tetrahydro-1,2-diaza-benzo[e]azulen-8-yl)-2-oxo-oxazolidin-5-ylmethyl]-acetamide (Step 2)

The title compound was prepared according to general procedure II using N-{3-[6(R,S)-(furan-3-carbonyl)-5-oxo-6,7,8,9-tetrahydro-5H-benzocyclohepten-2-yl]-2-oxo-oxazolidin-5(S)-ylmethyl}-acetamide (0.200 g, 0.49 mmol) and hydrazine hydrate (0.039 g, 1.22 mmol, 2.5 eq.) in ethanol (10 mL). The isolated residue was triturated with ethyl acetate/dichloromethane mixture. The resulting solids were filtered and washed with Ethyl acetate and Et2O to afford the title compound. Isolated yield: 0.140 g (71%). MS-APCI (m/z+): 363, 407.

### (S)-Furan-3-carboxylic acid [3-(3-furan-3-yl-1,4,5,6-tetrahydro-1,2-diaza-benzo[e]azulen-8-yl)-2-oxo-oxazolidin-5-ylmethyl]-amide (Step 2′)

The title compound was prepared according to general procedure II using furan-3-carboxylic acid {3-[6(R,S)-(furan-3-carbonyl)-5-oxo-6,7,8,9-tetrahydro-5H-benzocyclohepten-2-yl]-2-oxo-oxazolidin-5(S)-ylmethyl}-amide (0.200 g, 0.43 mmol) and hydrazine hydrate (0.035 g, 1.08 mmol, 2.5 eq.) in ethanol (10 mL). The isolated residue was triturated with ethyl acetate/dichloromethane mixture. The resulting solids were filtered and washed with Ethyl acetate and Et₂O to afford the title compound. Isolated yield: 0.075 g (38%). MS-APCI (m/z+): 415, 459.

### Example 105

### (S)-N-{3-[3-(4-Methyl-[1,2,3]thiadiazol-5-yl)-1,4,5,6-tetrahydro-1,2-diaza-benzo[e]azulen-8-yl]-2-oxo-oxazolidin-5-ylmethyl}-acetamide

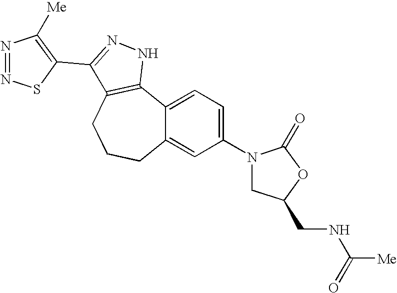

### N-[3-[6(R,S)-(4-Methyl-[1,2,3]thiadiazole-5-carbonyl)-5-oxo-6,7,8,9-tetrahydro-5H-benzocyclohepten-2-yl]-2-oxo-oxazolidin-5(S)-ylmethyl]-acetamide (Step 1)

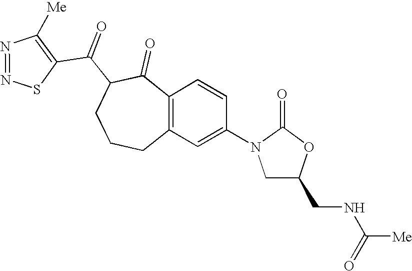

The title compound was prepared according to general procedure HH using (S)-N-[2-oxo-3-(5-oxo-6,7,8,9-tetrahydro-5H-benzocyclohepten-2-yl)-oxazolidin-5-ylmethyl]-acetamide (0.50 g, 1.58 mmol) dissolved in THF (14 mL), LiHMDS (1M, 2.1 eq., 3.32 mL) and 4-Methyl-[1,2,3]thiadiazole-5-carbonyl chloride (1.1 eq., 0.283 g, 1.74 mmol) in THF (3 mL). The isolated residue was subjected to silica gel flash chromatography, eluting with MeOH/dichloromethane gradient (0-6% MeOH over 1 hour and 10 minutes) to afford the title compounds. Isolated yield: 36%. MS-APCI (m/z+): 399, 443.

### (S)-N-[3-[3-(4-Methyl-[1,2,3]thiadiazol-5-yl)-1,4,5,6-tetrahydro-1,2-diaza-benzo[e]azulen-8-yl]-2-oxo-oxazolidin-5-ylmethyl]-acetamide (Step 2)

The title compound was prepared according to general procedure II using N-{3-[6(R,S)-(4-Methyl-[1,2,3]thiadiazole-5-carbonyl)-5-oxo-6,7,8,9-tetrahydro-5H-benzocyclohepten-2-yl]-2-oxo-oxazolidin-5(S)-ylmethyl}-acetamide (0.250 g, 0.57 mmol) and hydrazine hydrate (0.045 g, 1.41 mmol, 2.5 eq.) in Ethanol (12 mL). The isolated residue was subjected to silica gel flash chromatography, eluting with MeOH/dichloromethane gradient (0-7% MeOH over 1 hour, 10 minutes, then 7-9% over 30 minutes.) to afford the title compound. Isolated yield: 0.120 g (48%). MS-APCI (m/z+): 395, 439.

### Example 106

### (S)-N-{3-[3-(5-Methyl-isoxazol-3-yl)-1,4,5,6-tetrahydro-1,2-diaza-benzo[e]azulen-8-yl]-2-oxo-oxazolidin-5-ylmethyl}-acetamide and (S)-5-Methyl-isoxazole-3-carboxylic acid {3-[3-(5-methyl-isoxazol-3-yl)-1,4,5,6-tetrahydro-1,2-diaza-benzo[e]azulen-8-yl]-2-oxo-oxazolidin-5-ylmethyl}-amide

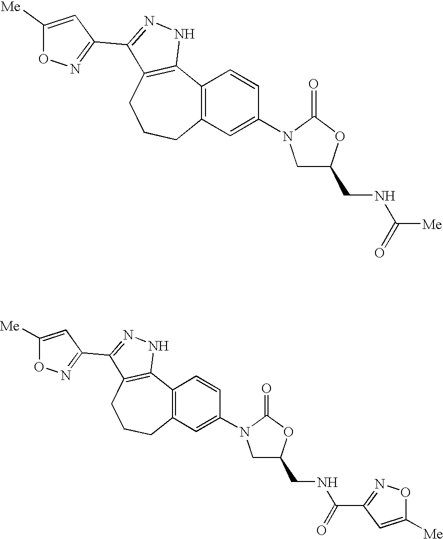

### N-{3-[6(R,S)-(5-Methyl-isoxazole-3-carbonyl)-5-oxo-6,7,8,9-tetrahydro-5H-benzocyclohepten-2-yl]-2-oxo-oxazolidin-5(S)-ylmethyl}-acetamide and 5-Methyl-isoxazole-3-carboxylic acid {3-[6(R,S)-(5-methyl-isoxazole-3-carbonyl)-5-oxo-6,7,8,9-tetrahydro-5H-benzocyclohepten-2-yl]-2-oxo-oxazolidin-5(S)-ylmethyl}-amide (Step 1)

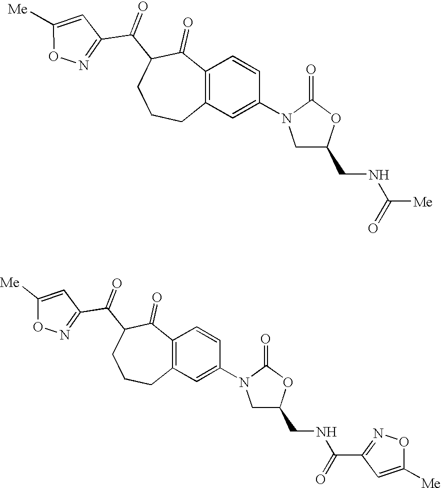

The title compounds were prepared according to general procedure HH using (S)-N-[2-oxo-3-(5-oxo-6,7,8,9-tetrahydro-5H-benzocyclohepten-2-yl)-oxazolidin-5-ylmethyl]-acetamide (0.50 g, 1.58 mmol) dissolved in THF (14 mL), LiHMDS (1M, 2.1 eq., 3.32 mL) and 5-methyl-isoxazole-3-carbonyl chloride (1.2 eq., 0.276 g, 1.90 mmol) in THF (4 mL). The isolated residue was subjected to silica gel flash chromatography, eluting with MeOH/dichloromethane gradient (0-6% MeOH over 1 hour and 10 minutes) to afford the title compounds.

For N-{3-[6(R,S)-(5-Methyl-isoxazole-3-carbonyl)-5-oxo-6,7,8,9-tetrahydro-5H-benzocyclohepten-2-yl]-2-oxo-oxazolidin-5(S)-ylmethyl}-acetamide: Isolated yield: 18%. MS-APCI (m/z+): 426;

For (S)-5-Methyl-isoxazole-3-carboxylic acid {3-[6(R,S)-(5-methyl-isoxazole-3-carbonyl)-5-oxo-6,7,8,9-tetrahydro-5H-benzocyclohepten-2-yl]-2-oxo-oxazolidin-5(S)-ylmethyl}-amide: Isolated yield: 36%. MS-APCI (m/z+): 493.

### (S)-N-{3-[3-(5-Methyl-isoxazol-3-yl)-1,4,5,6-tetrahydro-1,2-diaza-benzo[e]azulen-8-yl]-2-oxo-oxazolidin-5-ylmethyl}-acetamide (Step 2)

The title compound was prepared according to general procedure II using N-{3-[6(R,S)-(5-methyl-isoxazole-3-carbonyl)-5-oxo-6,7,8,9-tetrahydro-5H-benzocyclohepten-2-yl]-2-oxo-oxazolidin-5(S)-ylmethyl}-acetamide (0.120 g, 0.28 mmol) and hydrazine hydrate (0.023 g, 0.71 mmol, 2.5 eq.) in ethanol (10 mL). The isolated residue was subjected to silica gel flash chromatography, eluting with MeOH/dichloromethane gradient (0-6% MeOH over 1 hour, 10 minutes, then 6-8% over 30 minutes.) to afford the title compound. Isolated yield: 0.050 g (42%). MS-APCI (m/z+): 378, 422.

### (S)-5-Methyl-isoxazole-3-carboxylic acid {3-[3-(5-methyl-isoxazol-3-yl)-1,4,5,6-tetrahydro-1,2-diaza-benzo[e]azulen-8-yl]-2-oxo-oxazolidin-5-ylmethyl}-amide (Step 2′)

The title compound was prepared according to general procedure II using 5-methyl-isoxazole-3-carboxylic acid {3-[6(R,S)-(5-methyl-isoxazole-3-carbonyl)-5-oxo-6,7,8,9-tetrahydro-5H-benzocyclohepten-2-yl]-2-oxo-oxazolidin-5(S)-ylmethyl}-amide (0.280 g, 0.57 mmol) and hydrazine hydrate (0.046 g, 1.42 mmol, 2.5 eq.) in ethanol (14 mL). The isolated residue was subjected to silica gel flash chromatography, eluting with MeOH/dichloromethane gradient (0-6% MeOH over 1 hour, 10 minutes, then 6-8% over 30 minutes.) to afford the title compound. Isolated yield: 0.088 g (32%). MS-APCI (m/z+): 445, 489.

### Example 107

### (S)-N-[2-Oxo-3-(3-pyridin-3-yl-1,4,5,6-tetrahydro-1,2-diaza-benzo[e]azulen-8-yl)-oxazolidin-5-ylmethyl]-acetamide and (S)-N-[2-Oxo-3-(3-pyridin-3-yl-1,4,5,6-tetrahydro-1,2-diaza-benzo[e]azulen-8-yl)-oxazolidin-5-ylmethyl]-nicotinamide

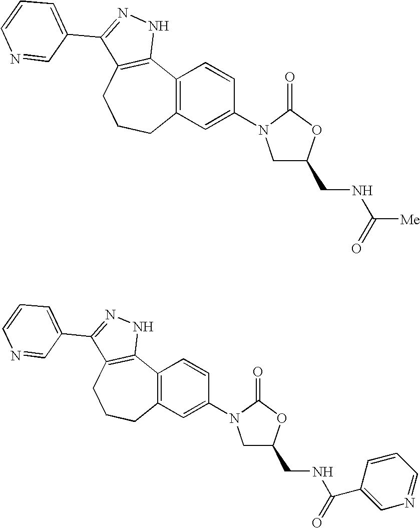

### N-{2-Oxo-3-[5-oxo-6(R,S)-(Pyridine-3-carbonyl)-6,7,8,9-tetrahydro-5H-benzocyclohepten-2-yl]-oxazolidin-5(S)-ylmethyl}-acetamide and N-{2-Oxo-3-[5-oxo-6(R,S)-(pyridine-3-carbonyl)-6,7,8,9-tetrahydro-5H-benzocyclohepten-2-yl]-oxazolidin-5(S)-ylmethyl}-nicotinamide (Step 1)

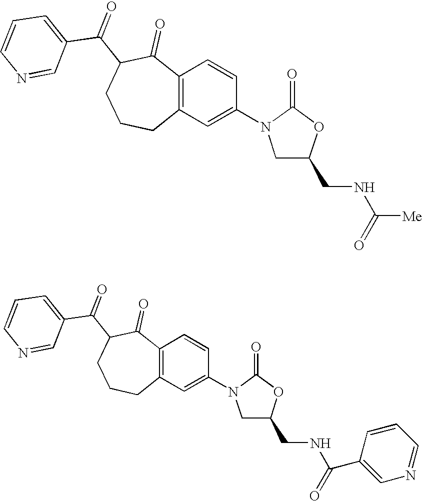

The title compounds were prepared according to general procedure HH using (S)-N-[2-oxo-3-(5-oxo-6,7,8,9-tetrahydro-5H-benzocyclohepten-2-yl)-oxazolidin-5-ylmethyl]-acetamide (0.50 g, 1.58 mmol) dissolved in THF (14 mL), LiHMDS (1M, 3.15 eq., 4.98 mL) and nicotinoyl chloride hydrochloride (1.0 eq., 0.282 g, 1.58 mmol) as a solid. The isolated residue was subjected to silica gel flash chromatography, eluting with MeOH/dichloromethane gradient (0-7% MeOH over 1 hour and 10 minutes) to afford the title compounds.

For N-{2-Oxo-3-[5-oxo-6(R,S)-(pyridine-3-carbonyl)-6,7,8,9-tetrahydro-5H-benzocyclohepten-2-yl]-oxazolidin-5 (S)-ylmethyl}-acetamide, Isolated yield: 26%. MS-APCI (m/z+): 378, 422;

For N-{2-Oxo-3-[5-oxo-6(R,S)-(pyridine-3-carbonyl)-6,7,8,9-tetrahydro-5H-benzocyclohepten-2-yl]-oxazolidin-5(S)-ylmethyl}-nicotinamide: Isolated yield: 16%. MS-APCI (m/z+): 441, 485.

### (S)-N-[2-Oxo-3-(3-pyridin-3-yl-1,4,5,6-tetrahydro-1,2-diaza-benzo[e]azulen-8-yl)-oxazolidin-5-ylmethyl]-acetamide (Step 2)

The title compound was prepared according to general procedure II using N-{2-oxo-3-[5-oxo-6(R,S)-(pyridine-3-carbonyl)-6,7,8,9-tetrahydro-5H-benzocyclohepten-2-yl]-oxazolidin-5(S)-ylmethyl}-acetamide (0.170 g, 0.40 mmol) and hydrazine hydrate (0.032 g, 1.01 mmol, 2.5 eq.) in ethanol (12 mL). The isolated residue was subjected to silica gel flash chromatography, eluting with MeOH/dichloromethane gradient (0-7% MeOH over 1 hour, 10 minutes, then 7-9% over 30 minutes) to afford the title compound. Isolated yield: 0.112 g (67%). MS-APCI (m/z+): 374, 418.

### (S)-N-[2-Oxo-3-(3-pyridin-3-yl-1,4,5,6-tetrahydro-1,2-diaza-benzo[e]azulen-8-yl)-oxazolidin-5-ylmethyl]-nicotinamide (Step 2′)

The title compound was prepared according to general procedure II using N-{2-oxo-3-[5-oxo-6(R,S)-(pyridine-3-carbonyl)-6,7,8,9-tetrahydro-5H-benzocyclohepten-2-yl]-oxazolidin-5(S)-ylmethyl}-nicotinamide (0.120 g, 0.25 mmol) and hydrazine hydrate (0.020 g, 0.62 mmol, 2.5 eq.) in Ethanol (10 mL). The isolated residue was subjected to silica gel flash chromatography, eluting with MeOH/dichloromethane gradient (1-7% MeOH over 1 hour, 10 minutes, then 7-9% over 30 minutes.) to afford the title compound. Isolated yield: 0.055 g (46%). MS-APCI (m/z+): 437, 481.

### Example 108

### (S)-N-{2-Oxo-3-[3-(2-phenyl-thiazol-4-yl)-1,4,5,6-tetrahydro-1,2-diaza-benzo[e]azulen-8-yl]-oxazolidin-5-ylmethyl}-acetamide

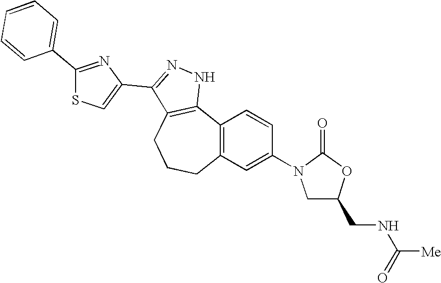

### N-{2-Oxo-3-[5-oxo-6(R,S)-(2-phenyl-thiazole-4-carbonyl)-6,7,8,9-tetrahydro-5H-benzocyclohepten-2-yl]-oxazolidin-5(S)-ylmethyl}-acetamide (Step 1)

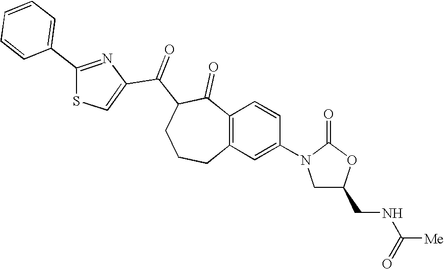

The title compound was prepared according to general procedure HH using (S)-N-[2-oxo-3-(5-oxo-6,7,8,9-tetrahydro-5H-benzocyclohepten-2-yl)-oxazolidin-5-ylmethyl]-acetamide (0.50 g, 1.58 mmol) dissolved in THF (16 mL), LiHMDS (1M, 2.1 eq., 3.32 mL) and 2-phenyl-thiazole-4-carbonyl chloride (1.1 eq., 0.389 g, 1.74 mmol) as a solid. The isolated residue was subjected to silica gel flash chromatography, eluting with MeOH/dichloromethane gradient (0-7% MeOH over 1 hour and 10 minutes) to afford the title compounds. Isolated yield: 29%. MS-APCI (m/z+): 460, 504.

### (S)-N-{2-Oxo-3-[3-(2-phenyl-thiazol-4-yl)-1,4,5,6-tetrahydro-1,2-diaza-benzo[e]azulen-8-yl]-oxazolidin-5-ylmethyl}-acetamide (Step 2)

The title compound was prepared according to general procedure II using N-{2-oxo-3-[5-oxo-6(R,S)-(2-phenyl-thiazole-4-carbonyl)-6,7,8,9-tetrahydro-H-benzocyclohepten-2-yl]-oxazolidin-5(S)-ylmethyl}-acetamide (0.230 g, 0.46 mmol) and hydrazine hydrate (0.037 g, 1.14 mmol, 2.5 eq.) in Ethanol (10 mL). The isolated residue was triturated with ethyl acetate/dichloromethane mixture. The resulting solids were filtered and washed with ethyl acetate and cold Ethanol to afford the title compound. Isolated yield: 0.200 g (88%). MS-APCI (m/z+): 456, 500.

### Example 109

### (S)-N-{2-Oxo-3-[3-(5-phenyl-[1,3,4]oxadiazol-2-yl)-1,4,5,6-tetrahydro-1,2-diaza-benzo[e]azulen-8-yl]-oxazolidin-5-ylmethyl}-acetamide

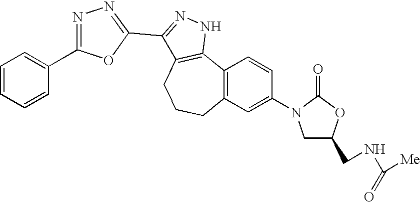

### N-{2-Oxo-3-[5-oxo-6(R,S)-(5-phenyl-[1,3,4]oxadiazole-2-carbonyl)-6,7,8,9-tetrahydro-5H-benzocyclohepten-2-yl]-oxazolidin-5(S)-ylmethyl}-acetamide (Step 1)

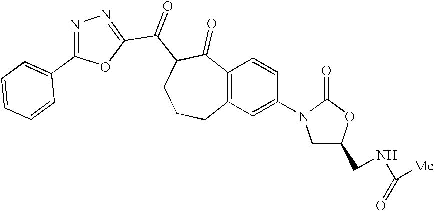

The title compound was prepared according to general procedure HH using (S)-N-[2-oxo-3-(5-oxo-6,7,8,9-tetrahydro-5H-benzocyclohepten-2-yl)-oxazolidin-5-ylmethyl]-acetamide (0.50 g, 1.58 mmol) dissolved in THF (14 mL), LiHMDS (1M, 2.1 eq., 3.32 mL) and 5-phenyl-[1,3,4]oxadiazole-2-carbonyl chloride (1.2 eq., 0.396 g, 1.90 mmol) in THF (8 mL). The isolated residue was subjected to silica gel flash chromatography, eluting with MeOH/dichloromethane gradient (0-6% MeOH over 1 hour and 10 minutes) to afford the title compounds. Isolated yield: 34%. MS-APCI (m/z+): 445, 489.

### (S)-N-{2-Oxo-3-[3-(5-phenyl-[1,3,4]oxadiazol-2-yl)-1,4,5,6-tetrahydro-1,2-diaza-benzo[e]azulen-8-yl]-oxazolidin-5-ylmethyl}-acetamide (Step 2)

The title compound was prepared according to general method II using N-{2-oxo-3-[5-oxo-6(R,S)-(5-phenyl-[1,3,4]oxadiazole-2-carbonyl)-6,7,8,9-tetrahydro-5H-benzocyclohepten-2-yl]-oxazolidin-5(S)-ylmethyl}-acetamide (0.260 g, 053 mmol) and hydrazine hydrate (0.043 g, 1.33 mmol, 2.5 eq.) in ethanol (10 mL). The isolated residue was triturated with ethyl acetate. The resulting solids were filtered and washed with ethyl acetate to afford the title compound. Isolated yield: 0.085 g (33%). MS-APCI (m/z+): 441, 485.

### Example 110

### (S)-N-{3-[3-(1,5-Dimethyl-1H-pyrazol-3-yl)-1,4,5,6-tetrahydro-1,2-diaza-benzo[e]azulen-8-yl]-2-oxo-oxazolidin-5-ylmethyl}-acetamide

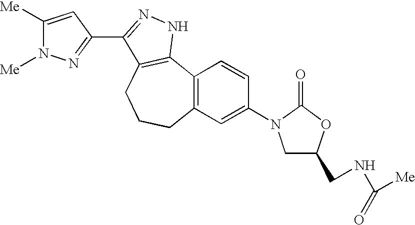

### N-[3-[6(R,S)-(1,5-Dimethyl-1H-pyrazole-3-carbonyl)-5-oxo-6,7,8,9-tetrahydro-5H-benzocyclohepten-2-yl]-2-oxo-oxazolidin-5(S)-ylmethyl]-acetamide (Step 1)

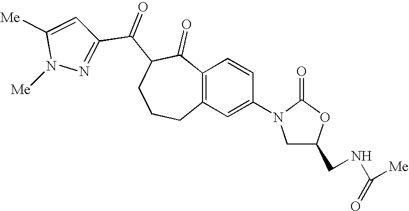

The title compound was prepared according to general procedure HH using (S)-N-[2-oxo-3-(5-oxo-6,7,8,9-tetrahydro-5H-benzocyclohepten-2-yl)-oxazolidin-5-ylmethyl]-acetamide (0.50 g, 1.58 mmol) dissolved in THF (14 mL), LiHMDS (1M, 2.1 eq., 3.32 mL) and 1,5-dimethyl-1H-pyrazole-3-carbonyl chloride (1.1 eq., 0.276 g, 1.74 mmol) in THF (8 mL). The isolated residue was subjected to silica gel flash chromatography, eluting with MeOH/dichloromethane gradient (0-6% MeOH over 1 hour and 10 minutes) to afford the title compound. Isolated yield: 22%. MS-APCI (m/z+): 395, 439.

### (S)-N-{3-[3-(1,5-Dimethyl-1H-pyrazol-3-yl)-1,4,5,6-tetrahydro-1,2-diaza-benzo[e]azulen-8-yl]-2-oxo-oxazolidin-5-ylmethyl}-acetamide (Step 2)

The title compound was prepared according to general procedure II using N-{3-[6(R,S)-(1,5-dimethyl-1H-pyrazole-3-carbonyl)-5-oxo-6,7,8,9-tetrahydro-5H-benzocyclohepten-2-yl]-2-oxo-oxazolidin-5(S)-ylmethyl}-acetamide (0.150 g, 0.34 mmol) and hydrazine hydrate (0.027 g, 0.86 mmol, 2.5 eq.) in ethanol (10 mL). The isolated residue was triturated with ethanol. The resulting solids were filtered and washed with ethanol and ethyl acetate to afford the title compound. Isolated yield: 0.085 g (57%). MS-APCI (m/z+): 391, 435.

### Example 111

### (S)-[3-(3-Isoxazol-5-yl-1,4,5,6-tetrahydro-1,2-diaza-benzo[e]azulen-8-yl)-2-oxo-oxazolidin-5-ylmethyl]-carbamic acid tert-butyl ester

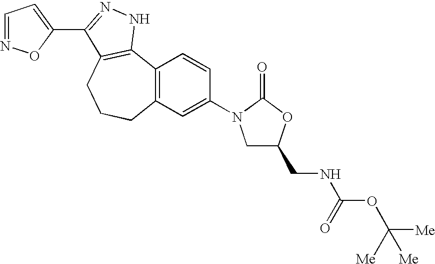

### {3-[6(R,S)-(Isoxazole-5-carbonyl)-5-oxo-6,7,8,9-tetrahydro-5H-benzocyclohepten-2-yl]-2-oxo-oxazolidin-5(S)-ylmethyl}-carbamic acid tert-butyl ester (Step 1)

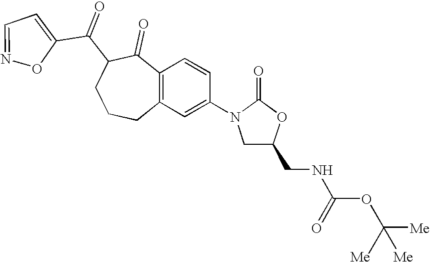

The title compound was prepared according to general procedure HH using (S)-[2-oxo-3-(5-oxo-6,7,8,9-tetrahydro-5H-benzocyclohepten-2-yl)-oxazolidin-5-ylmethyl]-carbamic acid tert-butyl ester (1.00 g, 2.67 mmol) dissolved in THF (25 mL), LiHMDS (1M, 2.1 eq., 5.61 mL) and isoxazole-5-carbonyl chloride (1.2 eq., 0.422 g, 3.21 mmol) in THF (10 mL). The isolated residue was subjected to silica gel flash chromatography, eluting with MeOH/dichloromethane gradient (0-6% MeOH over 1 hour and 10 minutes) to afford the title compounds. Isolated yield: 69%. MS-APCI (m/z−): 468.

### (S)-[3-(3-Isoxazol-5-yl-1,4,5,6-tetrahydro-1,2-diaza-benzo[e]azulen-8-yl)-2-oxo-oxazolidin-5-ylmethyl]-carbamic acid tert-butyl ester (Step 2)

The title compound was prepared according to general procedure II using {3-[6(R,S)-(isoxazole-5-carbonyl)-5-oxo-6,7,8,9-tetrahydro-5H-benzocyclohepten-2-yl]-2-oxo-oxazolidin-5(S)-ylmethyl}-carbamic acid tert-butyl ester (0.310 g, 0.66 mmol) and hydrazine hydrate (0.053 g, 1.65 mmol, 2.5 eq.) in ethanol (25 mL). The isolated residue was subjected to silica gel flash chromatography, eluting with MeOH/dichloromethane gradient (0-6% MeOH over 1 hour, 10 minutes) to afford the title compound. Isolated yield: 0.200 g (65%) MS-APCI (m/z−): 364, 464.

### Example 112

### (S)-N-[3-(3-Benzofuran-2-yl-1,4,5,6-tetrahydro-1,2-diaza-benzo[e]azulen-8-yl)-2-oxo-oxazolidin-5-ylmethyl]-acetamide and (S)-Benzofuran-2-carboxylic acid [3-(3-benzofuran-2-yl-1,4,5,6-tetrahydro-1,2-diaza-benzo[e]azulen-8-yl)-2-oxo-oxazolidin-5-ylmethyl]-amide

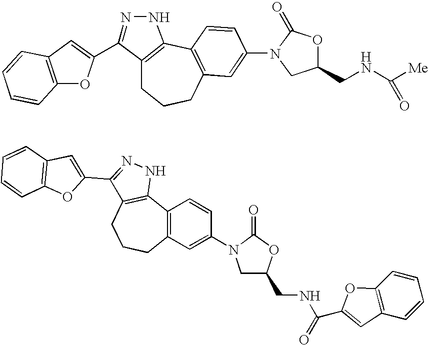

### N-{3-[6(R,S)-(Benzofuran-2-carbonyl)-5-oxo-6,7,8,9-tetrahydro-5H-benzocyclohepten-2-yl]-2-oxo-oxazolidin-5(S)-ylmethyl}-acetamide; and Benzofuran-2-carboxylic acid {3-[6(R,S)-(benzofuran-2-carbonyl)-5-oxo-6,7,8,9-tetrahydro-5H-benzocyclohepten-2-yl]-2-oxo-oxazolidin-5(S)-ylmethyl}-amide (Step 1)

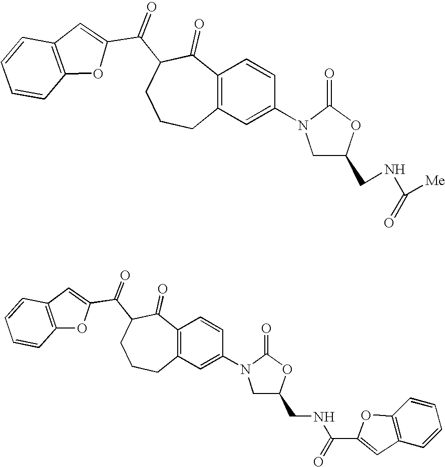

The title compounds were prepared according to general procedure HH using (S)-N-[2-oxo-3-(5-oxo-6,7,8,9-tetrahydro-5H-benzocyclohepten-2-yl)-oxazolidin-5-ylmethyl]-acetamide (0.50 g, 1.58 mmol) dissolved in THF (14 mL), LiHMDS (1 M, 2.1 eq., 3.32 mL) and benzofuran-2-carbonyl chloride (1.2 eq., 0.340 g, 1.90 mmol) as a solid. The isolated residue was subjected to silica gel flash chromatography, eluting with MeOH/dichloromethane gradient (0-7% MeOH over 1 hour and 10 minutes) to afford the title compounds.

For N-{3-[6(R,S)-(Benzofuran-2-carbonyl)-5-oxo-6,7,8,9-tetrahydro-5H-benzocyclohepten-2-yl]-2-oxo-oxazolidin-5(S)-ylmethyl}-acetamide: Isolated yield: 18%. MS-APCI (m/z+): 417, 461;

For Benzofuran-2-carboxylic acid {3-[6(R,S)-(benzofuran-2-carbonyl)-5-oxo-6,7,8,9-tetrahydro-5H-benzocyclohepten-2-yl]-2-oxo-oxazolidin-5(S)-ylmethyl}-amide: Isolated yield: 28%. MS-APCI (m/z+): 519, 563.

### (S)-N-[3-(3-Benzofuran-2-yl-1,4,5,6-tetrahydro-1,2-diaza-benzo[e]azulen-8-yl)-2-oxo-oxazolidin-5-ylmethyl]-acetamide (Step 2)

The title compound was prepared according to general procedure II using N-{3-[6(R,S)-(benzofuran-2-carbonyl)-5-oxo-6,7,8,9-tetrahydro-5H-benzocyclohepten-2-yl]-2-oxo-oxazolidin-5(S)-ylmethyl}-acetamide (0.130 g, 0.28 mmol) and hydrazine hydrate (0.023 g, 0.71 mmol, 2.5 eq.) in ethanol (18 mL). The isolated residue was triturated with dichloromethane and a trace amount of MeOH. The resulting solids were filtered and washed with ethyl acetate then with dichloromethane and a trace amount of MeOH to afford the title compound. Isolated yield: 0.075 g (58%). MS-APCI (m/z+): 413, 457.

### (S)-Benzofuran-2-carboxylic acid [3-(3-benzofuran-2-yl-1,4,5,6-tetrahydro-1,2-diaza-benzo[e]azulen-8-yl)-2-oxo-oxazolidin-5-ylmethyl]-amide (Step 2′)

The title compound was prepared according to general method HH using benzofuran-2-carboxylic acid {3-[6(R,S)-(benzofuran-2-carbonyl)-5-oxo-6,7,8,9-tetrahydro-5H-benzocyclohepten-2-yl]-2-oxo-oxazolidin-5(S)-ylmethyl}-amide (0.230 g, 0.41 mmol) and hydrazine hydrate (0.033 g, 1.03 mmol, 2.5 eq.) in ethanol (18 mL). The isolated residue was triturated with ethyl acetate and a trace amount of ethanol. The resulting solids were filtered and washed with Ethyl acetate to afford the title compound. Isolated yield: 0.130 g (57%). MS-APCI (m/z+): 515, 559.

### Example 113

### (S)-2,5-Dimethyl-2H-pyrazole-3-carboxylic acid {3-[3-(2,5-dimethyl-2H-pyrazol-3-yl)-1,4,5,6-tetrahydro-1,2-diaza-benzo[e]azulen-8-yl]-2-oxo-oxazolidin-5-ylmethyl}-amide

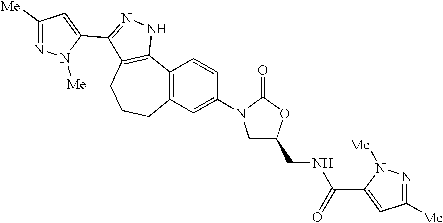

### N-{3-[6(R,S)-(2,5-Dimethyl-2H-Pyrazole-3-carbonyl)-5-oxo-6,7,8,9-tetrahydro-5H-benzocyclohepten-2-yl]-2-oxo-oxazolidin-5(S)-ylmethyl}-acetamide and 2,5-Dimethyl-2H-pyrazole-3-carboxylic acid {3-[6(R,S)-(2,5-dimethyl-2H-pyrazole-3-carbonyl)-5-oxo-6,7,8,9-tetrahydro-5H-benzocyclohepten-2-yl]-2-oxo-oxazolidin-5(S)-ylmethyl}-amide (Step 1)

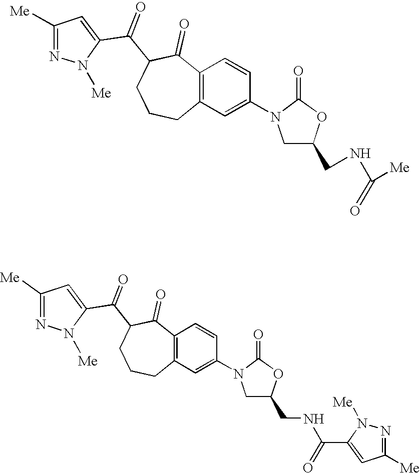

The title compounds were prepared according to general procedure HH using (S)-N-[2-oxo-3-(5-oxo-6,7,8,9-tetrahydro-5H-benzocyclohepten-2-yl)-oxazolidin-5-ylmethyl]-acetamide (0.50 g, 1.58 mmol) dissolved in THF (14 mL), LiHMDS (1M, 2.1 eq., 3.32 mL) and 2,5-dimethyl-2H-pyrazole-3-carbonyl chloride (1.1 eq., 0.276 g, 1.74 mmol) as a solid. The isolated residue was subjected to silica gel flash chromatography, eluting with ethyl acetate/dichloromethane gradient (0-7% MeOH over 1 hour and 10 minutes) to afford the title compounds.

For N-{3-[6(R,S)-(2,5-Dimethyl-2H-pyrazole-3-carbonyl)-5-oxo-6,7,8,9-tetrahydro-5H-benzocyclohepten-2-yl]-2-oxo-oxazolidin-5(S)-ylmethyl}-acetamide: Isolated yield: 20%. MS-APCI (m/z+): 395, 439;

For 2,5-Dimethyl-2H-pyrazole-3-carboxylic acid {3-[6(R,S)-(2,5-dimethyl-2H-pyrazole-3-carbonyl)-5-oxo-6,7,8,9-tetrahydro-5H-benzocyclohepten-2-yl]-2-oxo-oxazolidin-5(S)-ylmethyl}-amide: Isolated yield: 34%. MS-APCI (m/z+): 475, 519.

### (S)-2,5-Dimethyl-2H-pyrazole-3-carboxylic acid {3-[3-(2,5-dimethyl-2H-pyrazol-3-yl)-1,4,5,6-tetrahydro-1,2-diaza-benzo[e]azulen-8-yl]-2-oxo-oxazolidin-5-ylmethyl}-amide (Step 2)

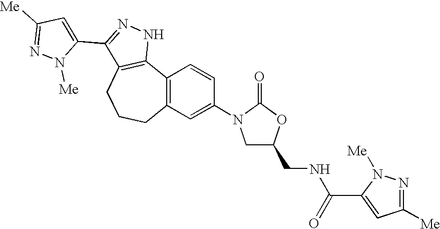

The title compound was prepared according to general procedure II using 2,5-dimethyl-2H-pyrazole-3-carboxylic acid {3-[6(R,S)-(2,5-dimethyl-2H-pyrazole-3-carbonyl)-5-oxo-6,7,8,9-tetrahydro-5H-benzocyclohepten-2-yl]-2-oxo-oxazolidin-5(S)-ylmethyl}-amide (0.170 g, 0.33 mmol) and hydrazine hydrate (0.026, 0.82 mmol, 2.5 eq.) in ethanol (14 mL). The isolated residue was subjected to silica gel flash chromatography, eluting with MeOH/dichloromethane gradient (1-7% MeOH over 1 hour, 10 minutes) to afford the title compound. Isolated yield: 0.052 g (31%). MS-APCI (m/z+): 471, 515.

### Example 114

### (S)-N-[3-(10-Fluoro-3-isoxazol-5-yl-1,4,5,6-tetrahydro-1,2-diaza-benzo[e]azulen-8-yl)-2-oxo-oxazolidin-5-ylmethyl]-acetamide

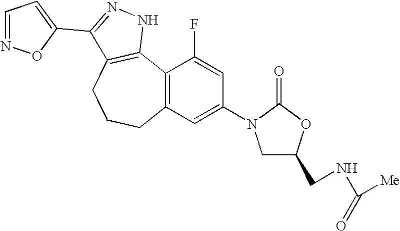

### N-{3-[4-Fluoro-6(R,S)-(isoxazole-5-carbonyl)-5-oxo-6,7,8,9-tetrahydro-5H-benzocyclohepten-2-yl]-2-oxo-oxazolidin-5(S)-ylmethyl}-acetamide (Step 1)

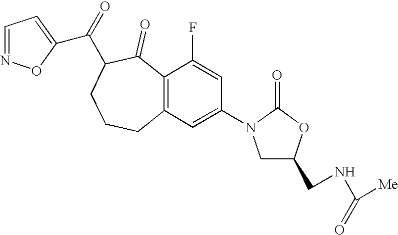

The title compound was prepared according to general procedure HH using (S)-N-[3-(4-fluoro-5-oxo-6,7,8,9-tetrahydro-5H-benzocyclohepten-2-yl)-2-oxo-oxazolidin-5-ylmethyl]-acetamide (0.50 g, 1.50 mmol) dissolved in THF (12 mL), LiHMDS (1M, 2.1 eq., 3.14 mL) and isoxazole-5-carbonyl chloride (1.1 eq., 0.220 g, 1.67 mmol) in THF (10 mL). The isolated residue was subjected to silica gel flash chromatography, eluting with MeOH/dichloromethane gradient (0-7% MeOH over 1 hour and 10 minutes) to afford the title compound. Isolated yield: 55%. MS-APCI (m/z+): 430.

### (S)-N-[3-(10-Fluoro-3-isoxazol-5-yl-1,4,5,6-tetrahydro-1,2-diaza-benzo[e]azulen-8-yl)-2-oxo-oxazolidin-5-ylmethyl]-acetamide (Step 2)

The title compound was prepared according to general procedure II using N-{3-[4-fluoro-6(R,S)-(isoxazole-5-carbonyl)-5-oxo-6,7,8,9-tetrahydro-5H-benzocyclohepten-2-yl]-2-oxo-oxazolidin-5(S)-ylmethyl}-acetamide (0.350 g, 0.82 mmol) and hydrazine hydrate (0.065, 2.04 mmol, 2.5 eq.) in ethanol (18 mL). The isolated residue was subjected to silica gel flash chromatography, eluting with MeOH/dichloromethane gradient (1-7% MeOH over 1 hour, 10 minutes.) to afford the title compound. Isolated yield: 0.100 g (29%). MS-APCI (m/z+): 382, 426.

### Example 115

### (S)-N-[3-(3-Benzothiazol-2-yl-1,4,5,6-tetrahydro-1,2-diaza-benzo[e]azulen-8-yl)-2-oxo-oxazolidin-5-ylmethyl]-acetamide

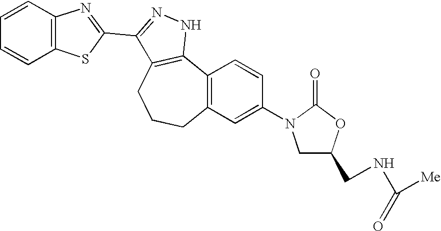

### N-{3-[6(R,S)-(Benzothiazole-2-carbonyl)-5-oxo-6,7,8,9-tetrahydro-5H-benzocyclohepten-2-yl]-2-oxo-oxazolidin-5(S)-ylmethyl}-acetamide (Step 1)

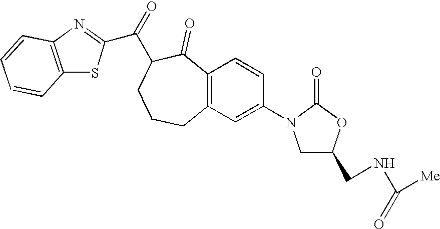

The title compound was prepared according to general procedure HH using (S)-N-[2-oxo-3-(5-oxo-6,7,8,9-tetrahydro-5H-benzocyclohepten-2-yl)-oxazolidin-5-ylmethyl]-acetamide (0.50 g, 1.58 mmol) dissolved in THF (14 mL), LiHMDS (1M, 2.1 eq., 3.32 mL) and benzothiazole-2-carbonyl chloride (1.1 eq., 0.344 g, 1.74 mmol) in THF (8 mL). The isolated residue was subjected to silica gel flash chromatography, eluting with MeOH/dichloromethane gradient (0-6% MeOH over 1 hour and 10 minutes) to afford the title compound. Isolated yield: 29%. MS-APCI (m/z+): 434, 478.

### (S)-N-[3-(3-Benzothiazol-2-yl-1,4,5,6-tetrahydro-1,2-diaza-benzo[e]azulen-8-yl)-2-oxo-oxazolidin-5-ylmethyl]-acetamide (Step 2)

The title compound was prepared according to general procedure II using N-{3-[6(R,S)-(benzothiazole-2-carbonyl)-5-oxo-6,7,8,9-tetrahydro-5H-benzocyclohepten-2-yl]-2-oxo-oxazolidin-5(S)-ylmethyl}-acetamide (0.240 g, 0.50 mmol) and hydrazine hydrate (0.040, 1.26 mmol, 2.5 eq.) in Ethanol (18 mL). The isolated residue was subjected to silica gel flash chromatography, eluting with MeOH/dichloromethane gradient (0-7% MeOH over 1 hour, 10 minutes, then 7-9% MeOH over 30 minutes) to afford the title compound. Isolated yield: 0.125 g (53%). MS-APCI (m/z+): 430, 474.

### Example 116

### (S)-5-Aminomethyl-3-(3-isoxazol-5-yl-1,4,5,6-tetrahydro-1,2-diaza-benzo[e]azulen-8-yl)-oxazolidin-2-one hydrochloride

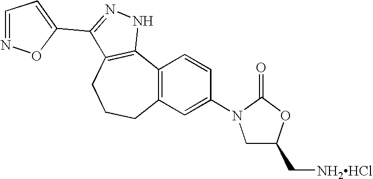

(S)-[3-(3-Isoxazol-5-yl-1,4,5,6-tetrahydro-1,2-diaza-benzo[e]azulen-8-yl)-2-oxo-oxazolidin-5-ylmethyl]-carbamic acid tert-butyl ester (0.50 g, 1.07 mmol) was dissolved in dioxane (20 mL) and 4N HCl/dioxane added (10 eq., 2.66 mL) and the reaction mixture stirred at room temperature overnight. Ether was then added and resulting solid precipitate filtered off and washed with ether and ethyl acetate to afford the title compound. Isolated yield: 0.390 g (91%). MS-APCI (m/z+): 366.

### Example 117

### (S)-N-[3-(3-Isoxazol-5-yl-1,4,5,6-tetrahydro-1,2-diaza-benzo[e]azulen-8-yl)-2-oxo-oxazolidin-5-ylmethyl]-benzamide

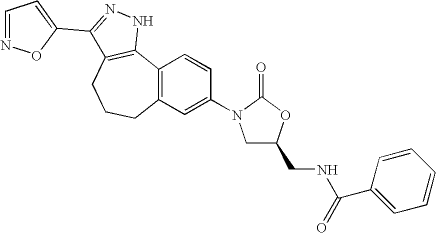

(S)-5-Aminomethyl-3-(3-isoxazol-5-yl-1,4,5,6-tetrahydro-1,2-diaza-benzo[e]azulen-8-yl)-oxazolidin-2-one hydrochloride (0.180 g, 0.45 mmol) was dissolved in dichloromethane (5 mL) and cooled to 0° C. in an ice bath. To this mixture was added triethylamine (0.181 g, 1.79 mmol, 4.0 eq.) followed by benzoyl chloride (0.157 g, 1.12 mmol, 2.5 eq.). The reaction mixture stirred under nitrogen allowing the reaction to warm to room temperature over night. The reaction mixture was then diluted with ethyl acetate, washed with saturated sodium bicarbonate solution, then with brine, dried over magnesium sulfate, filtered and concentrated. The isolated residue was subjected to silica gel flash chromatography, eluting with MeOH/dichloromethane gradient (0-6% MeOH over 1 hour, 10 minutes.) to afford the title compound. Isolated yield: 0.115 g (55%). MS-APCI (m/z+): 426, 470.

### Example 118

### (S)-Cyclopropanecarboxylic acid [3-(3-isoxazol-5-yl-1,4,5,6-tetrahydro-1,2-diaza-benzo[e]azulen-8-yl)-2-oxo-oxazolidin-5-ylmethyl]-amide

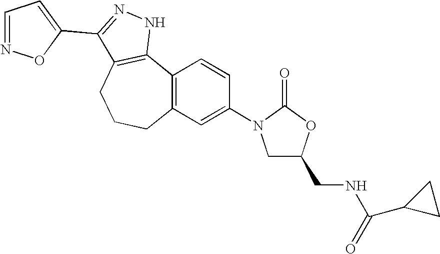

(S)-5-Aminomethyl-3-(3-isoxazol-5-yl-1,4,5,6-tetrahydro-1,2-diaza-benzo[e]azulen-8-yl)-oxazolidin-2-one hydrochloride (0.170 g, 0.42 mmol) was dissolved in dichloromethane (5 mL) and cooled to 0° C. in an ice bath. To this was added triethylamine (0.171 g, 1.69 mmol, 4.0 eq.) followed by cyclopropane carbonyl chloride (0.111 g, 1.06 mmol, 2.5 eq.). The reaction mixture stirred under nitrogen allowing the reaction to warm to room temperature over night. The reaction mixture was then diluted with ethyl acetate, washed with saturated sodium bicarbonate solution, then with brine, dried over magnesium sulfate, filtered and concentrated. The isolated residue was subjected to silica gel flash chromatography, eluting with MeOH/dichloromethane gradient (0-6% MeOH over 1 hour, 10 minutes) to afford the title compound. Isolated yield: 0.098 g (54%). MS-APCI (m/z+): 390, 434.

### Example 119

### (S)-N-[2-Oxo-3-(5-oxo-6-thiazol-2-ylmethylene-6,7,8,9-tetrahydro-5H-benzocyclohepten-2-yl)-oxazolidin-5-ylmethyl]-acetamide

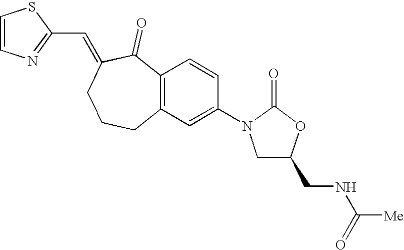

To (S)-N-[2-oxo-3-(5-oxo-6,7,8,9-tetrahydro-5H-benzocyclohepten-2-yl)-oxazolidin-5-ylmethyl]-acetamide (0.40 g, 1.26 mmol) and thiazole-2-carbaldehyde (0.57 g, 5.06 mmol, 4.0 eq.) was added acetic acid (2.5 mL) and piperidine (2.5 mL) and the mixture heated to 90-95° C. overnight. Heat was then removed, 0.5 N HCl added, extracted with ethyl acetate, washed organic phase with saturated sodium bicarbonate, then with brine, dried over magnesium sulfate, filtered and concentrated. The isolated residue was subjected to silica gel flash chromatography, eluting with MeOH/dichloromethane gradient (0-7% MeOH over 1 hour, 10 minutes) to afford the title compound. Isolated yield: 0.380 g (73%). MS-APCI (m/z+): 368, 412.

### Example 120

### (S)-N-{3-[3-(4-Hydroxy-phenyl)-1,4,5,6-tetrahydro-1,2-diaza-benzo[e]azulen-8-yl]-2-oxo-oxazolidin-5-ylmethyl}-acetamide

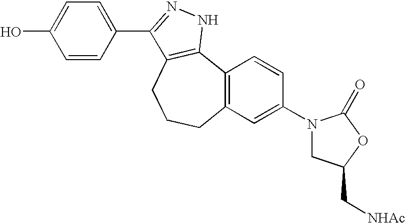

### (S)-N-{3-[6-(4-Hydroxy-benzylidene)-5-oxo-6,7,8,9-tetrahydro-5H-benzocyclohepten-2-yl]-2-oxo-oxazolidin-5-ylmethyl}-acetamide (Step 1)

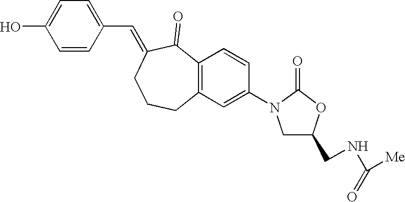

To (S)-N-[2-oxo-3-(5-oxo-6,7,8,9-tetrahydro-5H-benzocyclohepten-2-yl)-oxazolidin-5-ylmethyl]-acetamide (0.35 g, 1.11 mmol) and 4-hydroxy-benzaldehyde (0.54 g, 4.43 mmol, 4.0 eq.) was added acetic acid (2.0 mL) and piperidine (2.0 mL) and the mixture was heated to 90-95° C. overnight. Heat was then removed, 0.5 N HCl added and the reaction was extracted with ethyl acetate. The organic phase was washed with saturated sodium bicarbonate and then with brine, dried over magnesium sulfate, filtered and concentrated. The isolated residue was subjected to silica gel flash chromatography, eluting with MeOH/dichloromethane gradient (0-7% MeOH over 1 hour, 10 minutes, then 7-9% over 30 minutes) to afford the title compound. Isolated yield: 0.350 g (75%). MS-APCI (m/z+): 377, 421.

### (S)-N-{3-[3-(4-Hydroxy-phenyl)-1,4,5,6-tetrahydro-1,2-diaza-benzo[e]azulen-8-yl]-2-oxo-oxazolidin-5-ylmethyl}-acetamide (Step 2)

To (S)-N-{3-[6-(4-hydroxy-benzylidene)-5-oxo-6,7,8,9-tetrahydro-5H-benzocyclohepten-2-yl]-2-oxo-oxazolidin-5-ylmethyl}-acetamide (0.130 g, 0.31 mmol) in ethanol (5 mL) was added tosyl hydrazide (0.069 g, 0.37 mmol, 1.2 eq.) and p-toluene sulfonic acid (0.012 g, 0.06 mmol, 0.2 eq) and mixture was heated to reflux in an oil bath for 72 hours. Solvent was then removed in vacuo; saturated sodium bicarbonate was added and the reaction was extracted with dichloromethane. The organic phase was washed with brine, dried over magnesium sulfate, filtered and concentrated. The isolated residue was subjected to silica gel flash chromatography, eluting with MeOH/dichloromethane gradient (0-7% MeOH over 1 hour, 10 minutes, then 7-9% over 30 minutes) to afford the title compound. Isolated yield: 0.042 g (31%). MS-APCI (m/z+): 389, 433.

### Example 121

### (S)-N-{3-[6-(1-Methyl-1H-pyrrol-2-ylmethylene)-5-oxo-6,7,8,9-tetrahydro-5H-benzocyclohepten-2-yl]-2-oxo-oxazolidin-5-ylmethyl}-acetamide

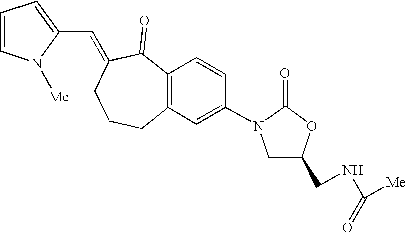

To (S)-N-[2-oxo-3-(5-oxo-6,7,8,9-tetrahydro-5H-benzocyclohepten-2-yl)-oxazolidin-5-ylmethyl]-acetamide (0.40 g, 1.26 mmol) and 1-methyl-1H-pyrrole-2-carbaldehyde (0.55 g, 5.06 mmol, 4.0 eq.) was added acetic acid (2.5 mL) and piperidine (2.5 mL) and the mixture heated to 90-95° C. overnight. Heat was then removed, saturated sodium bicarbonate was added and the reaction was extracted with ethyl acetate. The organic phase was washed with brine, dried over magnesium sulfate, filtered and concentrated. The isolated residue was subjected to silica gel flash chromatography, eluting with MeOH/dichloromethane gradient (0-7% MeOH over 1 hour, 10 minutes, then 7-9% over 30 minutes) to afford the title compound. Isolated yield: 0.200 g (39%). MS-APCI (m/z+): 364, 408.

### Example 122

### (S)-N-[3-(2-Methyl-2,4,5,6-tetrahydro-1,2-diaza-benzo[e]azulen-8-yl)-2-oxo-oxazolidin-5-ylmethyl]-acetamide

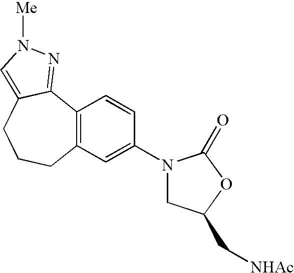

The title compound was prepared according to general procedure II using (S)-N-[3-(6-dimethyaminomethylene-5-oxo-6,7,8,9-tetrahydro-5H-benzocyclohepten-2-yl)-2-oxo-oxalidin-5-ylmethyl]acetamide (0.20 g, 0.54 mmol) and methyl hydrazine (0.099 g, 2.15 mmol, 4.0 eq.) in Ethanol (9 mL). The isolated residue was subjected to silica gel flash chromatography, eluting with MeOH/Ethyl acetate gradient (0-8% MeOH over 1 hour.) to afford the title compound. Isolated yield: 0.060 g (31%). MS-APCI (m/z+): 311, 355.

### Example 123

### (S)-N-[3-(2-Benzyl-2,4,5,6-tetrahydro-1,2-diaza-benzo[e]azulen-8-yl)-2-oxo-oxazolidin-5-ylmethyl]-acetamide and (S)-N-[3-(1-Benzyl-1,4,5,6-tetrahydro-1,2-diaza-benzo[e]azulen-8-yl)-2-oxo-oxazolidin-5-ylmethyl]-acetamide

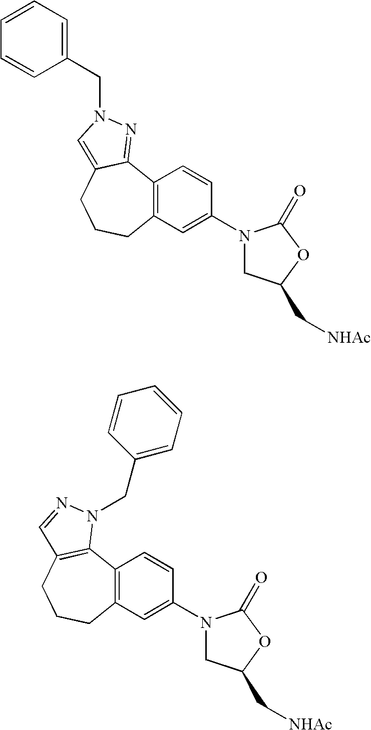

To (S)-N-[3-(6-dimethyaminomethylene-5-oxo-6,7,8,9-tetrahydro-5H-benzocyclohepten-2-yl)-2-oxo-oxalidin-5-ylmethyl]acetamide (0.20 g, 0.54 mmol) in Ethanol (9 mL) was added benzylhydrazine dihydrochloride (0.260 g, 1.33 mmol, 2.5 eq.) and triethylamine (0.270 mL, 2.67 mmol, 5.0 eq.) and the resulting mixture stirred at room temperature overnight. The solvent was then removed in vacuo and saturated sodium bicarbonate was added. The reaction was extracted with dichloromethane then organic phase was washed with brine, dried over sodium sulfate, filtered and concentrated. The isolated residue was subjected to silica gel flash chromatography eluting with MeOH/Ethyl acetate gradient (0-8% MeOH over 1 hour) to afford the title compounds as a 1:1 mixture of the two regioisomers. Isolated yield: 0.080 g (35%). MS-APCI (m/z+): 387, 431.

### Example 124

### (S)-N-[3-(2-Ethyl-2,4,5,6-tetrahydro-1,2-diaza-benzo[e]azulen-8-yl)-2-oxo-oxazolidin-5-ylmethyl]-acetamide and (S)-N-[3-(1-Ethyl-1,4,5,6-tetrahydro-1,2-diaza-benzo[e]azulen-8-yl)-2-oxo-oxazolidin-5-ylmethyl]-acetamide

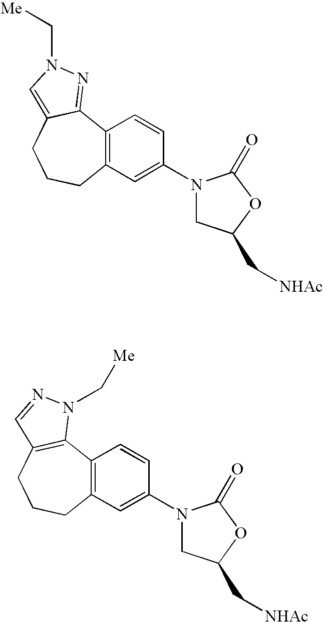

To (S)-N-[3-(6-dimethyaminomethylene-5-oxo-6,7,8,9-tetrahydro-5H-benzocyclohepten-2-yl)-2-oxo-oxalidin-5-ylmethyl]acetamide (0.20 g, 0.54 mmol) in Ethanol (9 mL) was added ethyl hydrazine oxalate (0.320 g, 2.13 mmol, 4.0 eq.) and triethylamine (0.44, 4.35 mmol, 8.0 eq.) and the resulting mixture stirred at room temperature overnight. The solvent was then removed in vacuo and saturated sodium bicarbonate was added. The reaction was extracted with dichloromethane then organic phase was washed with brine, dried over sodium sulfate, filtered and concentrated. The isolated residue was subjected to silica gel flash chromatography eluting with MeOH/Ethyl acetate gradient (0-8% MeOH over 1 hour.) to afford the title compounds as a 1:1 mixture of the two regioisomers. Isolated yield: 0.055 g (28%). MS-APCI (m/z+): 325, 369.

### Example 125

### N-[3-(6(R,S)-Bromo-5-oxo-6,7,8,9-tetrahydro-5H-benzocyclohepten-2-yl)-2-oxo-oxazolidin-5(S)-ylmethyl]-acetamide

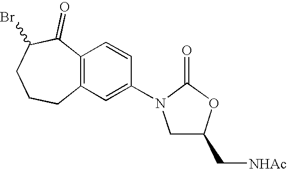

To compound (S)-N-[2-oxo-3-(5-oxo-6,7,8,9-tetrahydro-5H-benzocyclohepten-2-yl)-oxazolidin-5-ylmethyl]-acetamide (4.2 g, 13 mmol) in 414 mL of dichloromethane was added 69 mL of glacial acetic acid. Pyridinium bromide perbromide (4.7 g, 15 mmol) was added in one portion and the solution was stirred for 72 hours. The solution was concentrated in vacuo to give an oil. The oil was diluted with dichloromethane and washed with water (twice), sat sodium bicarbonate, brine, dried over sodium sulfate, and concentrated in vacuo. Purification by silica gel chromatography afforded the title compound in quantitative yield (5.24 g). MS-APCI (m/z+): 395, 396 (M+H).

### Example 126

### (S)-N-[3-(3-Methylamino-1,4,5,6-tetrahydro-1,2-diaza-benzo[e]azulen-8-yl)-2-oxo-oxazolidin-5-ylmethyl]-acetamide

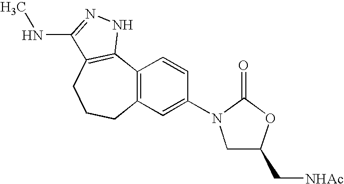

Following general procedure JJ, N-[3-(6(R,S)-bromo-5-oxo-6,7,8,9-tetrahydro-5H-benzocyclohepten-2-yl)-2-oxo-oxazolidin-5(S)-ylmethyl]-acetamide (0.395 g, 1 mmol) and 4-methylthiosemicarbazide (0.105 g, 1 mmol) gave the title compound in 8% yield (0.030 g). MS-APCI (m/z+): 326, 370 (M+H).

### Example 127

### (S)-N-[3-(3-Ethylamino-1,4,5,6-tetrahydro-1,2-diaza-benzo[e]azulen-8-yl)-2-oxo-oxazolidin-5-ylmethyl]-acetamide

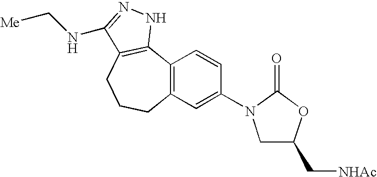

Following general procedure JJ, N-[3-(6(R,S)-bromo-5-oxo-6,7,8,9-tetrahydro-5H-benzocyclohepten-2-yl)-2-oxo-oxazolidin-5 (S)-ylmethyl]-acetamide (0.395 g, 1 mmol) and 4-ethylthiosemicarbazide (0.119 g, 1 mmol) gave the title compound in 21% yield (0.090 g). MS-APCI (m/z+): 340, 384 (M+H).

### Example 128

### (S)-N-[2-Oxo-3-(3-propylamino-1,4,5,6-tetrahydro-1,2-diaza-benzo[e]azulen-8-yl)-oxazolidin-5-ylmethyl]-acetamide

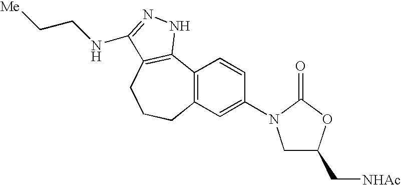

Following general procedure JJ, N-[3-(6(R,S)-bromo-5-oxo-6,7,8,9-tetrahydro-5H-benzocyclohepten-2-yl)-2-oxo-oxazolidin-5(S)-ylmethyl]-acetamide (0.395 g, 1 mmol) and 4-propylthiosemicarbazide (0.133 g, 1 mmol) gave the title compound in 28% yield (0.110 g). MS-APCI (m/z+): 354, 398 (M+H).

### Example 129

### (S)-N-[3-(3-Isopropylamino-1,4,5,6-tetrahydro-1,2-diaza-benzo[e]azulen-8-yl)-2-oxo-oxazolidin-5-ylmethyl]-acetamide

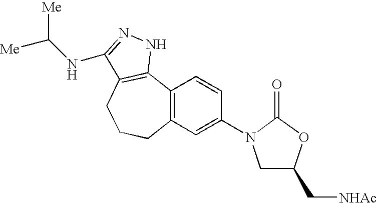

Following general procedure JJ, N-[3-(6(R,S)-bromo-5-oxo-6,7,8,9-tetrahydro-5H-benzocyclohepten-2-yl)-2-oxo-oxazolidin-5(S)-ylmethyl]-acetamide (0.395 g, 1 mmol) and 4-isopropylthiosemicarbazide (0.133 g, 1 mmol) gave the title compound in 18% yield (0.070 g). MS-APCI (m/z+): 354, 398 (M+H).

### Example 130

### (S)-(2-{8-[5-(Acetylamino-methyl)-2-oxo-oxazolidin-3-yl]-1,4,5,6-tetrahydro-1,2-diaza-benzo[e]azulen-3-ylamino}-ethyl)-carbamic acid tert-butyl ester

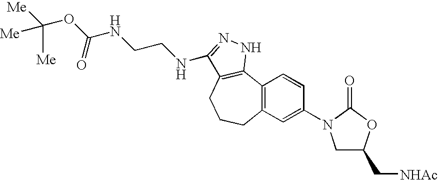

Following general procedure JJ, N-[3-(6(R,S)-bromo-5-oxo-6,7,8,9-tetrahydro-5H-benzocyclohepten-2-yl)-2-oxo-oxazolidin-5(S)-ylmethyl]-acetamide (0.395 g, 1 mmol) and 4-(2-t-butylcarbamoylethyl)-3-thiosemicarbazide (0.234 g, 1 mmol) gave the title compound in 31% yield (0.157 g). MS-APCI (m/z+): 355, 399, 499 (M+H).

### Example 131

### (S)-(3-{8-[5-(Acetylamino-methyl)-2-oxo-oxazolidin-3-yl]-1,4,5,6-tetrahydro-1,2-diaza-benzo[e]azulen-3-ylamino}-propyl)-carbamic acid tert-butyl ester

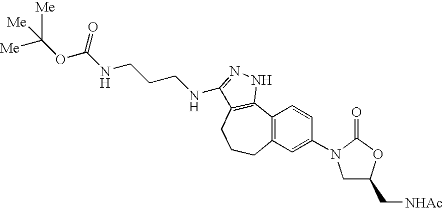

Following general procedure JJ, N-[3-(6(R,S)-bromo-5-oxo-6,7,8,9-tetrahydro-5H-benzocyclohepten-2-yl)-2-oxo-oxazolidin-5(S)-ylmethyl]-acetamide (0.395 g, 1 mmol) and 4-(3-t-butylcarbamoylpropyl)-3-thiosemicarbazide (0.248 g, 1 mmol) gave the title compound in 20% yield (0.105 g). MS-APCI (m/z+): 369, 413, 513 (M+H).

### Example 132

### (S)-N-{3-[3-(3-Diethylamino-propylamino)-1,4,5,6-tetrahydro-1,2-diaza-benzo[e]azulen-8-yl]-2-oxo-oxazolidin-5-ylmethyl}-acetamide

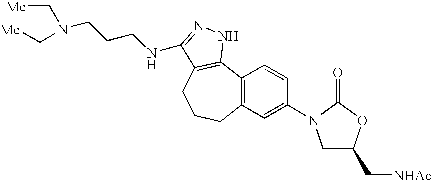

Following general procedure JJ, N-[3-(6(R,S)-bromo-5-oxo-6,7,8,9-tetrahydro-5H-benzocyclohepten-2-yl)-2-oxo-oxazolidin-5(S)-ylmethyl]-acetamide (0.395 g, 1 mmol) and 4-(3-N,N-diethylaminopropyl)-3-thiosemicarbazide (0.204 g, 1 mmol) gave the title compound in 28% yield (0.130 g). MS-APCI (m/z+): 425, 469 (M+H).

### Example 133

### (S)-N-{3-[3-(2-Hydroxy-ethylamino)-1,4,5,6-tetrahydro-1,2-diaza-benzo[e]azulen-8-yl]-2-oxo-oxazolidin-5-ylmethyl}-acetamide

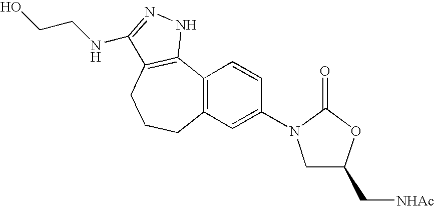

Following general procedure JJ, N-[3-(6(R,S)-bromo-5-oxo-6,7,8,9-tetrahydro-5H-benzocyclohepten-2-yl)-2-oxo-oxazolidin-5(S)-ylmethyl]-acetamide (0.395 g, 1 mmol) and 4-(2-t-butyldimethylsilanyloxyethyl)-3-thiosemicarbazide (0.249 g, 1 mmol) gave the title compound in 13% yield (0.054 g). MS-APCI (m/z+): 356, 400 (M+H).

### Example 134

### (S)-N-{3-[3-(3-Hydroxy-propylamino)-1,4,5,6-tetrahydro-1,2-diaza-benzo[e]azulen-8-yl]-2-oxo-oxazolidin-5-ylmethyl}-acetamide

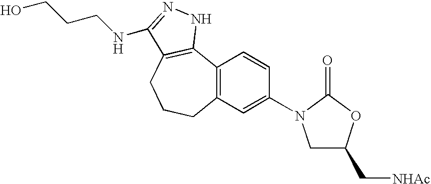

Following general procedure JJ, N-[3-(6(R,S)-bromo-5-oxo-6,7,8,9-tetrahydro-5H-benzocyclohepten-2-yl)-2-oxo-oxazolidin-5(S)-ylmethyl]-acetamide (0.395 g, 1 mmol) and 4-(3-t-butyldimethylsilanyloxypropyl)-3-thiosemicarbazide (0.263 g, 1 mmol) gave the title compound in 20% yield (0.086 g). MS-APCI (m/z+): 370, 414 (M+H).

### Example 135

### (S)-N-{3-[3-(2-Methoxy-ethylamino)-1,4,5,6-tetrahydro-1,2-diaza-benzo[e]azulen-8-yl]-2-oxo-oxazolidin-5-ylmethyl}-acetamide

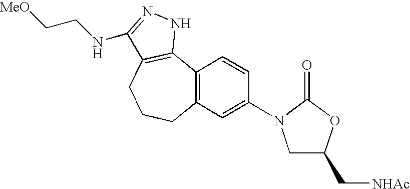

Following general procedure JJ, N-[3-(6(R,S)-bromo-5-oxo-6,7,8,9-tetrahydro-5H-benzocyclohepten-2-yl)-2-oxo-oxazolidin-5 (S)-ylmethyl]-acetamide (0.395 g, 1 mmol) and 4-(2-methoxyethyl)-3-thiosemicarbazide (0.149 g, 1 mmol) gave the title compound in 52% yield (0.213 g). MS-APCI (m/z+): 370, 414 (M+H).

### Example 136

### (S)-N-{3-[3-(3-Methoxy-propylamino)-1,4,5,6-tetrahydro-1,2-diaza-benzo[e]azulen-8-yl]-2-oxo-oxazolidin-5-ylmethyl}-acetamide

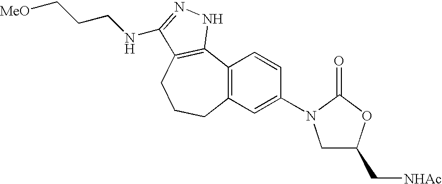

Following general procedure JJ, N-[3-(6(R,S)-bromo-5-oxo-6,7,8,9-tetrahydro-5H-benzocyclohepten-2-yl)-2-oxo-oxazolidin-5(S)-ylmethyl]-acetamide (0.395 g, 1 mmol) and 4-(3-methoxypropyl)-3-thiosemicarbazide (0.163 g, 1 mmol) gave the title compound in 50% yield (0.214 g). MS-APCI (m/z+): 384, 428 (M+H).

### Example 137

### (S)-N-[2-Oxo-3-(3-phenylamino-1,4,5,6-tetrahydro-1,2-diaza-benzo[e]azulen-8-yl)-oxazolidin-5-ylmethyl]-acetamide

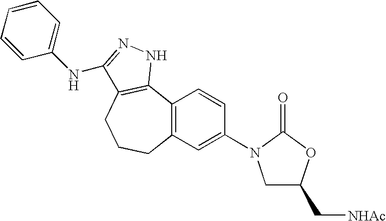

Following general procedure JJ, N-[3-(6(R,S)-bromo-5-oxo-6,7,8,9-tetrahydro-5H-benzocyclohepten-2-yl)-2-oxo-oxazolidin-5(S)-ylmethyl]-acetamide (0.395 g, 1 mmol) and 4-phenyl-3-thiosemicarbazide (0.167 g, 1 mmol) gave the title compound. MS-APCI (m/z+): 388, 432 (M+H).

### Example 138

### (S)-N-[3-(3-Benzylamino-1,4,5,6-tetrahydro-1,2-diaza-benzo[e]azulen-8-yl)-2-oxo-oxazolidin-5-ylmethyl]-acetamide

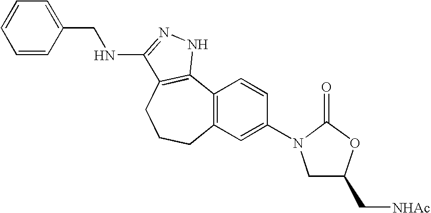

N-[3-(6(R,S)-bromo-5-oxo-6,7,8,9-tetrahydro-5H-benzocyclohepten-2-yl)-2-oxo-oxazolidin-5(S)-ylmethyl]-acetamide (0.198 g, 0.5 mmol), 4-benzyl-3-thiosemicarbazide (0.091 g, 0.5 mmol), 0.15 mL concentrated HCl, and 2.5 mL of absolute ethanol was heated to 88° C. overnight. The mixture was cooled to room temperature and filtered. The filtrate was washed with sat sodium bicarbonate and water, concentrated in vacuo and purified by silica gel chromatography to give the title compound in 23% yield (0.052 g). MS-APCI (m/z+): 402, 446 (M+H).

### Example 139

### (S)-N-{3-[3-(4-Methoxy-benzylamino)-1,4,5,6-tetrahydro-1,2-diaza-benzo[e]azulen-8-yl]-2-oxo-oxazolidin-5-ylmethyl}-acetamide

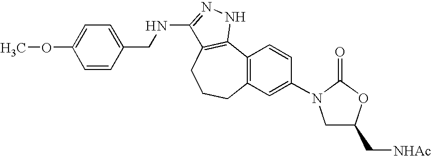

Following general procedure procedure JJ, N-[3-(6(R,S)-bromo-5-oxo-6,7,8,9-tetrahydro-H-benzocyclohepten-2-yl)-2-oxo-oxazolidin-5(S)-ylmethyl]-acetamide (0.201 g, 0.51 mmol), 4-(4-methoxybenzyl)-3-thiosemicarbazide (0.108 g, 0.51 mmol), and 5 mL of absolute ethanol gave the title compound in 31% yield (0.076 g). MS-APCI (m/z+): 432, 476 (M+H).

### Example 140

### (S)-N-{3-[3-(2-Morpholin-4-yl-ethylamino)-1,4,5,6-tetrahydro-1,2-diaza-benzo[e]azulen-8-yl]-2-oxo-oxazolidin-5-ylmethyl}-acetamide

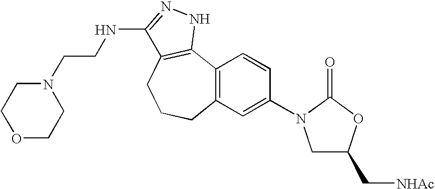

Following general procedure JJ, N-[3-(6(R,S)-bromo-5-oxo-6,7,8,9-tetrahydro-5H-benzocyclohepten-2-yl)-2-oxo-oxazolidin-5(S)-ylmethyl]-acetamide (0.395 g, 1 mmol) and 4-(2-morpholinoethyl)-3-thiosemicarbazide (0.204 g, 1 mmol) gave the title compound in 25% yield (0.130 g). MS-APCI (m/z+): 425, 469 (M+H).

### Example 141

### (S)-N-{2-Oxo-3-[3-(2-pyridin-2-yl-ethylamino)-1,4,5,6-tetrahydro-1,2-diaza-benzo[e]azulen-8-yl]-oxazolidin-5-ylmethyl}-acetamide

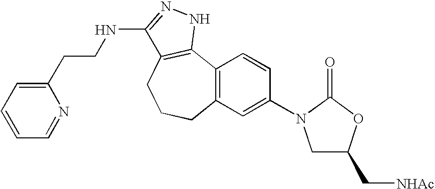

Following general procedure JJ, N-[3-(6(R,S)-bromo-5-oxo-6,7,8,9-tetrahydro-5H-benzocyclohepten-2-yl)-2-oxo-oxazolidin-5(S)-ylmethyl]-acetamide (0.367 g, 0.93 mmol) and 4-(2-(2-pyridyl)ethyl)-3-thiosemicarbazide (0.204 g, 1 mmol) gave the title compound in 31% yield (0.144 g). MS-APCI (m/z+): 417, 461 (M+H).

### Example 142

### (S)-N-(3-{3-[2-(4-Hydroxy-phenyl)-ethylamino]-1,4,5,6-tetrahydro-1,2-diaza-benzo[e]azulen-8-yl}-2-oxo-oxazolidin-5-ylmethyl)-acetamide

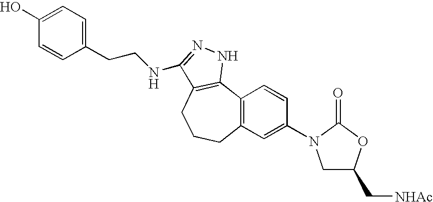

Following general procedure JJ, N-[3-(6(R,S)-bromo-5-oxo-6,7,8,9-tetrahydro-5H-benzocyclohepten-2-yl)-2-oxo-oxazolidin-5(S)-ylmethyl]-acetamide (0.395 g, 1.0 mmol) and 4-(2-(4-hydroxyphenyl)ethyl)-3-thiosemicarbazide (0.211 g, 1 mmol) gave the title compound in 25% yield (0.117 g). MS-APCI (m/z+): 432, 476 (M+H).

### Example 143

### (S)-N-{3-[3-(2-Amino-ethylamino)-1,4,5,6-tetrahydro-1,2-diaza-benzo[e]azulen-8-yl]-2-oxo-oxazolidin-5-ylmethyl}-acetamide

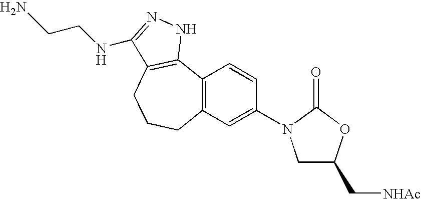

(S)-(2-{8-[5-(Acetylamino-methyl)-2-oxo-oxazolidin-3-yl]-1,4,5,6-tetrahydro-1,2-diaza-benzo[e]azulen-3-ylamino}-ethyl)-carbamic acid tert-butyl ester (0.120 g, 0.241 mmol) and 1.6 mL of anhydrous MeOH was cooled to 0° C. Acetyl chloride (0.17 mL, 2.41 mmol) was added dropwise and the mixture was stirred at 0° C. for 1 hour and then at room temperature overnight. The solution was concentrated in vacuo and then diluted with 10% MeOH/dichloromethane+2% NH₄OH. The solution was concentrated in vacuo to give a crude residue which was recrystallized from ethanol gave the title compound in 43% yield (0.041 g). MS-APCI (m/z+): 399 (M+H).

### Example 144

### (S)-N-{3-[3-(3-Amino-propylamino)-1,4,5,6-tetrahydro-1,2-diaza-benzo[e]azulen-8-yl]-2-oxo-oxazolidin-5-ylmethyl}-acetamide

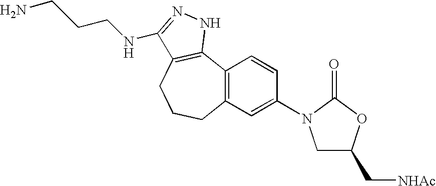

(S)-(3-{8-[5-(Acetylamino-methyl)-2-oxo-oxazolidin-3-yl]-1,4,5,6-tetrahydro-1,2-diaza-benzo[e]azulen-3-ylamino}-propyl)-carbamic acid tert-butyl ester (0.075 g, 0.146 mmol) and 1 mL of anhydrous MeOH was cooled to 0° C. Acetyl chloride (0.1 mL, 1.46 mmol) was added dropwise and the reaction was allowed to stir at room temperature overnight. The solution was concentrated in vacuo and then diluted with 10% MeOH/dichloromethane+2% NH₄OH. Purification by silica gel chromatography followed by recrystallization from ethanol gave the title compound in 54% yield (0.033 g). MS-APCI (m/z+): 369, 413 (M+H).

### Example 145

### (S)-N-[3-(3-Amino-1,4,5,6-tetrahydro-1,2-diaza-benzo[e]azulen-8-yl)-2-oxo-oxazolidin-5-ylmethyl]-acetamide

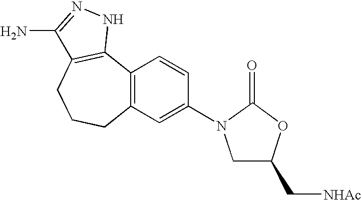

To a solution of (S)-N-{3-[3-(4-methoxy-benzylamino)-1,4,5,6-tetrahydro-1,2-diaza-benzo[e]azulen-8-yl]-2-oxo-oxazolidin-5-ylmethyl}-acetamide (0.197 g, 0.414 mmol) in 8 mL of dichloromethane was added triethylsilane (80 mL, 0.497 mmol), followed by the dropwise addition of trifluoroacetic acid (0.8 mL, 10.36 mmol). The mixture was stirred at room temperature for 3 hours and then concentrated in vacuo. The residue was diluted with ethyl acetate and then concentrated in vacuo a second time. The residue was washed with 75 mL of hexanes that had been heated to reflux. The residue was diluted with 20 mL of 20% MeOH/dichloromethane+2% NH₄OH and allowed to stir overnight. The mixture was concentrated in vacuo and then purified by silica gel chromatography to give the title compound in 87% yield (0.128 g). MS-APCI (m/z+): 312, 356 (M+H).

### Example 146

### (S)-N-{8-[5-(Acetylamino-methyl)-2-oxo-oxazolidin-3-yl]-1,4,5,6-tetrahydro-1,2-diaza-benzo[e]azulen-3-yl}-2-benzyloxy-acetamide

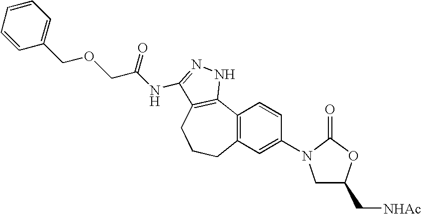

Pyridine (1.4 mL, 17.31 mmol), benzyloxyacetic acid (0.07 mL, 0.507 mmol), 1-(3-dimethylaminopropyl)-3-ethylcarbodiimide hydrochloride (EDC) (0.097 g, 0.51 mmol) and (S)-N-[3-(3-amino-1,4,5,6-tetrahydro-1,2-diaza-benzo[e]azulen-8-yl)-2-oxo-oxazolidin-5-ylmethyl]-acetamide (0.150 g, 0.422 mmol) was combined and allowed to stir overnight at room temperature. An additional 0.07 mL of benzyloxyacetic acid and 49 mg of EDC was added and the mixture was once again stirred overnight. The reaction was quenched with water and the aqueous layer was extracted twice with dichloromethane. The organic layer was concentrated in vacuo and then purified by silica gel chromatography to give the title compound in 47% yield (0.100 g). MS-APCI (m/z+): 460, 504 (M+H).

### Example 147

### (S)-N-{8-[5-(Acetylamino-methyl)-2-oxo-oxazolidin-3-yl]-1,4,5,6-tetrahydro-1,2-diaza-benzo[e]azulen-3-yl}-2-hydroxy-acetamide

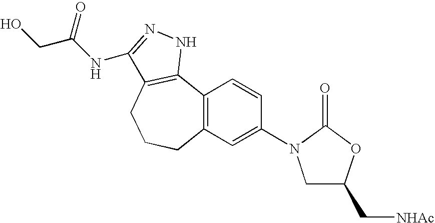

To (S)-N-{8-[5-(acetylamino-methyl)-2-oxo-oxazolidin-3-yl]-1,4,5,6-tetrahydro-1,2-diaza-benzo[e]azulen-3-yl}-2-benzyloxy-acetamide (0.086 g, 0.171 mmol) was added 2 mL of methanol, 28 mL of THF and 5% Pd/C (0.1 g). The reaction was placed under a H₂ pressure of 4295 psi/mol and monitored for progress. After 16 hours, an additional 20 mL of MeOH and 0.1 g of 5% Pd/C was added. After 37 hours, another 0.1 g of 5% Pd/C was added. After 55 hours, the mixture was filtered and concentrated in vacuo to give the title compound in 73% yield (0.051 g). MS-APCI (m/z+): 370, 414 (M+H).

### Example 148

### (S,S)-2,2-Dimethyl-[1,3]dioxolane-4-carboxylic acid {8-[5-(acetylamino-methyl)-2-oxo-oxazolidin-3-yl]-1,4,5,6-tetrahydro-1,2-diaza-benzo[e]azulen-3-yl}-amide

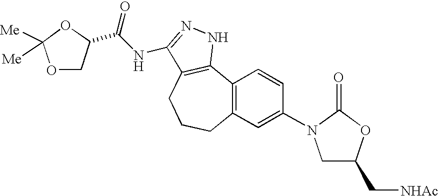

To (S)-N-[3-(3-amino-1,4,5,6-tetrahydro-1,2-diaza-benzo[e]azulen-8-yl)-2-oxo-oxazolidin-5-ylmethyl]-acetamide (0.073 g, 0.206 mmol) was added (S)-2,2-dimethyl-1,3-dioxolane-4-carboxylic acid (0.033 g, 0.23 mmol), pyridine (0.5 mL, 12.36 mmol), and 1-(3-dimethylaminopropyl)-3-ethylcarbodiimide hydrochloride (EDC) (0.047 g, 0.25 mmol). The reaction was stirred overnight at room temperature. An additional 24 mg of (S)-2,2-dimethyl-1,3-dioxolane-4-carboxylic acid, 0.4 mL of pyridine, and 22 mg of EDC was added and the mixture was then stirred for 72 hours. The reaction was quenched with water, the aqueous layer was extracted twice with dichloromethane, and the organic layer was concentrated in vacuo. Purification by silica gel chromatography afforded the title compound in 25% yield (0.025 g). MS-APCI (m/z+): 440, 484 (M+H).

### Example 149

### (S,S)-N-{8-[5-(Acetylamino-methyl)-2-oxo-oxazolidin-3-yl]-1,4,5,6-tetrahydro-1,2-diaza-benzo[e]azulen-3-yl}-2,3-dihydroxy-propionamide

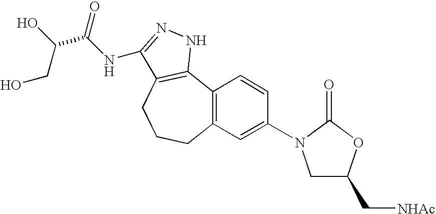

To (S,S)-2,2-dimethyl-[1,3]dioxolane-4-carboxylic acid {8-[5-(acetylamino-methyl)-2-oxo-oxazolidin-3-yl]-1,4,5,6-tetrahydro-1,2-diaza-benzo[e]azulen-3-yl}-amide (0.04 g, 0.083 mmol) suspended in 1 mL of THF was added 0.3 mL of 1M aqueous HCl. The solution was stirred overnight. An additional 0.3 mL of 1M aqueous HCl was added and the solution was stirred for another 6 hours. The solution was diluted with dichloromethane, concentrated in vacuo, and then this step was repeated. The solution was then diluted with toluene, concentrated in vacuo, and dried under vacuum at 40° C. to give the title compound in 85% yield (0.031 g). MS-APCI (m/z+): 444 (M+H).

### Example 150

### (S)-N-{3-[3-Amino-2-(furan-3-carbonyl)-2,4,5,6-tetrahydro-1,2-diaza-benzo[e]azulen-8-yl]-2-oxo-oxazolidin-5-ylmethyl}-acetamide

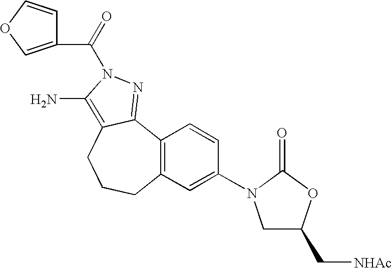

(S)-N-[3-(3-Amino-1,4,5,6-tetrahydro-1,2-diaza-benzo[e]azulen-8-yl)-2-oxo-oxazolidin-5-ylmethyl]-acetamide (0.075 g, 0.211 mmol), triethylamine (35 mL, 0.253 mmol) and 1 mL of dichloromethane was cooled to 0° C. To this suspension was added 3-furoyl chloride (0.033 g, 0.25 mmol) and the resultant clear solution was stirred for 45 minutes at 0° C. The reaction was quenched with saturated sodium bicarbonate and the aqueous layer was extracted twice with dichloromethane. The organic layer was washed with brine, dried over sodium sulfate and concentrated in vacuo. Purification by silica gel chromatography gave the title compound in 36% yield (0.034 g). MS-APCI (m/z+): 406, 450 (M+H).

### Example 151

### (S)-N-[3-(3-Benzylsulfanyl-1,4,5,6-tetrahydro-1,2-diaza-benzo[e]azulen-8-yl)-2-oxo-oxazolidin-5-ylmethyl]-acetamide

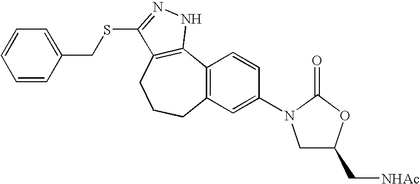

Following general procedure JJ, N-[3-(6(R,S)-bromo-5-oxo-6,7,8,9-tetrahydro-5H-benzocyclohepten-2-yl)-2-oxo-oxazolidin-5(S)-ylmethyl]-acetamide (0.395 g, 1 mmol) and hydrazinecarbodithioic acid benzyl ester (0.198 g, 1 mmol) gave the title compound in 13% yield (0.070 g). MS-APCI (m/z+): 419, 463 (M+H).

### Example 152

### (S)-N-[2-Oxo-3-(3-phenylmethanesulfonyl-1,4,5,6-tetrahydro-1,2-diaza-benzo[e]azulen-8-yl)-oxazolidin-5-ylmethyl]-acetamide

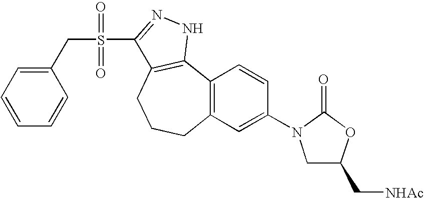

To a 0° C. suspension of (S)-N-[3-(3-benzylsulfanyl-1,4,5,6-tetrahydro-1,2-diaza-benzo[e]azulen-8-yl)-2-oxo-oxazolidin-5-ylmethyl]-acetamide 5 (0.130 g, 0.28 mmol) in 4 mL of chloroform was added mCPBA (0.130 g, 0.56 mmol) in one portion. The mixture was stirred for 1 hour at 0° C. and then stirred for 1 hour at room temperature. Saturated sodium bicarbonate and dichloromethane were added and the layers were separated. The organic layer was washed with water and the aqueous layer was back-extracted with dichloromethane. Purification by silica gel chromatography afforded the title compound in 34% yield (0.047 g). MS-APCI (m/z+): 451, 495 (M+H), 536 (M+CH₃CN).

### Example 153

### (S)-N-[3-(3-Benzyloxymethyl-1,4,5,6-tetrahydro-1,2-diaza-benzo[e]azulen-8-yl)-2-oxo-oxazolidin-5-ylmethyl]-acetamide

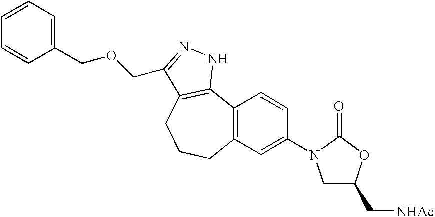

### N-{3-[6(R,S)-(2-Benzyloxy-acetyl)-5-oxo-6,7,8,9-tetrahydro-5H-benzocyclohepten-2-yl]-2-oxo-oxazolidin-5(S)-ylmethyl}-acetamide (Step 1)

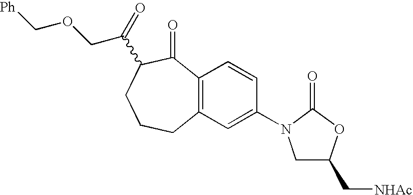

(S)-N-[2-Oxo-3-(5-oxo-6,7,8,9-tetrahydro-5H-benzocyclohepten-2-yl)-oxazolidin-5-ylmethyl]-acetamide (0.50 g, 1.58 mmol) in 11 mL of THF was cooled to ⁰° C. A 1 M solution of LiHMDS in THF (3.5 mL, 3.5 mmol) was added dropwise and the resultant solution was stirred for 30 minutes at 0° C. Benzyloxyacetyl chloride (0.5 mL, 3.16 mmol) in 8 mL of THF was added dropwise over the course of 30 minutes. The reaction was allowed to warm from 0 to 15° C. over the course of 1 hour and then saturated ammonium chloride was added. The mixture was concentrated in vacuo, diluted with dichloromethane, and the layers separated. The aqueous layer was extracted with dichloromethane and the organic layer was washed with brine, dried over sodium sulfate and concentrated in vacuo. Silica gel chromatography resulted in 0.700 g of the title compound. MS-APCI (m/z+): 417, 461 (M+H).

### (S)-N-[3-(3-Benzyloxymethyl-1,4,5,6-tetrahydro-1,2-diaza-benzo[e]azulen-8-yl)-2-oxo-oxazolidin-5-ylmethyl]-acetamide (Step 2)

To a suspension of N-{3-[6(R,S)-(2-benzyloxy-acetyl)-5-oxo-6,7,8,9-tetrahydro-5H-benzocyclohepten-2-yl]-2-oxo-oxazolidin-5(S)-ylmethyl}-acetamide (0.700 g, 1.51 mmol) in 12 mL of absolute ethanol was added hydrazine monohydrate (0.18 mL, 3.77 mmol). The reaction was stirred overnight at room temperature. Water and dichloromethane were added and the layers were separated. The organic layer was washed twice with water, dried over sodium sulfate and concentrated in vacuo. The residue was diluted with dichloromethane and allowed to stand for 5 days, during which time a solid precipitated from solution. The solid was collected and rinsed with ethyl acetate. Purification of the solid by silica gel chromatography afforded the title compound in 23% yield. MS-APCI (m/z+): 417, 461 (M+H).

### Example 154

### (S)-N-[3-(3-Hydroxymethyl-1,4,5,6-tetrahydro-1,2-diaza-benzo[e]azulen-8-yl)-2-oxo-oxazolidin-5-ylmethyl]-acetamide

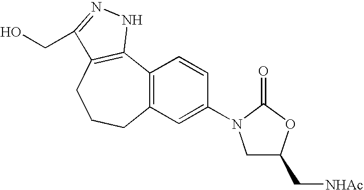

To (S)-N-[3-(3-benzyloxymethyl-1,4,5,6-tetrahydro-1,2-diaza-benzo[e]azulen-8-yl)-2-oxo-oxazolidin-5-ylmethyl]-acetamide (0.196 g, 0.426 mmol) was added 25 mL of MeOH, 25 mL of THF and 5% Pd/C (0.1 g). The reaction was placed under a H₂ pressure of 4295 psi/mol and monitored for progress. After 20 hours, an additional 0.1 g of 5% Pd/C was added. After 62 hours, an additional 0.05 g of 20% Pd/C was added. After a total of 82 hours, the mixture was filtered and concentrated in vacuo. Purification by silica gel chromatography afforded the title compound in 51% yield (0.08 g). MS-APCI (m/z+): 327, 371 (M+H).

### Example 155

### (R)-5-(Isoxazol-3-yloxymethyl)-3-(5-oxo-6,7,8,9-tetrahydro-5H-benzocyclohepten-2-yl)-oxazolidin-2-one

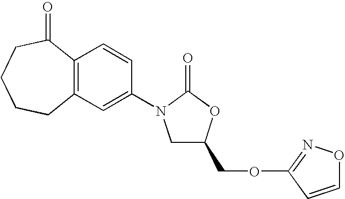

(R)-5-Hydroxymethyl-3-(5-oxo-6,7,8,9-tetrahydro-5H-benzocyclohepten-2-yl)-oxazolidin-2-one (0.275 g, 1 mmol), 3-hydroxyisoxazole (0.093 g, 1.1 mmol), 6 mL of THF, and polystyrene-bound triphenylphosphine (0.728 g, 1.15 mmol) was cooled to 0° C. Diisopropyl azodicarboxylate (DIAD) (0.22 mL, 1.1 mmol) was added dropwise and the mixture was stirred for 10 minutes at 0° C. and then allowed to warm to room temperature and stirred overnight in the dark. The solution was diluted with ethyl acetate, filtered through Celite, and rinsed with ethyl acetate. The filtrate was washed with water and brine, dried over sodium sulfate, and concentrated in vacuo. Purification by silica gel chromatography afforded the title compound in 66% yield (225 mg). MS-APCI (m/z+): 343 (M+H).

### Example 156

### 3-(5(R,S)-Hydroxy-6,7,8,9-tetrahydro-5H-benzocyclohepten-2-yl)-5(R)-(isoxazol-3-yloxymethyl)-oxazolidin-2-one

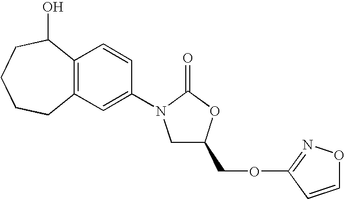

(R)-5-(Isoxazol-3-yloxymethyl)-3-(5-oxo-6,7,8,9-tetrahydro-5H-benzocyclohepten-2-yl)-oxazolidin-2-one (0.124 g, 0.362 mmol) and 2 mL of MeOH was cooled to 0° C. Sodium borohydride (4.5 mg, 0.12 mmol) was added in one portion. The cold bath was removed and the reaction was stirred at room temperature for 1 hour. An additional 10 mg of NaBH₄ was added and the reaction was stirred for another 2.5 hours. Water and ethyl acetate were added and the layers were separated. The organic layer was washed successively with water plus a few drops of 1 M aqueous HCl, water, saturated sodium bicarbonate and brine. The organic layer was dried over sodium sulfate, and concentrated in vacuo. Purification by silica gel chromatography gave the title compound in 52% yield (64.6 mg). MS-APCI (m/z+): 327, 343 (M−H), 344 (M).

### Example 157

### (R)-3-(6-Dimethylaminomethylene-5-oxo-6,7,8,9-tetrahydro-5H-benzocyclohepten-2-yl)-5-(isoxazol-3-yloxymethyl)-oxazolidin-2-one

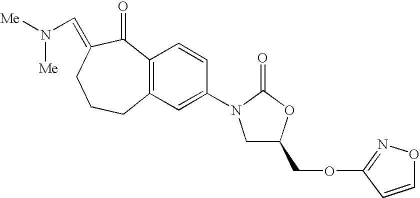

(R)-5-(Isoxazol-3-yloxymethyl)-3-(5-oxo-6,7,8,9-tetrahydro-5H-benzocyclohepten-2-yl)-oxazolidin-2-one (0.526 g, 1.54 mmol) in 15 mL of n-propanol was heated to just below reflux. N,N-dimethylformamide dimethyl acetal (0.82 mL, 6.14 mmol) was added and the resultant solution was then heated to reflux overnight. The solution was cooled to room temperature and concentrated in vacuo to give a solid. The solid was collected and rinsed with ether. Purification of the solid by silica gel chromatography gave the title compound in 67% yield (0.410 g). MS-APCI (m/z+): 398 (M+H).

### Example 158

### (R)-5-(Isoxazol-3-yloxymethyl)-3-(1,4,5,6-tetrahydro-1,2-diaza-benzo[e]azulen-8-yl)-oxazolidin-2-one

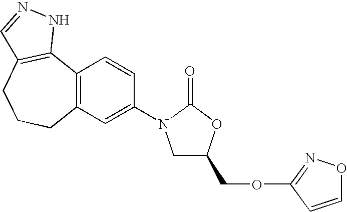

To a slurry of (R)-3-(6-dimethylaminomethylene-5-oxo-6,7,8,9-tetrahydro-5H-benzocyclohepten-2-yl)-5-(isoxazol-3-yloxymethyl)-oxazolidin-2-one (0.190 g, 0.48 mmol) in 6 mL of absolute ethanol was added hydrazine monohydrate (90 mL, 1.91 mmol). The reaction was stirred overnight at room temperature and the resultant precipitate was collected and washed with ethanol and ethyl acetate. Purification of the precipitate by silica gel chromatography afforded the title compound in 50% yield (87.6 mg). MS-APCI (m/z+): 367 (M+H).

### Example 159

### (R)-3-(5,6-Dihydro-4H-1-oxa-2-aza-benzo[e]azulen-8-yl)-5-(isoxazol-3-yloxymethyl)-oxazolidin-2-one

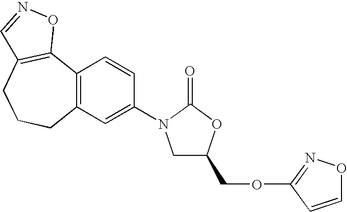

A slurry of (R)-3-(6-dimethylaminomethylene-5-oxo-6,7,8,9-tetrahydro-5H-benzocyclohepten-2-yl)-5-(isoxazol-3-yloxymethyl)-oxazolidin-2-one (0.436 g, 1.10 mmol) in 14 mL of MeOH was cooled to 0° C. Hydroxylamine-O-sulfonic acid (0.060 g, 0.53 mmol) in 7 mL of MeOH was added dropwise. After stirring for 10 minutes at 0° C., the cold bath was removed and the reaction was stirred at room temperature for 1 hour. Saturated sodium bicarbonate and ethyl acetate was added and the layers were separated. The aqueous layer was extracted twice with ethyl acetate. The organic layer was washed with brine, dried over sodium sulfate and concentrated in vacuo. Purification by silica gel chromatography gave the title compound in 44% yield (0.179 g). MS-APCI (m/z+): 368 (M+H).

### Example 160

### (R)-Methanesulfonic acid 2-oxo-3-(5-oxo-6,7,8,9-tetrahydro-5H-benzocyclohepten-2-yl)-oxazolidin-5-ylmethyl ester

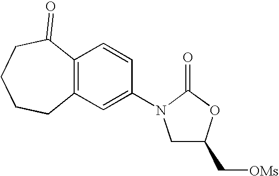

(R)-5-Hydroxymethyl-3-(5-oxo-6,7,8,9-tetrahydro-5H-benzocyclohepten-2-yl)-oxazolidin-2-one (1.00 g, 3.63 mmol) in 18 mL of dichloromethane was cooled to 0° C. Triethylamine (1 mL, 7.26 mmol) and methane sulfonyl chloride (0.3 mL, 3.63 mmol) was added dropwise. The mixture was stirred for 3 hours at 0° C. and then quenched with water. The layers were separated and the organic layer was washed with water. The aqueous layer was back-extracted with dichloromethane. The organic layers were combined, dried over sodium sulfate, and concentrated in vacuo to give the title compound in quantitative yield.

### Example 161

### (R)-3-(5-Oxo-6,7,8,9-tetrahydro-5H-benzocyclohepten-2-yl)-5-(pyridin-2-yloxymethyl)-oxazolidin-2-one

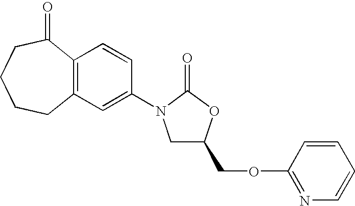

A solution of 2-hydroxypyridine (0.52 g, 5.45 mmol) in 3 mL of DMF was added dropwise to a 0° C. slurry of NaH (0.22 g, 5.45 mmol) in 18 mL of DMF. The cold bath was removed and the solution was stirred at room temperature for 30 minutes. A solution of (R)-methanesulfonic acid 2-oxo-3-(5-oxo-6,7,8,9-tetrahydro-5H-benzocyclohepten-2-yl)-oxazolidin-5-ylmethyl ester (1.28 g, 3.63 mmol) in 4 mL of DMF was added dropwise. The resultant mixture was stirred overnight at room temperature. The mixture was heated to 60° C. for 1.5 hours and then cooled to room temperature. Water, 1 M aqueous HCl and dichloromethane were added and the layers were separated. The organic layer was washed with several portions of water, dried over sodium sulfate, and concentrated in vacuo. Purification by silica gel chromatography afforded 0.362 g of the title compound, 0.392 g of Example 162 ((S)-1-[2-oxo-3-(5-oxo-6,7,8,9-tetrahydro-5H-benzocyclohepten-2-yl)-oxazolidin-5-ylmethyl]-1H-pyridin-2-one) and 0.124 g of starting material ((R)-methanesulfonic acid 2-oxo-3-(5-oxo-6,7,8,9-tetrahydro-5H-benzocyclohepten-2-yl)-oxazolidin-5-ylmethyl ester.) For the title compound: MS-APCI (m/z+): 353 (M+H).

### Example 162

### (S)-1-[2-Oxo-3-(5-oxo-6,7,8,9-tetrahydro-5H-benzocyclohepten-2-yl)-oxazolidin-5-ylmethyl]-1H-pyridin-2-one

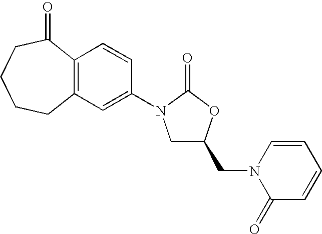

See Example 161 for procedure. MS-APCI (m/z+): 353 (M+H).

### Example 163

### (R)-3-(6-Dimethylaminomethylene-5-oxo-6,7,8,9-tetrahydro-5H-benzocyclohepten-2-yl)-5-(pyridin-2-yloxymethyl)-oxazolidin-2-one

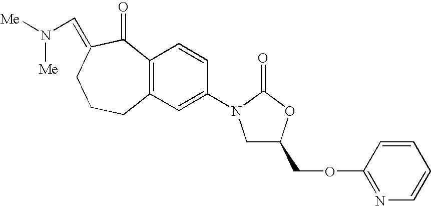

To (R)-3-(5-oxo-6,7,8,9-tetrahydro-5H-benzocyclohepten-2-yl)-5-(pyridin-2-yloxymethyl)-oxazolidin-2-one (0.333 g, 0.95 mmol) in 8 mL of n-propanol was added N,N-dimethylformamide dimethyl acetal (0.5 mL). The resultant solution was heated to reflux overnight. The solution was cooled to room temperature and concentrated in vacuo. Purification by silica gel chromatography gave the title compound in 65% yield (0.252 g). MS-APCI (m/z+): 408 (M+H).

### Example 164

### (R)-5-(Pyridin-2-yloxymethyl)-3-(1,4,5,6-tetrahydro-1,2-diaza-benzo[e]azulen-8-yl)-oxazolidin-2-one

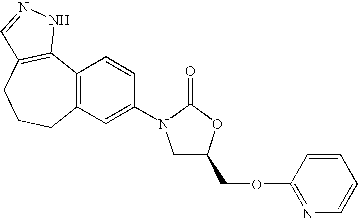

To a solution of (R)-3-(6-dimethylaminomethylene-5-oxo-6,7,8,9-tetrahydro-5H-benzocyclohepten-2-yl)-5-(pyridin-2-yloxymethyl)-oxazolidin-2-one (0.123 g, 0.302 mmol) in 3.8 mL of absolute ethanol was added hydrazine monohydrate (60 mL, 1.21 mmol). The solution was stirred overnight at room temperature. The reaction was diluted with dichloromethane and the organic layer was washed with 4×10 mL portions of water, brine, dried over sodium sulfate and concentrated in vacuo. Purification by silica gel chromatography gave the title compound in 68% yield (0.078 g). MS-APCI (m/z+): 377 (M+H).

### Example 165

### (R)-3-(5,6-Dihydro-4H-1-oxa-2-aza-benzo[e]azulen-8-yl)-5-(pyridin-2-yloxymethyl)-oxazolidin-2-one

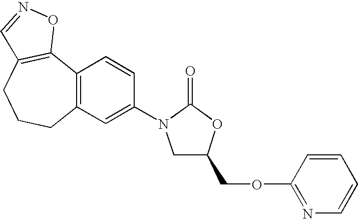

(R)-3-(6-Dimethylaminomethylene-5-oxo-6,7,8,9-tetrahydro-5H-benzocyclohepten-2-yl)-5-(pyridin-2-yloxymethyl)-oxazolidin-2-one (0.119 g, 0.29 mmol) in 3.7 mL of MeOH was cooled to 0° C. Hydroxylamine-O-sulfonic acid (0.036 g, 0.32 mmol) in 2 mL of MeOH was added and the solution was stirred for 10 minutes at 0° C. and then warmed to room temperature and stirred for 1 hour. Saturated sodium bicarbonate and ethyl acetate was added and the layers were separated. The aqueous layer was extracted with ethyl acetate and the organic layer was washed with water and brine, dried over sodium sulfate, and concentrated in vacuo. Purification by silica gel chromatography afforded the title compound in 31% yield (0.034 g). ¹H NMR (400 MHz, CDCl₃): δ 8.11 (m, 2H), 7.95 (d, 1H), 7.57 (m, 2H), 7.33 (dd, 1H), 6.90 (m, 1H), 6.75 (dt, 1H), 5.03 (m, 1H), 4.58 (d, 2H), 4.18 (t, 1H), 4.01 (dd, 1H), 2.92 (m, 2H), 2.76 (t, 2H), 1.99 (m, 2H).

### Example 166

### (S)-1-[3-(6-Dimethylaminomethylene-5-oxo-6,7,8,9-tetrahydro-5H-benzocyclohepten-2-yl)-2-oxo-oxazolidin-5-ylmethyl]-1H-pyridin-2-one

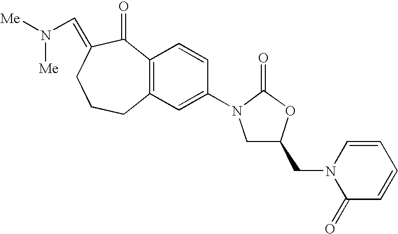

To (S)-1-[2-oxo-3-(5-oxo-6,7,8,9-tetrahydro-5H-benzocyclohepten-2-yl)-oxazolidin-5-ylmethyl]-1H-pyridin-2-one (0.364 g, 1.03 mmol) in 10 mL of n-propanol was added N,N-dimethylformamide dimethyl acetal (0.55 mL, 4.13 mmol). The resultant solution was heated to reflux overnight. The solution was cooled to room temperature and concentrated in vacuo. Purification by silica gel chromatography gave the title compound in quantitative yield (0.420 g).

### Example 167

### (S)-1-[2-Oxo-3-(1,4,5,6-tetrahydro-1,2-diaza-benzo[e]azulen-8-yl)-oxazolidin-5-ylmethyl]-1H-pyridin-2-one

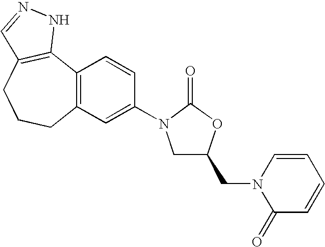

To a solution of (S)-1-[3-(6-dimethylaminomethylene-5-oxo-6,7,8,9-tetrahydro-5H-benzocyclohepten-2-yl)-2-oxo-oxazolidin-5-ylmethyl]-1H-pyridin-2-one (0.206 g, 0.506 mmol) in 6 mL of absolute ethanol was added hydrazine monohydrate (100 mL, 2.02 mmol). The solution was stirred overnight at room temperature. The reaction was diluted with dichloromethane and the organic layer was washed with 4×10 mL portions of water and brine, then dried over sodium sulfate and concentrated in vacuo. Purification by silica gel chromatography gave the title compound in 32% yield (0.060 g). MS-APCI (m/z+): 377 (M+H).

### Example 168

### (S)-1-[3-(5,6-Dihydro-4H-1-oxa-2-aza-benzo[e]azulen-8-yl)-2-oxo-oxazolidin-5-ylmethyl]-1H-pyridin-2-one

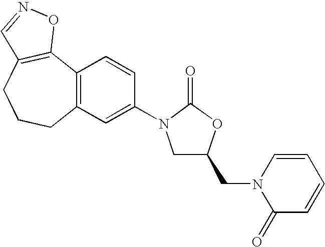

(S)-1-[3-(6-Dimethylaminomethylene-5-oxo-6,7,8,9-tetrahydro-5H-benzocyclohepten-2-yl)-2-oxo-oxazolidin-5-ylmethyl]-1H-pyridin-2-one (0.242 g, 0.59 mmol) in 7.4 mL of MeOH was cooled to 0° C. Hydroxylamine-O-sulfonic acid (0.074 g, 0.65 mmol) in 3 mL of MeOH was added and the solution was stirred for 45 minutes at 0° C. Saturated sodium bicarbonate and ethyl acetate were added and the layers were separated. The aqueous layer was extracted with ethyl acetate and the organic layer was washed with water and brine, dried over sodium sulfate, and concentrated in vacuo. Purification by silica gel chromatography afforded the title compound in 41% yield (0.092 g). MS-APCI (m/z+): 378 (M+H).

### Example 169

### (S)-N-[3-(3-Cyclopentyl-1,4,5,6-tetrahydro-1,2-diaza-benzo[e]azulen-8-yl)-2-oxo-oxazolidin-5-ylmethyl]-acetamide

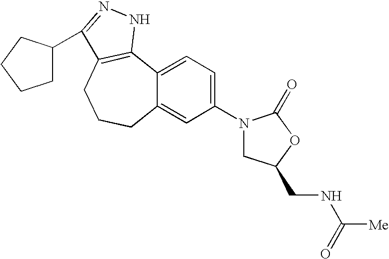

### N-[3-(6(R,S)-Cyclopentanecarbonyl-5-oxo-6,7,8,9-tetrahydro-5H-benzocyclohepten-2-yl)-2-oxo-oxazolidin-5(S)-ylmethyl]-acetamide (Step 1)

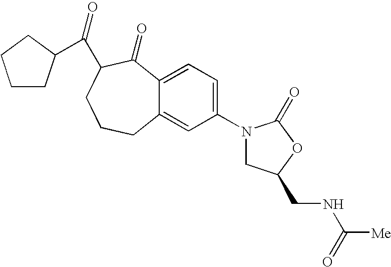

A solution of (S)-N-[2-oxo-3-(5-oxo-6,7,8,9-tetrahydro-5H-benzocyclohepten-2-yl)-oxazolidin-5-ylmethyl]-acetamide (501 mg. 1.58 mmol) in THF (12.7 mL) was cooled to ⁰° C. and 1 M solution of LiHMDS in THF (3.17 mL, 3.17 mmol) was added. The mixture was stirred at 0° C. for 30 minutes, then cyclopentanecarbonyl chloride (203 mL, 1.66 mmol) was added as a solution in THF (1.2 mL). After warming to room temperature overnight, the mixture was worked up with 0.5 N HCl, extracted with dichloromethane, dried over magnesium sulfate and concentrated in vacuo. The resulting oil was purified with column chromatography to result in the title compound (84.9 mg, 0.206 mmol, 13% Y). MS-APCI (m/z+): 369, 413 (M+H).

### (S)-N-[3-(3-Cyclopentyl-1,4,5,6-tetrahydro-1,2-diaza-benzo[e]azulen-8-yl)-2-oxo-oxazolidin-5-ylmethyl]-acetamide (Step 2)

To N-[3-(6(R,S)-cyclopentanecarbonyl-5-oxo-6,7,8,9-tetrahydro-5H-benzocyclohepten-2-yl)-2-oxo-oxazolidin-5(S)-ylmethyl]-acetamide (84.9 mg, 0.206 mmol) in ethanol (6 mL) was added hydrazine hydrochloride (16 mg, 0.23 mmol). The reaction mixture was then refluxed overnight. The resulting solution was cooled to room temperature and sodium bicarbonate was added to work up the reaction. The aqueous layer was extracted with dichloromethane; the organic layer was dried over sodium sulfate and concentrated in vacuo. The title compound was obtained by recrystallization from ethyl acetate. Isolated yield: 13 mg (15%). MS-APCI (m/z+): 365, 409 (M+H).

### Example 170

### (S)-Cyclopentanecarboxylic acid [2-oxo-3-(5-oxo-6,7,8,9-tetrahydro-5H-benzocyclohepten-2-yl)-oxazolidin-5-ylmethyl]-amide

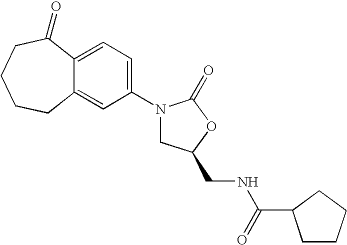

The title compound was isolated as another product in the synthesis of N-[3-(6(R,S)-cyclopentanecarbonyl-5-oxo-6,7,8,9-tetrahydro-5H-benzocyclohepten-2-yl)-2-oxo-oxazolidin-5(S)-ylmethyl]-acetamide. Isolated yield: 50 mg (8.5%). MS-APCI (m/z+): 327, 371 (M+H).

### Example 171

### (S)-N-[3-(3-Isopropyl-1,4,5,6-tetrahydro-1,2-diaza-benzo[e]azulen-8-yl)-2-oxo-oxazolidin-5-ylmethyl]-acetamide

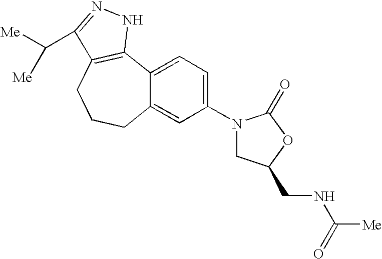

### N-[3-(6(R,S)-Isobutyryl-5-oxo-6,7,8,9-tetrahydro-5H-benzocyclohepten-2-yl)-2-oxo-oxazolidin-5(S)-ylmethyl]-acetamide (Step 1)

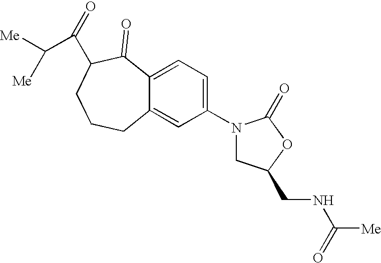

A solution of (S)-N-[2-oxo-3-(5-oxo-6,7,8,9-tetrahydro-5H-benzocyclohepten-2-yl)-oxazolidin-5-ylmethyl]-acetamide (799 mg, 2.52 mmol) in THF (18 mL) was cooled to 0° C. and a 1 M solution of LiHMDS in THF (5.05 mL, 5.05 mmol) was added. The mixture was stirred at 0° C. for 30 minutes then isobutyryl chloride (278 mL, 2.65 mmol) was added as a solution in THF (2 mL). After warming to room temperature overnight, the mixture was worked up with 0.5 N HCl, extracted with dichloromethane, dried over sodium sulfate and concentrated in vacuo. The resulting oil was purified with column chromatography to result in the title compound (226 mg, 0.585 mmol, 23% Y). MS-APCI (m/z+): 343, 387 (M+H).

### (S)-N-[3-(3-Isopropyl-1,4,5,6-tetrahydro-1,2-diaza-benzo[e]azulen-8-yl)-2=oxo-oxazolidin-5-ylmethyl]-acetamide (Step 2)

To N-[3-(6(R,S)-isobutyryl-5-oxo-6,7,8,9-tetrahydro-5H-benzocyclohepten-2-yl)-2-oxo-oxazolidin-5(S)-ylmethyl]-acetamide (226 mg, 0.585 mmol) in ethanol (16 mL) was added hydrazine hydrochloride (61 mg, 0.88 mmol). The reaction mixture was then refluxed overnight. The solution was cooled to room temperature and sodium bicarbonate was added. The aqueous layer was extracted with dichloromethane; the organic layer was dried over sodium sulfate and concentrated in vacuo. The titled compound was obtained by recrystallization from ethyl acetate followed by column chromatography and preparatory HPLC. Isolated yield: 14 mg (6%). MS-APCI (m/z+): 339, 383 (M+H).

### Example 172

### (S)-N-[3-(3-,5,6-tetrahydro-1,2-diaza-benzo[e]azulen-8-yl)-2-oxo-oxazolidin-5-ylmethyl]-isobutyramide

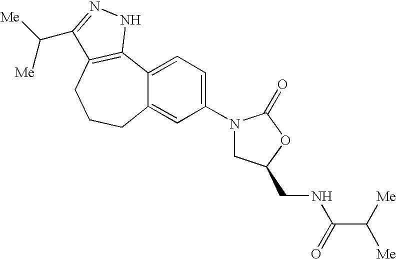

### N-[3-(6(R,S)-Isobutyryl-5-oxo-6,7,8,9-tetrahydro-5H-benzocyclohepten-2-oxo-oxazolidin-5(S)-ylmethyl]-isobutyramide (Step 1)

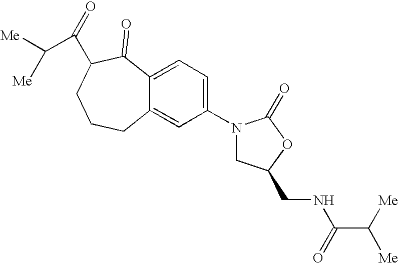

The title compound was isolated as another product in the synthesis of N-[3-(6(R,S)-isobutyryl-5-oxo-6,7,8,9-tetrahydro-5H-benzocyclohepten-2-yl)-2-oxo-oxazolidin-5(S)-ylmethyl]-acetamide (described above.) Isolated yield 510 mg (48%). MS-APCI (m/z+): 415 (M+H).

### (S)-N-[3-(3-,5,6-tetrahydro-1,2-diaza-benzo[e]azulen-8-yl)-2-oxo-oxazolidin-5-ylmethyl]-isobutyramide (Step 2)

To N-[3-(6(R,S)-isobutyryl-5-oxo-6,7,8,9-tetrahydro-5H-benzocyclohepten-2-yl)-2-oxo-oxazolidin-5(S)-ylmethyl]-isobutyramide (510 mg, 1.23 mmol) in ethanol (35 mL) was added hydrazine hydrochloride (130 mg, 1.8 mmol). The reaction mixture was then refluxed overnight. The resulting solution was cooled and sodium bicarbonate was added. The aqueous layer was extracted with dichloromethane; the organic layer was dried over sodium sulfate and concentrated in vacuo. The titled compound was obtained by recrystallization from ethyl acetate. Isolated yield: 20 mg (4%). MS-APCI (m/z+): 411 (M+H).

### Example 173

### (S)-N-[3-(3-Ethyl-1,4,5,6-tetrahydro-1,2-diaza-benzo[e]azulen-8-yl)-2-oxo-oxazolidin-5-ylmethyl]-acetamide

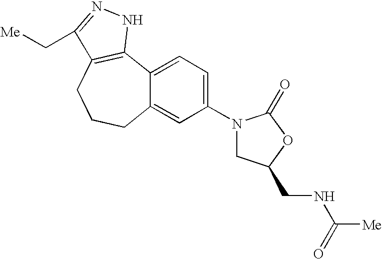

### N-[2-Oxo-3-(5-oxo-6(R,S)-propionyl-6,7,8,9-tetrahydro-5H-benzocyclohepten-2-yl)-oxazolidin-5(S)-ylmethyl]-acetamide (Step 1)

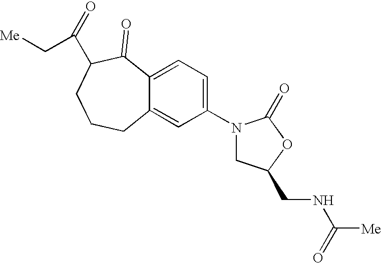

A solution of (S)-N-[2-oxo-3-(5-oxo-6,7,8,9-tetrahydro-5H-benzocyclohepten-2-yl)-oxazolidin-5-ylmethyl]-acetamide (501 mg, 1.58 mmol) in THF (15 mL) was cooled to 0° C. and 1 M solution of LiHMDS in THF (3.17 mL, 3.17 mmol) was added. The mixture was stirred at 0° C. for 30 minutes then propionyl chloride (145 mL, 1.66 mmol) was added as a solution in THF (10 mL). After warming to room temperature overnight, the mixture was worked up with saturated ammonium chloride, extracted with dichloromethane, dried over sodium sulfate and concentrated in vacuo. The resulting residue was purified with column chromatography to result in the title compound. Isolated yield: 199 mg (34% Y). MS-APCI (m/z+): 373 (M+H).

### (S)-N-[3-(3-Ethyl-1,4,5,6-tetrahydro-1,2-diaza-benzo[e]azulen-8-yl)-2-oxo-oxazolidin-5-ylmethyl]-acetamide (Step 2)

To N-[2-oxo-3-(5-oxo-6(R,S)-propionyl-6,7,8,9-tetrahydro-5H-benzocyclohepten-2-yl)-oxazolidin-5(S)-ylmethyl]-acetamide (199 mg, 0.53 mmol) in ethanol (10 mL) was added hydrazine hydrochloride (37 mg, 0.53 mmol). The reaction mixture was then refluxed overnight. The resulting solution was cooled and sodium bicarbonate was added. The aqueous layer was extracted with dichloromethane; the organic layer was dried over sodium sulfate and concentrated in vacuo. The title compound was obtained by recrystallization from ethyl acetate followed by column chromatography and preparatory HPLC. Isolated yield: 46 mg (23%). ¹H NMR (400 MHz, CD₃OD): δ 7.67 (br s, 1H), 7.40 (d, 1H), 7.34 (d, 1H), 4.72-4.77 (m, 1H), 4.12 (t, 1H), 3.81 (dd, 1H), 3.53 (d, 2H), 2.77 (br s, 2H), 2.66 (br s, 2H), 2.58 (q, 2H), 2.00 )br m, 2H), 1.93 (s, 3H), 1.21 (t, 3H)

### Example 174

### (S)-N-[2-Oxo-3-(3-propyl-1,4,5,6-tetrahydro-1,2-diaza-benzo[e]azulen-8-yl)-oxazolidin-5-ylmethyl]-acetamide

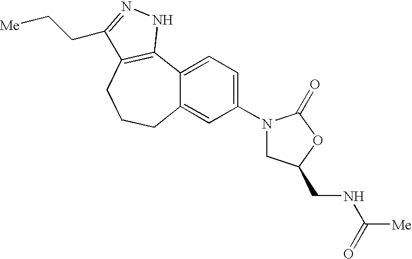

### N-[3-(6(R,S)-Butyryl-5-oxo-6,7,8,9-tetrahydro-5H-benzocyclohepten-2-yl)-2-oxo-oxazolidin-5(S)-ylmethyl]-acetamide (Step 1)

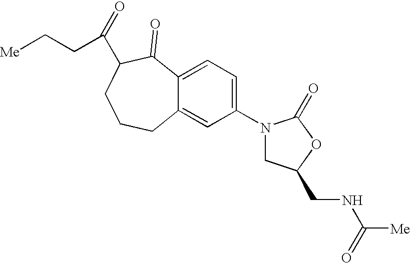

A solution of (S)-N-[2-oxo-3-(5-oxo-6,7,8,9-tetrahydro-5H-benzocyclohepten-2-yl)-oxazolidin-5-ylmethyl]-acetamide (502 mg, 1.59 mmol) in THF (15 mL) was cooled to 0° C. and 1 M solution of LiHMDS in THF (3.17 mL, 3.17 mmol) was added. The mixture was stirred at 0° C. for 30 minutes then butyryl chloride (173 mL, 1.67 mmol) was added dropwise as a solution in THF (10 mL) over 50 minutes. After warming to room temperature overnight, the mixture was worked up with saturated ammonium chloride, extracted with dichloromethane, dried over sodium sulfate and concentrated in vacuo. The resulting residue was purified with column chromatography to result in the title compound. Isolated yield: 225 mg (37% Y). MS-APCI (m/z+): 343, 387 (M+H).

### (S)-N-[2-Oxo-3-(3-propyl-1,4,5,6-tetrahydro-1,2-diaza-benzo[e]azulen-8-yl)-oxazolidin-5-ylmethyl]-acetamide (Step 2)

To N-[3-(6(R,S)-butyryl-5-oxo-6,7,8,9-tetrahydro-5H-benzocyclohepten-2-yl)-2-oxo-oxazolidin-5(S)-ylmethyl]-acetamide (225 mg, 0.582 mmol) in ethanol (11 mL) was added hydrazine hydrochloride (48 mg, 0.70 mmol). The reaction mixture was then refluxed overnight. The resulting solution was cooled and sodium bicarbonate was added. The aqueous layer was extracted with dichloromethane; the organic layer was dried over sodium sulfate and concentrated in vacuo. The titled compound was obtained by recrystallization from ethyl acetate followed by column chromatography and preparatory HPLC. Isolated yield: 45 mg (20%). MS-APCI (m/z+): 339, 387 (M+H).

### Example 175

### (S)-N-[2-Oxo-3-(3-propyl-1,4,5,6-tetrahydro-1,2-diaza-benzo[e]azulen-8-yl)-oxazolidin-5-ylmethyl]-butyramide

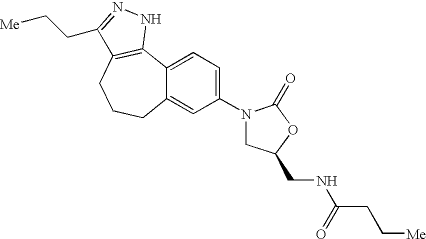

### N-[3-(6(R,S)-Butyryl-5-oxo-6,7,8,9-tetrahydro-5H-benzocyclohepten-2-yl)-2-oxo-oxazolidin-5(S)-ylmethyl]-butyramide (Step 1)

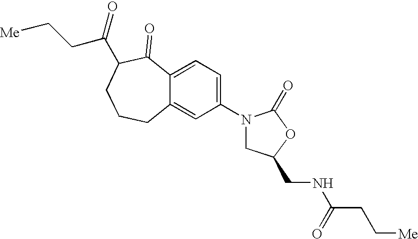

The title compound was isolated as another product in the synthesis of N-[3-(6(R,S)-butyryl-5-oxo-6,7,8,9-tetrahydro-5H-benzocyclohepten-2-yl)-2-oxo-oxazolidin-5(S)-ylmethyl]-acetamide. Isolated: 126 mg (19% Y). MS-APCI (m/z+): 371, 415 (M+H).

### (S)-N-[2-Oxo-3-(3-propyl-1,4,5,6-tetrahydro-1,2-diaza-benzo[e]azulen-8-yl)-oxazolidin-5-ylmethyl]-butyramide (Step 2)

To N-[3-(6(R,S)-butyryl-5-oxo-6,7,8,9-tetrahydro-5H-benzocyclohepten-2-yl)-2-oxo-oxazolidin-5(S)-ylmethyl]-butyramide (126 mg, 0.303 mmol) in ethanol (6 mL) was added hydrazine hydrate (23.6 mL, 0.758 mmol). The title compound formed as a precipitate and was isolated by filtering and washing with ethanol. Isolated yield: 22 mg (18%). MS-APCI (m/z+): 367, 411 (M+H).

### Example 176

### (S)-N-[2-Oxo-3-(3-thiophen-2-ylmethyl-1,4,5,6-tetrahydro-1,2-diaza-benzo[e]azulen-8-yl)-oxazolidin-5-ylmethyl]-acetamide

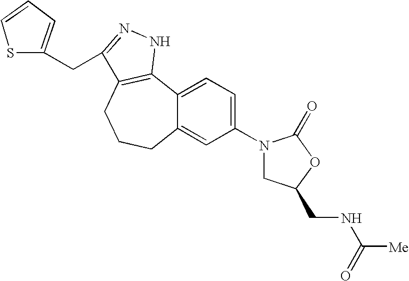

### N-{2-Oxo-3-[5-oxo-6(R,S)-(2-thiophen-2-yl-acetyl)-6,7,8,9-tetrahydro-5H-benzocyclohepten-2-yl]-oxazolidin-5(S)-ylmethyl}-acetamide (Step 1)

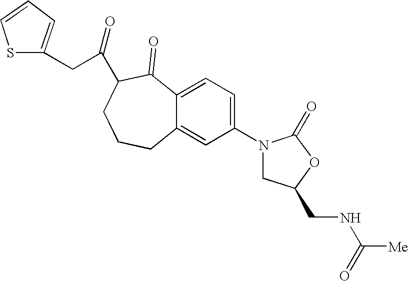

A solution of (S)-N-[2-oxo-3-(5-oxo-6,7,8,9-tetrahydro-5H-benzocyclohepten-2-yl)-oxazolidin-5-ylmethyl]-acetamide (400 mg, 1.26 mmol) in THF (15 mL) was cooled to 0° C., and 1 M solution of LiHMDS in THF (2.53 mL, 2.53 mmol) was added. The mixture was stirred at 0° C. for 30 minutes then thiophen-2-yl-acetyl chloride (164 mL, 1.33 mmol) was added dropwise as a solution in THF (10 mL) over 50 minutes. After warming to room temperature overnight, the mixture was worked up with saturated ammonium chloride, extracted with dichloromethane, dried over sodium sulfate and concentrated in vacuo. The resulting oil was purified with column chromatography to result in the title compound. Isolated yield: 221 mg (40% Y). MS-APCI (m/z+): 397, 441 (M+H).

### (S)-N-[2-Oxo-3-(3-thiophen-2-ylmethyl-1,4,5,6-tetrahydro-1,2-diaza-benzo[e]azulen-8-yl)-oxazolidin-5-ylmethyl]-acetamide (Step 2)

To N-{2-oxo-3-[5-oxo-6(R,S)-(2-thiophen-2-yl-acetyl)-6,7,8,9-tetrahydro-5H-benzocyclohepten-2-yl]-oxazolidin-5(S)-ylmethyl}-acetamide (221 mg) was added hydrazine hydrate (39.0 mL, 1.25 mmol). The title compound formed as a precipitate that was isolated by filtering and washing with ethanol. Isolated yield: 65 mg (30%). MS-APCI (m/z+): 393, 437 (M+H).

### Example 177

### (S)-N-{3-[10-Fluoro-3-(5-methyl-isoxazol-3-yl)-1,4,5,6-tetrahydro-1,2-diaza-benzo[e]azulen-8-yl]-2-oxo-oxazolidin-5-ylmethyl}-acetamide

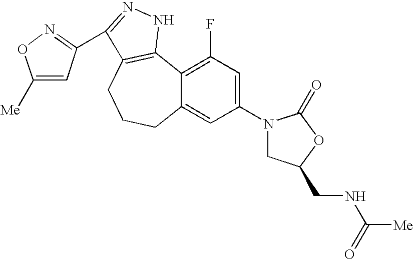

### N-{3-[4-Fluoro-6(R,S)-(5-methyl-isoxazole-3-carbonyl)-5-oxo-6,7,8,9-tetrahydro-5H-benzocyclohepten-2-yl]-2-oxo-oxazolidin-5(S)-ylmethyl}-acetamide (Step 1)

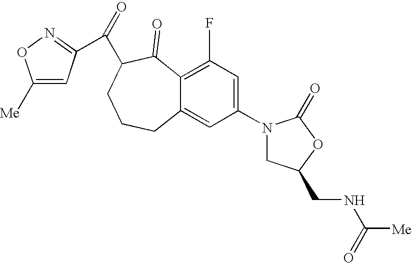

A solution of (S)-N-[3-(4-fluoro-5-oxo-6,7,8,9-tetrahydro-5H-benzocyclohepten-2-yl)-2-oxo-oxazolidin-5-ylmethyl]-acetamide (504 mg, 1.51 mmol) in THF (15 mL) was cooled to 0° C. and 1 M solution of LiHMDS in THF (2.70 mL, 2.70 mmol) was added. The mixture was stirred at 0° C. for 30 minutes then 5-methyl-isoxazole-3-carbonyl chloride (224 mg, 1.73 mmol) was added dropwise as a solution in THF (10 mL) over 1 hour. After warming to room temperature overnight, the mixture was worked up with saturated ammonium chloride, extracted with dichloromethane, dried over sodium sulfate and concentrated in vacuo. The resulting oil was purified with column chromatography to result in the title compound. Isolated yield: 388 mg (58% Y). MS-APCI (m/z+): 444 (M+H).

### (S)-N-{3-[10-Fluoro-3-(5-methyl-isoxazol-3-yl)-1,4,5,6-tetrahydro-1,2-diaza-benzo[e]azulen-8-yl]-2-oxo-oxazolidin-5-ylmethyl}-acetamide (Step 2)

To N-{3-[4-fluoro-6(R,S)-(5-methyl-isoxazole-3-carbonyl)-5-oxo-6,7,8,9-tetrahydro-5H-benzocyclohepten-2-yl]-2-oxo-oxazolidin-5(S)-ylmethyl}-acetamide (388 mg, 0.875 mmol) in ethanol (16 mL) hydrazine hydrate (68.1 mL, 2.19 mmol) was added. The reaction mixture was then stirred at room temperature overnight. The conversion was not complete after overnight stirring. Ethanol was removed in vacuo, added again and removed in vacuo. To the resulting residue, acetic acid (5 mL) was added. After stirring the reaction in acetic acid at room temperature, all the all the starting material was consumed. Acetic acid was removed in vacuo and sodium bicarbonate was added to work up the reaction. The aqueous layer was extracted with dichloromethane; the organic layer was dried over sodium sulfate and concentrated in vacuo. The titled compound was obtained by recrystallization from ethyl acetate. Isolated yield: 198 mg (51%). MS-APCI (m/z+): 396, 440 (M+H).

### Example 178

### (S)-5-Methyl-isoxazole-3-carboxylic acid {3-[10-fluoro-3-(5-methyl-isoxazol-3-yl)-1,4,5,6-tetrahydro-1,2-diaza-benzo[e]azulen-8-yl]-2-oxo-oxazolidin-5-ylmethyl}-amide

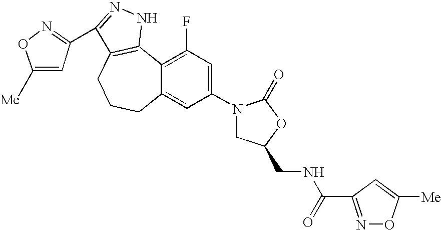

### 5-Methyl-isoxazole-3-carboxylic acid {3-[4-fluoro-6(R,S)-(5-methyl-isoxazole-3-carbonyl)-5-oxo-6,7,8,9-tetrahydro-5H-benzocyclohepten-2-yl]-2-oxo-oxazolidin-5(S)-ylmethyl}-amide (Step 1)

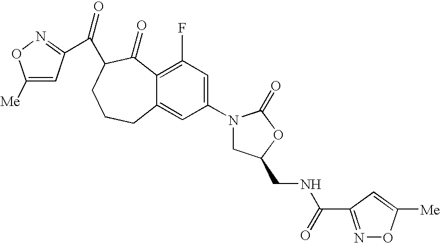

The title compound was isolated as another product in the synthesis of N-{3-[4-fluoro-6(R,S)-(5-methyl-isoxazole-3-carbonyl)-5-oxo-6,7,8,9-tetrahydro-5H-benzocyclohepten-2-yl]-2-oxo-oxazolidin-5(S)-ylmethyl}-acetamide. Isolated yield: 152 mg (20%). MS-APCI (m/z+): 511 (M+H).

### (S)-5-Methyl-isoxazole-3-carboxylic acid {3-[10-fluoro-3-(5-methyl-isoxazol-3-yl)-1,4,5,6-tetrahydro-1,2-diaza-benzo[e]azulen-8-yl]-2-oxo-oxazolidin-5-ylmethyl}-amide (Step 2)

To 5-methyl-isoxazole-3-carboxylic acid {3-[4-fluoro-6(R,S)-(5-methyl-isoxazole-3-carbonyl)-5-oxo-6,7,8,9-tetrahydro-5H-benzocyclohepten-2-yl]-2-oxo-oxazolidin-5(S)-ylmethyl}-amide (152 mg, 0.297 mmol) in ethanol (5 mL) was added hydrazine hydrate (23.1 mL, 0.742 mmol). The reaction mixture was then stirred at room temperature overnight. The conversion was not complete after overnight stirring. Ethanol was removed in vacuo, added again and removed in vacuo. To the resulting residue, acetic acid (5 mL) was added. After stirring the reaction in acetic acid at room temperature, all of the starting material was consumed. Acetic acid was removed in vacuo, and sodium bicarbonate was added; the aqueous layer was extracted with dichloromethane, and the organic extract was dried over sodium sulfate and concentrated in vacuo. The titled compound was obtained by recrystallization from ethyl acetate. Isolated yield: 55 mg (36%). MS-APCI (m/z+): 507 (M+H).

### Example 179

### (S)-N-{3-[3-(1,5-Dimethyl-1H-pyrazol-3-yl)-10-fluoro-1,4,5,6-tetrahydro-1,2-diaza-benzo[e]azulen-8-yl]-2-oxo-oxazolidin-5-ylmethyl}-acetamide

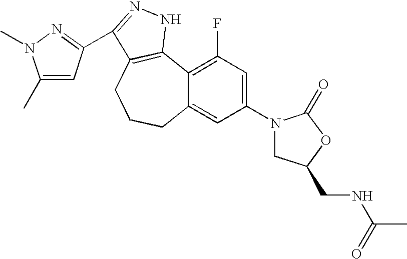

### N-{3-[6(R,S)-(1,5-Dimethyl-1H-pyrazole-3-carbonyl)-4-fluoro-5-oxo-6,7,8,9-tetrahydro-5H-benzocyclohepten-2-yl]-2-oxo-oxazolidin-5(S)-ylmethyl}-acetamide (Step 1)

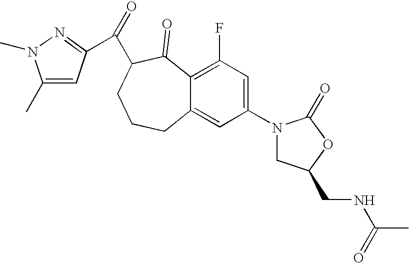

A solution of (S)-N-[3-(4-fluoro-5-oxo-6,7,8,9-tetrahydro-5H-benzocyclohepten-2-yl)-2-oxo-oxazolidin-5-ylmethyl]-acetamide (500 mg, 1.50 mmol) in THF (15 mL) was cooled to 0° C. and 1 M solution of LiHMDS in THF (2.68 mL, 2.68 mmol) was added. The mixture was stirred at 0° C. for 30 minutes then 1,5-dimethyl-1H-pyrazole-3-carbonyl chloride (243 mg, 1.53 mmol) was added dropwise as a solution in THF (10 mL) over 1 hour. After warming to room temperature overnight, the mixture was worked up with saturated ammonium chloride, extracted with dichloromethane, dried over sodium sulfate and concentrated in vacuo. The resulting oil was purified with column chromatography to result in the title compound. Isolated yield: 336 mg (49% Y). MS-APCI (m/z+): 413, 457 (M+H).

### (S)-N-{3-[3-(1,5-Dimethyl-1H-pyrazol-3-yl)-10-fluoro-1,4,5,6-tetrahydro-1,2-diaza-benzo[e]azulen-8-yl]-2-oxo-oxazolidin-5-ylmethyl}-acetamide (Step 2)

To N-{3-[6(R,S)-(1,5-dimethyl-1H-pyrazole-3-carbonyl)-4-fluoro-5-oxo-6,7,8,9-tetrahydro-5H-benzocyclohepten-2-yl]-2-oxo-oxazolidin-5 (S)-ylmethyl}-acetamide (336 mg, 0.736 mmol) in ethanol (13 mL) was added hydrazine hydrate (67.3 mL, 1.84 mmol). The reaction mixture was then stirred at room temperature overnight. Saturated ammonium chloride was added, and the aqueous layer was extracted with dichloromethane; the organic layer was dried over sodium sulfate and concentrated in vacuo. The resulting residue was purified by chromatography. Isolated yield: 84 mg (25%). MS-APCI (m/z+): 409, 453 (M+H).

### Example 180

### (S)-1,5-Dimethyl-1H-pyrazole-3-carboxylic acid {3-[3-(1,5-dimethyl-1H-pyrazol-3-yl)-10-fluoro-1,4,5,6-tetrahydro-1,2-diaza-benzo[e]azulen-8-yl]-2-oxo-oxazolidin-5-ylmethyl}-amide

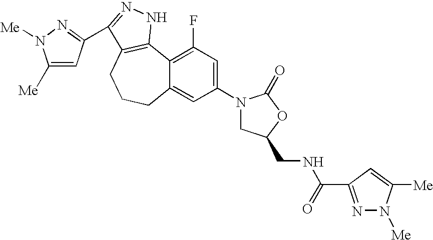

### 1,5-Dimethyl-1H-pyrazole-3-carboxylic acid {3-[6(R,S)-(1,5-dimethyl-1H-pyrazole-3-carbonyl)-4-fluoro-5-oxo-6,7,8,9-tetrahydro-5H-benzocyclohepten-2-yl]-2-oxo-oxazolidin-5(S)-ylmethyl}-amide (Step 1)

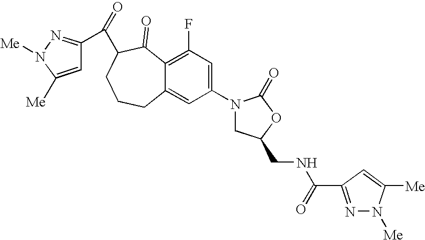

The title compound was isolated as another product in the synthesis of N-{3-[6(R,S)-(1,5-dimethyl-1H-pyrazole-3-carbonyl)-4-fluoro-5-oxo-6,7,8,9-tetrahydro-5H-benzocyclohepten-2-yl]-2-oxo-oxazolidin-5 (S)-ylmethyl}-acetamide. Isolated yield: 143 mg (18%). MS-APCI (m/z+): 493, 537 (M+H).

### (S)-1,5-Dimethyl-1H-pyrazole-3-carboxylic acid {3-[3-(1,5-dimethyl-1H-pyrazol-3-yl)-10-fluoro-1,4,5,6-tetrahydro-1,2-diaza-benzo[e]azulen-8-yl]-2-oxo-oxazolidin-5-ylmethyl}-amide (Step 2)

To 1,5-dimethyl-1H-pyrazole-3-carboxylic acid {3-[6(R,S)-(1,5-dimethyl-1H-pyrazole-3-carbonyl)-4-fluoro-5-oxo-6,7,8,9-tetrahydro-5H-benzocyclohepten-2-yl]-2-oxo-oxazolidin-5(S)-ylmethyl}-amide (143 mg, 0.266 mmol) in ethanol (5 mL) was added hydrazine hydrate (17.4 mL, 0.558 mmol). The reaction mixture was then stirred at room temperature overnight. After overnight stirring, the reaction was still not complete. Ethanol was removed in vacuo and acetic acid (5 mL) was added to the reaction. After stirring the reaction in acetic acid at room temperature, all of the starting material was consumed. Acetic acid was removed in vacuo, and sodium bicarbonate was added; the aqueous layer was extracted with dichloromethane, and the organic layer was dried over sodium sulfate and concentrated in vacuo. The title compound was obtained by recrystallization from ethyl acetate. Isolated yield: 29 mg (20%). MS-APCI (m/z+): 533 (M+H).

### Example 181

### (S)-N-[3-(10-Fluoro-3-furan-2-yl-1,4,5,6-tetrahydro-1,2-diaza-benzo[e]azulen-8-yl)-2-oxo-oxazolidin-5-ylmethyl]-acetamide

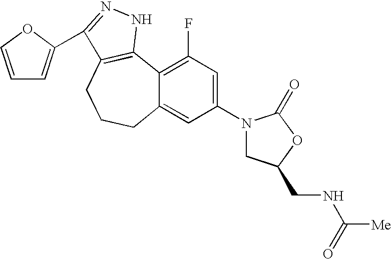

### N-{3-[4-Fluoro-6(R,S)-(furan-2-carbonyl)-5-oxo-6,7,8,9-tetrahydro-5H-benzocyclohepten-2-yl]-2-oxo-oxazolidin-5(S)-ylmethyl}-acetamide (Step 1)

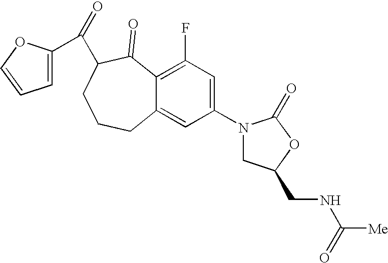

A solution of (S)-N-[3-(4-fluoro-5-oxo-6,7,8,9-tetrahydro-5H-benzocyclohepten-2-yl)-2-oxo-oxazolidin-5-ylmethyl]-acetamide (501 mg, 1.50 mmol) in THF (15 mL) was cooled to 0° C. and 1 M solution of LiHMDS in THF (3.15 mL, 3.15 mmol) was added. The mixture was stirred at 0° C. for 30 minutes, then furan-2-carbonyl chloride (235 mg, 1.80 mmol) was added dropwise as a solution in THF (10 mL) over 1 hour. After warming to room temperature overnight, the mixture was worked up with saturated ammonium chloride, extracted with dichloromethane, dried over sodium sulfate and concentrated in vacuo. The resulting oil was purified with column chromatography to result in the title compound. Isolated yield: 459 mg (71% Y). MS-APCI (m/z+): 385, 429 (M+H).

### (S)-N-[3-(10-Fluoro-3-furan-2-yl-1,4,5,6-tetrahydro-1,2-diaza-benzo[e]azulen-8-yl)-2-oxo-oxazolidin-5-ylmethyl]-acetamide (Step 2)

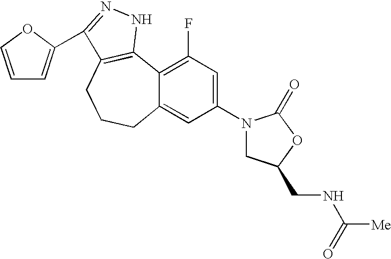

To N-{3-[4-fluoro-6(R,S)-(furan-2-carbonyl)-5-oxo-6,7,8,9-tetrahydro-5H-benzocyclohepten-2-yl]-2-oxo-oxazolidin-5(S)-ylmethyl}-acetamide (459 mg, 1.07 mmol) in ethanol (19 mL) was added hydrazine hydrate (83.3 mL, 2.68 mmol). The reaction mixture was then stirred at room temperature overnight. The title compound was formed in the reaction mixture as a precipitate and was isolated by filtration and washing with ethanol. Isolated yield: 125 mg (28%). MS-APCI (m/z+): 381, 425 (M+H).

### Example 182

### (S)-N-{3-[1-(5-Methyl-isoxazol-3-yl)-3,4,5,6-tetrahydro-2,3-diaza-benzo[e]azulen-8-yl]-2-oxo-oxazolidin-5-ylmethyl}-acetamide

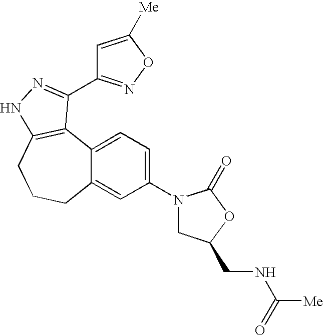

### N-[3-[5(R,S)-(5-Methyl-isoxazole-3-carbonyl)-6-oxo-6,7,8,9-tetrahydro-5H-benzocyclohepten-2-yl]-2-oxo-oxazolidin-5(S)-ylmethyl]-acetamide (Step 1)

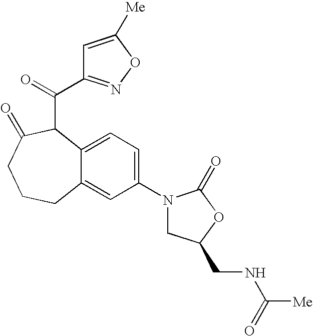

A solution of (S)-N-[2-oxo-3-(6-oxo-6,7,8,9-tetrahydro-5H-benzocyclohepten-2-yl)-oxazolidin-5-ylmethyl]-acetamide (46) (500 mg, 1.58 mmol) in THF (15 mL) was cooled to 0° C. and 1 M solution of LiHMDS in THF (3.32 mL, 3.32 mmol) was added. The mixture was stirred at 0° C. for 30 minutes, and then 5-methyl-isoxazole-3-carbonyl chloride (276 mg, 1.90 mmol) was added dropwise as a solution in THF (10 mL) over 50 minutes. After warming to room temperature overnight, the mixture was worked up with saturated ammonium chloride, extracted with dichloromethane, dried over sodium sulfate and concentrated in vacuo. The resulting oil was purified with column chromatography to result in the title compound. Isolated yield: 269 mg (40% Y). MS-APCI (m/z+): 426 (M+H).

### (S)-N-[3-[1-(5-Methyl-isoxazol-3-yl)-3,4,5,6-tetrahydro-2,3-diaza-benzo[e]azulen-8-yl]-2-oxo-oxazolidin-5-ylmethyl]-acetamide (48) (Step 2)

To N-{3-[5(R,S)-(5-methyl-isoxazole-3-carbonyl)-6-oxo-6,7,8,9-tetrahydro-5H-benzocyclohepten-2-yl]-2-oxo-oxazolidin-5(S)-ylmethyl}-acetamide (269 mg, 0.632 mmol) in ethanol (13 mL) was added hydrazine hydrate (49.2 mL, 1.58 mmol). The reaction mixture was then stirred at room temperature overnight. Since the reaction was not complete, ethanol was removed in vacuo and the reaction mixture was heated in acetic acid (10 mL) at 40-45° C. for 30 minutes. The acetic acid was then removed in vacuo, and sodium bicarbonate was added. The aqueous layer was extracted with dichloromethane, and the organic layer was dried over sodium sulfate and concentrated in vacuo. The resulting residue was purified by column chromatography. Isolated yield: 140 mg (53%). MS-APCI (m/z+): 378, 422 (M+H).

### Examples 183 and 184

### (S)-N-[2-Oxo-3-(1-pyridin-3-yl-3,4,5,6-tetrahydro-2,3-diaza-benzo[e]azulen-8-yl)-oxazolidin-5-ylmethyl]-acetamide and (S)-N-[2-Oxo-3-(1-pyridin-3-yl-3,4,5,6-tetrahydro-2,3-diaza-benzo[e]azulen-8-yl)-oxazolidin-5-ylmethyl]-nicotinamide

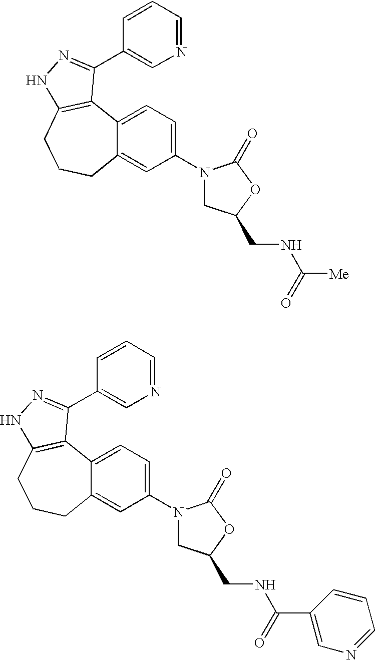

### N-{2-Oxo-3-[6-oxo-5(R,S)-(pyridine-3-carbonyl)-6,7,8,9-tetrahydro-5H-benzocyclohepten-2-yl]-oxazolidin-5(S)-ylmethyl}-acetamide and N-{2-Oxo-3-[6-oxo-5(R,S)-(pyridine-3-carbonyl)-6,7,8,9-tetrahydro-5H-benzocyclohepten-2-yl]-oxazolidin-5(S)-ylmethyl}-nicotinamide (Step 1)

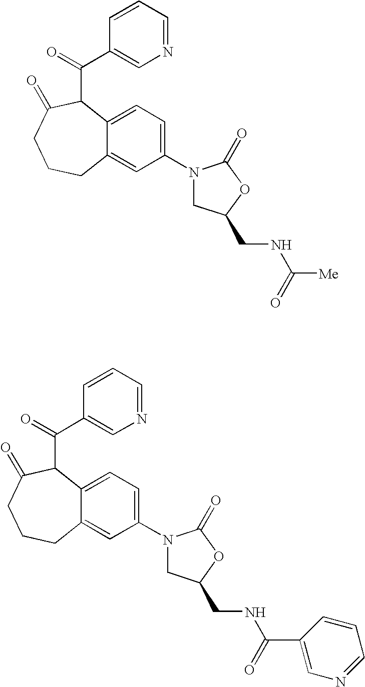

A solution of (S)-N-[2-oxo-3-(6-oxo-6,7,8,9-tetrahydro-5H-benzocyclohepten-2-yl)-oxazolidin-5-ylmethyl]-acetamide (500 mg, 1.58 mmol) in THF (15 mL) was cooled to 0° C. and 1 M solution of LiHMDS in THF (4.98 mL, 4.98 mmol) was added. The mixture was stirred at 0° C. for 30 minutes, and then nicotinoyl chloride hydrochloride salt (310 mg, 1.74 mmol) was added dropwise as a slurry in THF (10 mL) over 50 minutes After warming to room temperature overnight, the mixture was worked up with saturated ammonium chloride, extracted with dichloromethane, dried over sodium sulfate and concentrated in vacuo. The resulting oil was purified with column chromatography to result in the title compounds as a mixture. Isolated yield: 110 mg. (49) MS-APCI (m/z+): 379, 422 (M+H). (50) MS-APCI (m/z+): 485 (M+H).

### (S)-N-[2-Oxo-3-(1′-pyridin-3-yl-3,4,5,6-tetrahydro-2,3-diaza-benzo[e]azulen-8-yl)-oxazolidin-5-ylmethyl]-acetamide and (S)-N-[2-Oxo-3-(1-pyridin-3-yl-3,4,5,6-tetrahydro-2,3-diaza-benzo[e]azulen-8-yl)-oxazolidin-5-ylmethyl]-nicotinamide (Step 2)

To the mixture of N-{2-oxo-3-[6-oxo-5(R,S)-(pyridine-3-carbonyl)-6,7,8,9-tetrahydro-5H-benzocyclohepten-2-yl]-oxazolidin-5(S)-ylmethyl}-acetamide and N-{2-oxo-3-[6-oxo-5 (R,S)-(pyridine-3-carbonyl)-6,7,8,9-tetrahydro-5H-benzocyclohepten-2-yl]-oxazolidin-5(S)-ylmethyl}-nicotinamide (110 mg, approximately 260 mmol) in ethanol (13 mL) was added hydrazine hydrate (20.0 mL, 0.65 mmol). The reaction mixture was then stirred at room temperature overnight. Ethanol was removed in vacuo and water was added. The aqueous layer was extracted with dichloromethane, and the organic layer was dried over sodium sulfate and concentrated in vacuo. The resulting residue was purified by column chromatography.

For (S)-N-[2-Oxo-3-(1-pyridin-3-yl-3,4,5,6-tetrahydro-2,3-diaza-benzo[e]azulen-8-yl)-oxazolidin-5-ylmethyl]-acetamide: Isolated yield, 25 mg (23%). MS-APCI (m/z+): 374, 418 (M+H).

For (S)-N-[2-Oxo-3-(1-pyridin-3-yl-3,4,5,6-tetrahydro-2,3-diaza-benzo[e]azulen-8-yl)-oxazolidin-5-ylmethyl]-nicotinamide Isolated yield: 40 mg (32%). MS-APCI (m/z+): 437, 481 (M+H).

### Example 185

### (S)-N-[3-(1-Isoxazol-5-yl-3,4,5,6-tetrahydro-2,3-diaza-benzo[e]azulen-8-yl)-2-oxo-oxazolidin-5-ylmethyl]-acetamide

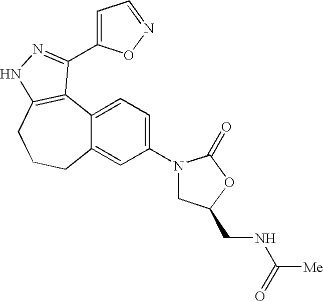

### N-{3-[5(R,S)-(Isoxazole-5-carbonyl)-6-oxo-6,7,8,9-tetrahydro-5H-benzocyclohepten-2-yl]-2-oxo-oxazolidin-5(S)-ylmethyl}-acetamide (Step 1)

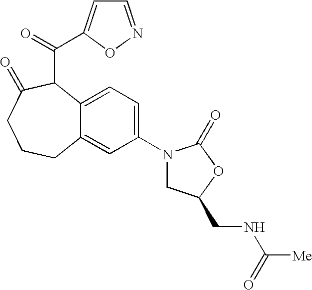

A solution of (S)-N-[2-oxo-3-(6-oxo-6,7,8,9-tetrahydro-5H-benzocyclohepten-2-yl)-oxazolidin-5-ylmethyl]-acetamide (500 mg, 1.58 mmol) in THF (15 mL) was cooled to 0° C. and 1 M solution of LiHMDS in THF (3.32 mL, 3.32 mmol) was added. The mixture was stirred at 0° C. for 30 minutes then isoxazole-5-carbonyl chloride (257 mg, 1.90 mmol) was added dropwise as a solution in THF (10 mL) over 50 minutes After warming to room temperature overnight, the mixture was worked up with saturated ammonium chloride, extracted with dichloromethane, dried over sodium sulfate and concentrated in vacuo. The resulting oil was purified with column chromatography to result in the title compound. Isolated yield: 269 mg (41% Y). MS-APCI (m/z+): 412 (M+H).

### (S)-N-[3-(1-Isoxazol-5-yl-3,4,5,6-tetrahydro-2,3-diaza-benzo[e]azulen-8-yl)-2-oxo-oxazolidin-5-ylmethyl]-acetamide (Step 2)

To N-{3-[5(R,S)-(isoxazole-5-carbonyl)-6-oxo-6,7,8,9-tetrahydro-5H-benzocyclohepten-2-yl]-2-oxo-oxazolidin-5(S)-ylmethyl}-acetamide (269 mg, 0.653 mmol) in ethanol (19 mL) was added hydrazine hydrate (50.9 mL, 1.63 mmol). The reaction mixture was then stirred at room temperature overnight. Since the reaction was not complete, ethanol was removed in vacuo and the reaction mixture was stirred in acetic acid (10 mL) at room temperature for 30 minutes Acetic acid was then removed in vacuo and water was added to work up the reaction. The aqueous layer was extracted with dichloromethane, and the organic layer was dried over sodium sulfate and concentrated in vacuo. The resulting oil was purified by column chromatography. Isolated yield: 257 mg (97%). MS-APCI (m/z+): 364, 408 (M+H).

### Example 186

### (S)-N-[3-(2-Amino-5,6-dihydro-4H-3-thia-1-aza-benzo[e]azulen-8-yl)-2-oxo-oxazolidin-5-ylmethyl]-acetamide hydrobromic acid salt

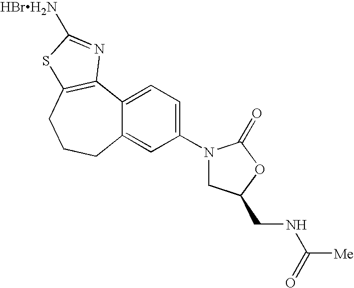

To N-[3-(6(R,S)-bromo-5-oxo-6,7,8,9-tetrahydro-5H-benzocyclohepten-2-yl)-2-oxo-oxazolidin-5(S)-ylmethyl]-acetamide (199.7 mg, 0.559 mmol) dissolved in warm ethanol (2 mL) was added thiourea (42.6 mg, 0.559 mmol). The resulting mixture was heated in microwave reactor at 120° C. for 4 minutes. The solid was filtered, washed with ethanol (2 mL) and ethyl acetate (2 mL) and dried to afford the title compound. Isolated yield: 174.6 mg (69%). MS-APCI (m/z+): 329, 373 (M+H).

### Example 187

### (S)-N-[3-(2-Methylamino-5,6-dihydro-4H-3-thia-1-aza-benzo[e]azulen-8-yl)-2-oxo-oxazolidin-5-ylmethyl]-acetamide hydrobromic acid salt

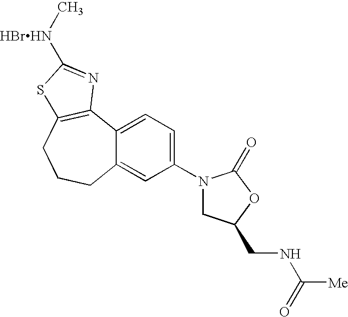

To N-[3-(6(R,S)-bromo-5-oxo-6,7,8,9-tetrahydro-5H-benzocyclohepten-2-yl)-2-oxo-oxazolidin-5(S)-ylmethyl]-acetamide (196.3 mg, 0.497 mmol) dissolved in warm ethanol (1.5 mL) was added methyl thiourea (44.8 mg, 0.497 mmol). The resulting mixture was heated in microwave reactor at 120° C. for 4 minutes. The solid was filtered, washed with ethanol (2 mL) and ethyl acetate (2 mL) and dried to afford the title compound. Isolated yield: 77.3 mg (33%). MS-APCI (m/z+): 343, 387 (M+H).

### Example 188

### (S)-N-{3-[2-(2-Morpholin-4-yl-ethylamino)-5,6-dihydro-4H-3-thia-1-aza-benzo[e]azulen-8-yl]-2-oxo-oxazolidin-5-ylmethyl}-acetamide hydrobromic acid salt

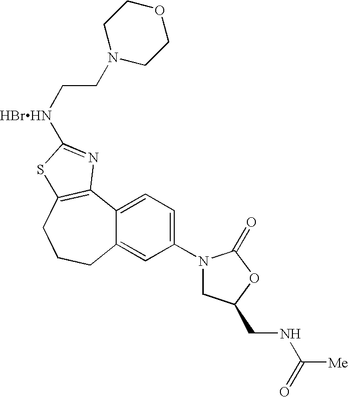

To N-[3-(6(R,S)-bromo-5-oxo-6,7,8,9-tetrahydro-5H-benzocyclohepten-2-yl)-2-oxo-oxazolidin-5(S)-ylmethyl]-acetamide (202.4 mg, 0.512 mmol) dissolved in warm ethanol (0.6 mL) was added (2-morpholin-4-yl-ethyl)-thiourea (96.9 mg, 0.512 mmol). The resulting mixture was heated in microwave reactor at 120° C. for 4 minutes. The solvent was removed from the reaction in vacuo; to the resulting crude was added ethanol (0.2 mL) and ethyl acetate (2 mL). A solid crashed out of the crude which was filtered, washed with ethyl acetate (2 mL) and dried to afford the title compound. Isolated yield: 228.1 mg (95%). MS-APCI (m/z+): 442, 486 (M+H).

### Example 189

### (S)-N-{2-Oxo-3-[2-(2-piperidin-1-yl-ethylamino)-5,6-dihydro-4H-3-thia-1-aza-benzo[e]azulen-8-yl]-oxazolidin-5-ylmethyl}-acetamide hydrobromic acid salt

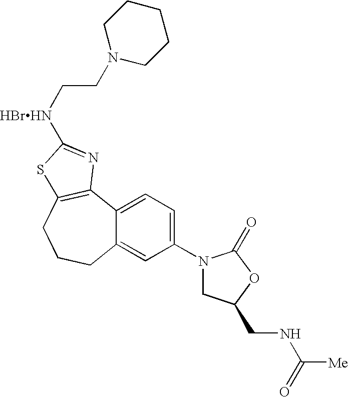

To N-[3-(6(R,S)-bromo-5-oxo-6,7,8,9-tetrahydro-5H-benzocyclohepten-2-yl)-2-oxo-oxazolidin-5(S)-ylmethyl]-acetamide (202.4 mg, 0.512 mmol) dissolved in warm ethanol (0.6 mL) was added (2-piperidin-1-yl-ethyl)-thiourea (95.9 mg, 0.512 mmol). The resulting mixture was heated in microwave reactor at 120° C. for 4 minutes. The solvent was removed from the reaction in vacuo; to the resulting crude was added ethanol (0.2 mL) and ethyl acetate (2 mL). The solid that formed was filtered, washed ethyl acetate (2 mL), and dried to afford the title compound. Isolated yield: 267.5 mg (99%). MS-APCI (m/z+): 440, 484 (M+H).

### Example 190

### (S)-N-[3-(2-Methyl-5,6-dihydro-4H-3-thia-1-aza-benzo[e]azulen-8-yl)-2-oxo-oxazolidin-5-ylmethyl]-acetamide

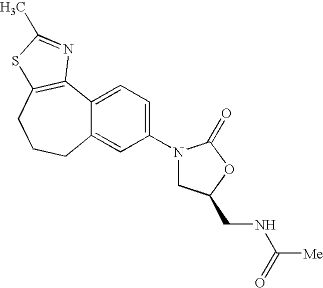

To N-[3-(6(R,S)-bromo-5-oxo-6,7,8,9-tetrahydro-5H-benzocyclohepten-2-yl)-2-oxo-oxazolidin-5(S)-ylmethyl]-acetamide (300.0 mg, 0.760 mmol) dissolved in warm ethanol (1.3 mL) was added thioacetamide (75.1 mg, 0.760 mmol). The resulting mixture was heated in microwave reactor at 150° C. for 1 hour. The solid was filtered out, washed with ethanol (2 mL) and ethyl acetate (2 mL) and dried to afford the title compound. Isolated yield: 80.6 mg (29%). MS-APCI (m/z+): 328, 372 (M+H).

### Example 191

### (S)-N-[2-Oxo-3-(2-pyridin-4-yl-5,6-dihydro-4H-3-thia-1-aza-benzo[e]azulen-8-yl)-oxazolidin-5-ylmethyl]-acetamide

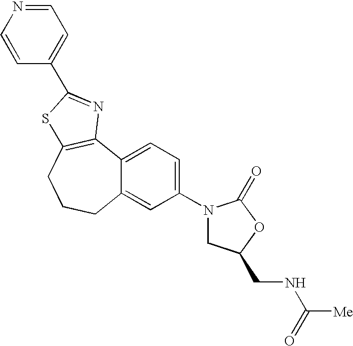

To N-[3-(6(R,S)-bromo-5-oxo-6,7,8,9-tetrahydro-5H-benzocyclohepten-2-yl)-2-oxo-oxazolidin-5(S)-ylmethyl]-acetamide (180.4 mg, 0.456 mmol) dissolved in warm ethanol (1.3 mL) was added thioisonicotinamide (138.2 mg, 0.456 mmol). The resulting mixture was heated in microwave reactor at 150° C. for 10 minutes. The solid was filtered away, washed with ethanol (2 mL) and ethyl acetate (2 mL) and dried to afford the title compound. Isolated yield: 72.3 mg (36%). MS-APCI (m/z+): 391, 435 (M+H).

### Example 192

### (S)-N-[3-(2-Morpholin-4-yl-5,6-dihydro-4H-3-thia-1-aza-benzo[e]azulen-8-yl)-2-oxo-oxazolidin-5-ylmethyl]-acetamide

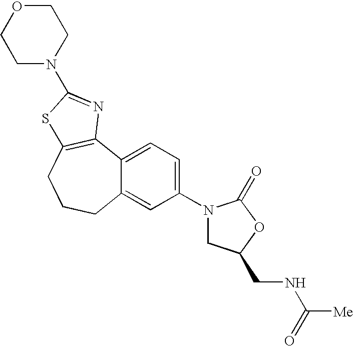

To N-[3-(6(R,S)-bromo-5-oxo-6,7,8,9-tetrahydro-5H-benzocyclohepten-2-yl)-2-oxo-oxazolidin-5(S)-ylmethyl]-acetamide (251.2 mg, 0.636 mmol) dissolved in warm ethanol (1 mL) was added morpholine-4-carbothioic acid amide (92.9 mg, 0.636 mmol). The resulting mixture was heated in microwave reactor at 120° C. for 14 minutes. The solid was filtered out, washed with ethyl acetate (2 mL) and dried to give the title compound. Isolated yield: 170.8 mg. MS-APCI (m/z+): 399, 443 (M+H).

### Example 193

### (S)-N-(3-{6-[4-(3-Dimethylamino-propoxy)-benzylidene]-5-oxo-6,7,8,9-tetrahydro-5H-benzocyclohepten-2-yl}-2-oxo-oxazolidin-5-ylmethyl)-acetamide

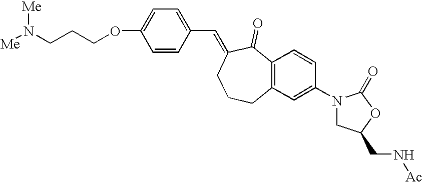

The title compound was prepared as described in the general procedure KK using (S)-N-[2-oxo-3-(5-oxo-6,7,8,9-tetrahydro-5H-benzocyclohepten-2-yl)-oxazolidin-5-ylmethyl]-acetamide (0.5 g), 4-(3-dimethylaminopropoxy) benzaldehyde (1.3 g), acetic acid (3 mL) and piperidine (3 mL). The crude product was purified by flash silica gel chromatography using dichloromethane/methanol as gradient system. Yield: 0.55 g. ¹H NMR (400 MHz, DMSOD-d₆): δ 1.78 (s, 3H), 1.83 (m, 2H), 2.0 (t, 2H), 2.13 (s, 6H), 2.30 (t, 2H), 2.48 (m, 2H), 2.83 (t, 2H), 3.26 (d, 2H), 3.35 (t, 2H), 3.74 (dd, 1H), 4.0 (t, 2H), 4.09 (t, 1H), 4.74 (m, 1H), 6.96 (d, 2H), 7.39-7.65 (m, 6H), 8.22 (t, 1H).

### Example 194

### (S)-N-[3-(6-Benzylidene-5-oxo-6,7,8,9-tetrahydro-5H-benzocyclohepten-2-yl)-2-oxo-oxazolidin-5-ylmethyl]-acetamide

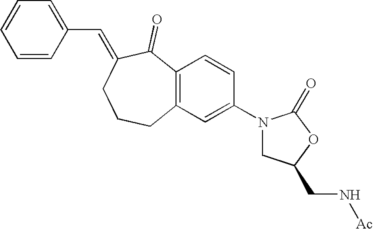

The title compound was prepared as described in the general procedure KK using (S)-N-[2-oxo-3-(5-oxo-6,7,8,9-tetrahydro-5H-benzocyclohepten-2-yl)-oxazolidin-5-ylmethyl]-acetamide (0.317 g), benzaldehyde (0.43 g), acetic acid (2 mL) and piperidine (2 mL). The crude product was purified by flash silica gel chromatography using dichloromethane/methanol as gradient system. Yield: 0.25 g. ¹H NMR (400 MHz, DMSO-d₆): δ 1.78 (s, 3H), 2.26 (t, 2H), 2.83 (t, 2H), 3.26 (t, 2H), 3.39 (dd, 1H), 4.13 (t, 1H), 4.74 (m, 1H), 7.30-7.70 (m, 9H), 8.2 (t, 1H).

### Example 195

### (S)-N-(3-{6-[4-(2-Hydroxy-ethoxy)-benzylidene]-5-oxo-6,7,8,9-tetrahydro-5H-benzocyclohepten-2-yl}-2-oxo-oxazolidin-5-ylmethyl)-acetamide

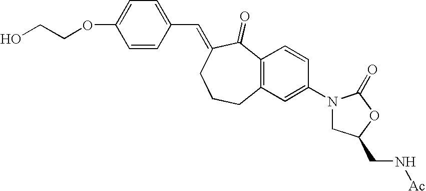

The title compound was prepared as described in the general procedure KK using (S)-N-[2-oxo-3-(5-oxo-6,7,8,9-tetrahydro-5H-benzocyclohepten-2-yl)-oxazolidin-5-ylmethyl]-acetamide (0.67 g), 4-(2-hydroxyethoxy)benzaldehyde (0.32 g), acetic acid (2 mL) and piperidine (2 mL). The crude product was purified by flash silica gel chromatography using dichloromethane/methanol as gradient system. Yield: 0.38 g. ¹H NMR (400 MHz, DMSO-d₆): δ 1.8 (s, 3H), 2.0 (t, 2H), 2.48 (m, 2H), 2.78 (t, 2H), 3.39 (m, 2H), 3.65 (m, 2H), 3.78 (dd, 1H), 4.0 (t, 2H), 4.13 (t, 1H), 4.70 (m, 1H), 4.87 (t, 1H), 6.98 (d, 2H), 7.39-7.65 (m, 6H), 8.17 (t, 1H).

### Example 196

### (S)-N-{3-[6-(3-Cyano-benzylidene)-5-oxo-6,7,8,9-tetrahydro-5H-benzocyclohepten-2-yl]-2-oxo-oxazolidin-5-ylmethyl}-acetamide

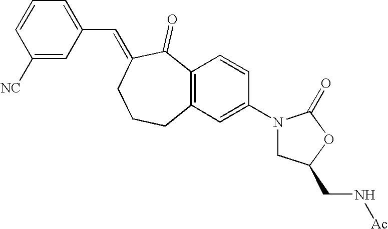

The title compound was prepared as described in the general procedure KK using (S)-N-[2-oxo-3-(5-oxo-6,7,8,9-tetrahydro-5H-benzocyclohepten-2-yl)-oxazolidin-5-ylmethyl]-acetamide (0.5 g), 3-cyanobenzaldehyde (0.83 g), acetic acid (2 mL) and piperidine (2 mL). The crude product was purified by flash silica gel chromatography using dichloromethane/methanol as gradient system. Yield: 0.5 g. APCI (m/z): 430 (M+H).

### Example 197

### (S)-N-{3-[3-(3-Cyano-phenyl)-1,4,5,6-tetrahydro-1,2-diaza-benzo[e]azulen-8-yl]-2-oxo-oxazolidin-5-ylmethyl}-acetamide

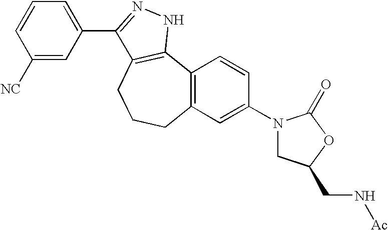

The title compound was prepared as described in the general procedure LL using (S)-N-{3-[6-(3-cyano-benzylidene)-5-oxo-6,7,8,9-tetrahydro-5H-benzocyclohepten-2-yl]-2-oxo-oxazolidin-5-ylmethyl}-acetamide (0.4 g), para-toluenesulfonyl hydrazide (0.38 g), and para-touene sulfonic acid (0.35 g). Yield: 0.2 g. MS-APCI (m/z): 398.

### Example 198

### (S)-N-(3-{6-[4-(2-Diethylamino-ethoxy)-benzylidene]-5-oxo-6,7,8,9-tetrahydro-5H-benzocyclohepten-2-yl}-2-oxo-oxazolidin-5-ylmethyl)-acetamide

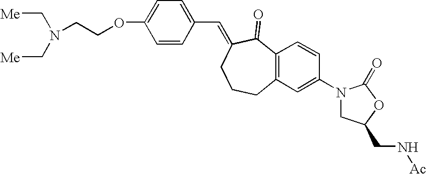

The title compound was prepared as described in the general procedure KK using (S)-N-[2-oxo-3-(5-oxo-6,7,8,9-tetrahydro-5H-benzocyclohepten-2-yl)-oxazolidin-5-ylmethyl]-acetamide (0.32 g), 4-(N,N-diethylamino)ethoxy benzaldehyde (0.9 g), acetic acid (2 mL) and piperidine (2 mL). The crude product was purified by flash silica gel chromatography using dichloromethane/methanol as gradient system. Yield: 0.22 g. MS-APCI (m/z): 520 (M+H).

### Example 199

### (S)-N-(3-{3-[4-(2-Diethylamino-ethoxy)-phenyl]-1,4,5,6-tetrahydro-1,2-diaza-benzo[e]azulen-8-yl}-2-oxo-oxazolidin-5-ylmethyl)-acetamide

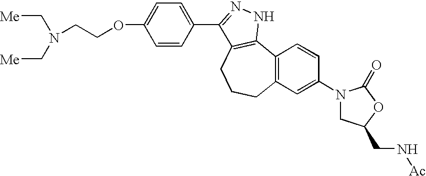

The title compound was prepared as described in the general procedure LL using (S)-N-{3-[6-(3-cyano-benzylidene)-5-oxo-6,7,8,9-tetrahydro-5H-benzocyclohepten-2-yl]-2-oxo-oxazolidin-5-ylmethyl}-acetamide (0.15 g), para-toluenesulfonyl hydrazide (0.12 g), and para-touene sulfonic acid (0.11 g). Yield: 0.089 g. APCI (m/z): 532 (M+H).

### Example 200

### (S)-N-[3-(6-Benzo[1,3]dioxol-5-ylmethylene-5-oxo-6,7,8,9-tetrahydro-5H-benzocyclohepten-2-yl)-2-oxo-oxazolidin-5-ylmethyl]-acetamide

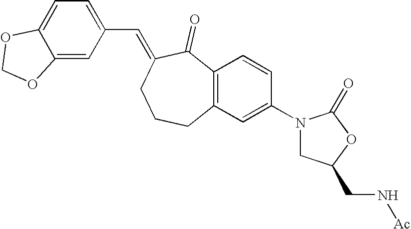

The title compound was prepared as described in the general procedure KK using (S)-N-[2-oxo-3-(5-oxo-6,7,8,9-tetrahydro-5H-benzocyclohepten-2-yl)-oxazolidin-5-ylmethyl]-acetamide (0.32 g), 4-(N,N-diethylamino)ethoxy benzaldehyde (0.6 g), acetic acid (2 mL) and piperidine (2 mL). The crude product was purified by flash silica gel chromatography using dichloromethane/methanol as gradient system. Yield: 0.26 g. APCI (m/z): 449 (M+H).

### Example 201

### (S)-N-[3-(3-Benzo[1,3]dioxol-5-yl-1,4,5,6-tetrahydro-1,2-diaza-benzo[e]azulen-8-yl)-2-oxo-oxazolidin-5-ylmethyl]-acetamide

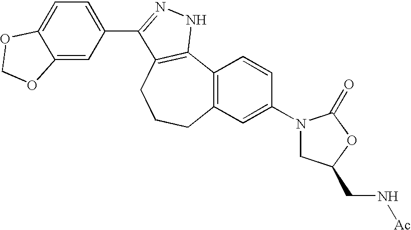

The title compound was prepared as described in the general procedure LL using (S)-N-[3-(6-benzo[1,3]dioxol-5-ylmethylene-5-oxo-6,7,8,9-tetrahydro-5H-benzocyclohepten-2-yl)-2-oxo-oxazolidin-5-ylmethyl]-acetamide (0.15 g), para-toluenesulfonyl hydrazide (0.12 g), and para-touene sulfonic acid (0.11 g). Yield: 0.089 g. APCI (m/z): 461 (M+H).

### Example 202

### (S)-N-[2-Oxo-3-(5-oxo-6-thiophen-3-ylmethylene-6,7,8,9-tetrahydro-5H-benzocyclohepten-2-yl)-oxazolidin-5-ylmethyl]-acetamide

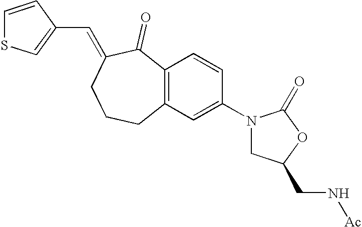

The title compound was prepared as described in the general procedure KK using (S)-N-[2-oxo-3-(5-oxo-6,7,8,9-tetrahydro-5H-benzocyclohepten-2-yl)-oxazolidin-5-ylmethyl]-acetamide (0.25 g), thiophene-3-carboxaldehyde (0.45 g), acetic acid (2 mL) and piperidine (2 mL). The crude product was purified by flash silica gel chromatography using dichloromethane/methanol as gradient system. Yield: 0.3 g. APCI (m/z): 411 (M+H).

### Example 203

### (S)-N-[2-Oxo-3-(3-thiophen-3-yl-1,4,5,6-tetrahydro-1,2-diaza-benzo[e]azulen-8-yl)-oxazolidin-5-ylmethyl]-acetamide

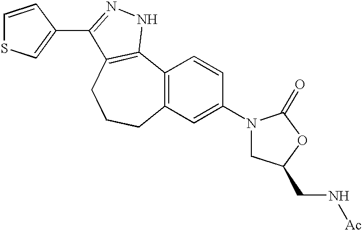

The title compound was prepared as described in the general procedure LL using (S)-N-[2-oxo-3-(5-oxo-6-thiophen-3-ylmethylene-6,7,8,9-tetrahydro-5H-benzocyclohepten-2-yl)-oxazolidin-5-ylmethyl]-acetamide (0.25 g), para-toluenesulfonyl hydrazide (0.25 g), and para-toluene sulfonic acid (0.2 g). Yield: 0.1 g. APCI (m/z): 423 (M+H).

### Example 204

### (S)-N-{3-[6-(4-Methanesulfonyl-benzylidene)-5-oxo-6,7,8,9-tetrahydro-5H-benzocyclohepten-2-yl]-2-oxo-oxazolidin-5-ylmethyl}-acetamide

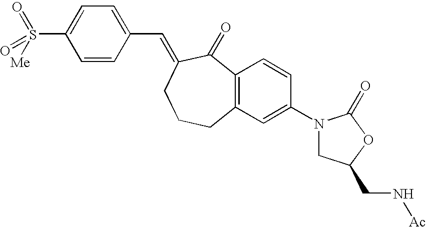

The title compound was prepared as described in the general procedure KK using (S)-N-[2-oxo-3-(5-oxo-6,7,8,9-tetrahydro-5H-benzocyclohepten-2-yl)-oxazolidin-5-ylmethyl]-acetamide (0.316 g), 4-methylsulfonylbenzaldehyde (0.79 g), acetic acid (2 mL) and piperidine (2 mL). The crude product was purified by flash silica gel chromatography using dichloromethane/methanol as gradient system. Yield: 0.4 g. APCI (m/z): 483 (M+H).

### Example 205

### (S)-N-{3-[6-(4-Fluoro-benzylidene)-5-oxo-6,7,8,9-tetrahydro-5H-benzocyclohepten-2-yl]-2-oxo-oxazolidin-5-ylmethyl}-acetamide

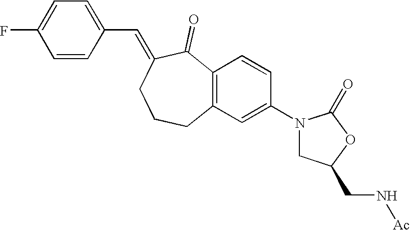

The title compound was prepared as described in the general procedure KK using (S)-N-[2-oxo-3-(5-oxo-6,7,8,9-tetrahydro-5H-benzocyclohepten-2-yl)-oxazolidin-5-ylmethyl]-acetamide (0.316 g), 4-fluorobenzaldehyde (0.5 g), acetic acid (2 mL) and piperidine (2 mL). The crude product was purified by flash silica gel chromatography using dichloromethane/methanol as gradient system. Yield: 0.22 g. APCI (m/z): 423 (M+H).

### Example 206

### (S)-N-{3-[3-(4-Fluoro-phenyl)-2-(2-hydroxy-ethyl)-2,4,5,6-tetrahydro-1,2-diaza-benzo[e]azulen-8-yl]-2-oxo-oxazolidin-5-ylmethyl}-acetamide

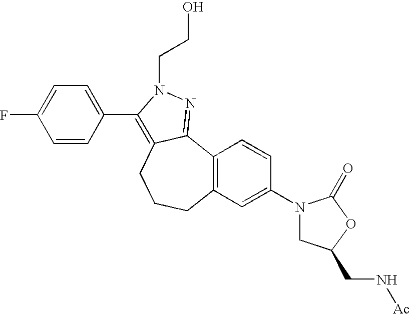

To the (S)-N-{3-[6-(4-fluoro-benzylidene)-5-oxo-6,7,8,9-tetrahydro-5H-benzocyclohepten-2-yl]-2-oxo-oxazolidin-5-ylmethyl}-acetamide (0.25 g) taken in 5 mL of acetic acid was added 2-hydroxyethylhydrazine. The reaction was kept at 80-100° C. for 3 hours. The crude product was purified by flash silica gel chromatography using dichloromethane/methanol as gradient system. Yield: 0.075 g. APCI (m/z): 481 (M+H).

### Example 207

### (S)-N-{3-[3-(4-Fluoro-phenyl)-2-(2-hydroxy-ethyl)-2,3,3a,4,5,6-hexahydro-1,2-diaza-benzo[e]azulen-8-yl]-2-oxo-oxazolidin-5-ylmethyl}-acetamide

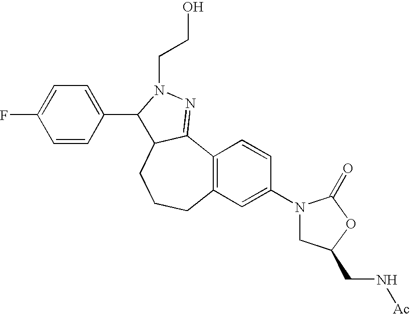

From the above reaction (Example 206) this product was also isolated. APCI (m/z): 483 (M+H).

### Example 208

### (S)-N-[2-Oxo-3-(5-oxo-6-pyridin-4-ylmethylene-6,7,8,9-tetrahydro-5H-benzocyclohepten-2-yl)-oxazolidin-5-ylmethyl]-acetamide

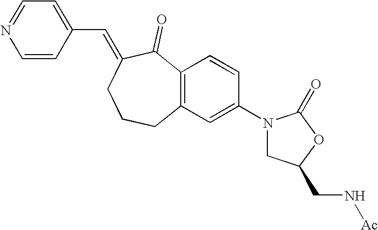

The title compound was prepared as described in the general procedure KK using (S)-N-[2-oxo-3-(5-oxo-6,7,8,9-tetrahydro-5H-benzocyclohepten-2-yl)-oxazolidin-5-ylmethyl]-acetamide (0.316 g), 4-pyridinecarboxaldehyde (0.48 g), acetic acid (2 mL) and piperidine (2 mL). The crude product was purified by flash silica gel chromatography using dichloromethane/methanol as gradient system. Yield: 0.3 g. APCI (m/z): 406 (M+H).

### Example 209

### (S)-N-{3-[2-(2-Hydroxy-ethyl)-3-pyridin-4-yl-2,4,5,6-tetrahydro-1,2-diaza-benzo[e]azulen-8-yl]-2-oxo-oxazolidin-5-ylmethyl}-acetamide

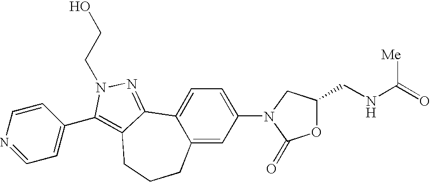

To the (S)-N-[2-oxo-3-(5-oxo-6-pyridin-4-ylmethylene-6,7,8,9-tetrahydro-5H-benzocyclohepten-2-yl)-oxazolidin-5-ylmethyl]-acetamide (0.30 g) taken in 5 mL of acetic acid was added 2-hydroxyethylhydrazine. The reaction was kept at 80-100° C. for 9 hours. The crude product was purified by flash silica gel chromatography using dichloromethane/methanol as gradient system. Yield: 0.075 g. APCI (m/z): 462 (M+H).

### Example 210

The following illustrates representative pharmaceutical dosage forms, containing a compound of Formula I (“Invention Compound”), for therapeutic or prophylactic use in humans.

| (i) Tablet | mg/tablet |
|---|---|
| ‘Invention Compound’ | 10-1000 |
| Lactose | 50.0 |
| Corn Starch (for mix) | 10.0 |
| Com Starch (paste) | 10.0 |
| Magnesium Stearate (1%) | 3.0 |
| | 300.0 |

The invention compound, lactose, and corn starch (for mix) are blended to uniformity. The corn starch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for treatment of pathogenic bacterial infections.

| (ii) Tablet | mg/capsule |
|---|---|
| ‘Invention Compound | 10-1000 |
| Colloidal Silicon Dioxide | 1.5 |
| Lactose | 465.5 |
| Pregelatinized Starch | 120.0 |
| Magnesium Stearate (1%) | 3.0 |
| | 600.0 |

| (iii) Preparation for | | |
|---|---|---|
| Oral Solution | Amount | |
| ‘Invention Compound’ | 10-1000 | |
| Sorbitol Solution (70% N.F.) | 40 | mL |
| Sodium Benzoate | 20 | mg |
| Saccharin | 5 | mg |
| Cherry Flavor | 20 | mg |
| Distilled Water q.s. | 100 | mL |

The sorbitol solution is added to 40 mL of distilled water, and the invention compound is dissolved therein. The saccharin, sodium benzoate, flavor, and dye are added and dissolved. The volume is adjusted to 100 mL with distilled water. Each milliliter of syrup contains 4 mg of invention compound.

(iv) Parenteral Solution

In a solution of 700 mL of propylene glycol and 200 mL of water for injection is suspended 20 g of an invention compound. After suspension is complete, the pH is adjusted to 6.5 with 1 N hydrochloric acid, and the volume is made up to 1000 mL with water for injection. The Formulation is sterilized, filled into 5.0 mL ampoules each containing 2.0 mL, and sealed under nitrogen.

| | Amount |
|---|---|
| (v) Injection 1 (1 mg/mL) | |
| ‘Invention Compound’ | 10-1000 |
| Dibasic Sodium Phosphate | 12.0 |
| Monobasic Sodium Phosphate | 0.7 |
| Sodium Chloride | 4.5 |
| 1.0 N Sodium hydroxide solution | q.s. |
| (pH adjustment to 7.0-7.5) | |
| Water for injection | q.s. ad 1 mL |
| (vi) Injection 2 (10 mg/mL) | |
| ‘Invention Compound’ | 10-1000 |
| Dibasic Sodium Phosphate | 1.1 |
| Monobasic Sodium Phosphate | 0.3 |
| Polyethylene glyco 400 | 200.0 |
| 0.1 N hydrochloric acid solution | q.s. |
| (pH adjustment to 7.0-7.5) | |
| Water for injection | q.s. ad 1 mL |
| (vii) Injection 2 (10 mg/mL) | |
| ‘Invention Compound’ | 10-1000 |
| Oleic Acid | 10.0 |
| Trichloromonofluoromethane | 5,000.0 |
| Dichlorodifluoromethane | 10,000.0 |
| Dichlorotetrafluoroethane | 5,000.0 |

All patents, and patent documents are incorporated by reference herein, as though individually incorporated by reference. The invention and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the spirit or scope of the present invention as set forth in the claims. To particularly point out and distinctly claim the subject matter regarded as invention, the following claims conclude this specification.

1. A compound of formula I: 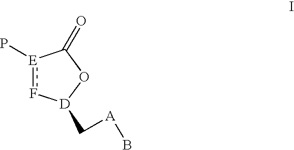
or a pharmaceutically acceptable salt thereof wherein:
A is O,
NH, or
S;
B is
C(═O)R₁,
C(═S)R₁,
heterocylco,
heteroaryl,
C(═O)-heterocyclo, or
C(═O)-heteteroaryl;
D is N when E is C and F is CH when “- - - - - -” is a bond, or D is CH when E is N and F is CH₂ when “- - - - - -” is absent;
P is 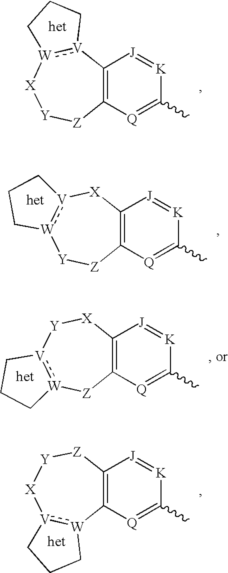
wherein [symbol] indicates the point of attachment; and 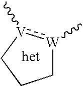
is 5-membered heterocycle or heteroaryl, wherein [symbol] indicates points of attachment, and wherein the 5-membered heterocyclo or heteroaryl is optionally substituted with one or more group selected from aryl, heteroaryl, heterocyclo, OR₅, OC(═O)R₁, NR₆R₇, NR₅, N(C═O)R₅, NH(C═O)OR₅, NHSO₂R₅, NHSO₂NR₅, aryl, heteroaryl, heterocyclo, wherein aryl or heteroaryl is optionally substituted with one or more halo, OH, CF₃, CN, NO₂, (C₁-C₈)alkyl, (C₃-C₆)cycloalkyl, S(C₁-C₄)alkyl, C(═O)R₁, OR₅, OC(═O)R₁, NR₆R₇, NHR₅, N(C═O)R₅, NH(C═O)OR₅, NHSO₂R₅, NHSO₂NR₅;
J, K, Q independently are CR₂ or N, with the proviso that when any one of J, K, or Q is N, then the other two are CR₂;
X, Y, Z independently are C═C—R₅, O═C,
CH₂,
CHR₃,
CHR₄,
CR₃R₄,
CH(OR₅), or
CHNR₆R₇;
R₁ is H,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl,
R₂ is H,
halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl;
R₃ and R₄ independently are halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂,
NH—(C₃-C₆)cycloalkyl;
aryl,
(CH₂)ₙ-aryl,
heterocyclo,
(CH₂)ₙ-heterocyclo,
heteroaryl, or
(CH₂)ₙ-heteroaryl;
wherein n is 0, 1, 2, or 3;
R₅ is H,
(C₁-C₈)alkyl,
(C₃-C₆)cycloalkyl,
aryl,
(CH₂)ₙ-aryl,
heterocyclo,
(CH₂)ₙ-heterocyclo,
heteroaryl, or
(CH₂)ₙ-heteroaryl;
wherein n is as defined above;
R₆ and R₇ independently are H,
(C₁-C₈)alkyl,
(C₃-C₆)cycloalkyl,
aryl,
(CH₂)ₙ-aryl,
heterocyclo,
(CH₂)ₙ-heterocyclo,
heteroaryl, or
(CH₂)ₙ-heteroaryl;
wherein n is 0, 1, 2, or 3; or R₆ and R₇ together can form a 5-7-membered ring containing 1, 2, or 3 heteroatoms which are N or S.

2. The compound of claim 1, wherein

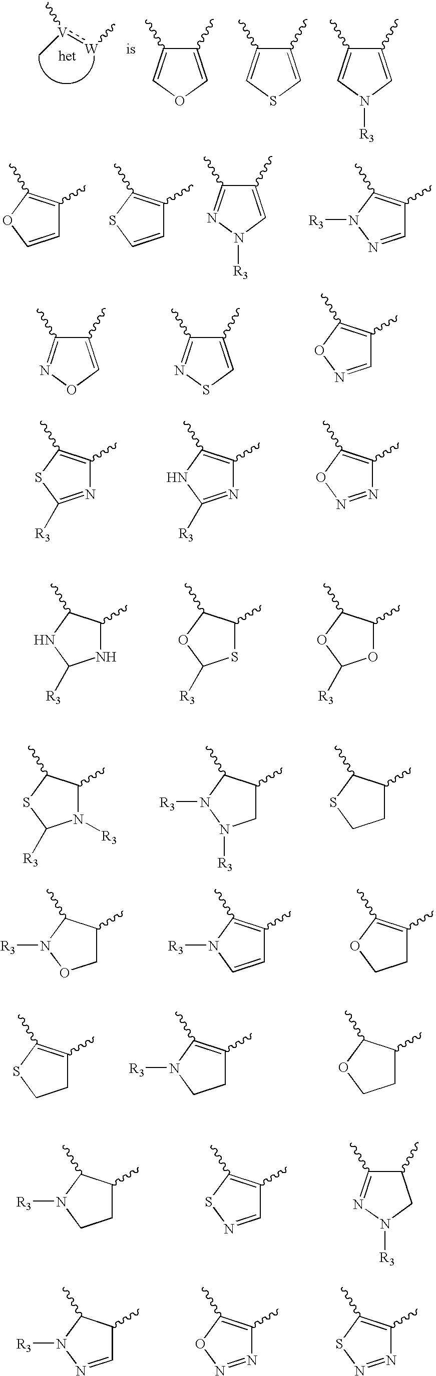

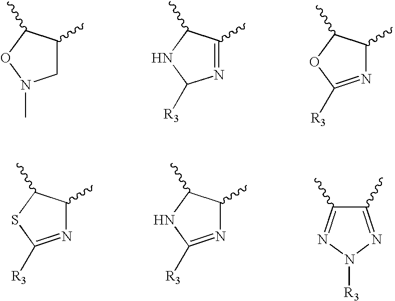

3. The compound of claim 1 as designated in formula IA.

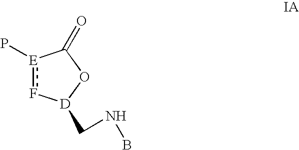

4. The compound of claim 1 as designated in formula IB.

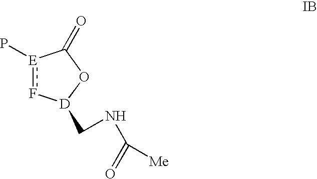

5. The compound of claim 1 as designated in formula IC.

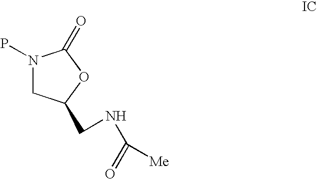

6. The compound of claim 5, wherein P is

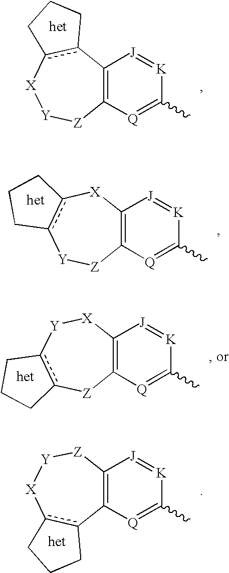

7. The compound of claim 6, wherein P is 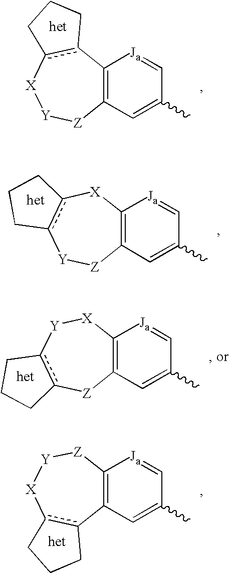
wherein Jₐ is N or CR₁₀, wherein R₁₀ is H or F, and wherein [symbol] indicates the point of attachment.

8. The compound of claim 7, wherein P is 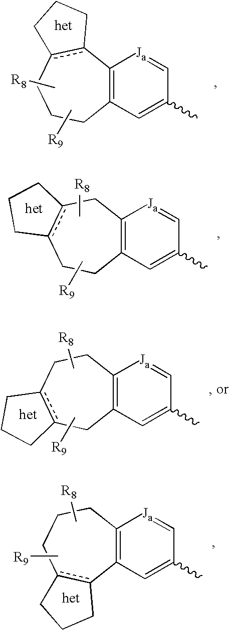
wherein [symbol] indicates the point of attachment; and wherein R₈ and R₉ are each independently H; halo, (C₁-C₈)alkyl, (C₃-C₆)cycloalkyl, O—(C₁-C₄) alkyl, S—(C₁-C₄) alkyl, aryl, (CH₂)ₙ-aryl, heterocyclo, (CH₂)ₙ-heterocyclo, heteroaryl, or (CH₂)ₙ-heteroaryl; wherein n is 0, 1, 2, or 3; or taken together R₈ and R₉ are bonded to the same C and form C═O.

9. A compound of formula II 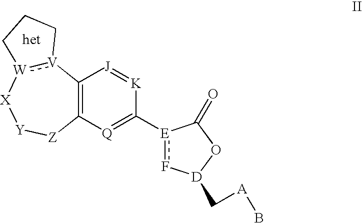
or a pharmaceutically acceptable salt thereof wherein
A is O,
NH, or
S;
B is
C(═O)R₁,
C(═S)R₁,
heterocylco,
heteroaryl,
C(═O)-heterocyclo, or
C(═O)-heteteroaryl;
D is N when E is C and F is CH when “- - - - - -” is a bond, or D is CH when E is N and F is CH₂ when “- - - - - -” is absent; 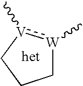
is 5-membered heterocyclo or heteroaryl, wherein [symbol] indicates points of attachment, and wherein the 5-membered heterocyclo or heteroaryl is optionally substituted with one or more group selected from aryl, heteroaryl, heterocyclo, OR₅, OC(═O)R₁, NR₆R₇, NR₅, N(C═O)R₅, NH(C═O)OR₅, NHSO₂R₅, NHSO₂NR₅, aryl, heteroaryl, heterocyclo, wherein aryl or heteroaryl is optionally substituted with one or more halo, OH, CF₃, CN, NO₂, (C₁-C₈)alkyl, (C₃-C₆)cycloalkyl, S(C₁-C₄)alkyl, C(═O)R₁, OR₅, OC(═O)R₁, NR₆R₇, NHR₅, N(C═O)R₅, NH(C═O)OR₅, NHSO₂R₅, NHSO₂NR₅;
J, K, Q independently are CR₂ or N, with the proviso that when any one of J, K, or Q is N, then the other two are CR₂;
X, Y, Z independently are C═C—R₅, O═C,
CH₂,
CHR₃,
CHR₄,
CR₃R₄,
CH(OR₅), or
CHNR₆R₇;
R₁ is H,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl,
R₂ is H,
halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl;
R₃ and R₄ independently are H, halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂,
NH—(C₃-C₆)cycloalkyl;
aryl,
(CH₂)ₙ-aryl,
heterocyclo,
(CH₂)ₙ-heterocyclo,
heteroaryl, or
(CH₂)ₙ-heteroaryl;
wherein n is 0, 1, 2, or 3;
R₅ is H,
(C₁-C₈)alkyl,
(C₃-C₆)cycloalkyl,
aryl,
(CH₂)ₙ-aryl,
heterocyclo,
(CH₂)ₙ-heterocyclo,
heteroaryl, or
(CH₂)ₙ-heteroaryl;
wherein n is 0, 1, 2, or 3;
R₆ and R₇ independently are H;
(C₁-C₈)alkyl,
(C₃-C₆)cycloalkyl,
aryl,
(CH₂)ₙ-aryl,
heterocyclo,
(CH₂)ᵣ-heterocyclo,
heteroaryl, or
(CH₂)ₙ-heteroaryl;
wherein n is 0, 1, 2, or 3; or R₆ and R₇ together can form a 5-7-membered ring containing 1, 2, or 3 heteroatoms which are N or S.

10. The compound of claim 9, wherein 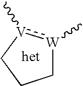
is as defined in claim 2.

11. The compound of claim 9 as designated in formula IIA.

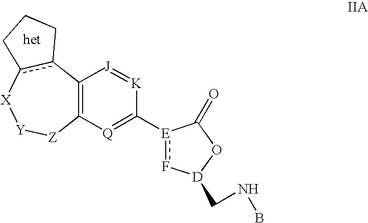

12. The compound of claim 9 as designated in formula IIB.

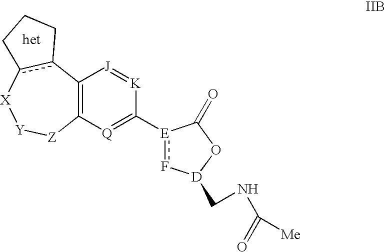

13. The compound of claim 9 as designated in formula IIC.

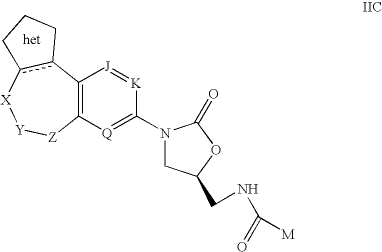

14. The compound of claim 9 as designated in formula IID 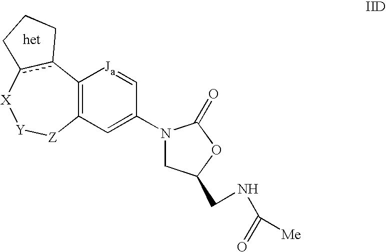
wherein Jₐ is N or CR₁₀, wherein R₁₀ is H or F.

15. The compound of claim 9 as designated in formula IIE 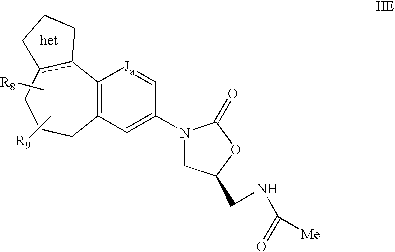
wherein R₈ and R₉ are each independently H; halo, (C₁-C₈)alkyl, (C₃-C₆)cycloalkyl, O—(C₁-C₄) alkyl, S—(C₁-C₄) alkyl, aryl, (CH₂)ₙ-aryl, heterocyclo, (CH₂)ₙ-heterocyclo, heteroaryl, or (CH₂)ₙ-heteroaryl, wherein n is 0, 1, 2, or 3; or taken together R₈ and R₉ are bonded to the same C and form C═O.

16. A compound of formula III 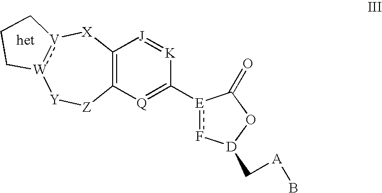
or a pharmaceutically acceptable salt thereof wherein:
A is O,
NH, or
S;
B is
C(═O)R₁,
C(═S)R₁,
heterocylco,
heteroaryl,
C(═O)-heterocyclo, or
C(═O)-heteteroaryl;
D is N when E is C and F is CH when “- - - - - -” is a bond, or D is CH when E is N and F is CH₂ when “- - - - - -” is absent; 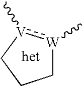
is 5-membered heterocyclo or heteroaryl, wherein [symbol] indicates points of attachment, and wherein the 5-membered heterocyclo or heteroaryl is optionally substituted with one or more group selected from aryl, heteroaryl, heterocyclo, OR₅, OC(═O)R₁, NR₆R₇, NR₅, N(C═O)R₅, NH(C═O)OR₅, NHSO₂R₅, NHSO₂NR₅, aryl, heteroaryl, heterocyclo, wherein aryl or heteroaryl is optionally substituted with one or more halo, OH, CF₃, CN, NO₂, (C₁-C₈)alkyl, (C₃-C₆)cycloalkyl, S(C₁-C₄)alkyl, C(═O)R₁, OR₅, OC(═O)R₁, NR₆R₇, NHR₅, N(C═O)R₅, NH(C═O)OR₅, NHSO₂R₅, NHSO₂NR₅;
J, K, Q independently are CR₂ or N, with the proviso that when any one of J, K, or Q is N, then the other two are CR₂;
X, Y, Z independently are C═C—R₅, O═C,
CH₂,
CHR₃,
CHR₄,
CR₃R₄,
CH(OR₅), or
CHNR₆R₇;
R₁ is H,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl,
R₂ is H,
halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl;
R₃ and R₄ independently are halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂,
NH—(C₃-C₆)cycloalkyl;
aryl,
(CH₂)ₙ-aryl,
heterocyclo,
(CH₂)ₙ-heterocyclo,
heteroaryl, or
(CH₂)ₙ-heteroaryl;
wherein n is 0, 1, 2, or 3;
R₅ is H,
(C₁-C₈)alkyl,
(C₃-C₆)cycloalkyl,
aryl,
(CH₂)ₙ-aryl,
heterocyclo,
(CH₂)ₙ-heterocyclo,
heteroaryl, or
(CH₂)ₙ-heteroaryl;
wherein n is 0, 1, 2, or 3;
R₆ and R₇ independently are H,
(C₁-C₈)alkyl,
(C₃-C₆)cycloalkyl,
aryl,
(CH₂)ₙ-aryl,
heterocyclo,
(CH₂)ₙ-heterocyclo,
heteroaryl, or
(CH₂)ₙ-heteroaryl;
wherein n is 0, 1, 2, or 3; or R₆ and R₇ together can form a 5-7-membered ring containing 1, 2, or 3 heteroatoms which are N or S.

17. The compound of claim 16, wherein 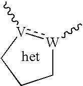
is as defined in claim 2.

18. The compound of claim 16 as designated in formula IIIA.

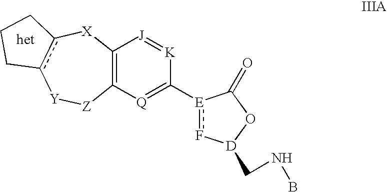

19. The compound of claim 16 as designated in formula IIIB.

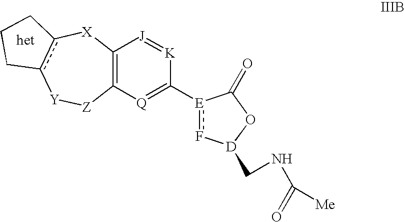

20. The compound of claim 16 as designated in formula IIIC.

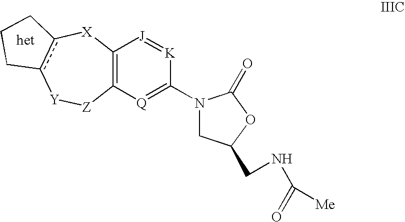

21. The compound of claim 16 as designated in formula IIID 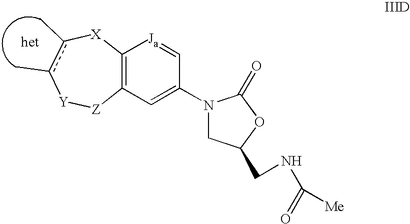
wherein Jₐ is N or CR₁₀, wherein R₁₀ is H or F.

22. The compound of claim 16 as designated in formula IIIE 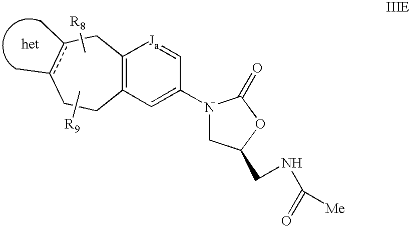
wherein R₈ and R₉ are each independently H; halo, (C₁-C₈)alkyl, (C₃-C₆)cycloalkyl, O—(C₁-C₄) alkyl, S—(C₁-C₄) alkyl, aryl, (CH₂)ₙ-aryl, heterocyclo, (CH₂)ₙ-heterocyclo, heteroaryl, or (CH₂)ₙ-heteroaryl, wherein n is 0, 1, 2, or 3; or taken together R₈ and R₉ are bonded to the same C and form C═O.

23. A compound of formula IV: 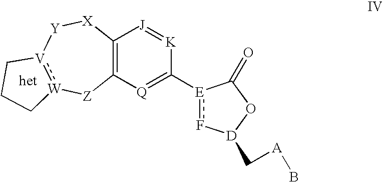
or a pharmaceutically acceptable salt thereof wherein:
A is O,
NH, or
S;
B is
C(═O)R₁,
C(═S)R₁,
heterocylco,
heteroaryl,
C(═O)-heterocyclo, or
C(═O)-heteteroaryl;
D is N when E is C and F is CH when “- - - - - -” is a bond, or D is CH when E is N and F is CH₂ when “- - - - - -” is absent; 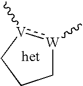
is 5-membered heterocyclo or heteroaryl, wherein [symbol] indicates points of attachment, and wherein the 5-membered heterocyclo or heteroaryl is optionally substituted with one or more group selected from aryl, heteroaryl, heterocyclo, OR₅, OC(═O)R₁, NR₆R₇, NR₅, N(C═O)R₅, NH(C═O)OR₅, NHSO₂R₅, NHSO₂NR₅, aryl, heteroaryl, heterocyclo, wherein aryl or heteroaryl is optionally substituted with one or more halo, OH, CF₃, CN, NO₂, (C₁-C₈)alkyl, (C₃-C₆)cycloalkyl, S(C₁-C₄)alkyl, C(═O)R₁, OR₅, OC(═O)R₁, NR₆R₇, NHR₅, N(C═O)R₅, NH(C═O)OR₅, NHSO₂R₅, NHSO₂NR₅;
J, K, Q independently are CR₂ or N, with the proviso that when any one of J, K, or Q is N, then the other two are CR₂;
X, Y, Z independently are C═C—R₅, O═C,
CH₂,
CHR₃,
CHR₄,
CR₃R₄,
CH(OR₅), or
CHNR₆R₇;
R₁ is H,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl,
R₂ is H,
halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl;
R₃ and R₄ independently are halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂,
NH—(C₃-C₆)cycloalkyl;
aryl,
(CH₂)ₙ-aryl,
heterocyclo,
(CH₂)ₙ-heterocyclo,
heteroaryl, or
(CH₂)ₙ-heteroaryl;
wherein n is 0, 1, 2, or 3;
R₅ is H,
(C₁-C₈)alkyl,
(C₃-C₆)cycloalkyl,
aryl,
(CH₂)ᵣ-aryl,
heterocyclo,
(CH₂)ₙ-heterocyclo,
heteroaryl, or
(CH₂)ᵣ-heteroaryl;
wherein n is 0, 1, 2, or 3;
R₆ and R₇ independently are H,
(C₁-C₈)alkyl,
(C₃-C₆)cycloalkyl,
aryl,
(CH₂)ₙ-aryl,
heterocyclo,
(CH₂)ₙ-heterocyclo,
heteroaryl, or
(CH₂)ₙ-heteroaryl;
wherein n is 0, 1, 2, or 3; or
R₆ and R₇ together can form a 5-7-membered ring containing 1, 2, or 3 heteroatoms which are N or S.

24. The compound of claim 23, wherein 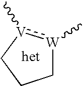
is as defined in claim 2.

25. The compound of claim 23 as designated in formula IVA.

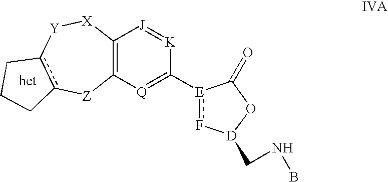

26. The compound of claim 23 as designated in formula IVB.

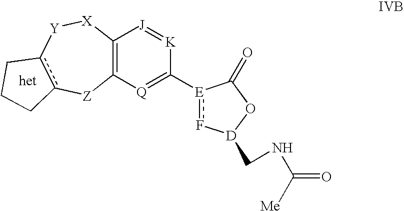

27. The compound of claim 23 as designated in formula IVC.

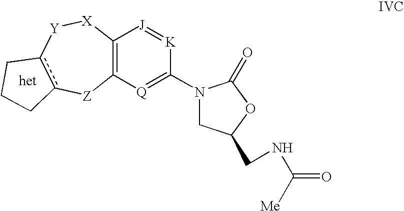

28. The compound of claim 23 as designated in formula IVD 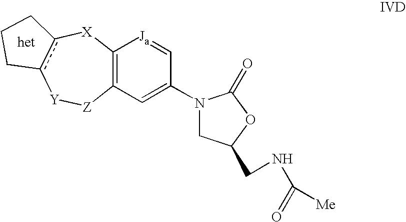
wherein Jₐ is N or CR₁₀, wherein R₁₀ is H or F. Specific calues for K and Q are CH, and CH, respectively.

29. The compound of claim 23 as designated in formula IVE 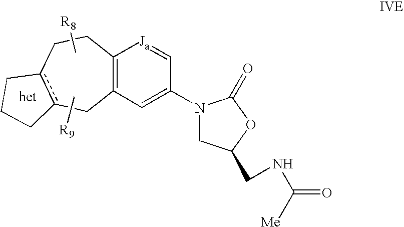
wherein R₈ and R₉ are each independently H; halo, (C₁-C₈)alkyl, (C₃-C₆)cycloalkyl, O—(C₁-C₄) alkyl, S—(C₁-C₄) alkyl, aryl, (CH₂)ₙ-aryl, heterocyclo, (CH₂)ₙ-heterocyclo, heteroaryl, or (CH₂)ₙ-heteroaryl, wherein n is 0, 1, 2, or 3; or taken together R₈ and R₉ are bonded to the same C and form C═O.

30. A compound of formula V: 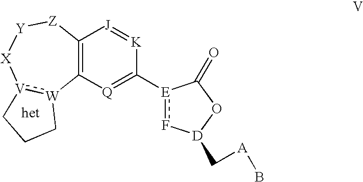
or a pharmaceutically acceptable salt thereof wherein:
A is O,
NH, or
S;
B is
C(═O)R₁,
C(═S)R₁,
heterocylco,
heteroaryl,
C(═O)-heterocyclo, or
C(═O)-heteteroaryl;
D is N when E is C and F is CH when “- - - - - -” is a bond, or D is CH when E is N and F is CH₂ when “- - - - - -” is absent; 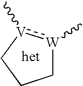
is 5-membered heterocyclo or heteroaryl, wherein [symbol] indicates points of attachment, and wherein the 5-membered heterocyclo or heteroaryl is optionally substituted with one or more group selected from aryl, heteroaryl, heterocyclo, OR₅, OC(═O)R₁, NR₆R₇, NR₅, N(C═O)R₅, NH(C═O)OR₅, NHSO₂R₅, NHSO₂NR₅, aryl, heteroaryl, heterocyclo, wherein aryl or heteroaryl is optionally substituted with one or more halo, OH, CF₃, CN, NO₂, (C₁-C₈)alkyl, (C₃-C₆)cycloalkyl, S(C₁-C₄)alkyl, C(═O)R₁, OR₅, OC(═O)R₁, NR₆R₇, NHR₅, N(C═O)R₅, NH(C═O)OR₅, NHSO₂R₅, NHSO₂NR₅;
J, K, Q independently are CR₂ or N, with the proviso that when any one of J, K, or Q is N, then the other two are CR₂;
X, Y, Z independently are C═C—R₅, O═C,
CH₂,
CHR₃,
CHR₄,
CR₃R₄,
CH(OR₅), or
CHNR₆R₇;
R₁ is H,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl,
R₂ is H,
halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂, or
NH—(C₃-C₆)cycloalkyl;
R₃ and R₄ independently are halo,
(C₁-C₈)alkyl,
(C₃-C₆)cycloalkyl,
O—(C₁-C₄)alkyl,
O—(C₃-C₆)cycloalkyl,
S—(C₁-C₄) alkyl,
S—(C₃-C₆)cycloalkyl,
NH₂,
NH(C₁-C₄)alkyl,
N((C₁-C₄)alkyl)₂,
NH—(C₃-C₆)cycloalkyl;
aryl,
(CH₂)ₙ-aryl,
heterocyclo,
(CH₂)ₙ-heterocyclo,
heteroaryl, or
(CH₂)ₙ-heteroaryl;
wherein n is 0, 1, 2, or 3;
R₅ is H,
(C₁-C₈)alkyl,
(C₃-C₆)cycloalkyl,
aryl,
(CH₂)ₙ-aryl,
heterocyclo,
(CH₂)ₙ-heterocyclo,
heteroaryl, or
(CH₂)ₙ-heteroaryl;
wherein n is 0, 1, 2, or 3;
R₆ and R₇ independently are H,
(C₁-C₈)alkyl,
(C₃-C₆)cycloalkyl,
aryl,
(CH₂)ₙ-aryl,
heterocyclo,
(CH₂)ₙ-heterocyclo,
heteroaryl, or
(CH₂)ₙ-heteroaryl;
wherein n is 0, 1, 2, or 3; or
R₆ and R₇ together can form a 5-7-membered ring containing 1, 2, or 3 heteroatoms which are N or S.

31. The compound of claim 30, wherein 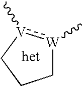
is as defined in claim 2.

32. The compound of claim 30 as designated in formula VA.

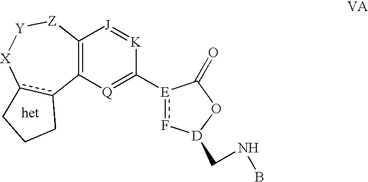

33. The compound of claim 30 as designated in formula VB.

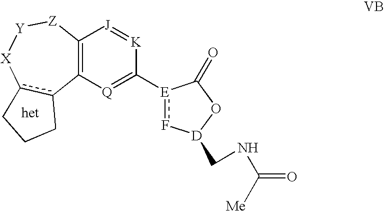

34. The compound of claim 30 as designated in formula VC.

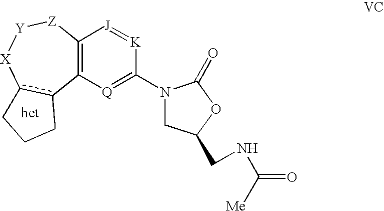

35. The compound of claim 30 as designated in formula VD 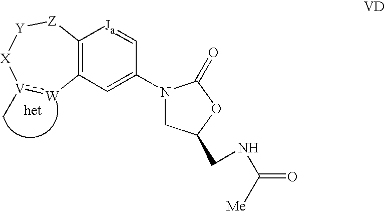
wherein Jₐ is N or CR₁₀, wherein R₁₀ is H or F.

36. The compound of claim 30 as designated in formula VE 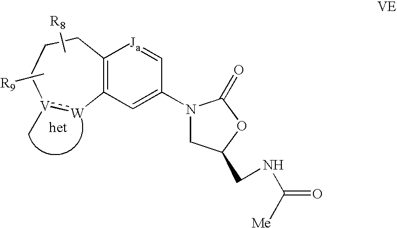
wherein R₈ and R₉ are each independently H; halo, (C₁-C₈)alkyl, (C₃-C₆)cycloalkyl, O—(C₁-C₄) alkyl, S—(C₁-C₄) alkyl, aryl, (CH₂)ₙ-aryl, heterocyclo, (CH₂)ₙ-heterocyclo, heteroaryl, or (CH₂)ₙ-heteroaryl, wherein n is 0, 1, 2, or 3; or taken together R₈ and R₉ are bonded to the same C and form C═O.

37. A compound which is:
(S)-N-[2-Oxo-3-(1,4,5,6-tetrahydro-1,2-diaza-benzo[e]azulen-8-yl)-oxazolidin-5-ylmethyl]-acetamide;
(S)-N-[2-Oxo-3-(3-phenyl-1,4,5,6-tetrahydro-1,2-diaza-benzo[e]azulen-8-yl)-oxazolidin-5-ylmethyl]-acetamide;
(S)-N-{3-[3-(2-Hydroxy-phenyl)-1,4,5,6-tetrahydro-1,2-diaza-benzo[e]azulen-8-yl]-2-oxo-oxazolidin-5-ylmethyl}-acetamide;
(S)-N-{3-[3-(2-Methoxy-phenyl)-1,4,5,6-tetrahydro-1,2-diaza-benzo[e]azulen-8-yl]-2-oxo-oxazolidin-5-ylmethyl}-acetamide;
(S)-N-{2-Oxo-3-[3-(2-trifluoromethoxy-phenyl)-1,4,5,6-tetrahydro-1,2-diaza-benzo[e]azulen-8-yl]-oxazolidin-5-ylmethyl}-acetamide;
(S)-N-{2-Oxo-3-[3-(2-trifluoromethyl-phenyl)-1,4,5,6-tetrahydro-1,2-diaza-benzo[e]azulen-8-yl]-oxazolidin-5-ylmethyl}-acetamide;
(S)-N-{3-[3-(2-Fluoro-phenyl)-1,4,5,6-tetrahydro-1,2-diaza-benzo[e]azulen-8-yl]-2-oxo-oxazolidin-5-ylmethyl}-acetamide;
(S)-N-{3-[3-(3-Hydroxy-phenyl)-1,4,5,6-tetrahydro-1,2-diaza-benzo[e]azulen-8-yl]-2-oxo-oxazolidin-5-ylmethyl}-acetamide;
(S)-N-{3-[3-(3-Methroxy-phenyl)-1,4,5,6-tetrahydro-1,2-diaza-benzo[e]azulen-8-yl]-2-oxo-oxazolidin-5-ylmethyl}-acetamide;
(S)-N-{2-Oxo-3-[3-(3-trifluoromethoxy-phenyl)-1,4,5,6-tetrahydro-1,2-diaza-benzo[e]azulen-8-yl]-oxazolidin-5-ylmethyl}-acetamide;
(S)-N-{2-Oxo-3-[3-(3-trifluormethyl-phenyl)-1,4,5,6-tetrahydro-1,2-diaza-benzo[e]azulen-8-yl]-oxazolidin-5-ylmethyl}-acetamide;
(S)-N-{3-[3-(3-Fluoro-phenyl)-1,4,5,6-tetrahydro-1,2-diaza-benzo[e]azulen-8-yl]-2-oxo-oxazolidin-5-ylmethyl}-acetamide;
(S)-N-{3-[3-(4-Hydroxy-phenyl)-1,4,5,6-tetrahydro-1,2-diaza-benzo[e]azulen-8-yl]-2-oxo-oxazolidin-5-ylmethyl}-acetamide;
(S)-N-{3-[3-(4-Methoxy-phenyl)-1,4,5,6-tetrahydro-1,2-diaza-benzo[e]azulen-8-yl]-2-oxo-oxazolidin-5-ylmethyl}-acetamide;
(S)-N-{2-Oxo-3-[3-(4-trifluoromethoxy-phenyl)-1,4,5,6-tetrahydro-1,2-diaza-benzo[e]azulen-8-yl]-oxazolidin-5-ylmethyl}-acetamide;
(S)-N-{2-Oxo-3-[3-(4-trifluoromethyl-phenyl)-1,4,5,6-tetrahydro-1,2-diaza-benzo[e]azulen-8-yl]-oxazolidin-5-ylmethyl}-acetamide;
(S)-N-{3-[3-(4-Fluoro-phenyl)-1,4,5,6-tetrahydro-1,2-diaza-benzo[e]azulen-8-yl]-2-oxo-oxazolidin-5-ylmethyl}-acetamide;
(S)-N-[2-Oxo-3-(3-thiophen-3-yl-1,4,5,6-tetrahydro-1,2-diaza-benzo[e]azulen-8-yl)-oxazolidin-5-ylmethyl]-acetamide;
(S)-N-{3-[3-(4-Hydroxy-thiophen-3-yl)-1,4,5,6-tetrahydro-1,2-diaza-benzo[e]azulen-8-yl]-2-oxo-oxazolidin-5-ylmethyl}-acetamide;
(S)-N-{3-[3-(4-Methoxy-thiophen-3-yl)-1,4,5,6-tetrahydro-1,2-diaza-benzo[e]azulen-8-yl]-2-oxo-oxazolidin-5-ylmethyl}-acetamide;
(S)-N-{2-Oxo-3-[3-(4-trifluoromethoxy-thiophen-3-yl)-1,4,5,6-tetrahydro-1,2-diaza-benzo[e]azulen-8-yl]-oxazolidin-5-ylmethyl}-acetamide;
(S)-N-{2-Oxo-3-[3-(4-trifluoromethyl-thiophen-3-yl)-1,4,5,6-tetrahydro-1,2-diaza-benzo[e]azulen-8-yl]-oxazolidin-5-ylmethyl}-acetamide;
(S)-N-{3-[3-(4-Fluoro-thiophen-3-yl)-1,4,5,6-tetrahydro-1,2-diaza-benzo[e]azulen-8-yl]-2-oxo-oxazolidin-5-ylmethyl}-acetamide;
(S)-N-{3-[3-(5-Hydroxy-thiophen-3-yl)-1,4,5,6-tetrahydro-1,2-diaza-benzo[e]azulen-8-yl]-2-oxo-oxazolidin-5-ylmethyl}-acetamide;
(S)-N-{3-[3-(5-Methoxy-thiophen-3-yl)-1,4,5,6-tetrahydro-1,2-diaza-benzo[e]azulen-8-yl]-2-oxo-oxazolidin-5-ylmethyl}-acetamide;
(S)-N-{2-Oxo-3-[3-(5-trifluoromethoxy-thiophen-3-yl)-1,4,5,6-tetrahydro-1,2-diaza-benzo[e]azulen-8-yl]-oxazolidin-5-ylmethyl}-acetamide;
(S)-N-{2-Oxo-3-[3-(5-trifluoromethyl-thiophen-3-yl)-1,4,5,6-tetrahydro-1,2-diaza-benzo[e]azulen-8-yl]-oxazolidin-5-ylmethyl}-acetamide;
(S)-N-{3-[3-(5-Fluoro-thiophen-3-yl)-1,4,5,6-tetrahydro-1,2-diaza-benzo[e]azulen-8-yl]-2-oxo-oxazolidin-5-ylmethyl}-acetamide;
(S)-N-[3-(3-Furan-3-yl-1,4,5,6-tetrahydro-1,2-diaza-benzo[e]azulen-8-yl)-2-oxo-oxazolidin-5-ylmethyl]-acetamide;
(S)-N-{3-[3-(4-Hydroxy-furan-3-yl)-1,4,5,6-tetrahydro-1,2-diaza-benzo[e]azulen-8-yl]-2-oxo-oxazolidin-5-ylmethyl}-acetamide;
(S)-N-{3-[3-(4-Methoxy-furan-3-yl)-1,4,5,6-tetrahydro-1,2-diaza-benzo[e]azulen-8-yl]-2-oxo-oxazolidin-5-ylmethyl}-acetamide;
(S)-N-{2-Oxo-3-[3-(4-trifluoromethoxy-furan-3-yl)-1,4,5,6-tetrahydro-1,2-diaza-benzo[e]azulen-8-yl]-oxazolidin-5-ylmethyl}-acetamide;
(S)-N-{2-Oxo-3-[3-(4-trifluoromethyl-furan-3-yl)-1,4,5,6-tetrahydro-1,2-diaza-benzo[e]azulen-8-yl]-oxazolidin-5-ylmethyl}-acetamide;
(S)-N-{3-[3-(4-Fluoro-furan-3-yl)-1,4,5,6-tetrahydro-1,2-diaza-benzo[e]azulen-8-yl]-2-oxo-oxazolidin-5-ylmethyl}-acetamide;
(S)-N-{3-[3-(5-Hydroxy-furan-3-yl)-1,4,5,6-tetrahydro-1,2-diaza-benzo[e]azulen-8-yl]-2-oxo-oxazolidin-5-ylmethyl}-acetamide;
(S)-N-{3-[3-(5-Methoxy-furan-3-yl)-1,4,5,6-tetrahydro-1,2-diaza-benzo[e]azulen-8-yl]-2-oxo-oxazolidin-5-ylmethyl}-acetamide;
(S)-N-{2-Oxo-3-[3-(5-trifluoromethoxy-furan-3-yl)-1,4,5,6-tetrahydro-1,2-diaza-benzo[e]azulen-8-yl]-oxazolidin-5-ylmethyl}-acetamide;
(S)-N-{2-Oxo-3-[3-(5-trifluoromethyl-furan-3-yl)-1,4,5,6-tetrahydro-1,2-diaza-benzo[e]azulen-8-yl]-oxazolidin-5-ylmethyl}-acetamide;
(S)-N-{3-[3-(5-Fluoro-furan-3-yl)-1,4,5,6-tetrahydro-1,2-diaza-benzo[e]azulen-8-yl]-2-oxo-oxazolidin-5-ylmethyl}-acetamide;
(S)-N-[2-Oxo-3-(3-pyridin-4-yl-1,4,5,6-tetrahydro-1,2-diaza-benzo[e]azulen-8-yl)-oxazolidin-5-ylmethyl]-acetamide;
(S)-N-{3-[3-(3-Hydroxy-pyridin-4-yl)-1,4,5,6-tetrahydro-1,2-diaza-benzo[e]azulen-8-yl]-2-oxo-oxazolidin-5-ylmethyl}-acetamide;
(S)-N-{3-[3-(3-Methoxy-pyridin-4-yl)-1,4,5,6-tetrahydro-1,2-diaza-benzo[e]azulen-8-yl]-2-oxo-oxazolidin-5-ylmethyl}-acetamide;
(S)-N-{2-Oxo-3-[3-(3-trifluoromethoxy-pyridin-4-yl)-1,4,5,6-tetrahydro-1,2-diaza-benzo[e]azulen-8-yl]-oxazolidin-5-ylmethyl}-acetamide;
(S)-N-{2-Oxo-3-[3-(3-trifluoromethyl-pyridin-4-yl)-1,4,5,6-tetrahydro-1,2-diaza-benzo[e]azulen-8-yl]-oxazolidin-5-ylmethyl}-acetamide;
(S)-N-{3-[3-(3-Fluoro-pyridin-4-yl)-1,4,5,6-tetrahydro-1,2-diaza-benzo[e]azulen-8-yl]-2-oxo-oxazolidin-5-ylmethyl}-acetamide;
(S)-N-{3-[3-(2-Hydroxy-pyridin-4-yl)-1,4,5,6-tetrahydro-1,2-diaza-benzo[e]azulen-8-yl]-2-oxo-oxazolidin-5-ylmethyl}-acetamide;
(S)-N-{3-[3-(2-Methoxy-pyridin-4-yl)-1,4,5,6-tetrahydro-1,2-diaza-benzo[e]azulen-8-yl]-2-oxo-oxazolidin-5-ylmethyl}-acetamide;
(S)-N-{2-Oxo-3-[3-(2-trifluoromethoxy-pyridin-4-yl)-1,4,5,6-tetrahydro-1,2-diaza-benzo[e]azulen-8-yl]-oxazolidin-5-ylmethyl}-acetamide;
(S)-N-{2-Oxo-3-[3-(2-trifluoromethyl-pyridin-4-yl)-1,4,5,6-tetrahydro-1,2-diaza-benzo[e]azulen-8-yl]-oxazolidin-5-ylmethyl}-acetamide;
(S)-N-{3-[3-(2-Fluoro-pyridin-4-yl)-1,4,5,6-tetrahydro-1,2-diaza-benzo[e]azulen-8-yl]-2-oxo-oxazolidin-5-ylmethyl}-acetamide;
(S)-N-[3-(5,6-Dihydro-4H-1-oxa-2-aza-benzo[e]azulen-8-yl)-2-oxo-oxazolidin-5-ylmethyl]-acetamide;
(S)-N-[2-Oxo-3-(3-phenyl-5,6-dihydro-4H-1-oxa-2-aza-benzo[e]azulen-8-yl)-oxazolidin-5-ylmethyl]-acetamide;
(S)-N-{3-[3-(2-Hydroxy-phenyl)-5,6-dihydro-4H-1-oxa-2-aza-benzo[e]azulen-8-yl]-2-oxo-oxazolidin-5-ylmethyl}-acetamide;
(S)-N-{3-[3-(2-Methoxy-phenyl)-5,6-dihydro-4H-1-oxa-2-aza-benzo[e]azulen-8-yl]-2-oxo-oxazolidin-5-ylmethyl}-acetamide;
(S)-N-{2-Oxo-3-[3-(2-trifluoromethoxy-phenyl)-5,6-dihydro-4H-1-oxa-2-aza-benzo[e]azulen-8-yl]-oxazolidin-5-ylmethyl}-acetamide;
(S)-N-{2-Oxo-3-[3-(2-trifluoromethyl-phenyl)-5,6-dihydro-4H-1-oxa-2-aza-benzo[e]azulen-8-yl]-oxazolidin-5-ylmethyl}-acetamide;
(S)-N-{3-[3-(2-Fluoro-phenyl)-5,6-dihydro-4H-1-oxa-2-aza-benzo[e]azulen-8-yl]-2-oxo-oxazolidin-5-ylmethyl}-acetamide;
(S)-N-{3-[3-(3-Hydroxy-phenyl)-5,6-dihydro-4H-1-oxa-2-aza-benzo[e]azulen-8-yl]-2-oxo-oxazolidin-5-ylmethyl}-acetamide;
(S)-N-{3-[3-(3-Methoxy-phenyl)-5,6-dihydro-4H-1-oxa-2-aza-benzo[e]azulen-8-yl]-2-oxo-oxazolidin-5-ylmethyl}-acetamide;
(S)-N-{2-Oxo-3-[3-(3-trifluoromethoxy-phenyl)-5,6-dihydro-4H-1-oxa-2-aza-benzo[e]azulen-8-yl]-oxazolidin-5-ylmethyl}-acetamide;
(S)-N-{2-Oxo-3-[3-(3-trifluoromethyl-phenyl)-5,6-dihydro-4H-1-oxa-2-aza-benzo[e]azulen-8-yl]-oxazolidin-5-ylmethyl}-acetamide;
(S)-N-{2-Oxo-3-[3-(3-trifluoromethyl-phenyl)-5,6-dihydro-4H-1-oxa-2-aza-benzo[e]azulen-8-yl]-oxazolidin-5-ylmethyl}-acetamide;
(S)-N-{3-[3-(3-Fluoro-phenyl)-5,6-dihydro-4H-1-oxa-2-aza-benzo[e]azulen-8-yl]-2-oxo-oxazolidin-5-ylmethyl}-acetamide;
(S)-N-{3-[3-(4-Hydroxy-phenyl)-5,6-dihydro-4H-1-oxa-2-aza-benzo[e]azulen-8-yl]-2-oxo-oxazolidin-5-ylmethyl}-acetamide;
(S)-N-{3-[3-(4-methoxy-phenyl)-5,6-dihydro-4H-1-oxa-2-aza-benzo[e]azulen-8-yl]-2-oxo-oxazolidin-5-ylmethyl}-acetamide;
(S)-N-{2-Oxo-3-[3-(4-trifluoromethoxy-phenyl)-5,6-dihydro-4H-1-oxa-2-aza-benzo[e]azulen-8-yl]-oxazolidin-5-ylmethyl}-acetamide;
(S)-N-{2-Oxo-3-[4-(3-trifluoromethyl-phenyl)-5,6-dihydro-4H-1-oxa-2-aza-benzo[e]azulen-8-yl]-oxazolidin-5-ylmethyl}-acetamide;
(S)-N-{3-[3-(4-Fluoro-phenyl)-5,6-dihydro-4H-1-oxa-2-aza-benzo[e]azulen-8-yl]-2-oxo-oxazolidin-5-ylmethyl}-acetamide;
(S)-N-[2-Oxo-3-(3-thiophen-3-yl-5,6-dihydro-4H-1-oxa-2-aza-benzo[e]azulen-8-yl)-oxazolidin-5-ylmethyl]-acetamide;
(S)-N-{3-[3-(4-Hydroxy-thiophen-3-yl)-5,6-dihydro-4H-1-oxa-2-aza-benzo[e]azulen-8-yl]-2-oxo-oxazolidin-5-ylmethyl}-acetamide;
(S)-N-{3-[3-(4-Methoxy-thiophen-3-yl)-5,6-dihydro-4H-1-oxa-2-aza-benzo[e]azulen-8-yl]-2-oxo-oxazolidin-5-ylmethyl}-acetamide;
(S)-N-{2-Oxo-3-[3-(4-trifluoromethoxy-thiophen-3-yl)-5,6-dihydro-4H-1-oxa-2-aza-benzo[e]azulen-8-yl]-oxazolidin-5-ylmethyl}-acetamide;
(S)-N-{2-Oxo-3-[3-(4-trifluoromethyl-thiophen-3-yl)-5,6-dihydro-4H-1-oxa-2-aza-benzo[e]azulen-8-yl]-oxazolidin-5-ylmethyl}-acetamide;
(S)-N-{3-[3-(4-Fluoro-thiophen-3-yl)-5,6-dihydro-4H-1-oxa-2-aza-benzo[e]azulen-8-yl]-2-oxo-oxazolidin-5-ylmethyl}-acetamide;
(S)-N-{3-[3-(5-Hydroxy-thiophen-3-yl)-5,6-dihydro-4H-1-oxa-2-aza-benzo[e]azulen-8-yl]-2-oxo-oxazolidin-5-ylmethyl}-acetamide;
(S)-N-{3-[3-(5-Methoxy-thiophen-3-yl)-5,6-dihydro-4H-1-oxa-2-aza-benzo[e]azulen-8-yl]-2-oxo-oxazolidin-5-ylmethyl}-acetamide;
(S)-N-{2-Oxo-3-[3-(5-trifluoromethoxy-thiophen-3-yl)-5,6-dihydro-4H-1-oxa-2-aza-benzo[e]azulen-8-yl]-oxazolidin-5-ylmethyl}-acetamide;
(S)-N-{2-Oxo-3-[3-(5-trifluoromethyl-thiophen-3-yl)-5,6-dihydro-4H-1-oxa-2-aza-benzo[e]azulen-8-yl]-oxazolidin-5-ylmethyl}-acetamide;
(S)-N-{3-[3-(5-Fluoro-thiophen-3-yl)-5,6-dihydro-4H-1-oxa-2-aza-benzo[e]azulen-8-yl]-2-oxo-oxazolidin-5-ylmethyl}-acetamide;
(S)-N-[3-(3-Furan-3-yl-5,6-dihydro-4H-1-oxa-2-aza-benzo[e]azulen-8-yl)-2-oxo-oxazolidin-5-ylmethyl]-acetamide;
(S)-N-{3-[3-(4-Hydroxy-furan-3-yl)-5,6-dihydro-4H-1-oxa-2-aza-benzo[e]azulen-8-yl]-2-oxo-oxazolidin-5-ylmethyl}-acetamide;
(S)-N-{3-[3-(4-Methoxy-furan-3-yl)-5,6-dihydro-4H-1-oxa-2-aza-benzo[e]azulen-8-yl]-2-oxo-oxazolidin-5-ylmethyl}-acetamide;
(S)-N-{2-Oxo-3-[3-(4-trifluoromethoxy-furan-3-yl)-5,6-dihydro-4H-1-oxa-2-aza-benzo[e]azulen-8-yl]-oxazolidin-5-ylmethyl}-acetamide;
(S)-N-{2-Oxo-3-[3-(4-trifluoromethyl-furan-3-yl)-5,6-dihydro-4H-1-oxa-2-aza-benzo[e]azulen-8-yl]-oxazolidin-5-ylmethyl}-acetamide;
(S)-N-{3-[3-(4-Fluoro-thiophen-3-yl)-5,6-dihydro-4H-1-oxa-2-aza-benzo[e]azulen-8-yl]-2-oxo-oxazolidin-5-ylmethyl}-acetamide;
(S)-N-{3-[3-(5-Hydroxy-furan-3-yl)-5,6-dihydro-4H-1-oxa-2-aza-benzo[e]azulen-8-yl]-2-oxo-oxazolidin-5-ylmethyl}-acetamide;
(S)-N-{3-[3-(5-Methoxy-furan-3-yl)-5,6-dihydro-4H-1-oxa-2-aza-benzo[e]azulen-8-yl]-2-oxo-oxazolidin-5-ylmethyl}-acetamide;
(S)-N-{2-Oxo-3-[3-(5-trifluoromethoxy-furan-3-yl)-5,6-dihydro-4H-1-oxa-2-aza-benzo[e]azulen-8-yl]-oxazolidin-5-ylmethyl}-acetamide;
(S)-N-{2-Oxo-3-[3-(5-trifluoromethyl-furan-3-yl)-5,6-dihydro-4H-1-oxa-2-aza-benzo[e]azulen-8-yl]-oxazolidin-5-ylmethyl}-acetamide;
(S)-N-{3-[3-(5-Fluoro-furan-3-yl)-5,6-dihydro-4H-1-oxa-2-aza-benzo[e]azulen-8-yl]-2-oxo-oxazolidin-5-ylmethyl}-acetamide;
(S)-N-[2-Oxo-3-(3-pyridin-4-yl-5,6-dihydro-4H-1-oxa-2-aza-benzo[e]azulen-8-yl)-oxazolidin-5-ylmethyl]-acetamide;
(S)-N-{3-[3-(3-Hydroxy-pyridin-4-yl)-5,6-dihydro-4H-1-oxa-2-aza-benzo[e]azulen-8-yl]-2-oxo-oxazolidin-5-ylmethyl}-acetamide;
(S)-N-{3-[3-(3-Methoxy-pyridin-4-yl)-5,6-dihydro-4H-1-oxa-2-aza-benzo[e]azulen-8-yl]-2-oxo-oxazolidin-5-ylmethyl}-acetamide;
(S)-N-{2-Oxo-3-[3-(3-trifluoromethoxy-pyridin-4-yl)-5,6-dihydro-4H-1-oxa-2-aza-benzo[e]azulen-8-yl]-oxazolidin-5-ylmethyl}-acetamide;
(S)-N-{2-Oxo-3-[3-(3-trifluoromethyl-pyridin-4-yl)-5,6-dihydro-4H-1-oxa-2-aza-benzo[e]azulen-8-yl]-oxazolidin-5-ylmethyl}-acetamide;
(S)-N-{3-[3-(3-Fluoro-pyridin-4-yl)-5,6-dihydro-4H-1-oxa-2-aza-benzo[e]azulen-8-yl]-2-oxo-oxazolidin-5-ylmethyl}-acetamide;
(S)-N-{3-[3-(2-Hydroxy-pyridin-4-yl)-5,6-dihydro-4H-1-oxa-2-aza-benzo[e]azulen-8-yl]-2-oxo-oxazolidin-5-ylmethyl}-acetamide;
(S)-N-{3-[3-(2-Methoxy-pyridin-4-yl)-5,6-dihydro-4H-1-oxa-2-aza-benzo[e]azulen-8-yl]-2-oxo-oxazolidin-5-ylmethyl}-acetamide;
(S)-N-{2-Oxo-3-[3-(2-trifluoromethoxy-pyridin-4-yl)-5,6-dihydro-4H-1-oxa-2-aza-benzo[e]azulen-8-yl]-oxazolidin-5-ylmethyl}-acetamide;
(S)-N-{2-Oxo-3-[3-(2-trifluoromethyl-pyridin-4-yl)-5,6-dihydro-4H-1-oxa-2-aza-benzo[e]azulen-8-yl]-oxazolidin-5-ylmethyl}-acetamide;
(S)-N-{3-[3-(2-Fluoro-pyridin-4-yl)-5,6-dihydro-4H-1-oxa-2-aza-benzo[e]azulen-8-yl]-2-oxo-oxazolidin-5-ylmethyl}-acetamide;
(S)-N-[2-Oxo-3-(1,4,5,6-tetrahydro-1,2-diaza-benzo[e]azulen-9-yl)-oxazolidin-5-ylmethyl]-acetamide;
(S)-N-[2-Oxo-3-(3-phenyl-1,4,5,6-tetrahydro-1,2-diaza-benzo[e]azulen-9-yl)-oxazolidin-5-ylmethyl]-acetamide;
(S)-N-{3-[3-(2-Hydroxy-phenyl)-1,4,5,6-tetrahydro-1,2-diaza-benzo[e]azulen-9-yl]-2-oxo-oxazolidin-5-ylmethyl}-acetamide;
(S)-N-{3-[3-(2-Methoxy-phenyl)-1,4,5,6-tetrahydro-1,2-diaza-benzo[e]azulen-9-yl]-2-oxo-oxazolidin-5-ylmethyl}-acetamide;
(S)-N-{2-Oxo-3-[3-(2-trifluoromethoxy-phenyl)-1,4,5,6-tetrahydro-1,2-diaza-benzo[e]azulen-9-yl]-oxazolidin-5-ylmethyl}-acetamide;
(S)-N-{2-Oxo-3-[3-(2-trifluoromethyl-phenyl)-1,4,5,6-tetrahydro-1,2-diaza-benzo[e]azulen-9-yl]-oxazolidin-5-ylmethyl}-acetamide;
(S)-N-{3-[3-(2-Fluoro-phenyl)-1,4,5,6-tetrahydro-1,2-diaza-benzo[e]azulen-9-yl]-2-oxo-oxazolidin-5-ylmethyl}-acetamide;
(S)-N-{3-[3-(3-Hydroxy-phenyl)-1,4,5,6-tetrahydro-1,2-diaza-benzo[e]azulen-9-yl]-2-oxo-oxazolidin-5-ylmethyl}-acetamide;
(S)-N-{3-[3-(3-Methoxy-phenyl)-1,4,5,6-tetrahydro-1,2-diaza-benzo[e]azulen-9-yl]-2-oxo-oxazolidin-5-ylmethyl}-acetamide;
(S)-N-{2-Oxo-3-[3-(3-trifluoromethoxy-phenyl)-1,4,5,6-tetrahydro-1,2-diaza-benzo[e]azulen-9-yl]-oxazolidin-5-ylmethyl}-acetamide;
(S)-N-{2-Oxo-3-[3-(3-trifluoromethyl-phenyl)-1,4,5,6-tetrahydro-1,2-diaza-benzo[e]azulen-9-yl]-oxazolidin-5-ylmethyl}-acetamide;
(S)-N-{3-[3-(3-Fluoro-phenyl)-1,4,5,6-tetrahydro-1,2-diaza-benzo[e]azulen-9-yl]-2-oxo-oxazolidin-5-ylmethyl}-acetamide;
(S)-N-{3-[3-(4-Hydroxy-phenyl)-1,4,5,6-tetrahydro-1,2-diaza-benzo[e]azulen-9-yl]-2-oxo-oxazolidin-5-ylmethyl}-acetamide;
(S)-N-{3-[3-(4-Methoxy-phenyl)-1,4,5,6-tetrahydro-1,2-diaza-benzo[e]azulen-9-yl]-2-oxo-oxazolidin-5-ylmethyl}-acetamide;
(S)-N-{2-Oxo-3-[3-(4-trifluoromethoxy-phenyl)-1,4,5,6-tetrahydro-1,2-diaza-benzo[e]azulen-9-yl]-oxazolidin-5-ylmethyl}-acetamide;
(S)-N-{2-Oxo-3-[3-(4-trifluoromethyl-phenyl)-1,4,5,6-tetrahydro-1,2-diaza-benzo[e]azulen-9-yl]-oxazolidin-5-ylmethyl}-acetamide;
(S)-N-{3-[3-(4-Fluoro-phenyl)-1,4,5,6-tetrahydro-1,2-diaza-benzo[e]azulen-9-yl]-2-oxo-oxazolidin-5-ylmethyl}-acetamide;
(S)-N-[3-(5,6-Dihydro-4H-1-oxa-2-aza-benzo[e]azulen-9-yl)-2-oxo-oxazolidin-5-ylmethyl]-acetamide;
(S)-N-[2-Oxo-3-(3-phenyl-5,6-dihydro-4H-1-oxa-2-aza-benzo[e]azulen-9-yl)-oxazolidin-5-ylmethyl]-acetamide;
(S)-N-{3-[3-(2-Hydroxy-phenyl)-5,6-dihydro-4H-1-oxa-2-aza-benzo[e]azulen-9-yl]-2-oxo-oxazolidin-5-ylmethyl}-acetamide;
(S)-N-{3-[3-(2-Methoxy-phenyl)-5,6-dihydro-4H-1-oxa-2-aza-benzo[e]azulen-9-yl]-2-oxo-oxazolidin-5-ylmethyl}-acetamide;
(S)-N-{2-Oxo-3-[3-(2-trifluoromethoxy-phenyl)-5,6-dihydro-4H-1-oxa-2-aza-benzo[e]azulen-9-yl]-oxazolidin-5-ylmethyl}-acetamide;
(S)-N-{2-Oxo-3-[3-(2-trifluoromethyl-phenyl)-5,6-dihydro-4H-1-oxa-2-aza-benzo[e]azulen-9-yl]-oxazolidin-5-ylmethyl}-acetamide;
(S)-N-{3-[3-(2-Fluoro-phenyl)-5,6-dihydro-4H-1-oxa-2-aza-benzo[e]azulen-9-yl]-2-oxo-oxazolidin-5-ylmethyl}-acetamide;
(S)-N-{3-[3-(3-Hydroxy-phenyl)-5,6-dihydro-4H-1-oxa-2-aza-benzo[e]azulen-9-yl]-2-oxo-oxazolidin-5-ylmethyl}-acetamide;
(S)-N-{3-[3-(3-Methoxy-phenyl)-5,6-dihydro-4H-1-oxa-2-aza-benzo[e]azulen-9-yl]-2-oxo-oxazolidin-5-ylmethyl}-acetamide;
(S)-N-{2-Oxo-3-[3-(3-trifluoromethoxy-phenyl)-5,6-dihydro-4H-1-oxa-2-aza-benzo[e]azulen-9-yl]-oxazolidin-5-ylmethyl}-acetamide;
(S)-N-{2-Oxo-3-[3-(3-trifluoromethyl-phenyl)-5,6-dihydro-4H-1-oxa-2-aza-benzo[e]azulen-9-yl]-oxazolidin-5-ylmethyl}-acetamide;
(S)-N-{3-[3-(3-Fluoro-phenyl)-5,6-dihydro-4H-1-oxa-2-aza-benzo[e]azulen-9-yl]-2-oxo-oxazolidin-5-ylmethyl}-acetamide;
(S)-N-{3-[3-(4-Hydroxy-phenyl)-5,6-dihydro-4H-1-oxa-2-aza-benzo[e]azulen-9-yl]-2-oxo-oxazolidin-5-ylmethyl}-acetamide;
(S)-N-{3-[3-(4-Methoxy-phenyl)-5,6-dihydro-4H-1-oxa-2-aza-benzo[e]azulen-9-yl]-2-oxo-oxazolidin-5-ylmethyl}-acetamide;
(S)-N-{2-Oxo-3-[3-(4-trifluoromethoxy-phenyl)-5,6-dihydro-4H-1-oxa-2-aza-benzo[e]azulen-9-yl]-oxazolidin-5-ylmethyl}-acetamide;
(S)-N-{2-Oxo-3-[3-(4-trifluoromethyl-phenyl)-5,6-dihydro-4H-1-oxa-2-aza-benzo[e]azulen-9-yl]-oxazolidin-5-ylmethyl}-acetamide;
(S)-N-{3-[3-(4-Fluoro-phenyl)-5,6-dihydro-4H-1-oxa-2-aza-benzo[e]azulen-9-yl]-2-oxo-oxazolidin-5-ylmethyl}-acetamide;
(S)-N-[3-(2-Methyl-9,10-dihydro-4H-3-thia-1-aza-benzo[f]azulen-7-yl)-2-oxo-oxazolidin-5-ylmethyl]-acetamide;
(S)-N-[3-(2-Methyl-9,10-dihydro-4H-3-thia-1-aza-benzo[f]azulen-6-yl)-2-oxo-oxazolidin-5-ylmethyl]-acetamide;
(S)-N-[3-(2-Methyl-5,6-dihydro-4H-3-thia-1-aza-benzo[e]azulen-8-yl)-2-oxo-oxazolidin-5ylmethyl] acetamide;
(S)-N-[3-(2-Amino-5,6-dihydro-4H-3-thia-1-aza-benzo[e]azulen-8-yl)-2-oxo-oxazolidin-5-ylmethyl]-acetamide;
(S)-N-[3-(2-Methyl-3,4,5,6-tetrahydro-1,3-diaza-benzo[e]azulen-8-yl)-2-oxo-oxazolidin-5-ylmethyl]-acetamide;
(S)-N-[2-Oxo-3-(2-trifluoromethyl-3,4,5,6-tetrahydro-1,3-diaza-benzo[e]azulen-8-yl)-oxazolidin-5-ylmethyl]-acetamide;
(S)-N-[2-Oxo-3-(3,4,5,6-tetrahydro-2,3-diaza-benzo[e]azulen-9-yl)-oxazolidin-5-ylmethyl]-acetamide; or
(S)-N-[3-(5,6-Dihydro-4H-3-oxa-2-aza-benzo[e]azulen-9-yl)-2-oxo-oxazolidin-5-ylmethyl]-acetamide.

38. A pharmaceutical formulation comprising a compound of claim 1 admixed with a pharmaceutically acceptable diluent, carrier, or excipient.

39. A method of treating a bacterial infection in a mammal, comprising administering to a mammal in need thereof an effective amount of a compound of claim 1.
